SEQUENCE LISTING (3 sequences)

SEQ ID NO: 1 - protein, 696 aa, synthetic construct
MEHHHHHHHHEYMPMEMAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIWPLQGWQATFGGGDHPPKSDLVPRHNQTSLYKKAGSAAAVLEENLYFQGTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 2 - protein, 659 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIANLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - protein, 10 aa, synthetic construct
  MOD_RES 1: note = pyro-Glutamic acid
  MOD_RES 6: note = O-t-butyl-D-Serine
  MOD_RES 10: note = Amidated azaglycine
EHWSYSLRPG

(19) United States
(12) Patent Application Publication
(10) Pub. No.: US 2024/0116877 A1
(43) Pub. Date: Apr. 11, 2024

(54) KIT KINASE INHIBITORS AND METHODS OF USE THEREOF

(71) Applicant: Deciphera Pharmaceuticals, LLC, Waltham, MA (US)

(72) Inventors: Yu Mi Ahn, Waltham, MA (US); Timothy Caldwell, Lawrence, KS (US); Daniel L. Flynn, Waltham, MA (US); Bertrand LeBourdonnec, Waltham, MA (US)

(21) Appl. No.: 18/073,721

(22) Filed: Dec. 2, 2022

Related U.S. Application Data
(60) Provisional application No. 63/329,674, filed on Apr. 11, 2022.
(60) Provisional application No. 63/287,857, filed on Dec. 9, 2021.

(51) Int. Cl. C07D 239/94; C07D 237/28; C07D 401/12; C07D 403/12; C07D 405/12; C07D 413/12; C07D 487/08

(52) U.S. Cl. CPC C07D 239/94; C07D 237/28; C07D 401/12; C07D 403/12; C07D 405/12; C07D 413/12; C07D 487/08

(57) ABSTRACT

Described herein are compounds that are inhibitors of wild type and oncogenic mutant c-KIT kinase and their use in the treatment of disorders such as cancers.

### CROSS-REFERENCE

This application claims priority to U.S. Provisional Application No. 63/287,857 filed Dec. 9, 2021, and U.S. Provisional Application No. 63/329,674 filed Apr. 11, 2022, the contents of each of which are incorporated herein by reference in their entireties.

### REFERENCE TO A SEQUENCE LISTING XML

This application contains a Sequence Listing which has been submitted electronically in XML format. The Sequence Listing XML is incorporated herein by reference. Said XML file, created on Jun. 15, 2023, is named DCP-113_SL.xml and is 6,127 bytes in size.

### BACKGROUND

Gain-of-function mutations in Type III receptor tyrosine kinases KIT and PDGFRA, are present in many cancers and myeloproliferative diseases. For example, gastrointestinal stromal tumors (GISTs) are driven by activating mutations in KIT (˜80%) and PDGFRA (˜5-10%). Mutations in KIT drive >90% of cases of systemic mastocytosis (SM) and mast cell leukemia (MCL), and a small but significant percentage of acute myeloid leukemia (AML), germ cell tumors, and melanoma. PDGFRA mutations or gene rearrangements are also observed in cancers such as hypereosinophilic leukemia and glioblastoma. KIT and/or PDGFRA gene amplifications are likely oncogenic in gliomas and lung cancers as well.

Most primary and secondary resistance mutations in KIT and PDGFRA are located within switch regions that reside within the intracellular kinase domain. KIT and PDGFRA are named “dual switch kinases” based on the observation that they each incorporate a) an autoinhibitory switch within the intracellular juxtamembrane domain (JMD) and b) a main activation loop (AL) switch within the kinase domain. Several studies have shown that this dual switch mechanism regulates KIT and PDGFRA cellular kinase activity by controlling kinase conformation. Indeed, phosphorylation of one or more amino acids triggers conformational change leading to activation of the kinase from an inactive (or “off”) state to an active (or “on”) state. Disruption of one or more of these switch control mechanisms can cause the kinase to aberrantly adopt an active Type I conformation. In approximately 70% of GIST patients, the primary activating mutation is found in KIT exon 11 which encodes the JMD inhibitory switch resulting in a loss-of-function in the inhibitory switch, shifting conformational equilibrium towards an active Type I form. A small percentage of primary activating mutations and almost all secondary resistance mutations in GIST are mutations located either in the main AL switch found on exons 17 and 18 of KIT and exon 18 of PDGFRA, respectively, or on exons 13 and 14 of KIT and exons 14 and 15 of PDGFRA in the switch pocket region adjacent to the ATP binding pocket. These gain-of-function mutations likewise stabilize an active Type I conformation. Specifically, the D816 switch residue of KIT (exon 17) and the analogous D842 residue of PDGFRA (exon 18) are essential for the AL switch to reside in a Type II off state. Mutation of these aspartic acid residues to valine (KIT D816V and PDGFRA D842V mutants) results in a change in protein conformation leading to a transition of the kinase from an inactive state (Type II conformation) to an active state (Type I conformation). Diseases caused by mast cell proliferation, such as systemic mastocytosis (SM) and mantle cell lymphoma (MCL), and a small percentage of acute myeloid leukemia (AML) are driven by primary mutations in the main AL switch of KIT (e.g., D816V or N822K). Similarly, a D842V mutation within the PDGFRA main AL switch is the most common primary mutation observed within a small subset of PDGFRA-mutant GIST.

Treatment of metastatic GIST was revolutionized with the advent of tyrosine kinase inhibitors (TKIs), led by imatinib, a potent inhibitor of primary exon 11 JMD KIT mutations, approved as first-line therapy for advanced GIST in 2002. While the majority of the GIST patients responded well to imatinib treatment, about 10-14% of treated patients were found to be insensitive to imatinib therapy. In addition, more than half of early imatinib treatment responders developed drug resistance within two years of imatinib therapy. This resistance was primarily due to the formation of secondary mutations within KIT (e.g., exon 13 V654A within the switch pocket region or various exon 17 mutations in the AL switch). Subsequently, sunitinib and regorafenib were approved as second- and third-line therapies for imatinib-resistant GIST. Sunitinib preferentially inhibits secondary KIT mutations in exons 13 and 14 but does not inhibit secondary mutations in the exon 17/18 AL switch. Conversely, regorafenib inhibits primarily a subset of secondary mutations in exon 17, while only weakly inhibiting secondary mutations in KIT exons 13 and 14.

Examination of individual GIST patient biopsies obtained from different progressing metastases indicates that a complex heterogenous array of primary and multiple secondary KIT mutations can exist within the same patient and in different metastatic lesions. This observation further complicates the selection of an appropriate TKI-based therapy. Furthermore, the off-target kinase activity of sunitinib and regorafenib leads to tolerability issues, which require significant clinical treatment management (e.g., drug holidays, dose reduction). The necessary adjustment in therapy treatment schedule may also exacerbate the development of additional secondary resistance mutations. From a safety perspective, the use of avapritinib is also limited due to central nervous system (CNS) adverse events that resulted in black box label restriction.

There is a clear unmet need for a tyrosine kinase inhibitor with potent inhibitory activity against a broad spectrum of clinically relevant KIT and PDGFRA mutants, and an improved safety and tolerability profile when compared to currently available GIST treatments.

### SUMMARY

Described herein, in part, are compounds that are inhibitors of wild type and oncogenic mutant c-KIT kinase and methods of use thereof, such as the treatment of cancers driven by c-KIT kinase mutations, e.g., primary (KIT exon 9 or 11) and secondary (exons 13, 14, 17 and 18) c-KIT mutations.

In one embodiment, described herein, is a compound represented by Formula I:

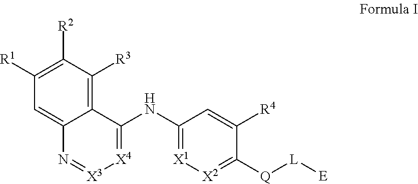

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
Q is selected from the group consisting of

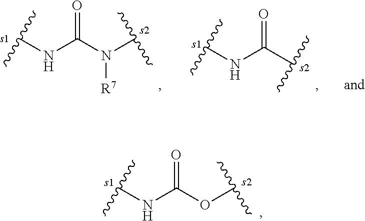

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

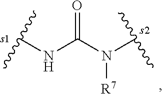

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

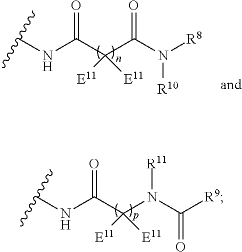

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3, provided that the compound is not:

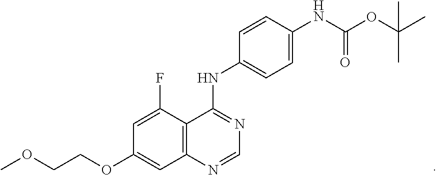 [SMILES: COCCOc1cc(F)c2c(Nc3ccc(NC(=O)OC(C)(C)C)cc3)ncnc2c1]

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The definitions set forth in this application are intended to clarify terms used throughout this application.

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used herein, the singular forms “a,” “an,” and, “the” encompass plural references unless the context clearly indicates otherwise. The terms “comprising,” “including,” and “having” are intended to be inclusive and mean that there may be additional elements other than the listed elements.

As used herein, the following definitions shall apply unless otherwise indicated. For purposes of this disclosure, the chemical elements are identified in accordance with the Periodic Table of the Elements, CAS version, and the Handbook of Chemistry and Physics, 75th Ed.1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference.

As used herein, the term “herein” means the entire application.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with the radical of a specified substituent including, but not limited to, hydroxyl, hydroxyalkyl, alkoxy, halogen, alkyl, aryl, alkoxy, cycloalkyl, heterocyclyl, amino, aminoalkyl, cyano, haloalkyl, haloalkoxy, and —OC(═O)—CH2-Oalkyl. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, an alkoxy, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN, and the like. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to a straight chained or branched non-aromatic hydrocarbon which is completely saturated. Typically, a straight chained or branched alkyl group has from 1 to about 20 carbon atoms, preferably from 1 to about 10, e.g., may be C₁-C₆alkyl or e.g., C₁-C₆ alkyl unless otherwise defined. Examples of straight chained and branched alkyl groups include, but are not limited to, methyl, ethyl, 1-propyl (n-propyl), 2-propyl, n-butyl, sec-butyl, tertbutyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl, or 4-octyl, and the like. Moreover, the term “alkyl” used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. The “alkyl” group may be optionally substituted.

The term “Cₓ-C_{y}” when used in conjunction with a chemical moiety, such as, acyl, acyloxy, alkyl, alkenyl, or alkoxy is meant to include groups that contain from x to y carbons in the chain. For example, the term “Cₓ-C_{y}” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. Co alkyl indicates a hydrogen where the group is in a terminal position, a bond if internal.

As used herein, the terms “amine” and “amino” refer to both unsubstituted and substituted amines and salts thereof, e.g., a moiety that can be represented by:

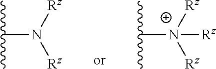

wherein R^{z} each independently represents hydrogen or a hydrocarbyl group, or R^{z} groups are taken together with the N atom to which they are attached complete a heterocyclyl having from 4 to 8 atoms in the ring structure.

As used herein, the terms “amide” and “amido” refers to a group represented by

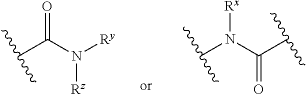

wherein R^{X}, R^{y}, and R^{z} each independently represents hydrogen or a hydrocarbyl group, or R^{y} and R^{z} are taken together with the N atom to which they are attached complete a heterocyclyl having from 4 to 8 atoms in the ring structure.

As used herein, the term “alkoxy” refers to a straight or branched, saturated aliphatic (alkyl) hydrocarbon radical bonded to an oxygen atom that is attached to a core structure. Preferably, alkoxy groups have one to six carbon atoms, i.e., may be C₁-C₆ alkoxy. Examples of alkoxy groups include but are not limited to methoxy, ethoxy, propoxy, isopropoxy, butoxy, isobutoxy, tert-butoxy, pentoxy, 3-methyl butoxy, and the like. The term “alkoxy” as used herein may be optionally substituted, as defined above.

As used herein, the term “haloalkyl” refers to alkyl group (as defined above) that is substituted with one or more halogens. A monohaloalkyl radical, for example, may have a chlorine, bromine, iodine, or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl, and the like.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃), or trifluoroethoxy (—OCH₂CF₃).

As used herein, the term “hydrocarbyl” refers to an aliphatic hydrocarbon group. The hydrocarbyl moiety may be a “saturated alkyl” group, which means that it does not contain any alkene or alkyne moieties. The hydrocarbyl moiety may also be an “unsaturated alkyl” moiety, which means that it contains at least one alkene or alkyne moiety. An “alkene” moiety refers to a straight or branched hydrocarbon chain group consisting of from two to eight carbon atoms and at least one carbon-carbon double bond, which is attached to the rest of the molecule by a single bond, e.g., ethenyl, prop-1-enyl, but-1-enyl, pent-1-enyl, pent-1,4-dienyl and the like. An “alkyne” moiety refers to a straight or branched hydrocarbon chain group consists of from two to eight carbon atoms and at least one carbon-carbon triple bond, which is attached to the rest of the molecule by a single bond. The hydrocarbyl moiety, whether saturated or unsaturated, may be branched chain or straight chain.

As used herein, the term “aminoalkyl” refers to an alkyl group substituted with an amino group.

As used herein, the term “hydroxyalkyl” refers to an alkyl group substituted with a hydroxy group.

As used herein, the term “cycloalkyl” alone or in combination with other term(s) refers to a cyclic hydrocarbon which is completely saturated. “Cycloalkyl” includes monocyclic, bicyclic, and tricyclic rings. Typically, a monocyclic cycloalkyl group has from 3 to about 10 carbon atoms, more typically 3 to 8 carbon atoms (e.g., C₃-C₁₀ cycloalkyl or e.g., C₃-C₆ cycloalkyl unless otherwise defined. Examples of monocyclic cycloalkyls include cyclopropyl, cyclobutyl, cyclopentyl, cyclohexyl, cycloheptyl, and the like. The second ring of a bicyclic cycloalkyl or, the second or third rings of a tricyclic cycloalkyl, may be selected from saturated, unsaturated, and aromatic rings. Cycloalkyl includes bicyclic and tricyclic molecules in which one, two or three or more atoms are shared between the two rings. The term “fused cycloalkyl” refers to a bicyclic or tricyclic cycloalkyl in which each of the rings shares two adjacent atoms with the other ring. The second ring of a fused bicyclic cycloalkyl or, the second or third rings of a fused tricyclic cycloalkyl, may be selected from saturated, unsaturated, and aromatic rings. A “cycloalkenyl” group is a cyclic hydrocarbon containing one or more double bonds. Cycloalkyls can be further substituted with alkyls, alkenyls, alkoxys, alkylthios, aminoalkyls, carbonyl-substituted alkyls, —CF₃, —CN, and the like. A cycloalkyl may alternatively be polycyclic with more than two rings. Examples of polycyclic cycloalkyls include bridged, fused, and spirocyclic carbocyclyls. The term “cycloalkyl” as used herein may be optionally substituted, as defined above.

As used herein, the term “cycloalkyloxy” refers to a cycloalkyl group bonded to an oxygen atom that is attached to a core structure. Exemplary cycloalkyloxy groups include, but are not limited to, cycloalkyloxy groups of 3-6 carbon atoms, referred to herein as C₃-C₆ cycloalkyloxy groups. Exemplary cycloalkyloxy groups include, but are not limited to, cyclopropyloxy, cyclobutoyloxy, cyclohexyloxy, etc.

As used herein, the term “cyano” refers to —CN group.

As used herein, the term “hydroxy” or “hydroxyl” refers to —OH group.

As used herein, the term “halo” or “halogen” alone or in combination with other term(s) means chloro, fluoro, bromo, and iodo.

As used herein, the term “heteroatom” refers to an atom of any element other than carbon or hydrogen. Exemplary heteroatoms are nitrogen (N), oxygen (O), sulfur (S), and silicon (Si).

As used herein, the terms “heterocyclyl”, “heterocycloalkyl”, “heterocyclyl”, and “heterocyclic” refer to substituted or unsubstituted non-aromatic ring structures, including monocyclic, polycyclic (e.g., bicyclic, tricyclic) bridged, or fused, ring structures preferably 3- to 10-membered rings, more preferably 3- to 7-membered rings, whose ring structures include at least one heteroatom, preferably one to four heteroatoms, more preferably one or two heteroatoms. Attachment of a heterocyclyl substituent can occur via either a carbon atom or a heteroatom. Examples of “heterocyclyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, 2-azabicyclo[2.2.2]octanyl, azocinyl, chromanyl, xanthenyl, lactones, lactams, and the like, and N-oxides thereof. A heterocyclyl group may be optionally substituted with one or more suitable groups. Examples of “heterocyclyl” include, but are not limited to, morpholine mimetics. Examples of morpholine mimetics include, but are not limited to:

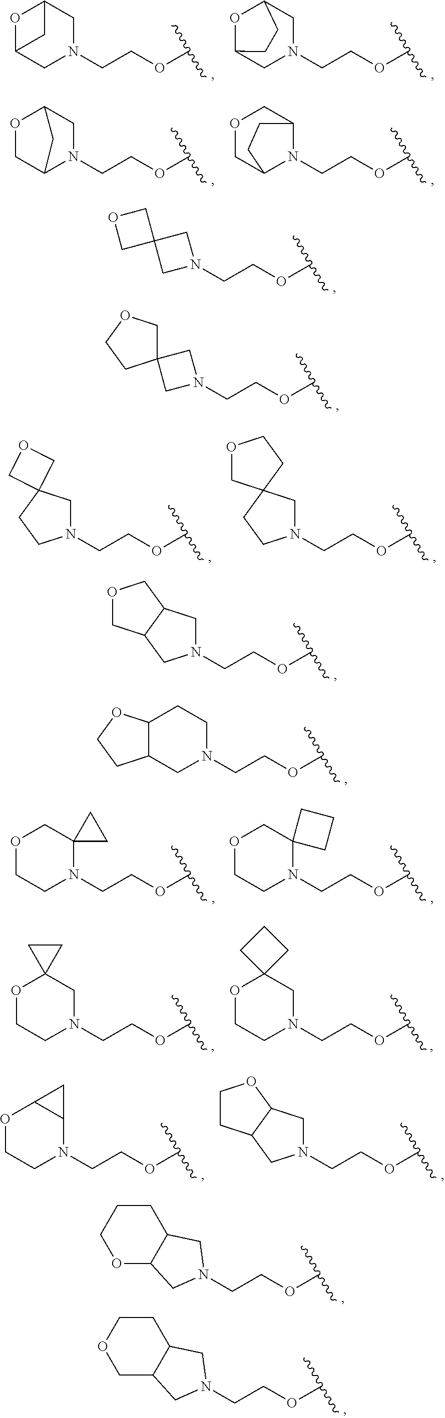

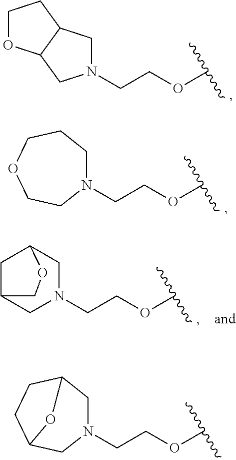 [SMILES: *OCCN1CC2CCOC2C1 ; *OCCN1CCCOCC1 ; *OCCN1CC2COC(C2)C1 ; *OCCN1CC2CCC(C1)O2]

As used herein, the term “heterocyclyloxy” refers to a heterocyclyl group bonded to an oxygen atom that is attached to a core structure. Exemplary heterocyclyloxy groups include, but are not limited to, heterocyclyloxy 3- to 10-membered rings, referred to herein as C₃-C₁₀ heterocyclyloxy groups. Exemplary heterocyclyloxy groups include, but are not limited to, oxetanyloxy, azetidinyloxy, tetrahydrofuranyloxy, piperidinyloxy, piperazinyloxy, etc.

A “combination therapy” is a treatment that includes the administration of two or more therapeutic agents, e.g., a compound of the disclosure and a MAPK pathway inhibitor, to a patient in need thereof.

“Disease,” “disorder,” and “condition” are used interchangeably herein.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like), and laboratory animals (e.g., rats, mice, guinea pigs, and the like).

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R,” or “S,” depending on the configuration of substituents around the stereogenic carbon atom, but the skilled artisan will recognize that a structure may denote a chiral center implicitly. The presently described compounds encompasses all stereoisomers of these compounds and mixtures thereof. Mixtures of enantiomers or diastereomers may be designated “(±)” in nomenclature, but the skilled artisan will recognize that a structure may denote a chiral center implicitly.

In the present specification, the term “therapeutically effective amount” means the amount of the subject compound that will elicit the biological or medical response of a tissue, system or animal, (e.g., mammal or human) that is being sought by the researcher, veterinarian, medical doctor, or other clinician. The compounds described herein are administered in therapeutically effective amounts to treat a disorder.

“Treating” includes any effect, e.g., lessening, reducing, modulating, or eliminating, that results in the improvement of the condition, disease, disorder, and the like.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

As used herein, “compounds of the disclosure”, comprise compounds of Formula I, Formula I-A, Formula I-B, Formula I-C, Formula I-D, Formula I-E, Formula I-F, Formula I-G, Formula I-H, Formula I-I, Formula I-J, Formula I-K, Formula I-L, Formula I-M, Formula I-N, Formula I-O, and Formula I-P, Formula I-Q, Formula I-R, Formula I-S, Formula I-T, Formula I-U, Formula I-V, Formula I-W, Formula I-X, Formula I-Y, Formula I-Z, Formula I-AA, Formula I-AB, and Formula I-AC, or a pharmaceutically acceptable salt, enantiomer, stereoisomer, or tautomer thereof.

### Compounds

Compounds of Formula I, Formula I-A, Formula I-B, Formula I-C, Formula I-D, Formula I-E, and Formula I-F

In one embodiment, described herein, is a compound represented by Formula I:

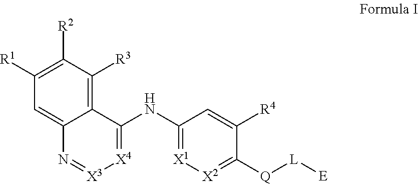

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
Q is selected from the group consisting of

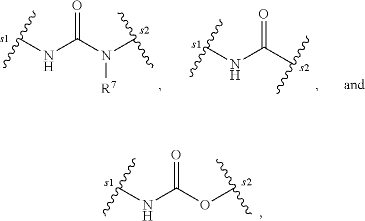

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

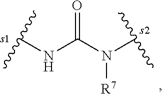

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

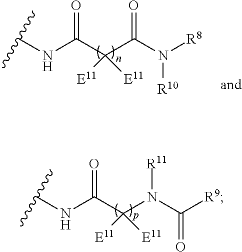

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3,
provided that the compound is not:

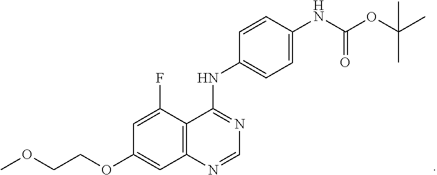 [SMILES: COCCOc1cc(F)c2c(Nc3ccc(NC(=O)OC(C)(C)C)cc3)ncnc2c1]

In one embodiment, described herein is a compound represented by Formula I-A:

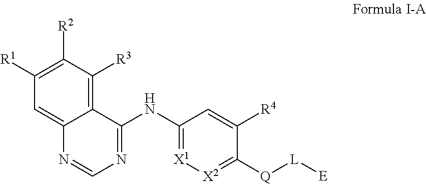

or a pharmaceutically acceptable salt, enantiomer, stereoisomer, or tautomer thereof, wherein:
X¹ and X² are each independently selected from the group consisting of N, CH, and CF;
Q is selected from the group consisting of

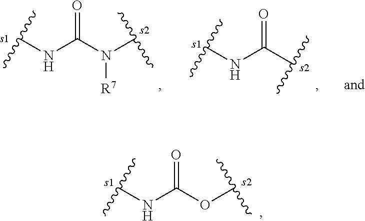

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

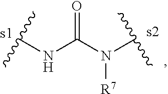

L when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, alkoxy, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

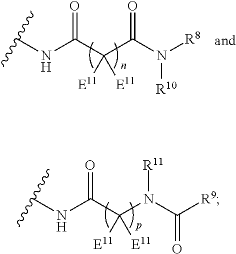

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of: H,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently, selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3,
provided that the compound is not:

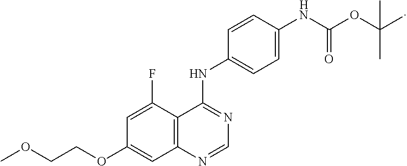

In one embodiment, described herein is a compound represented by Formula I-B:

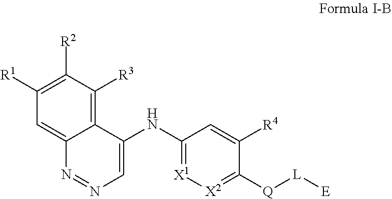

or a pharmaceutically acceptable salt, enantiomer, stereoisomer, or tautomer thereof, wherein:
X¹ and X² are each independently selected from the group consisting of N, CH, and CF;
Q is selected from the group consisting of

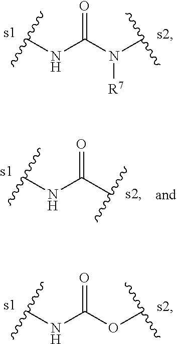

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

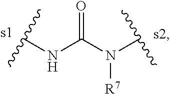

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

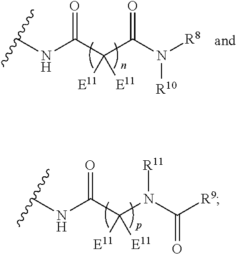

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
   R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3.

In one embodiment, described herein is a compound represented by Formula I-C:

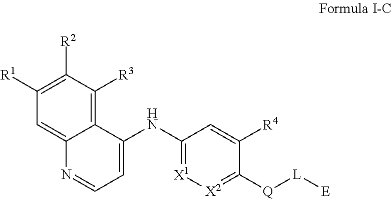

or a pharmaceutically acceptable salt, enantiomer, stereoisomer, or tautomer thereof, wherein:
X¹ and X² are each independently selected from the group consisting of N, CH, and CF;
Q is selected from the group consisting of

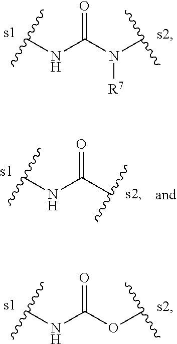

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

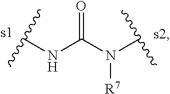

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

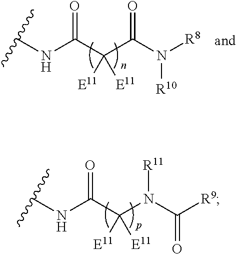

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F, and alkoxy;
R⁴ is selected from the group consisting of H, F, alkyl, and alkoxy;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3.

In one embodiment, described herein is a compound represented by Formula I-D:

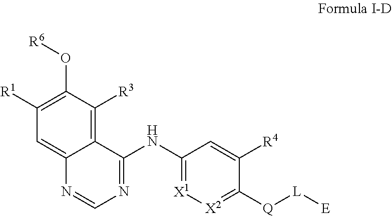

or a pharmaceutically acceptable salt, enantiomer, stereoisomer, or tautomer thereof, wherein:
X¹ and X² are each independently selected from the group consisting of N, CH, and CF;
Q is selected from the group consisting of

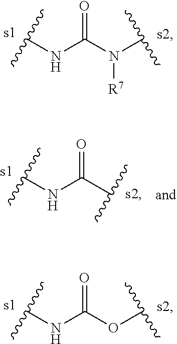

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

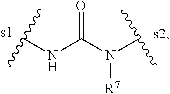

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

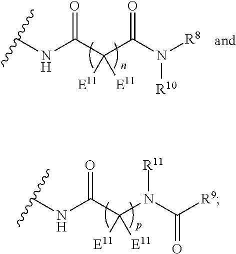

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R³ is selected from the group consisting of F and alkoxy;
R⁴ is selected from the group consisting of H, F, alkyl, and alkoxy;
R⁶ is optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, and cycloalkyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3.

In one embodiment, described herein is a compound represented by Formula I-E:

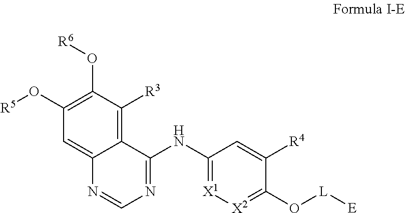

or a pharmaceutically acceptable salt, enantiomer, stereoisomer, or tautomer thereof, wherein:
X¹ and X² are each independently selected from the group consisting of N, CH, and CF;
Q is selected from the group consisting of

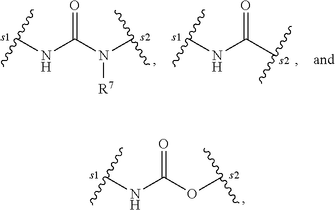

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

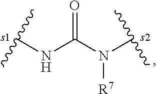

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

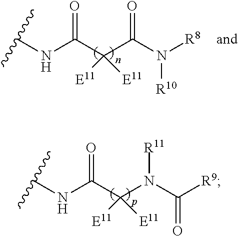

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R³ is selected from the group consisting of F and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁶ is optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, and cycloalkyl, or when taken together with R⁵ form an optionally substituted heterocyclyl having from 5 to 6 atoms in the ring structure wherein the optionally substituted substituent, at each occurrence, is independently selected from the group consisting of C₁-C₆ alkyl and halogen; R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3.

In one embodiment, described herein is a compound represented by Formula I-F:

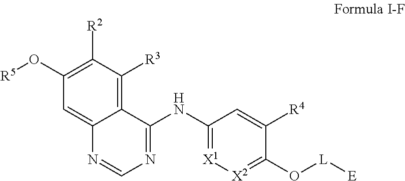

or a pharmaceutically acceptable salt, enantiomer, stereoisomer, or tautomer thereof, wherein:
X¹ and X² are each independently selected from the group consisting of N, CH, and CF;
Q is selected from the group consisting of

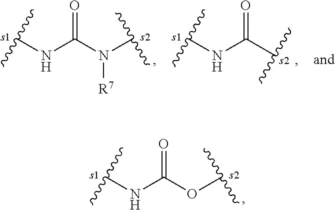

wherein s1 is the site covalently linked to the ring; and s2 is the site covalently linked to L;
L is selected from the group consisting of a direct bond and a C₁-C₆ alkyl optionally substituted with (E¹¹)ₘ, or when Q is

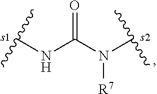

L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of

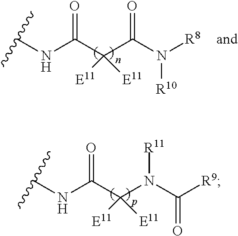

E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, optionally substituted amido, and
optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3,
provided that the compound is not:

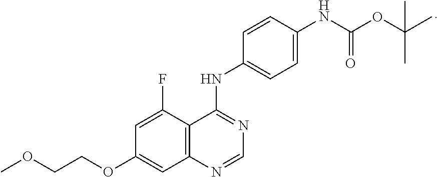 [SMILES: COCCOc1cc(F)c2c(Nc3ccc(NC(=O)OC(C)(C)C)cc3)ncnc2c1]

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

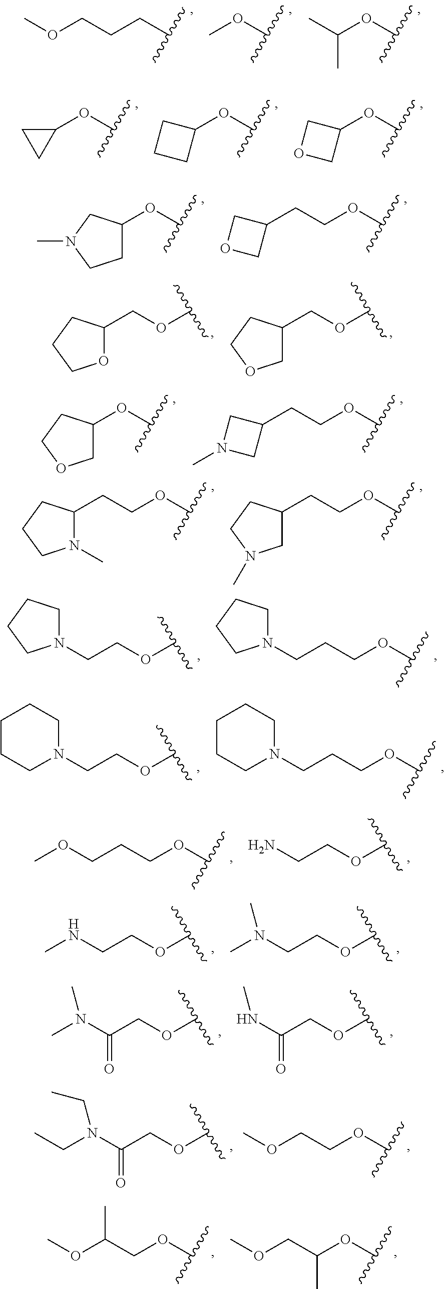

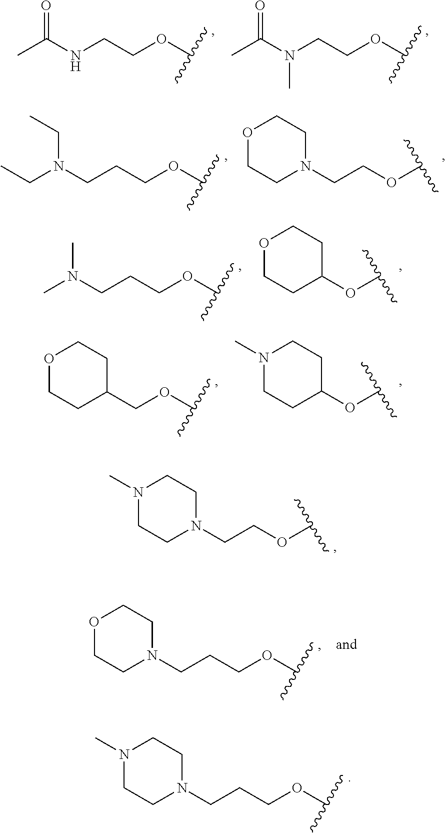

In some embodiments, R¹ is selected from the group consisting of H,

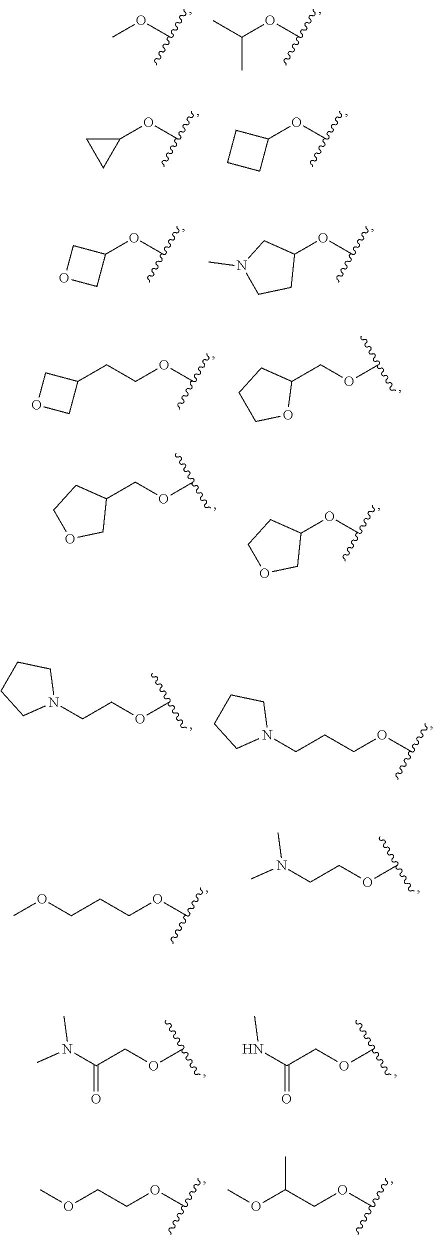

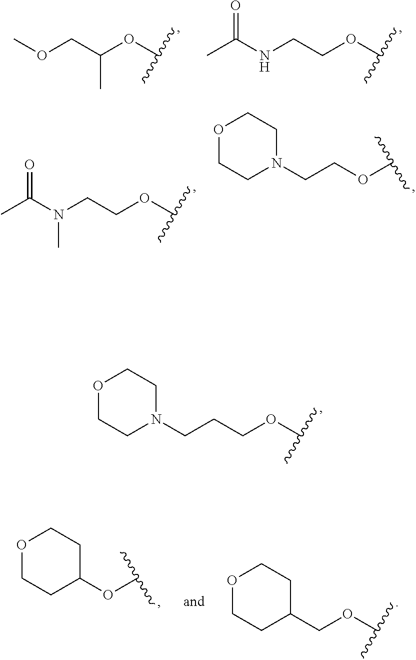

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃, CF₂CF₃,

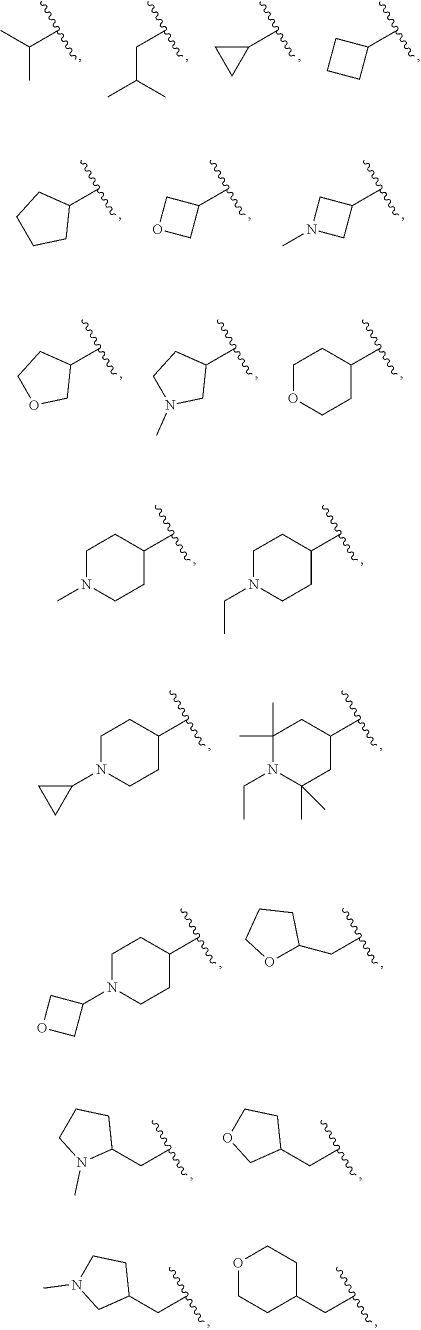

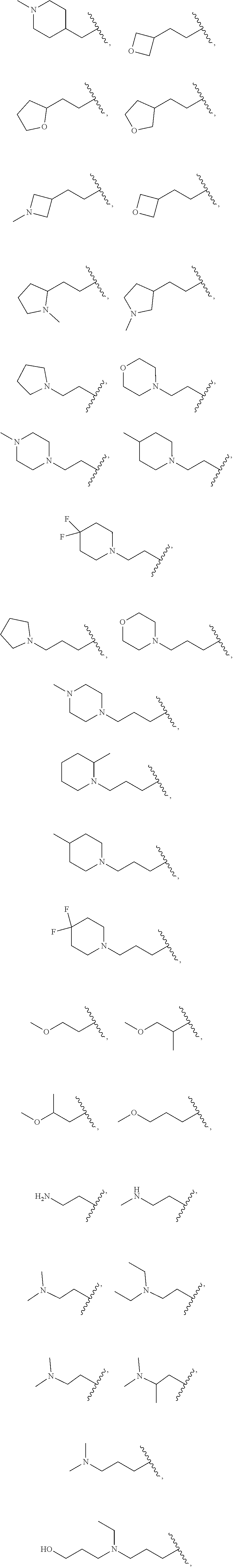

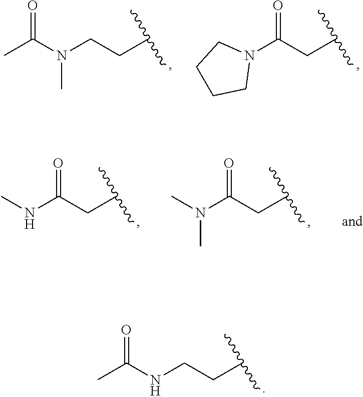

In some embodiments, R⁵ is selected from the group consisting of methyl,

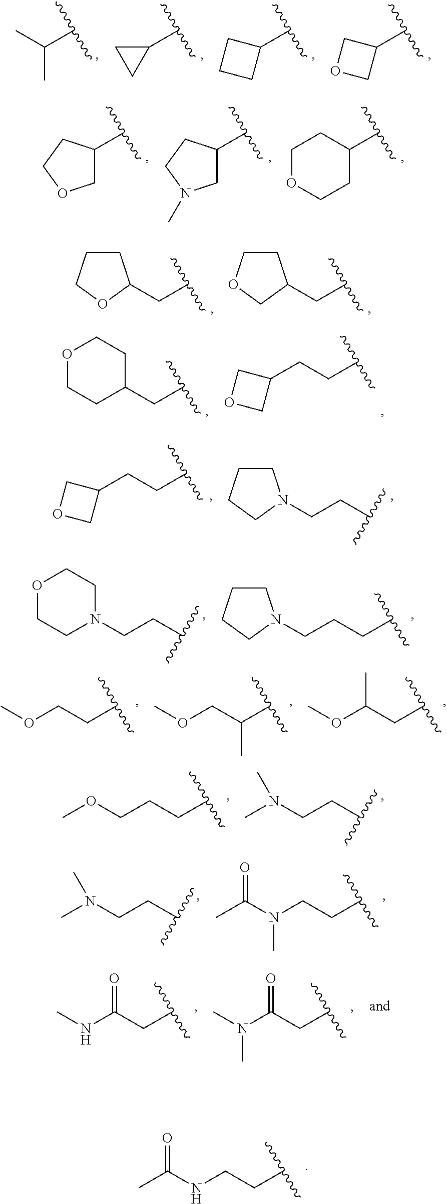

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

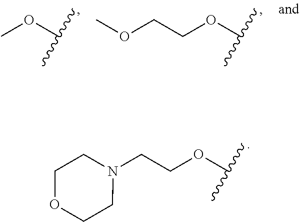

In some embodiments, R² is H.

In some embodiments, R¹ and R² taken together form a ring structure selected from the group consisting of

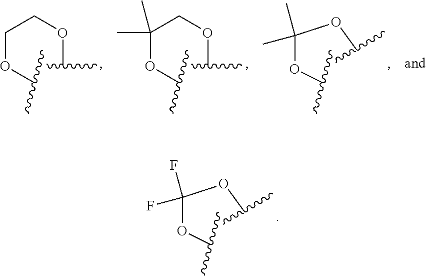

In some embodiments, R² and R⁵ taken together form a ring structure selected from the group consisting of consisting of

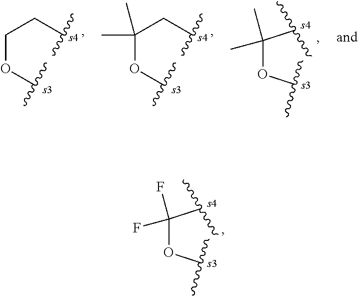

wherein s3 is the site covalently linked to the bicyclic ring structure and s4 is the site covalently linked to the oxygen atom.

In some embodiments, R⁶ is optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, and cycloalkyl.

In some embodiments, R⁶ is selected from the group consisting of methyl and

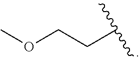 [SMILES: *CCOC]

In some embodiments, R¹ and R⁶ taken together form a ring structure selected from the group consisting of consisting of

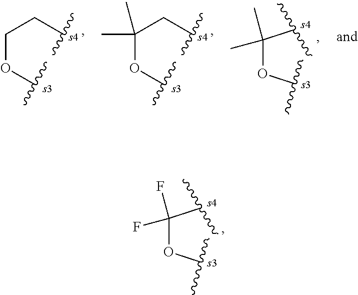

wherein s3 is the site covalently linked to the bicyclic ring structure and s4 is the site covalently linked to the oxygen atom.

In some embodiments, R⁵ and R⁶ taken together form a ring structure selected from the group consisting of

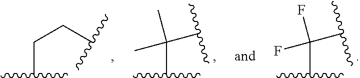

In some embodiments, R³ is F or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹, X², X³, and X⁴ are independently CH or N, provided that not more that one of X³ and X⁴ is N.

In some embodiments, X¹ is CH and X² is CH.

In some embodiments, X¹ is CH and X² is N.

In some embodiments, X³ is CH and X⁴ is N.

In some embodiments, X¹ is N, X² is CH, X³ is CH, and X⁴ is CH; X¹ is CH, X² is N, X³ is CH, and X⁴ is CH; X¹ is CH, X² is CH, X³ is N, and X⁴ is CH; X¹ is CH, X² is CH, X³ is CH, and X⁴ is N; X¹ is N, X² is N, X³ is CH, and X⁴ is CH; X¹ is N, X² is N, X³ is N, and X⁴ is CH; or X¹ is N, X² is N, X³ is CH, and X⁴ is N.

In some embodiments, L is selected from the group consisting of direct bond and C₁-C₆ alkyl optionally substituted with (E¹¹)ₘ.

In some embodiments,

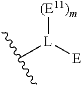

is selected from the group consisting of

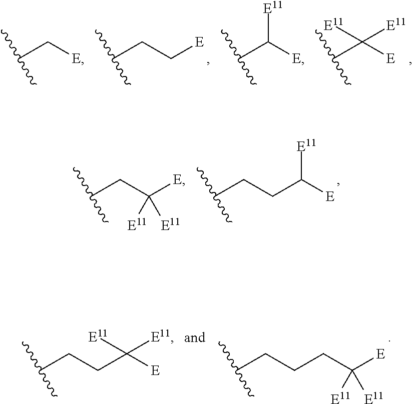

In some embodiments, E¹¹, at each occurrence, is independently selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

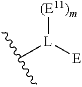

is selected from the group consisting of

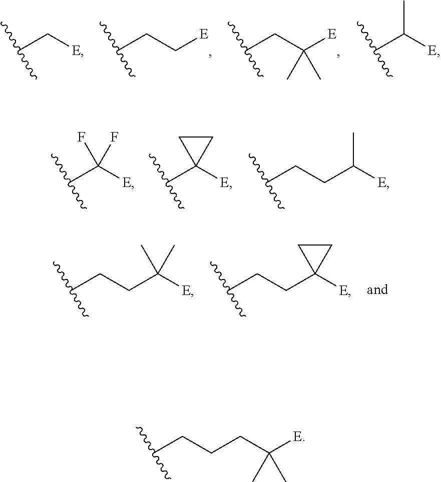

In some embodiments, E is selected from the group consisting of H; C₁-C₆ alkenyl; cyano; haloalkoxy; haloalkyl; halogen; optionally substituted C₁-C₆ alkyl wherein the optionally substituted substituent, at each occurrence, is independently selected from the group consisting of C₁-C₆ alkyl, alkoxy, amine, halogen, haloalkyl, haloalkoxy, hydroxy, and cyano; optionally substituted C₃-C₆ cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano; optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone; and optionally substituted alkoxy wherein the optionally substituted substituent, at each occurrence, is independently, selected from the group consisting of cycloalkyl, and heterocyclyl.

In some embodiments, E is selected from the group consisting of H, fluoro, methyl, trifluoromethyl, methoxy, tert-butoxy, trifluoromethoxy, cyano, alkenyl,

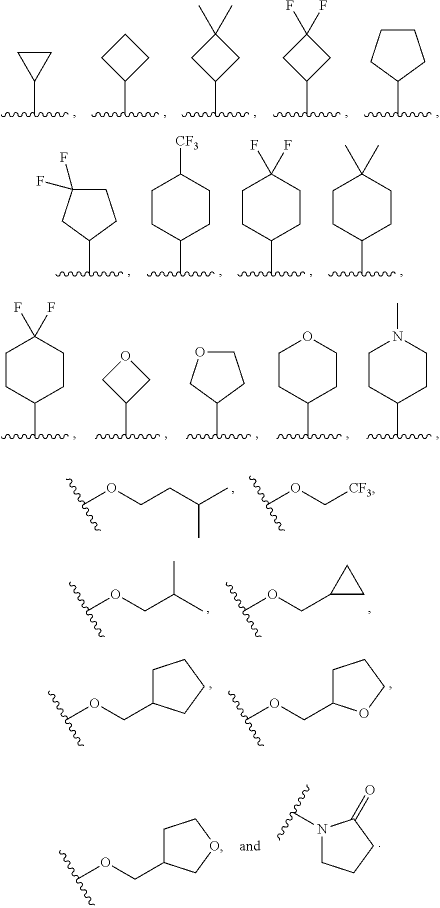

In some embodiments,

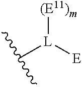

is selected from the group consisting of

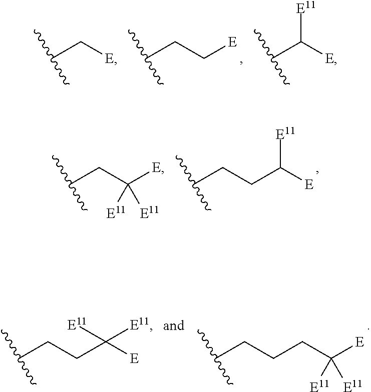

In some embodiments,

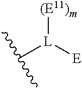

is selected from the group consisting of

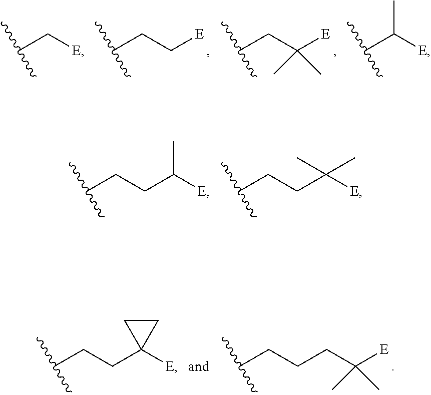

In some embodiments, L is not a direct bond, E is selected from the group consisting of H, fluoro, methyl, trifluoromethyl, methoxy, tert-butoxy, trifluoromethoxy, cyano, alkenyl,

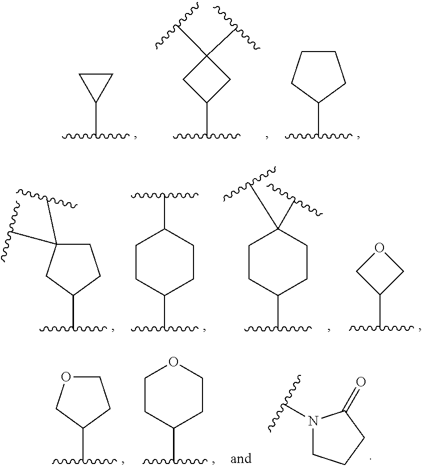

In some embodiments, L is not a direct bond, E is selected from the group consisting of H, fluoro, methyl, trifluoromethyl, methoxy, tert-butoxy, trifluoromethoxy, cyano, alkenyl,

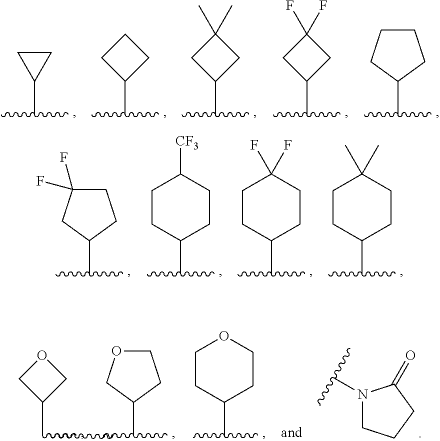

In some embodiments,

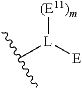

is selected from the group consisting of

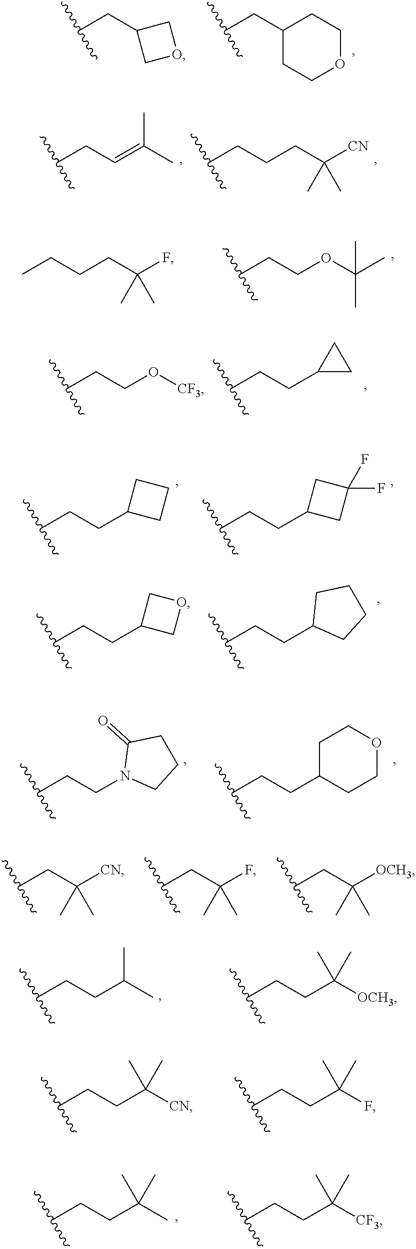

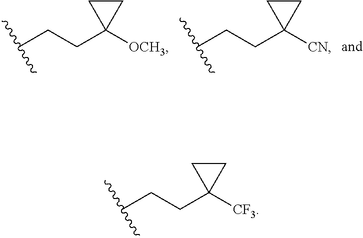 [SMILES: *CCC1(OC)CC1 ; *CCC1(C#N)CC1 ; *CCC1(C(F)(F)F)CC1]

In some embodiments, when L is a direct bond,

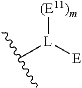

is selected from the group consisting of

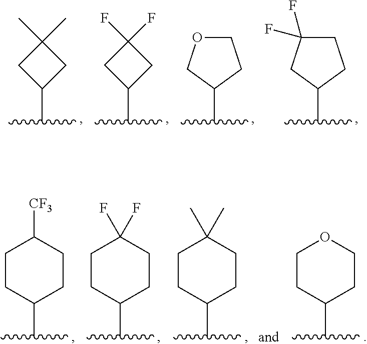

In some embodiments, R⁷ is H, alkyl, or haloalkyl.

In some embodiments, R⁷ is H.

In some embodiments,

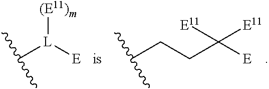

In some embodiments,

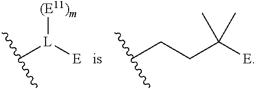

In some embodiments, E is selected from the group consisting of H, fluoro, methoxy, and cyano.

In some embodiments,

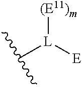

is selected from the group consisting of

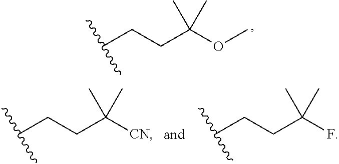

In some embodiments,

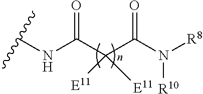

is selected from the group

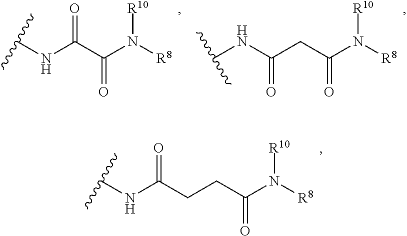

consisting of

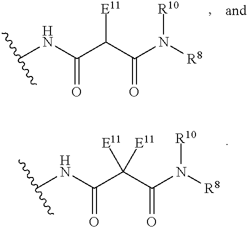

In some embodiments,

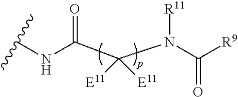

is selected from the group consisting

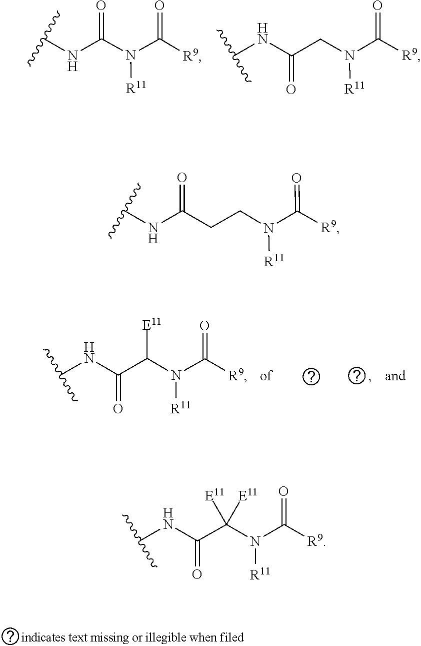

occurrence, is independently selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments, E is selected from the group consisting of H, fluoro, methyl, trifluoromethyl, methoxy, cyano,

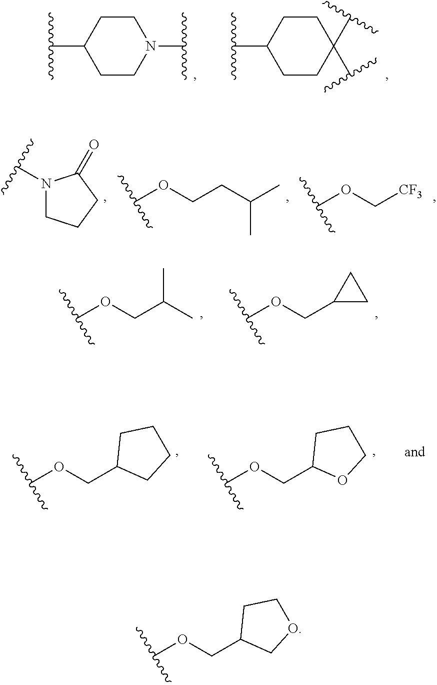

In some embodiments, E is selected from the group consisting of H, fluoro, methyl, trifluoromethyl, methoxy, cyano,

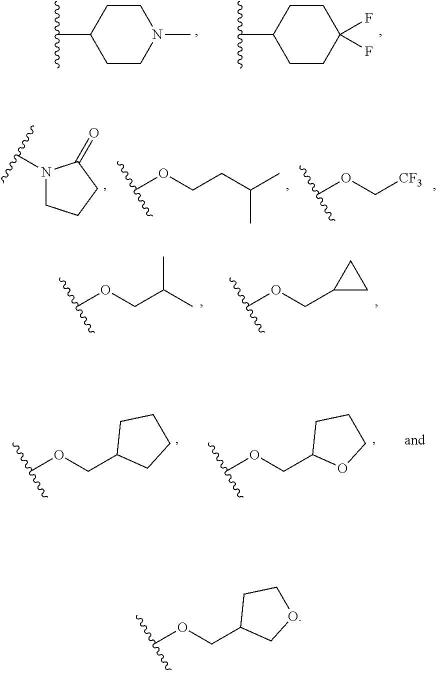

In some embodiments,

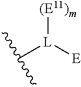

is selected from the group consisting of

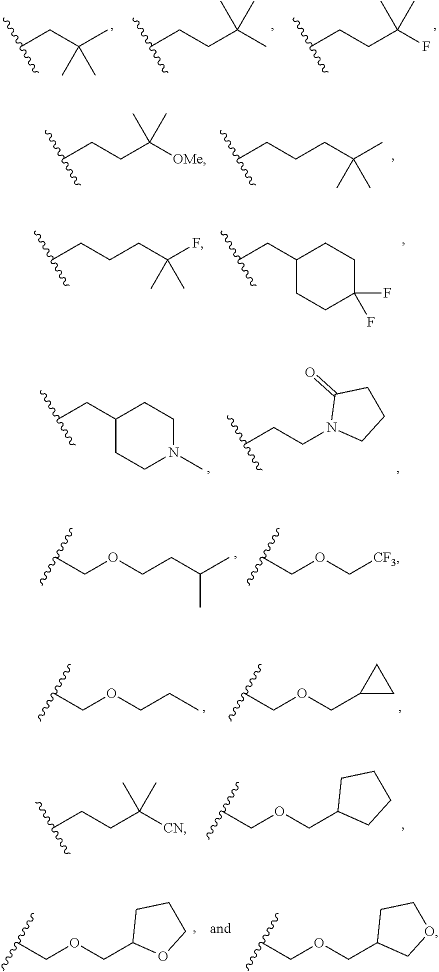

In some embodiments, R⁸ is selected from the group consisting of alkyl, cycloalkyl, and heterocyclyl, and R¹⁰ is selected from the group consisting of H and alkyl.

In some embodiments, R⁸ is selected from the group consisting of

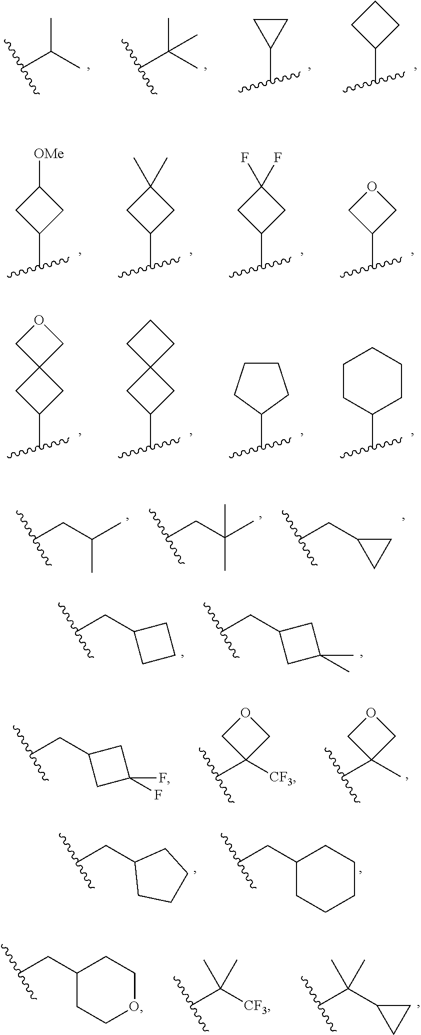

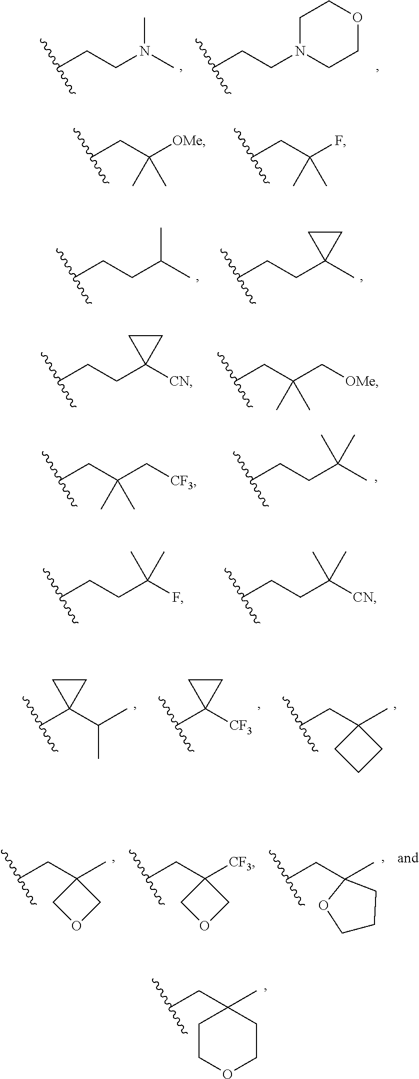

and R¹⁰ is selected from the group consisting of H, methyl, ethyl, and isopropyl.

In some embodiments, R⁸ taken together with R¹⁰ and the N atom to which R⁸ and R are attached form a ring structure selected from the group consisting of

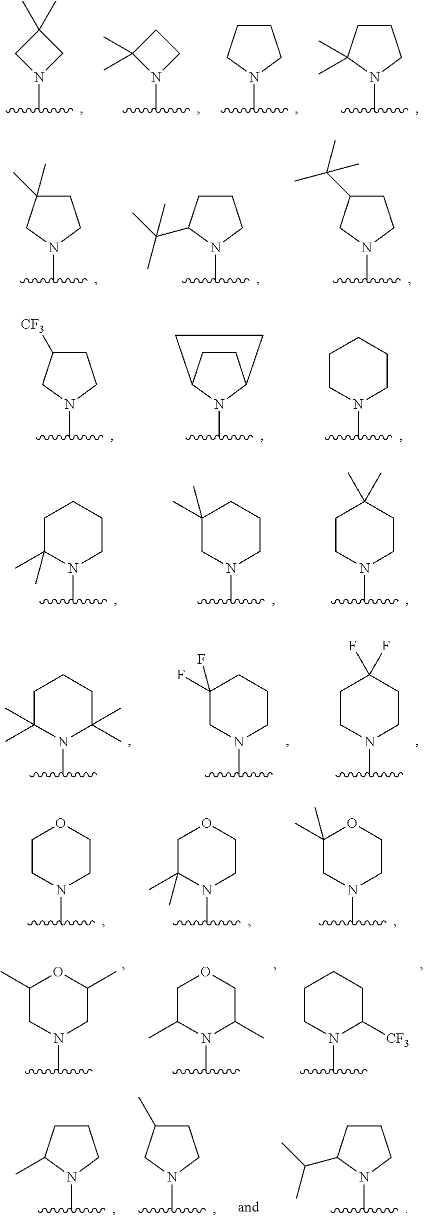

In some embodiments, L is a direct bond, R⁸ is selected from the group consisting of

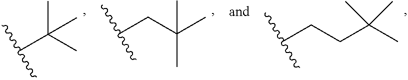

### and R¹⁰ is H.

In some embodiments, R⁹ is selected from the group consisting of

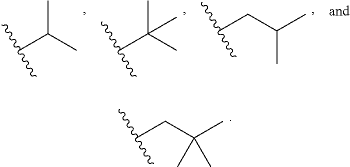

In some embodiments, R¹¹ is selected from the group consisting of H and C₁-C₆ alkyl.

In some embodiments, R¹¹ is selected from the group consisting of H and methyl.

In some embodiments, R¹¹ taken together with R⁹ and the atoms to which they are respectively attached form an optionally substituted ring wherein the substituent at each occurrence, is independently selected from the group consisting of alkyl, haloalkyl, and cycloalkyl.

In some embodiments, R¹¹ taken together with R⁹ and the atoms to which they are respectively attached form an optionally substituted ring wherein two instances of the substituent taken together with the atom to which they are attached form a 4 to 6-membered ring.

In some embodiments, R¹¹ taken together with R⁹ and the atoms to which they are respectively attached form an optionally substituted ring selected from the group consisting of

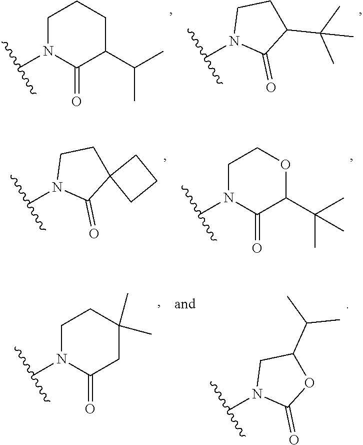

In some embodiments, E¹¹, at each occurrence, is independently selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

Compounds of Formula I-G, Formula I-H, Formula I-I, Formula I-J, Formula I-K, Formula I-L, and Formula I-M

In one embodiment, described herein is a compound represented by Formula I-G:

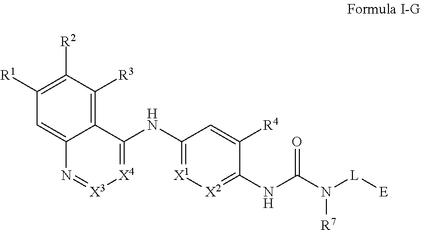

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-H:

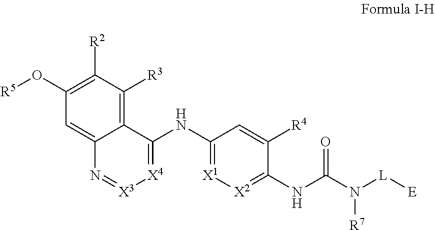

or a pharmaceutically acceptable salt, enantiomer, stereoisomer, or tautomer thereof, wherein:
X¹ and X² are each independently selected from the group consisting of N, CH, and CF; X³ and X⁴ are each independently CH or N, provided that not more that one of X³ and X⁴ is N;
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-I:

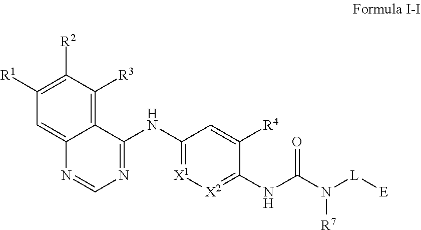

or a pharmaceutically acceptable salt, enantiomer, stereoisomer, or tautomer thereof, wherein:
X¹ and X² are each independently selected from the group consisting of N, CH, and CF;
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-J:

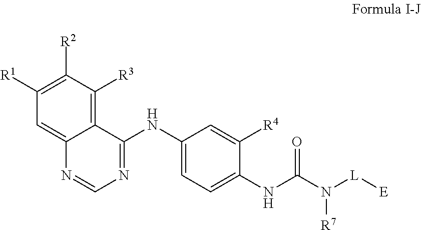

or a pharmaceutically acceptable salt, enantiomer, stereoisomer, or tautomer thereof, wherein:
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-K:

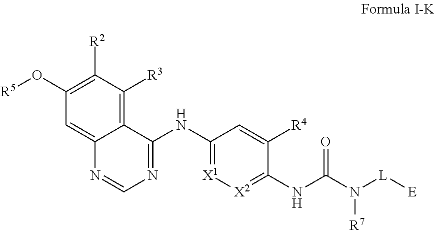

or a pharmaceutically acceptable salt, enantiomer, stereoisomer, or tautomer thereof, wherein:
X¹ and X² are each independently selected from the group consisting of N, CH, and CF;
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-L:

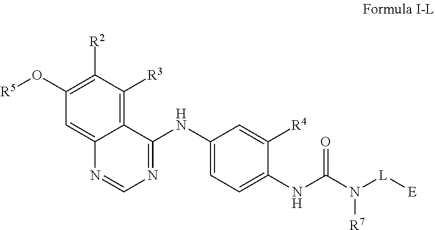

or a pharmaceutically acceptable salt, enantiomer, stereoisomer, or tautomer thereof, wherein:
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁷ is selected independently from the group consisting of H, alkyl, haloalkyl, or cycloalkyl; and
each occurrence of m is independently 0, 1, 2, 3, or 4.

In one embodiment, described herein is a compound represented by Formula I-M:

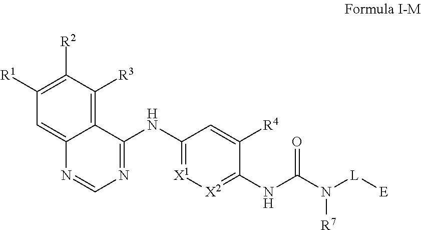

or a pharmaceutically acceptable salt, enantiomer, stereoisomer, or tautomer thereof, wherein:
X¹ and X² are each independently selected from the group consisting of N, CH, and CF;
L is selected from the group consisting of a direct bond and a C₁-C₆ alkyl optionally substituted with (E¹¹)ₘ, or when taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R⁴ is selected from the group consisting of H, F, alkyl, and alkoxy;
R⁷ is selected independently from the group consisting of H, alkyl, haloalkyl, or cycloalkyl; and
each occurrence of m is independently 0, 1, 2, 3, or 4;
with the proviso that the compound is not:

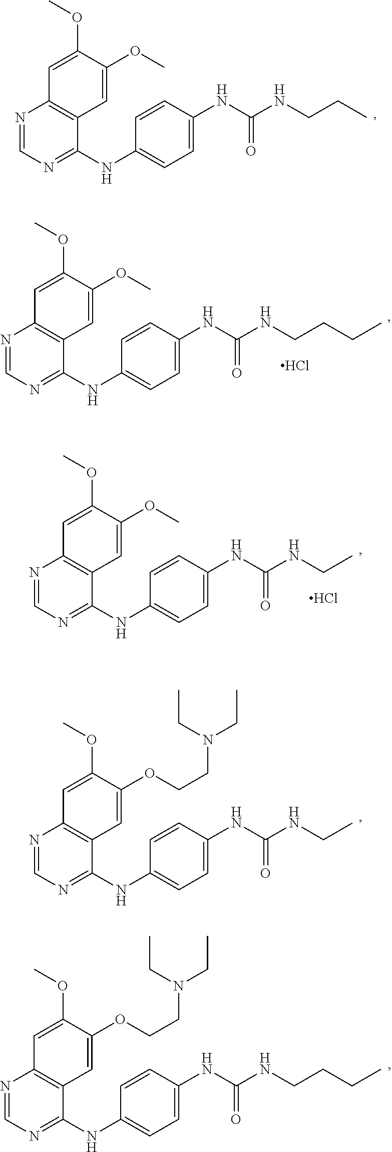

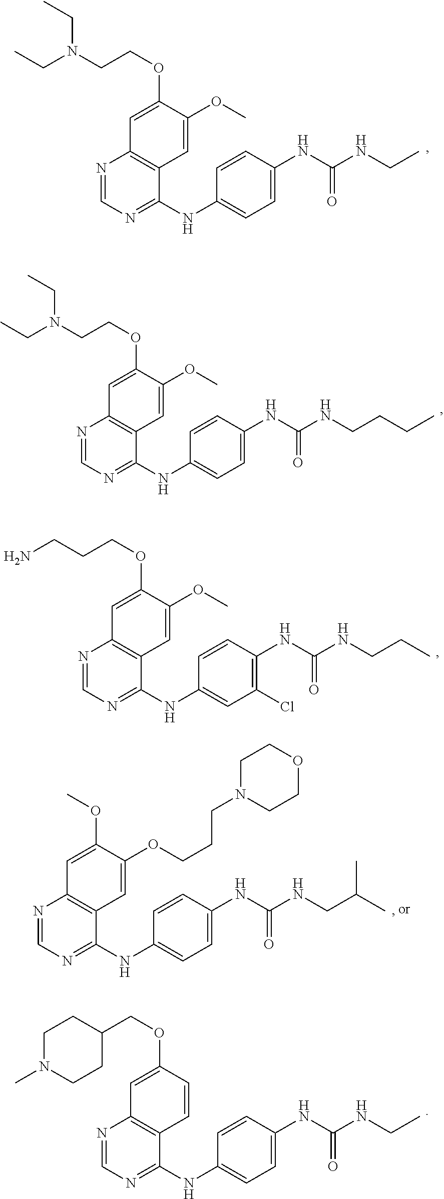

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

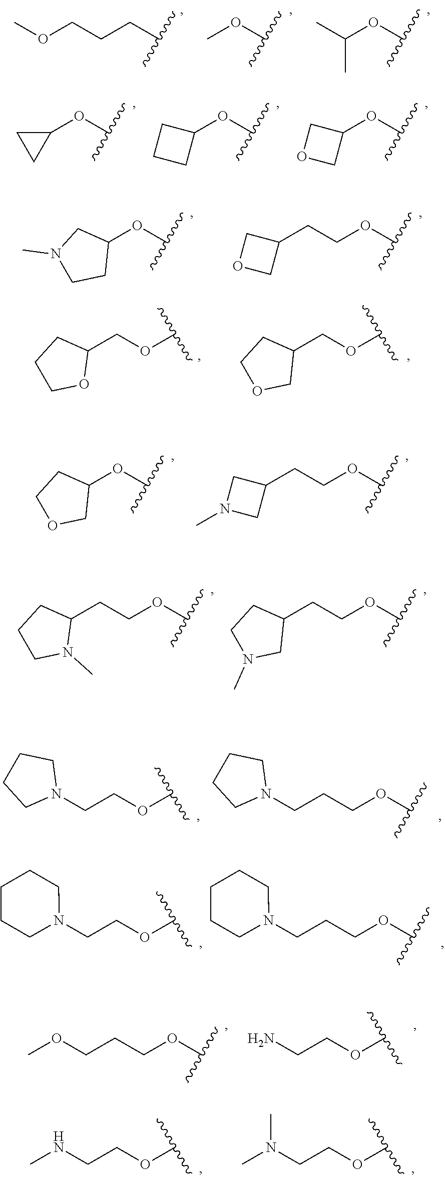

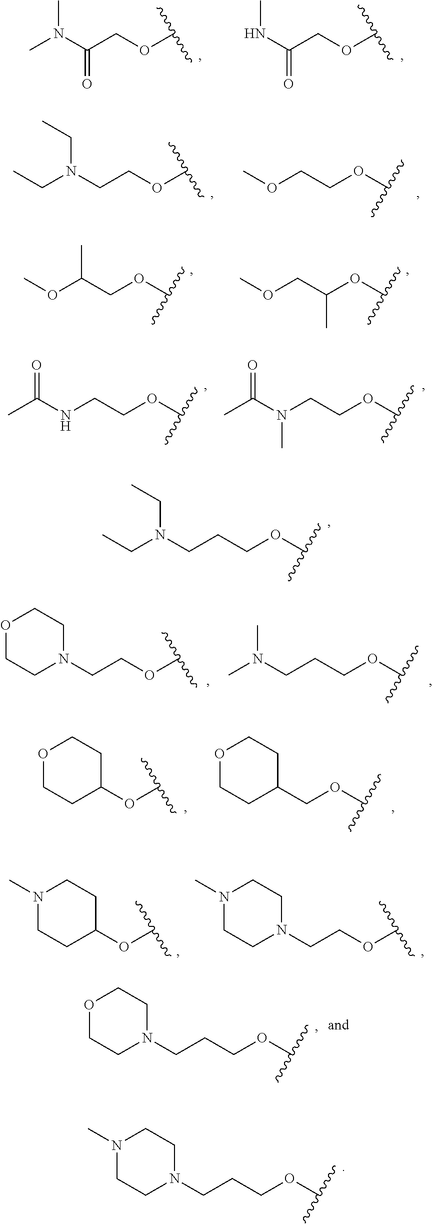

In some embodiments, R¹ is selected from the group consisting of H,

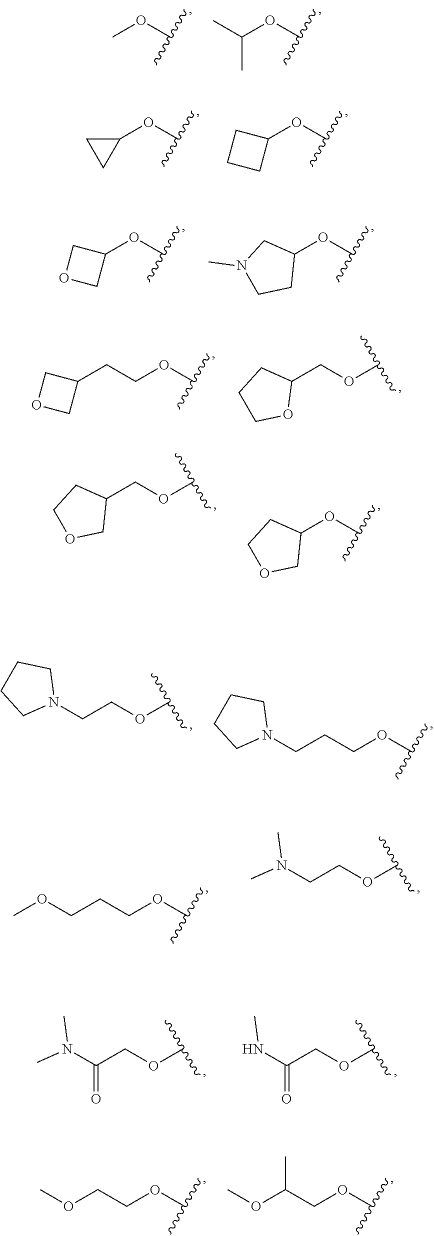

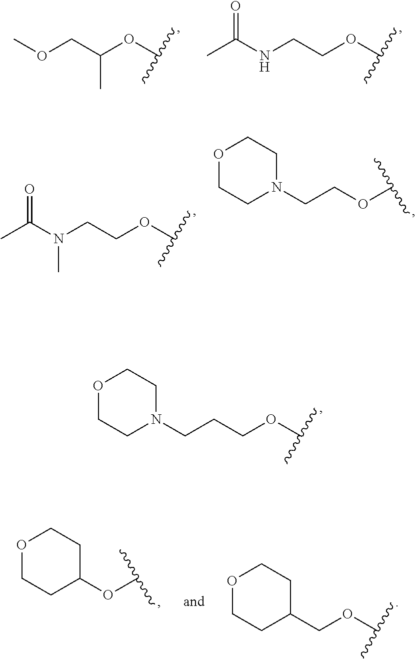

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃, CF₂CF₃

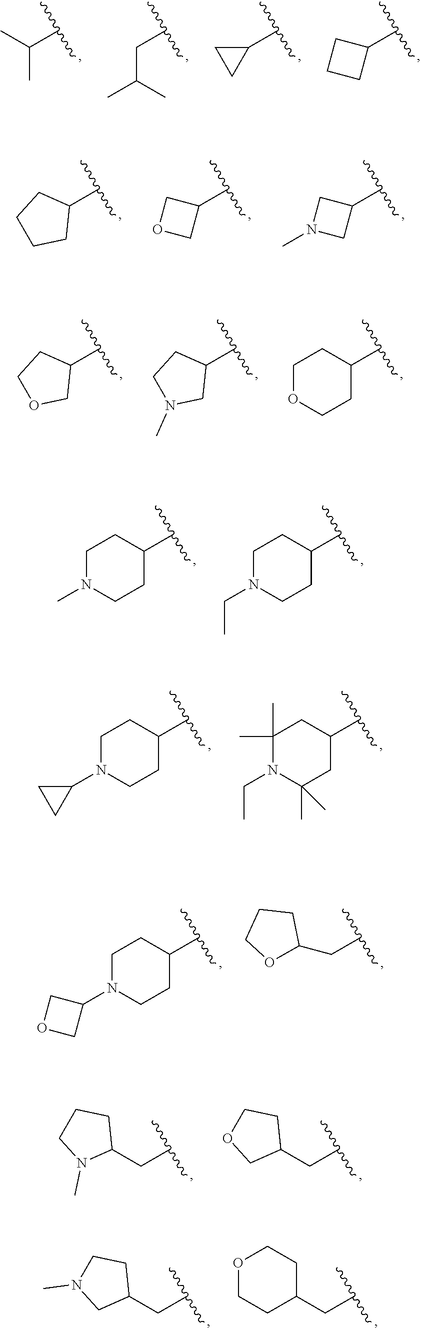

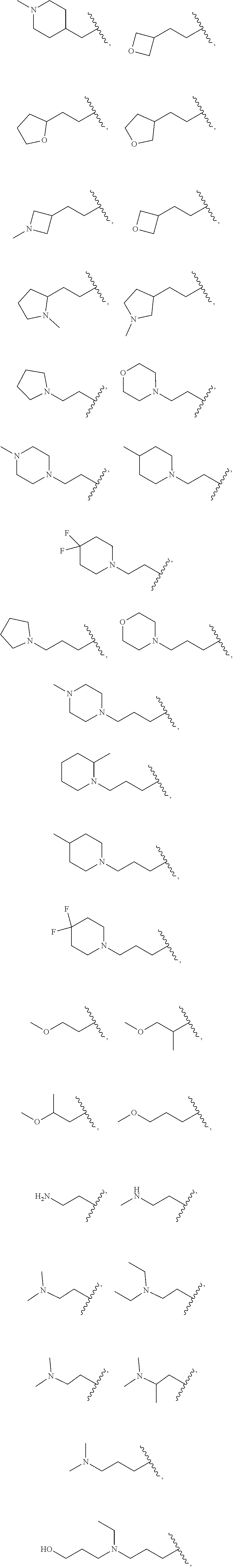

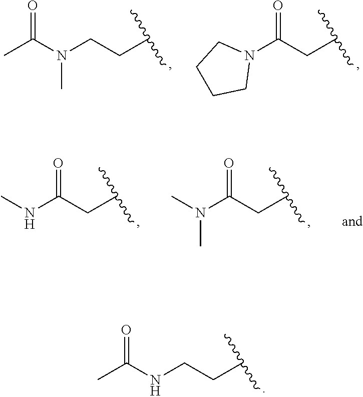

In some embodiments, R⁵ is selected from the group consisting of methyl,

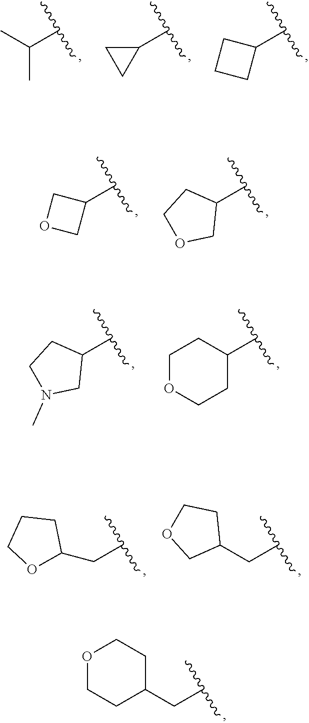

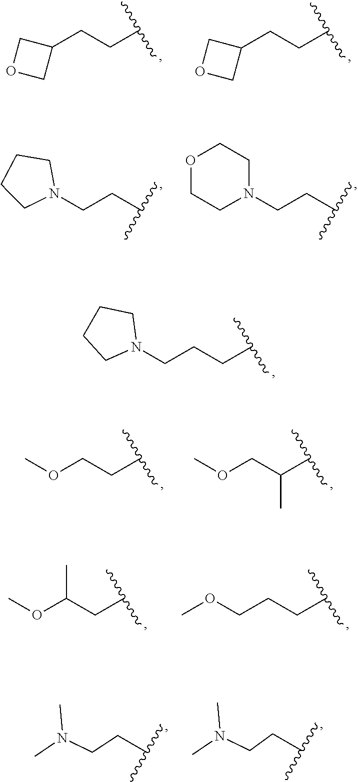

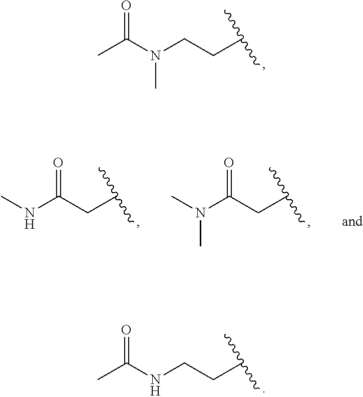

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

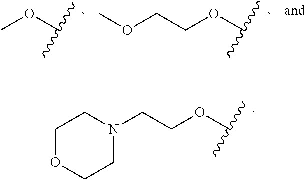

In some embodiments, R is H.

In some embodiments, R³ is F or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹, X², X³, and X⁴ are independently CH or N, provided that not more that one of X³ and X⁴ is N.

In some embodiments, X¹ is CH and X² is CH.

In some embodiments, X¹ is CH and X² is N.

In some embodiments, X³ is CH and X⁴ is N.

In some embodiments, L is selected from the group consisting of direct bond and C₁-C₆ alkyl optionally substituted with (E¹¹)ₘ.

In some embodiments,

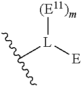

is selected from the group consisting of

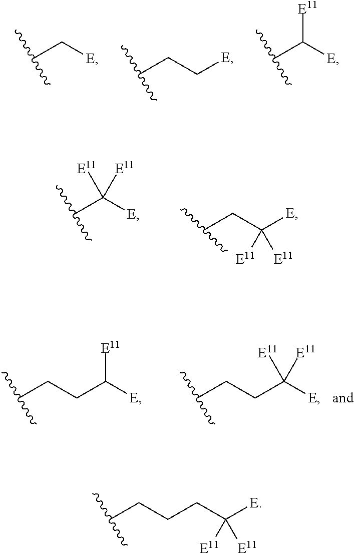

In some embodiments,

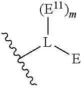

is selected from the group consisting of

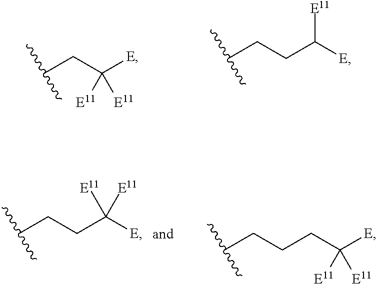

In some embodiments, E¹¹, at each occurrence, is independently, selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

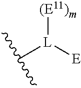

is selected from the group consisting of

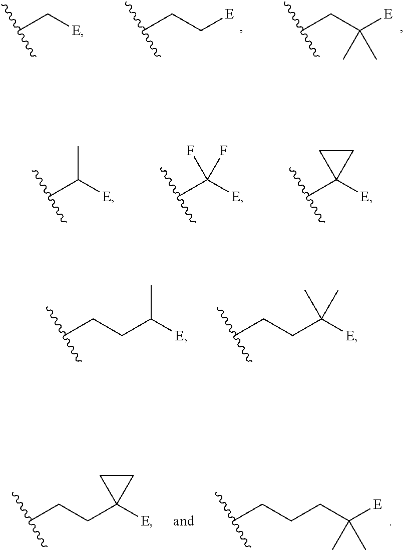

In some embodiments,

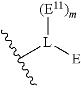

is selected from the group consisting of

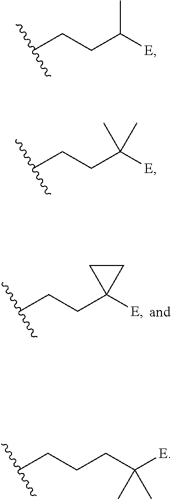

In some embodiments, R⁷ is H, alkyl, or haloalkyl.

In some embodiments, R⁷ is H.

In some embodiments, E is selected from the group consisting of: optionally substituted C₁-C₆ alkyl wherein the optionally substituted substituent, at each occurrence, is independently, selected from the group consisting of C₁-C₆ alkyl, alkoxy, amine, halogen, haloalkyl, haloalkoxy, hydroxy, and cyano, optionally substituted C₃-C₆ cycloalkyl wherein the optionally substituted substituent, at each occurrence, is independently, selected from the group consisting of C₁-C₆ alkyl, alkoxy, amine, halogen, haloalkyl, haloalkoxy, hydroxy, and cyano, H, C₁-C₆ alkenyl, cyano, haloalkoxy, haloalkyl, and halogen.

In some embodiments, when L is alkyl optionally substituted with (E¹¹)ₘ, E is selected from the group consisting of H, fluoro, methyl, trifluoromethyl, methoxy, tert-butoxy, trifluoromethoxy, cyano, alkenyl,

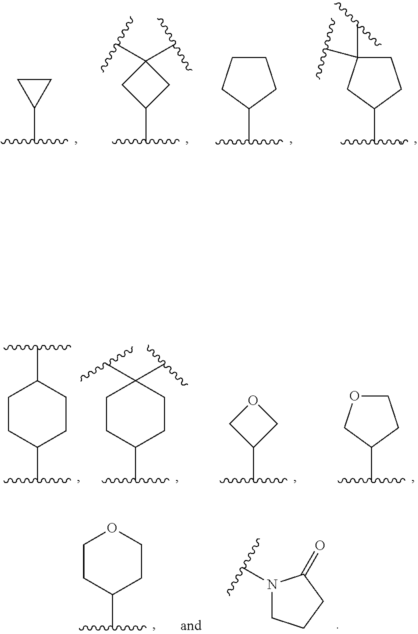

In some embodiments, when L is alkyl optionally substituted with (E¹¹)ₘ, E is selected from the group consisting of H, fluoro, methyl, trifluoromethyl, methoxy, tert-butoxy, trifluoromethoxy, cyano, alkenyl,

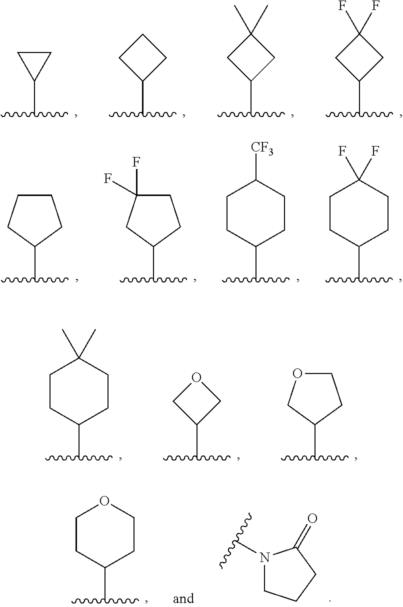

In some embodiments,

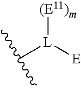

is selected from the group consisting of

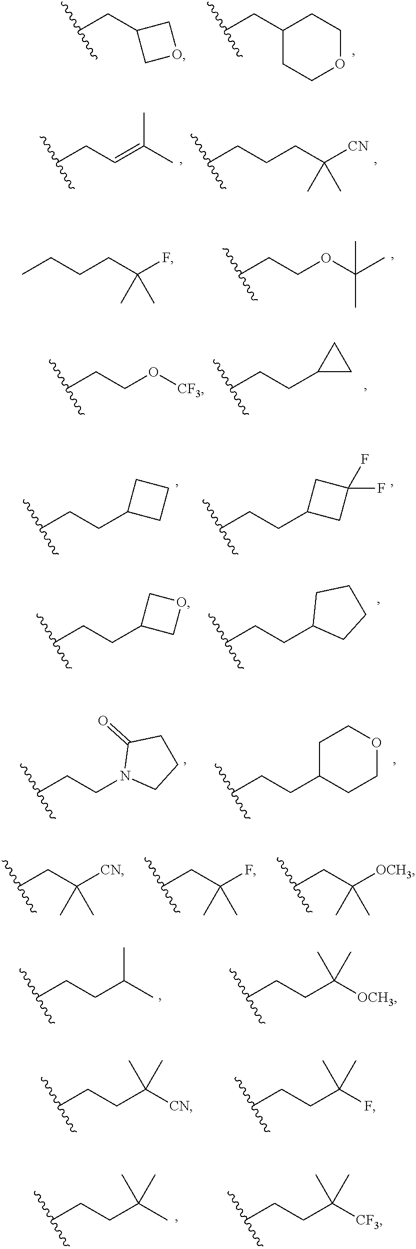

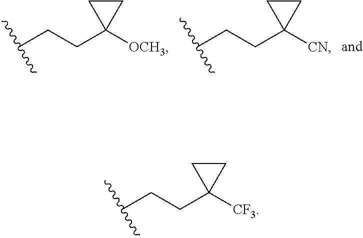 [SMILES: *CCC1(OC)CC1 ; *CCC1(C#N)CC1 ; *CCC1(C(F)(F)F)CC1]

In some embodiments,

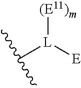

is selected from the group consisting of

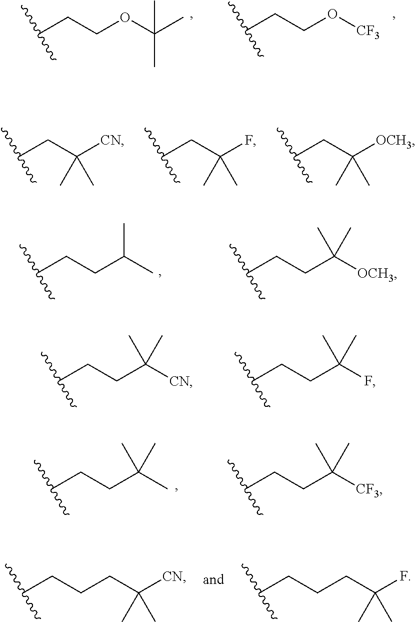

In some embodiments,

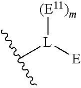

is selected from the group consisting of

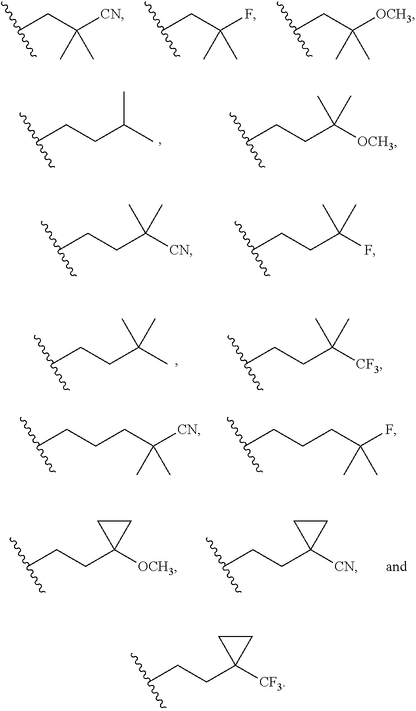

In some embodiments,

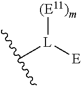

is selected from the group consisting of

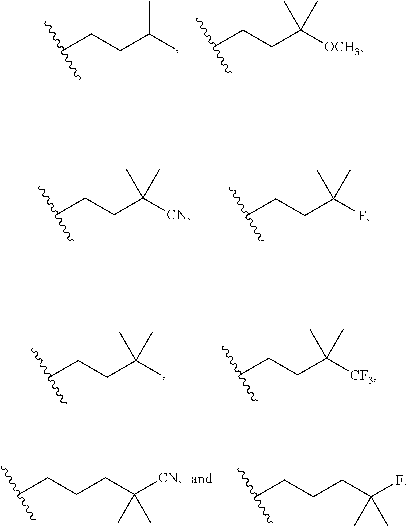

In some embodiments,

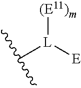

is selected from the group consisting of

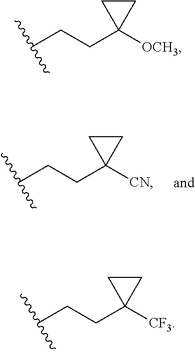 [SMILES: *CCC1(OC)CC1 ; *CCC1(C#N)CC1 ; *CCC1(C(F)(F)F)CC1]

In some embodiments, when L is a direct bond,

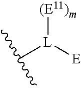

is selected from the group consisting of

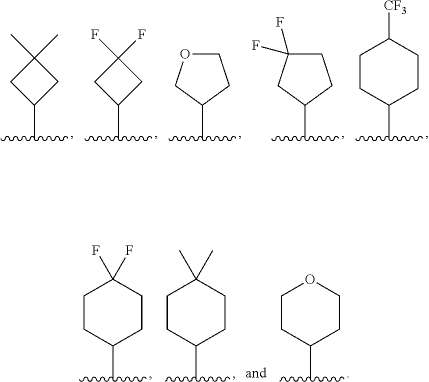

In some embodiments, R⁷ taken together with L and the N atom to which R⁷ and L are attached form an optionally substituted ring wherein the substituent, at each occurrence, is independently selected from the group consisting of alkyl, cycloalkyl, and haloalkyl.

In some embodiments, R⁷ taken together with L and the N atom to which R⁷ and L are attached form an optionally substituted ring wherein two instances of the substituent taken together with the atom to which they are attached form a 4 to 6-membered ring.

In some embodiments, R⁷ taken together with L and the N atom to which R⁷ and L are attached form a ring structure selected from the group consisting of

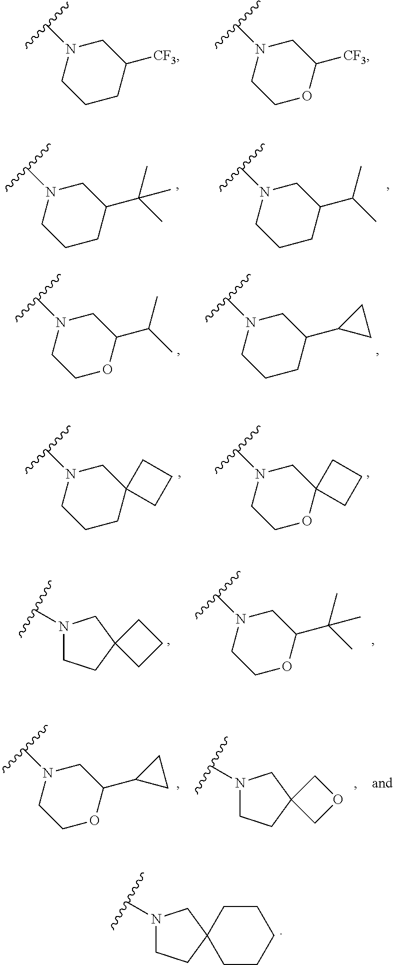

Compounds of Formula I-N, Formula I-O, Formula I-P, and Formula I-Q

In one embodiment, described herein is a compound represented by Formula I-N:

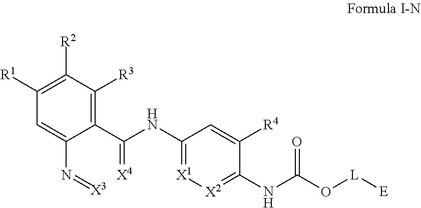

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy; and
each occurrence of m is independently 0, 1, 2, 3, or 4,
provided that the compound is not:

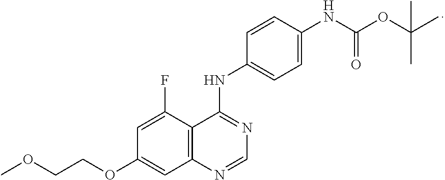 [SMILES: COCCOc1cc(F)c2c(Nc3ccc(NC(=O)OC(C)(C)C)cc3)ncnc2c1]

In one embodiment, described herein is a compound represented by Formula I-O:

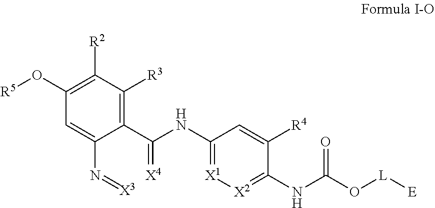

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R4 is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4,
provided that the compound is not:

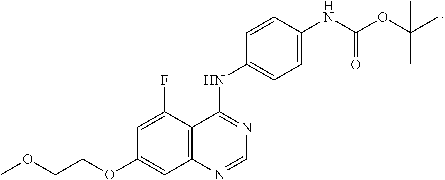 [SMILES: COCCOc1cc(F)c2c(Nc3ccc(NC(=O)OC(C)(C)C)cc3)ncnc2c1]

In one embodiment, described herein is a compound represented by Formula I-P:

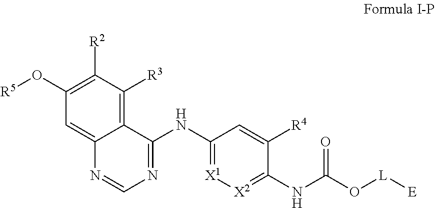

or a pharmaceutically acceptable salt, enantiomer, stereoisomer, or tautomer thereof, wherein:
X¹ and X² are each independently selected from the group consisting of N, CH, and CF;
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4,
provided that the compound is not:

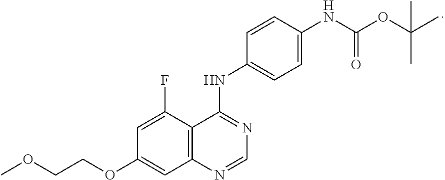 [SMILES: COCCOc1cc(F)c2c(Nc3ccc(NC(=O)OC(C)(C)C)cc3)ncnc2c1]

In one embodiment, described herein is a compound represented by Formula I-Q:

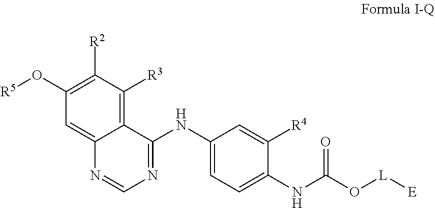

or a pharmaceutically acceptable salt, enantiomer, stereoisomer, or tautomer thereof, wherein:
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen, and
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4, provided that the compound is not:

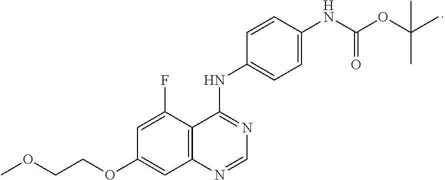 [SMILES: COCCOc1cc(F)c2c(Nc3ccc(NC(=O)OC(C)(C)C)cc3)ncnc2c1]

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

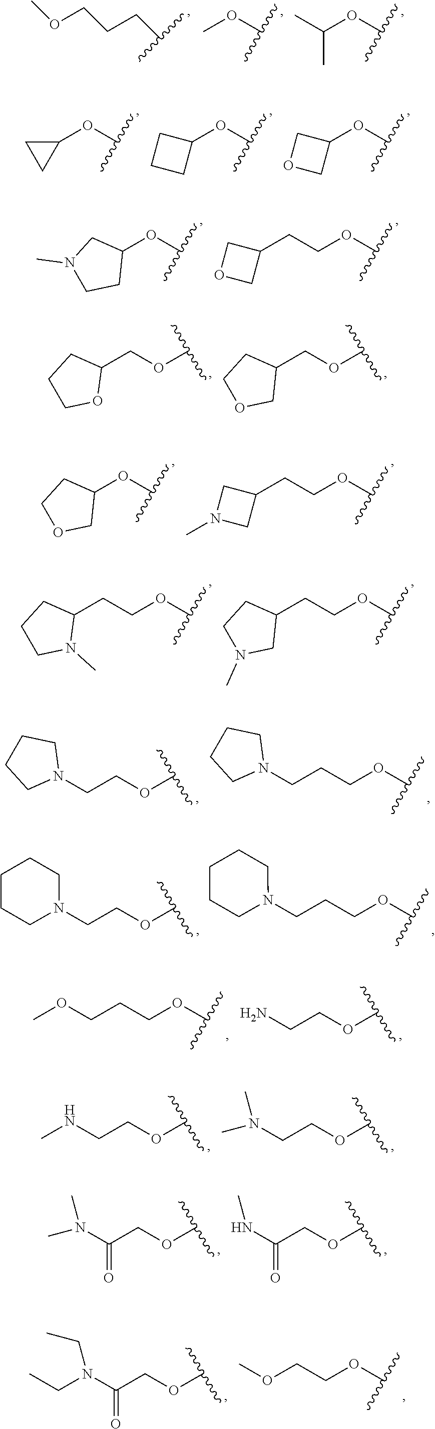

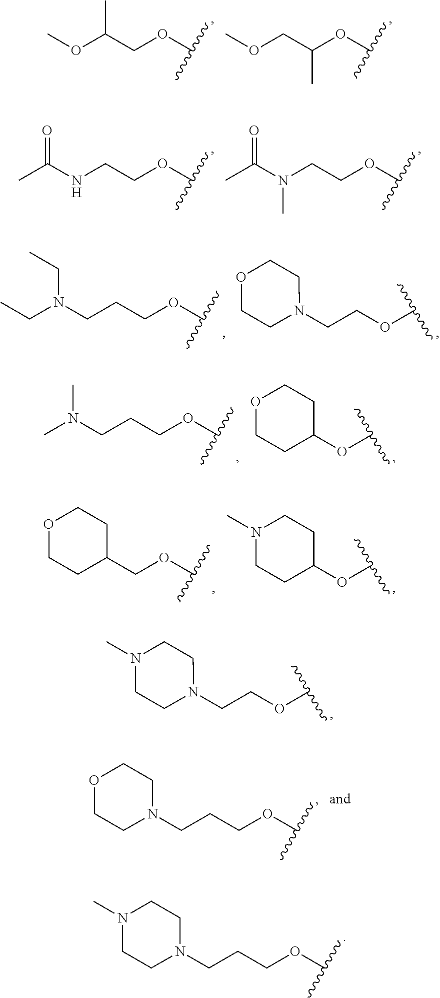

In some embodiments, R¹ is selected from the group consisting of H,

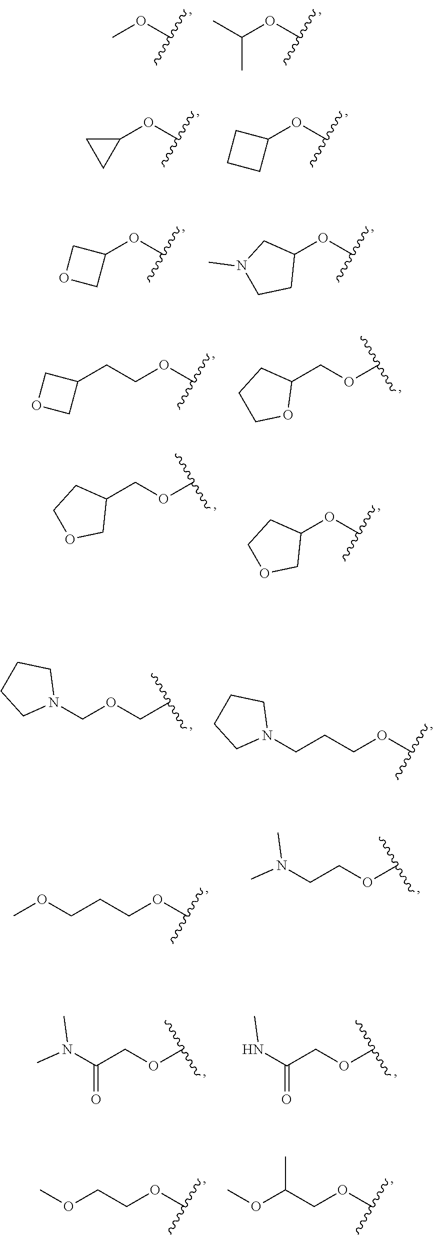

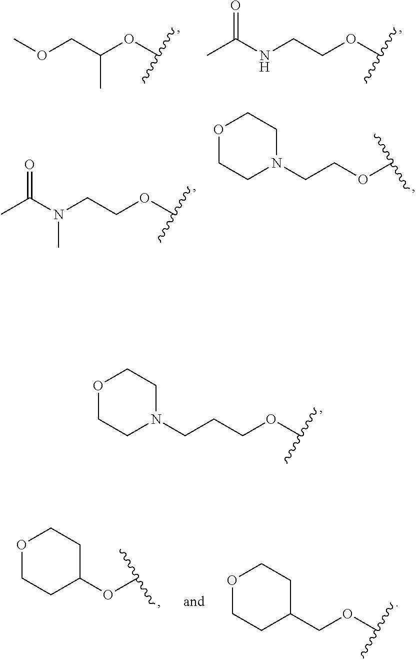

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃,

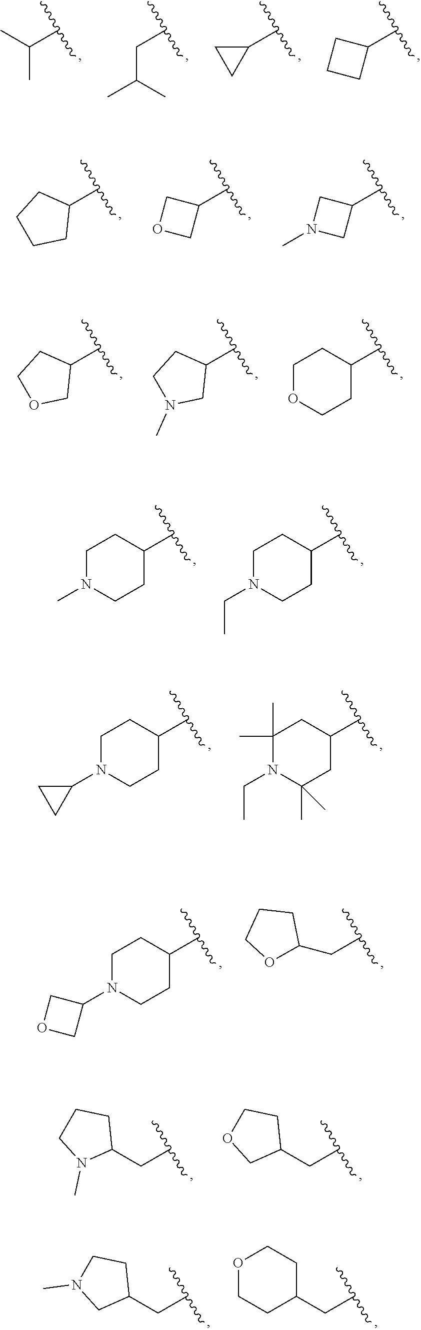

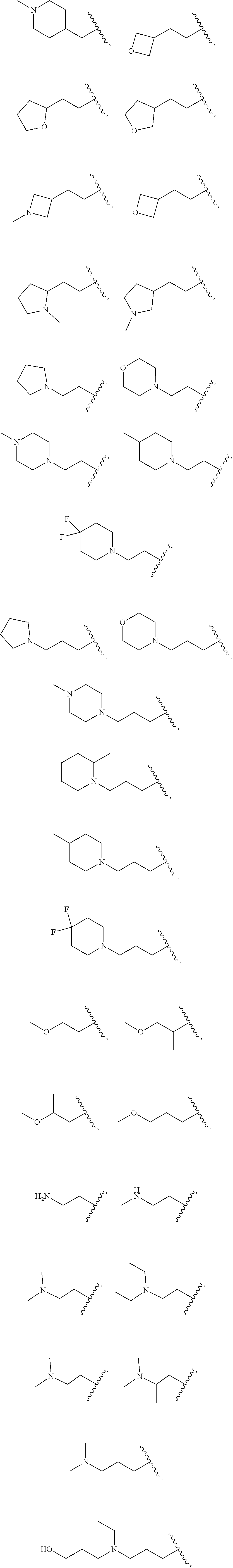

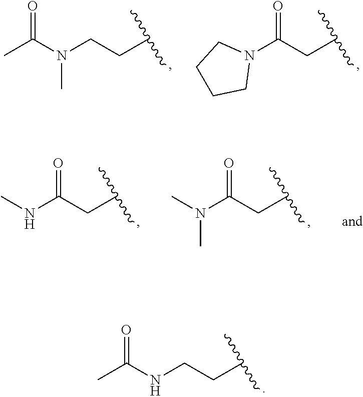

In some embodiments, R⁵ is selected from the group consisting of methyl,

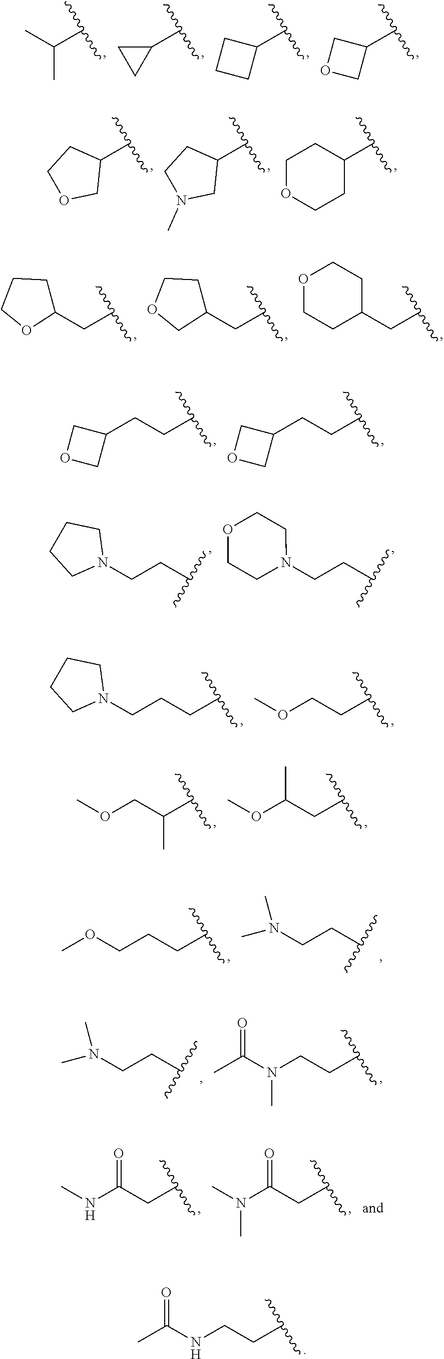

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

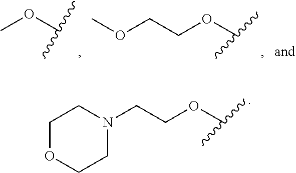

In some embodiments, R² is H.

In some embodiments, R³ is F or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹, X², X³, and X⁴ are independently CH or N, provided that not more that one of X³ and X⁴ is N.

In some embodiments, X¹ is CH and X² is CH.

In some embodiments, X¹ is CH and X² is N.

In some embodiments, X³ is CH and X⁴ is N.

In some embodiments, X¹ is N, X² is CH, X³ is CH, and X⁴ is CH; X¹ is CH, X² is N, X³ is CH, and X⁴ is CH; X¹ is CH, X² is CH, X³ is N, and X⁴ is CH; X¹ is CH, X² is CH, X³ is CH, and X⁴ is N; X¹ is N, X² is N, X³ is CH, and X⁴ is CH; X¹ is N, X² is N, X³ is N, and X⁴ is CH; or X¹ is N, X² is N, X³ is CH, and X⁴ is N.

In some embodiments, L is selected from the group consisting of direct bond and C₁-C₆ alkyl optionally substituted with (E¹¹)ₘ.

In some embodiments,

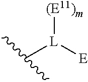

is selected from the group consisting of

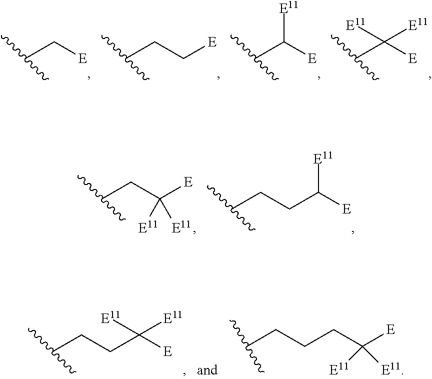

In some embodiments, E¹¹, at each occurrence, is independently, selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

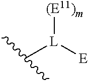

is selected from the group consisting of

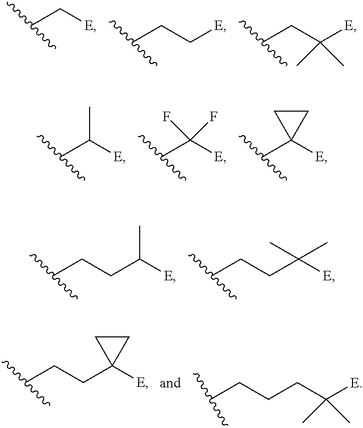

In some embodiments,

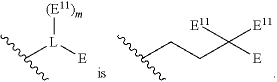

In some embodiments,

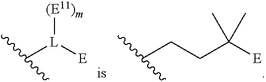

In some embodiments, E is selected from the group consisting of optionally substituted amido wherein the substituent, at each occurrence, is independently, selected from the group consisting of optionally substituted C₁-C₆ alkyl wherein the optionally substituted substituent, at each occurrence, is independently, selected from the group consisting of C₁-C₆ alkyl, alkoxy, amine, halogen, haloalkyl, haloalkoxy, hydroxy, and cyano, H, cyano, haloalkoxy, haloalkyl, and halogen.

In some embodiments, E is selected from the group consisting of H, fluoro, methoxy, and cyano.

In some embodiments,

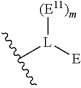

is selected from the group consisting of

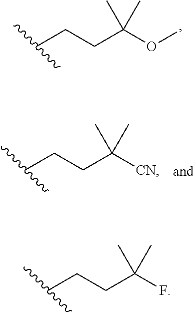

Compounds of Formula I-R, Formula I-S, Formula I-T, and Formula I-U

In one embodiment, described herein is a compound represented by Formula I-R:

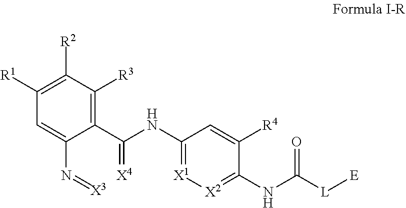

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy; and
each occurrence of m is independently 0, 1, 2, 3, or 4.

In one embodiment, described herein is a compound represented by Formula I-S:

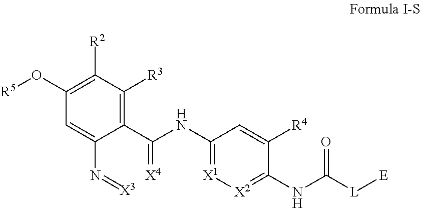

or a pharmaceutically acceptable salt, enantiomer, stereoisomer, or tautomer thereof, wherein:
X¹ and X² are each independently selected from the group consisting of N, CH, and CF; X³ and X⁴ are each independently CH or N, provided that not more that one of X³ and X⁴ is N;
L is selected from the group consisting of a direct bond and a C₁-C₆ alkyl optionally substituted with (E¹¹)ₘ;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4.

In one embodiment, described herein is a compound represented by Formula I-T:

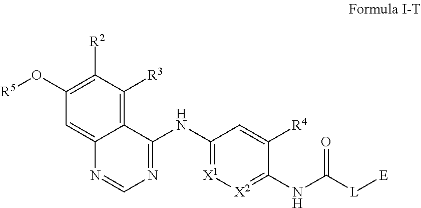

or a pharmaceutically acceptable salt, enantiomer, stereoisomer, or tautomer thereof, wherein:
X¹ and X² are each independently selected from the group consisting of N, CH, and CF;
L is selected from the group consisting of a direct bond and a C₁-C₆ alkyl optionally substituted with (E¹¹)ₘ;
E is selected from the group consisting of H, cyano, alkoxy, haloalkoxy, haloalkyl, halogen,
optionally substituted amido wherein the substituent, at each occurrence, is independently, selected from the group consisting of optionally substituted alkyl, optionally substituted heterocyclyl, and optionally substituted cycloalkyl, wherein
   the substituent of the optionally substituted alkyl, at each occurrence, is independently, selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently, selected from the group consisting of C₁-C₆ alkyl, alkoxy, amine, halogen, haloalkyl, haloalkoxy, hydroxy, oxo, and cyano,
   the substituent of the optionally substituted cycloalkyl at each occurrence, is independently, selected from the group consisting of C₁-C₆ alkyl, alkoxy, amine, halogen, haloalkyl, haloalkoxy, hydroxy, and cyano,
optionally substituted alkyl wherein the optionally substituted substituent, at each occurrence, is independently, selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently, selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently, selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently, selected from the group consisting of cycloalkyl and heterocyclyl;
E¹¹, at each occurrence, is independently, selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl,
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4.

In one embodiment, described herein is a compound represented by Formula I-U

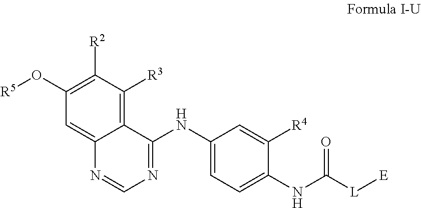

or a pharmaceutically acceptable salt, enantiomer, stereoisomer, or tautomer thereof, wherein:
L is selected from the group consisting of a direct bond and a C₁-C₆ alkyl optionally substituted with (E¹¹)ₘ;
E is selected from the group consisting of H, cyano, alkoxy, haloalkoxy, haloalkyl, halogen,
optionally substituted amido wherein the substituent, at each occurrence, is independently, selected from the group consisting of optionally substituted alkyl, optionally substituted heterocyclyl, and optionally substituted cycloalkyl, wherein
   the substituent of the optionally substituted alkyl, at each occurrence, is independently, selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently, selected from the group consisting of C₁-C₆ alkyl, alkoxy, amine, halogen, haloalkyl, haloalkoxy, hydroxy, oxo, and cyano,
   the substituent of the optionally substituted cycloalkyl at each occurrence, is independently, selected from the group consisting of C₁-C₆ alkyl, alkoxy, amine, halogen, haloalkyl, haloalkoxy, hydroxy, and cyano,
optionally substituted alkyl wherein the optionally substituted substituent, at each occurrence, is independently, selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently, selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently, selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently, selected from the group consisting of cycloalkyl heterocyclyl;
E¹¹, at each occurrence, is independently, selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of F and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl,
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen; and
each occurrence of m is independently 0, 1, 2, 3, or 4.

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

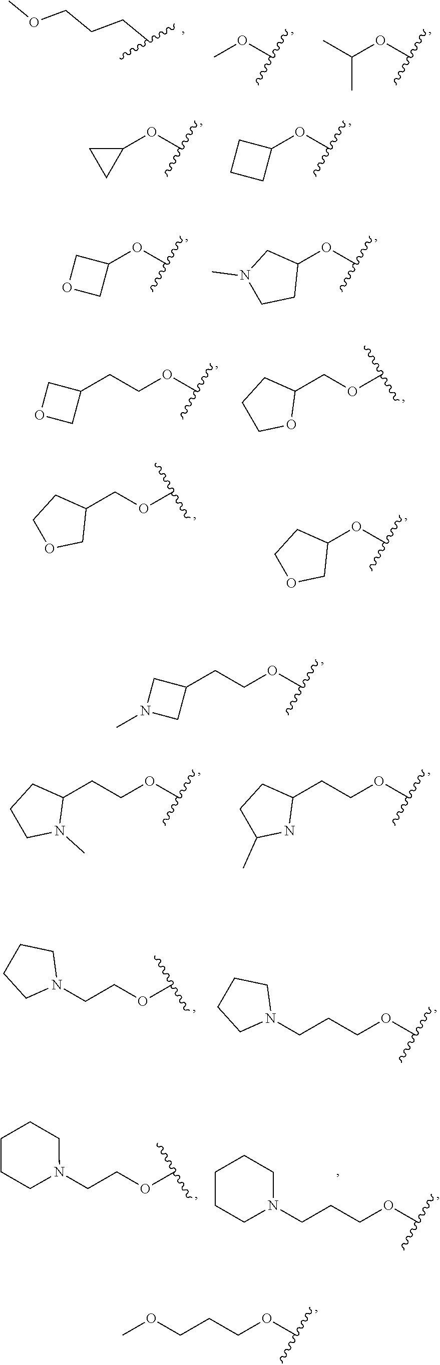

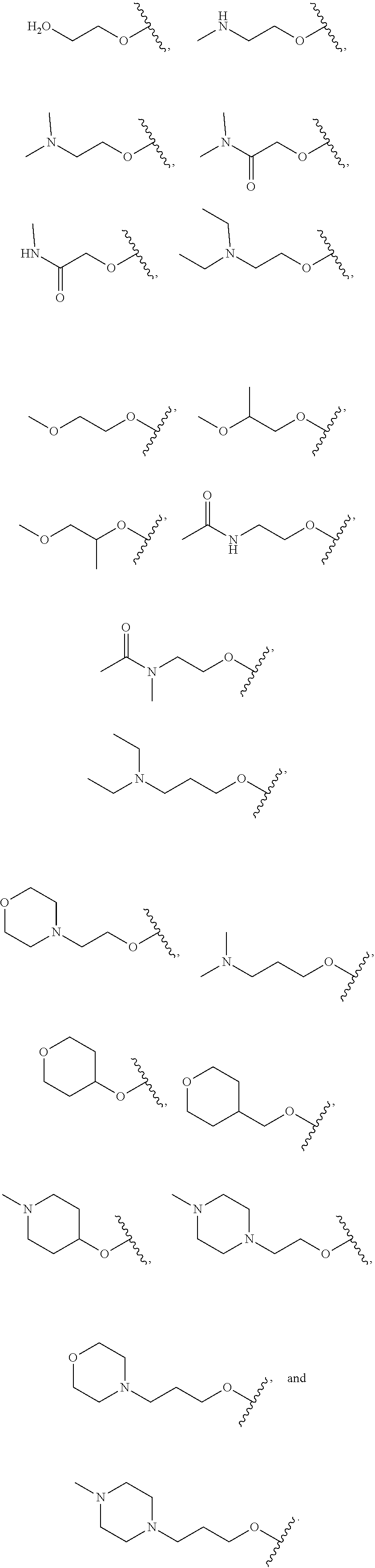

In some embodiments, R¹ is selected from the group consisting of H,

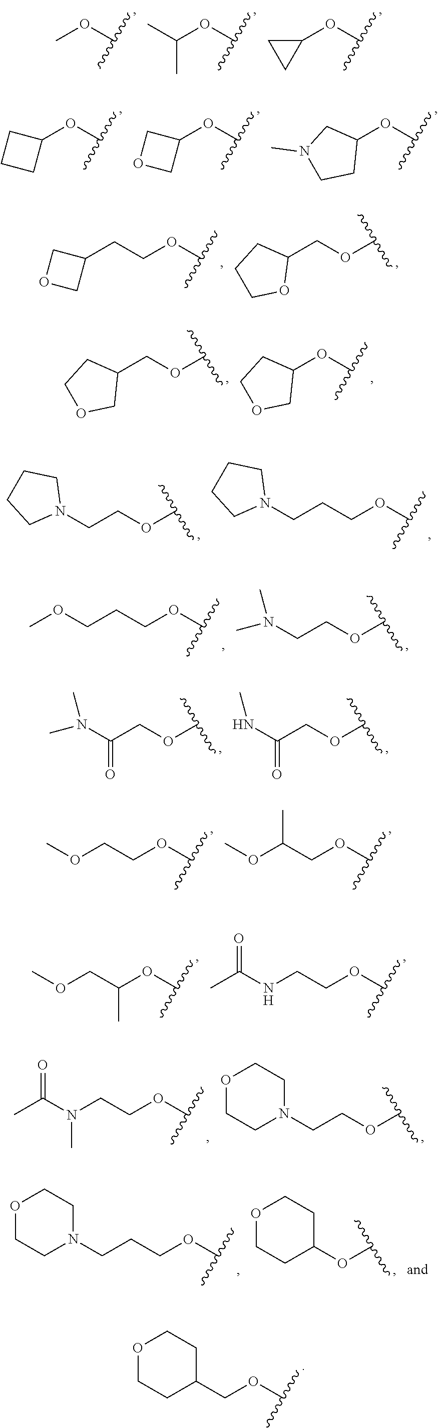

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃

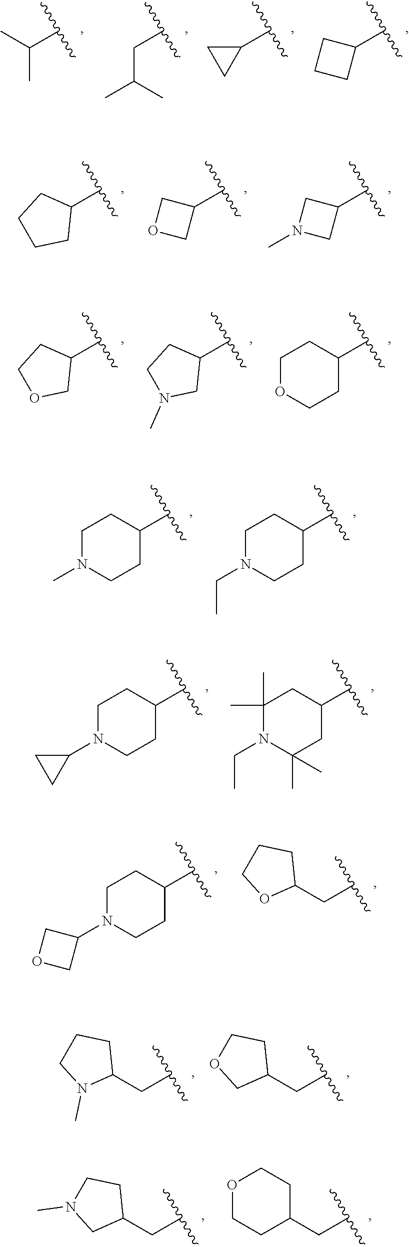

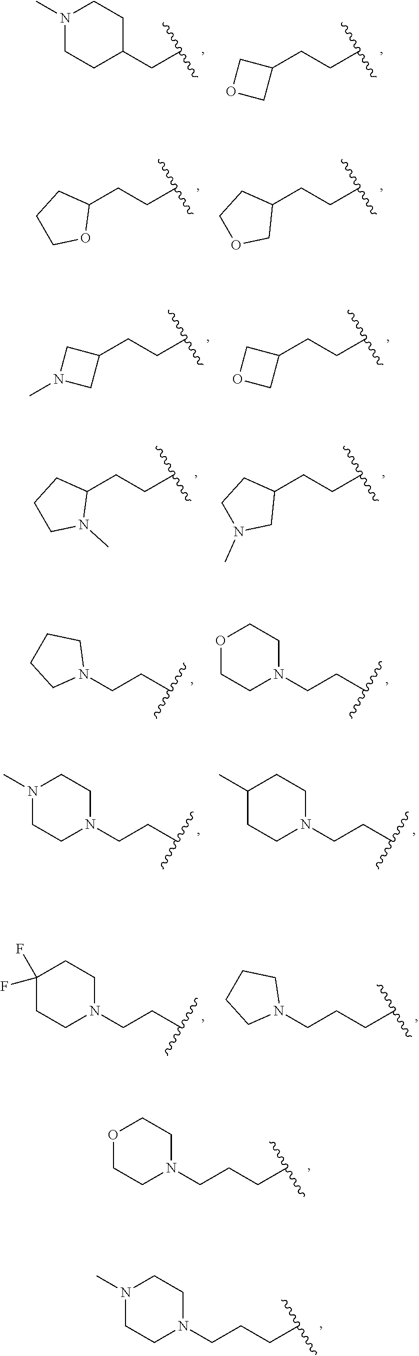

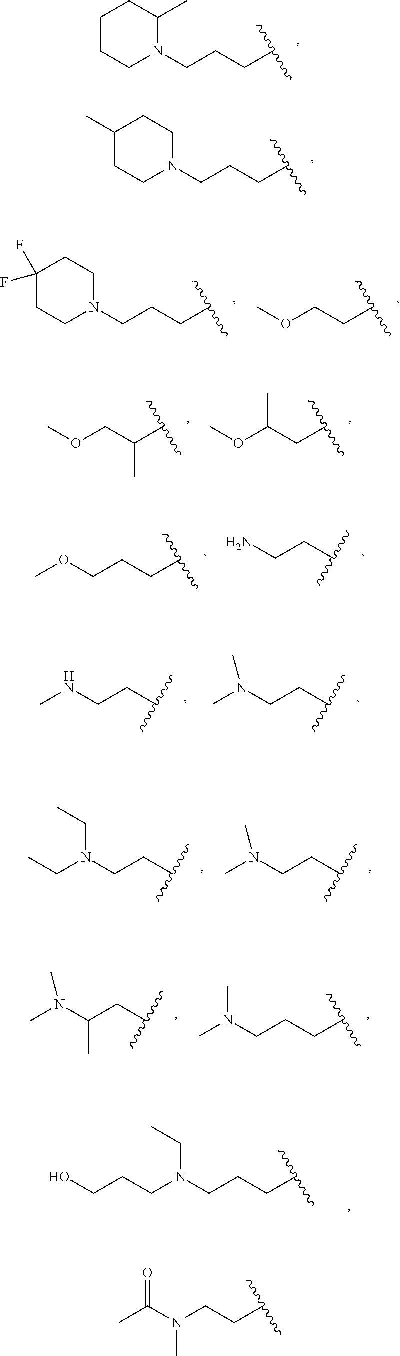

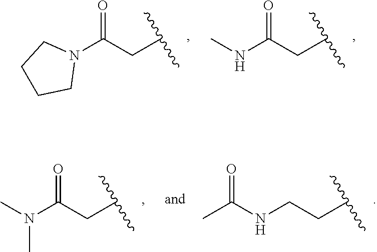

In some embodiments, R⁵ is selected from the group consisting of methyl,

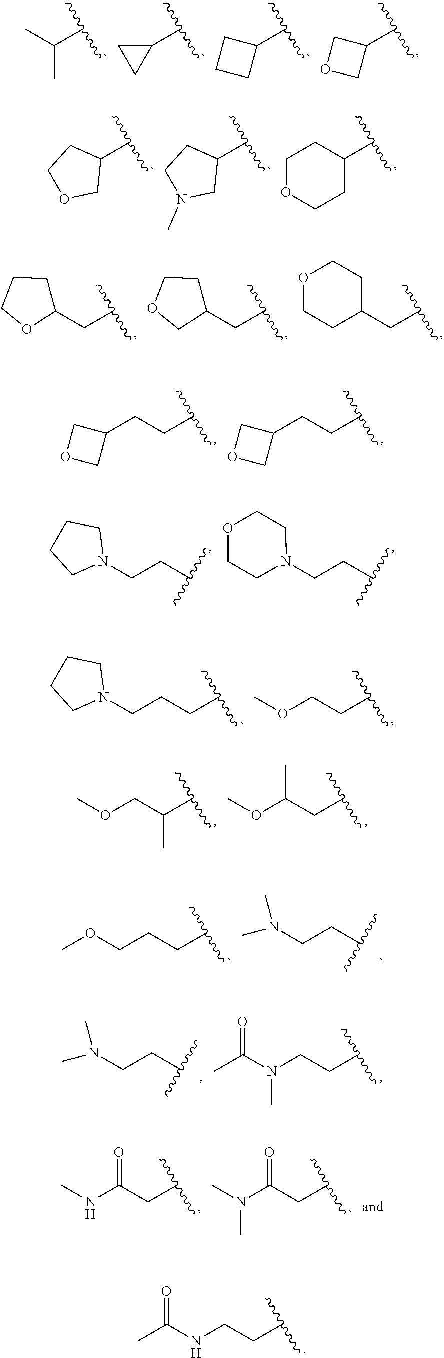

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

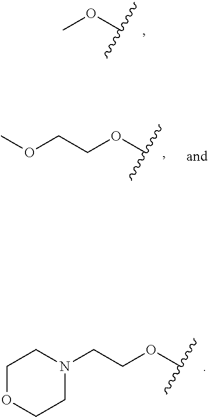

In some embodiments, R2 is H.

In some embodiments, R³ is F or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹, X², X³, and X⁴ are independently CH or N, provided that not more that one of X³ and X⁴ is N.

In some embodiments, X¹ is CH and X² is CH.

In some embodiments, X¹ is CH and X² is N.

In some embodiments, X³ is CH and X⁴ is N.

In some embodiments, X¹ is N, X² is CH, X³ is CH, and X⁴ is CH; X¹ is CH, X² is N, X³ is CH, and X⁴ is CH; X¹ is CH, X² is CH, X³ is N, and X⁴ is CH; X¹ is CH, X² is CH, X³ is CH, and X⁴ is N; X¹ is N, X² is N, X³ is CH, and X⁴ is CH; X¹ is N, X² is N, X³ is N, and X⁴ is CH; or X¹ is N, X² is N, X³ is CH, and X⁴ is N.

In some embodiments, L is selected from the group consisting of direct bond and C₁-C₆ alkyl optionally substituted with (E¹¹)ₘ.

In some embodiments,

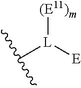

is selected from the group consisting of

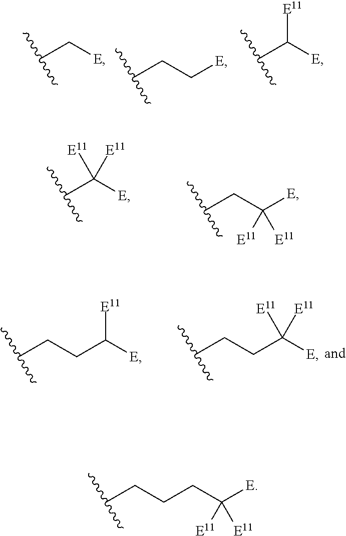

In some embodiments, E¹¹, at each occurrence, is independently, selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

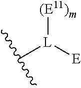

is selected from the group consisting of

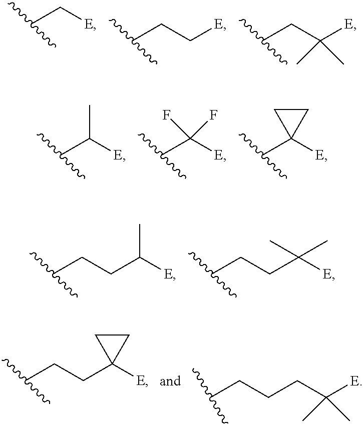

In some embodiments,

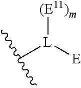

is selected from the group consisting of

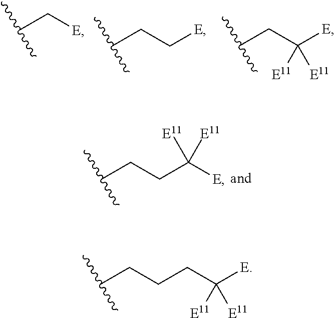

In some embodiments, wherein

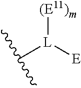

is selected from the group consisting

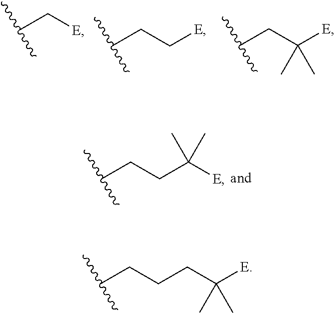

In some embodiments, E is selected from the group consisting of: optionally substituted amido wherein the substituent, at each occurrence, is independently, selected from the group consisting of optionally substituted C₁-C₆ alkyl, optionally substituted C₃-C₆ heterocyclyl, and optionally substituted C₃-C₆ cycloalkyl, wherein the substituent of the optionally substituted C₁-C₆ alkyl, at each occurrence, is independently, selected from the group consisting of C₁-C₆ alkyl, alkoxy, amine, halogen, haloalkyl, haloalkoxy, hydroxy, cyano, cycloalkyl, and heterocyclyl, the substituent of the optionally substituted C₃-C₆ heterocyclyl, at each occurrence, is independently, selected from the group consisting of C₁-C₆ alkyl, alkoxy, amine, halogen, haloalkyl, haloalkoxy, hydroxy, and cyano, the substituent of the optionally substituted cycloalkyl at each occurrence, is independently, selected from the group consisting of C₁-C₆ alkyl, alkoxy, amine, halogen, haloalkyl, haloalkoxy, hydroxy, and cyano, optionally substituted C₁-C₆ alkyl wherein the optionally substituted substituent, at each occurrence, is independently, selected from the group consisting of C₁-C₆ alkyl, alkoxy, amine, halogen, haloalkyl, haloalkoxy, hydroxy, and cyano, optionally substituted C₃-C₆ cycloalkyl wherein the optionally substituted substituent, at each occurrence, is independently, selected from the group consisting of C₁-C₆ alkyl, alkoxy, amine, halogen, haloalkyl, haloalkoxy, hydroxy, and cyano, optionally substituted heterocyclyl wherein the optionally substituted substituent, at each occurrence, is independently, selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, optionally substituted alkoxy wherein the optionally substituted substituent, at each occurrence, is independently, selected from the group consisting of cycloalkyl, heterocyclyl, and H, cyano, alkoxy, haloalkoxy, haloalkyl, and halogen.

In some embodiments, E is selected from the group consisting of H, fluoro, methyl, trifluoromethyl, methoxy, cyano,

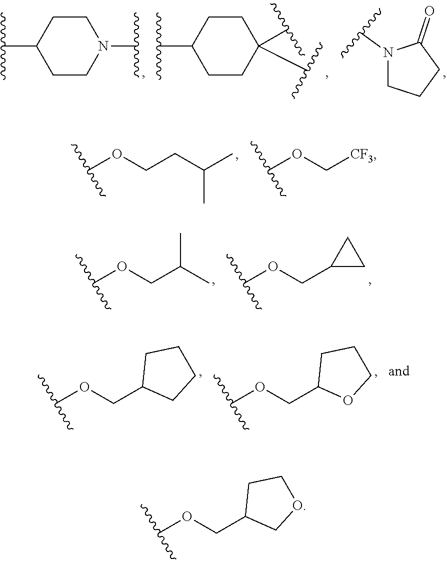

In some embodiments, E is selected from the group consisting of H, fluoro, methyl, trifluoromethyl, methoxy, cyano,

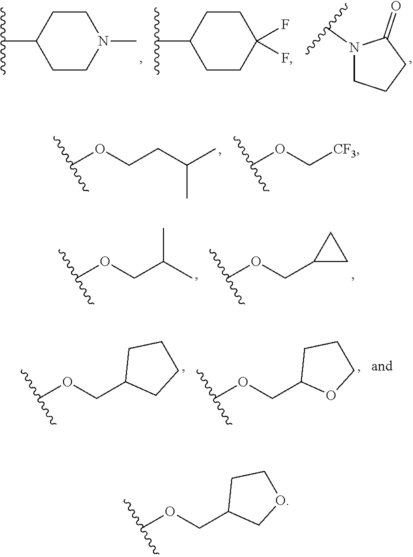

In some embodiments,

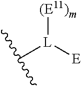

is selected from the group consisting of

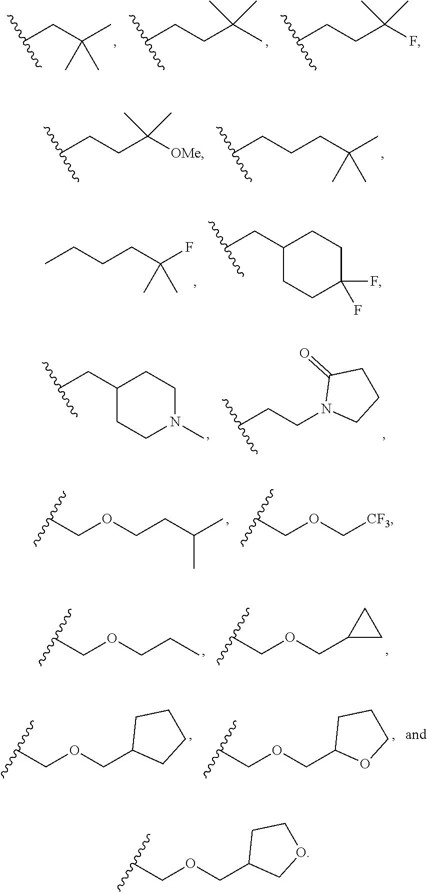

Compounds of Formula I-V, Formula I-W, Formula I-X, and Formula I-Y

In one embodiment, described herein is a compound represented by Formula I-V

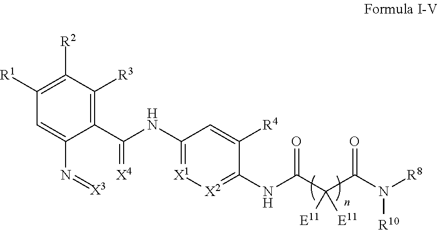

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of H, F and alkoxy;
R⁴ is selected from the group consisting of H, F, alkyl, and alkoxy;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo; and
n is 0, 1, 2, 3, or 4.

In one embodiment, described herein is a compound represented by Formula I-W

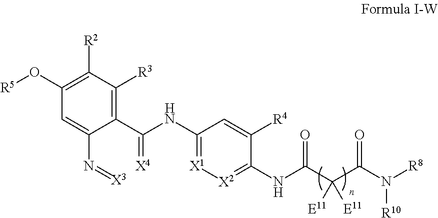

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of H, F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo; and
n is 0, 1, 2, 3, or 4.

In one embodiment, described herein is a compound represented by Formula I-X:

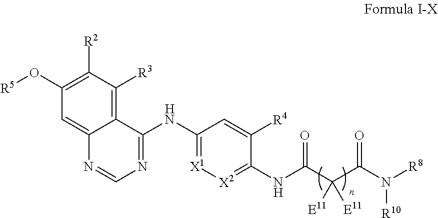

or a pharmaceutically acceptable salt, enantiomer, stereoisomer, or tautomer thereof, wherein:
X¹ and X² are each independently selected from the group consisting of N, CH, and CF;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of H, F, and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen;
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent is, at each occurrence, is independently selected from the group consisting of alkyl, halogen, haloalkyl, hydroxyl, alkoxy, haloalkoxy, and oxo; and
n is 0, 1, 2, 3, or 4.

In one embodiment, described herein is a compound represented by Formula I-Y:

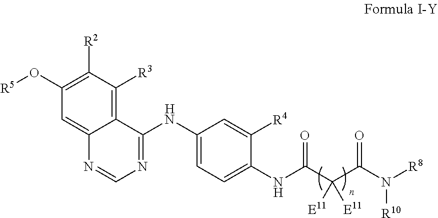

or a pharmaceutically acceptable salt, enantiomer, stereoisomer, or tautomer thereof, wherein:
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of H, F, and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen;
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁸ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent is, at each occurrence, is independently selected from the group consisting of alkyl, halogen, haloalkyl, hydroxyl, alkoxy, haloalkoxy, and oxo; and
n is 0, 1, 2, 3, or 4.

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

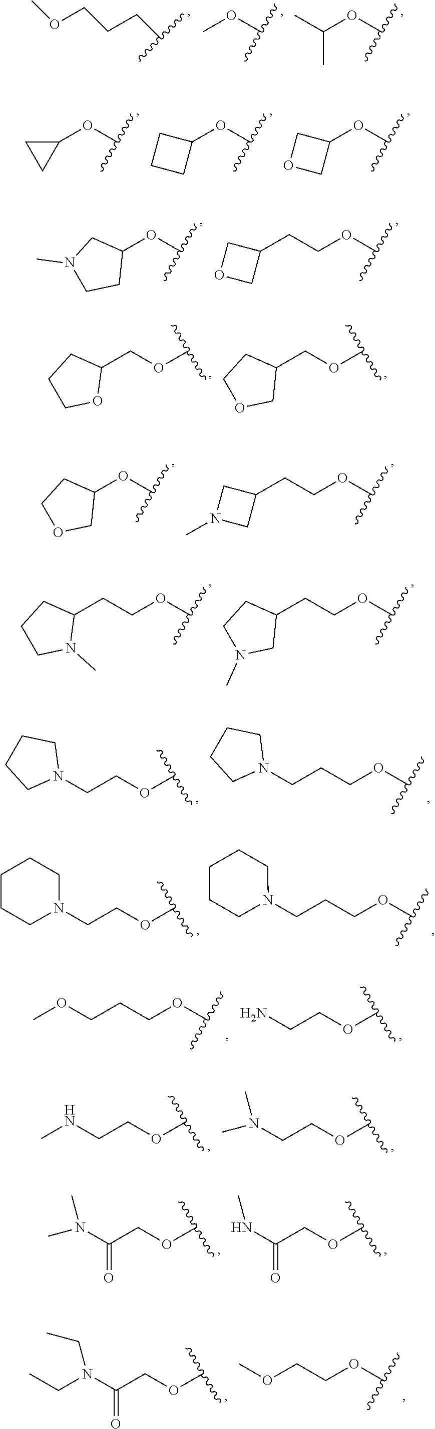

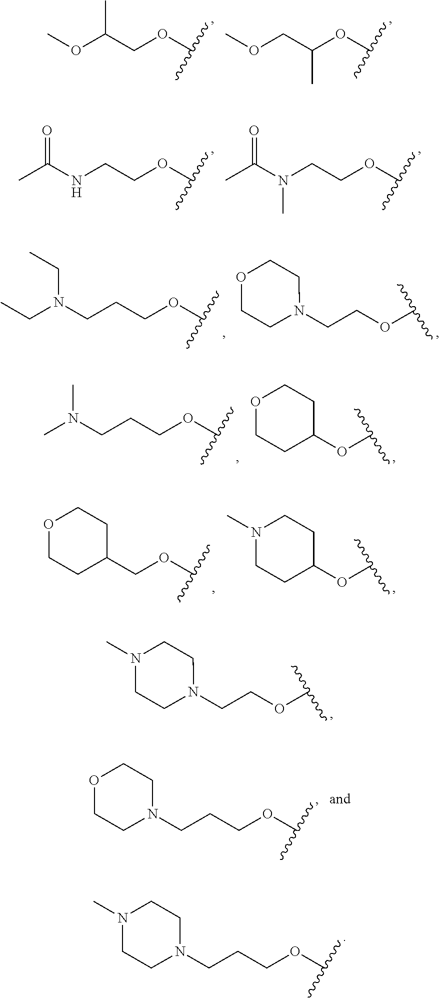

In some embodiments, R¹ is selected from the group consisting of H,

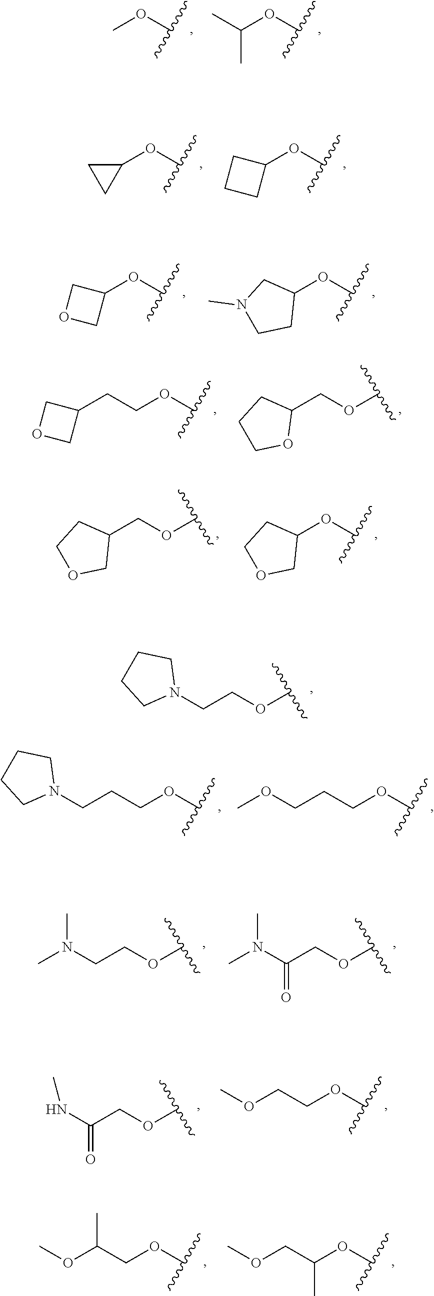

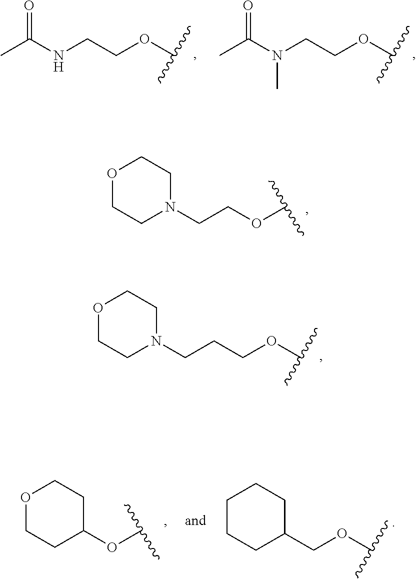

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃

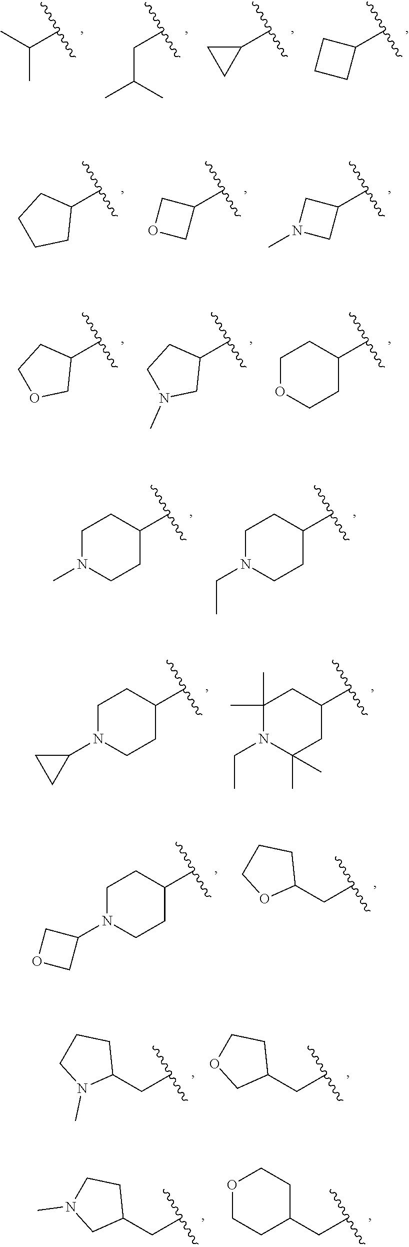

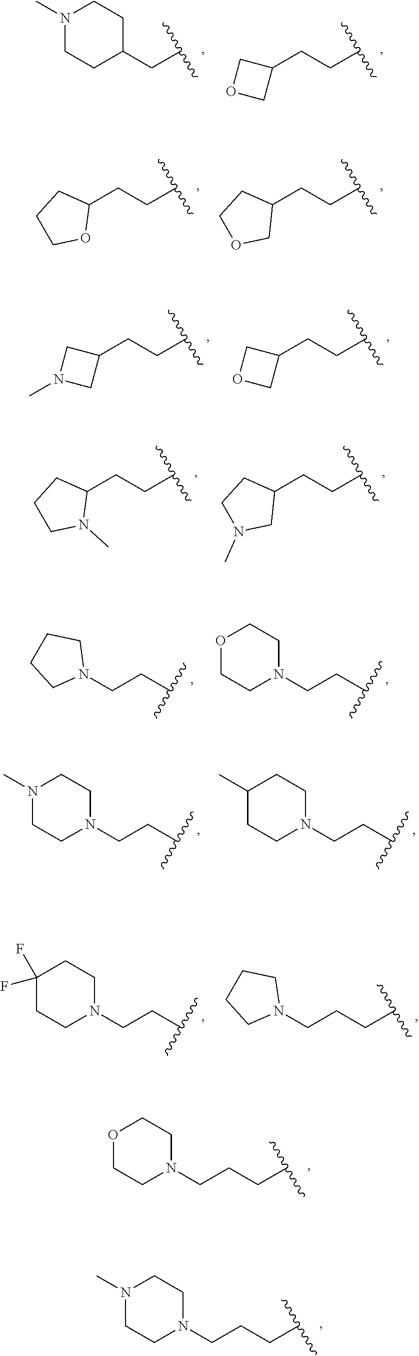

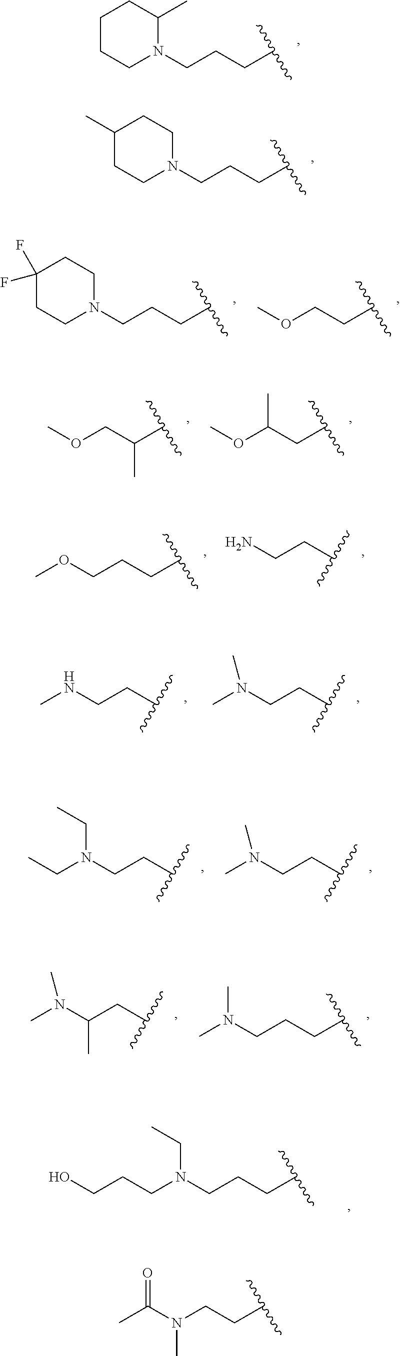

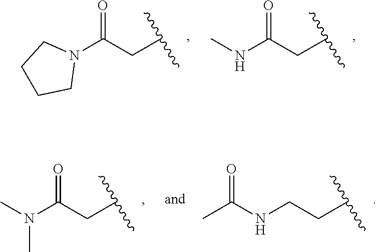

In some embodiments, R⁵ is selected from the group consisting of methyl,

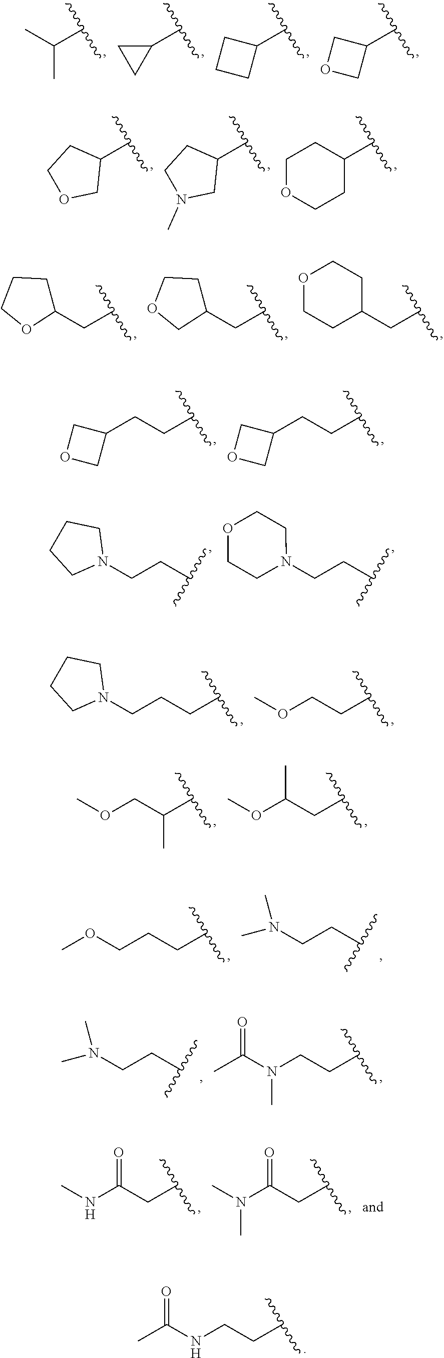

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

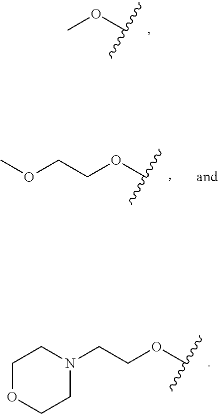

In some embodiments, R² is H.

In some embodiments, R³ is F, H, or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹ and X² are independently CH or N.

In some embodiments, X¹ is CH and X² is CH.

In some embodiments, X¹ is CH and X² is N.

In some embodiments, L is selected from the group consisting of direct bond and C₁-C₆ alkyl optionally substituted with (E¹¹)ₘ.

In some embodiments,

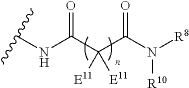

is selected from the group

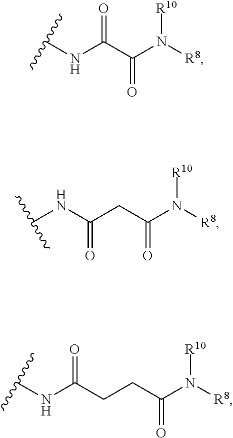

consisting of

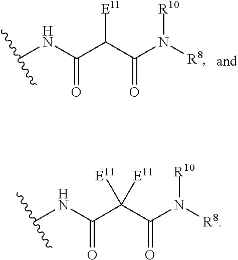

In some embodiments,

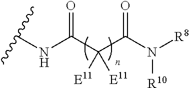

is selected from the group consisting of

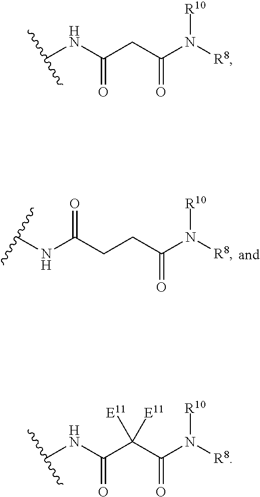

In some embodiments, E¹¹, at each occurrence, is independently, selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

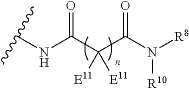

is selected from the group consisting of

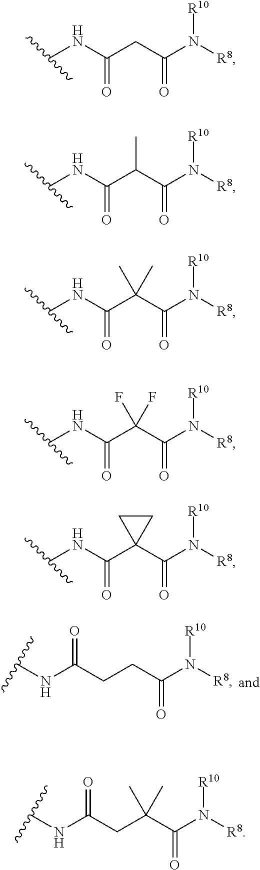

In some embodiments,

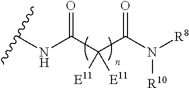

is selected from the group consisting of

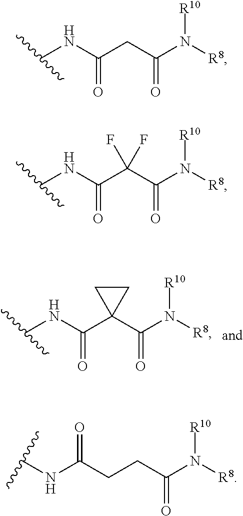

In some embodiments, R⁸ is selected from the group consisting of alkyl, cycloalkyl, and heterocyclyl.

In some embodiments, R⁸ is selected from the group consisting of C₁-C₆ alkyl, cycloalkyl, and heterocyclyl.

In some embodiments, R¹⁰ is selected from the group consisting of H and alkyl.

In some embodiments, R¹⁰ is selected from the group consisting of H and C₁-C₆ alkyl.

In some embodiments, R⁸ is selected from the group consisting of alkyl, cycloalkyl, and heterocyclyl, and R¹⁰ is selected from the group consisting of H and alkyl.

In some embodiments, R⁸ is selected from the group consisting of

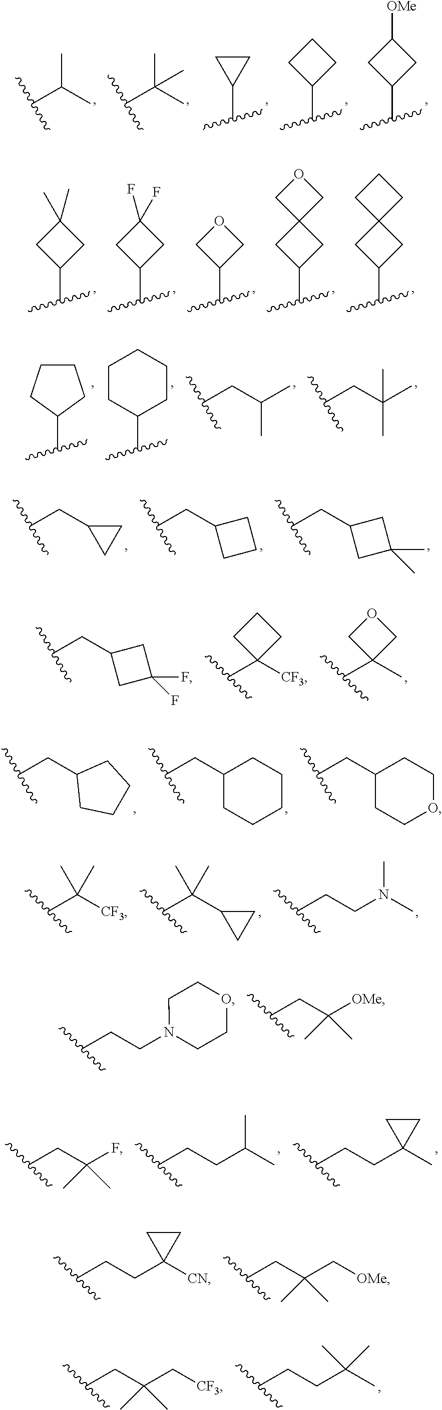

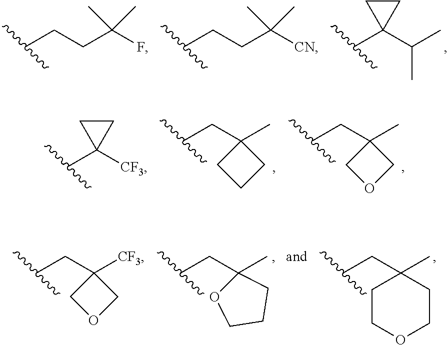

and R¹⁰ is selected from the group consisting of H, methyl, ethyl, and isopropyl.

In some embodiments, R⁸ taken together with R¹⁰ and the N atom to which R⁸ and R¹⁰ are attached form a ring structure selected from the group consisting of

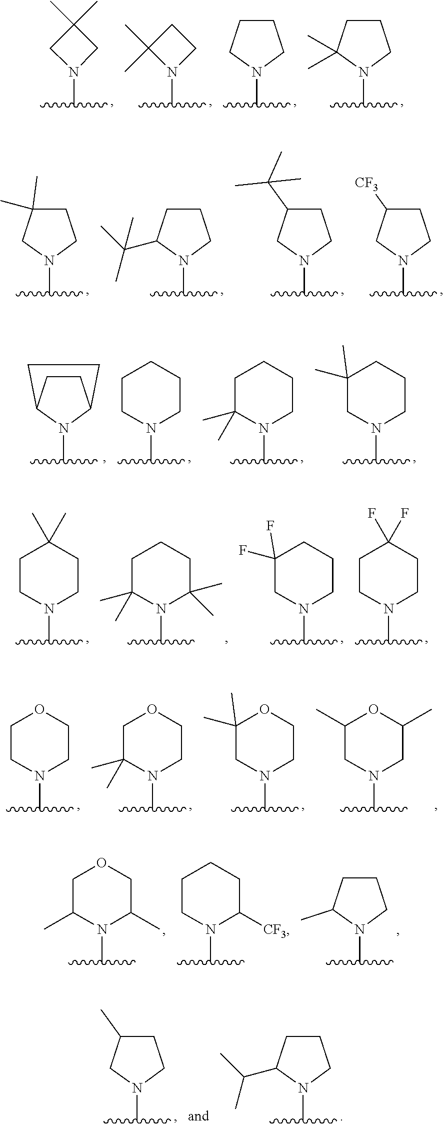

In some embodiments, L is a direct bond, R⁸ is selected from the group consisting of

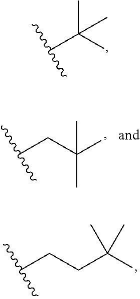

### and R¹⁰ is H.

Compounds of Formula I-Z, Formula I-AA, Formula I-AB, and Formula I-AC

In one embodiment, described herein is a compound represented by Formula I-Z

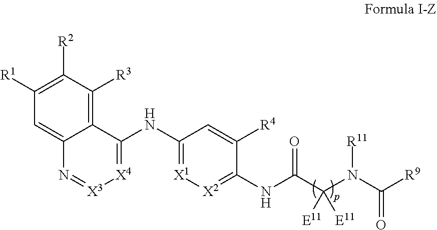

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
   the substituent of the optionally substituted amido, at each occurrence, is independently, C₁-C₆ alkyl, and
   the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of H, F and alkoxy;
R4 is selected from the group consisting of H, F, alkyl, and alkoxy;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy; and
p is 1, 2, or 3,
provided that the compound is not:

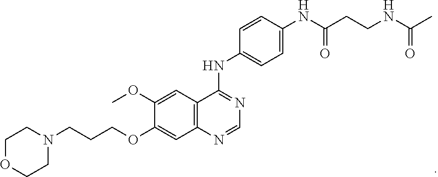 [SMILES: COc1cc2c(Nc3ccc(NC(=O)CCNC(C)=O)cc3)ncnc2cc1OCCCN1CCOCC1]

In one embodiment, described herein is a compound represented by Formula I-AA

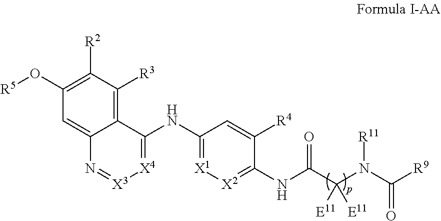

or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently, alkoxy, amine, heterocyclyl, or when taken together with R⁵ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of H, F and alkoxy;
R⁴ is selected from the group consisting of H, F, alkyl, and alkoxy;
R⁵ is selected from the group consisting of optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy; and
p is 1, 2, or 3,
provided that the compound is not:

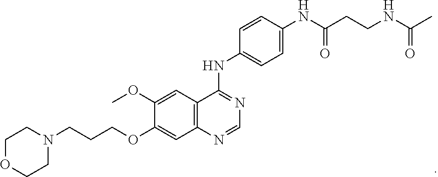 [SMILES: COc1cc2c(Nc3ccc(NC(=O)CCNC(C)=O)cc3)ncnc2cc1OCCCN1CCOCC1]

In one embodiment, described herein is a compound represented by Formula I-AB:

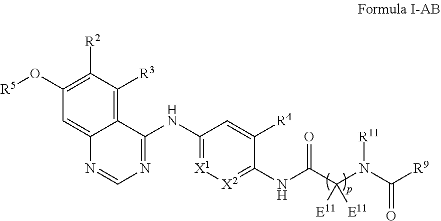

or a pharmaceutically acceptable salt, enantiomer, stereoisomer, or tautomer thereof, wherein:
X¹ and X² are each independently selected from the group consisting of N, CH, and CF;
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of H, F, and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy; and
p is 1, 2, or 3,
provided that the compound is not

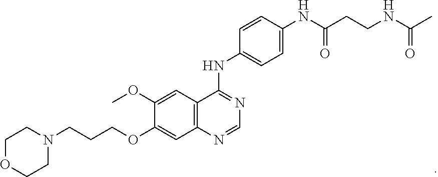 [SMILES: COc1cc2c(Nc3ccc(NC(=O)CCNC(C)=O)cc3)ncnc2cc1OCCCN1CCOCC1]

In one embodiment, described herein is a compound represented by Formula I-AC:

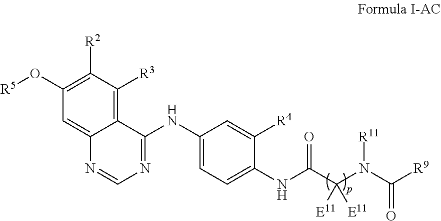

or a pharmaceutically acceptable salt, enantiomer, stereoisomer, or tautomer thereof, wherein:
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆haloalkyl, C₁-C₆ alkoxy, C₁-C₆ haloalkoxy, hydroxy, and oxo;
R² is selected from the group consisting of H and optionally substituted alkoxy wherein the optionally substituted substituent, at each occurrence, is independently alkoxy, amine, heterocyclyl, or when taken together with R⁵ form an optionally substituted heterocyclyl having from 5 to 6 atoms in the ring structure wherein the optionally substituted substituent, at each occurrence, is independently selecting from the group consisting of C₁-C₆ alkyl and halogen;
R³ is selected from the group consisting of H, F, and alkoxy;
R⁴ is selected from the group consisting of H, F, alkyl, and alkoxy;
R⁵ is selected from the group consisting of
optionally substituted alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, wherein
   the substituent of the optionally substituted amido, at each occurrence, is independently C₁-C₆ alkyl, and
   the substituent of the optionally substituted heterocyclyl, at each occurrence, is independently selected from the group consisting of C₁-C₆ alkyl, alkoxy, and halogen,
optionally substituted heterocyclyl wherein the optionally substituted substituent, at each occurrence, is independently selected from the group consisting of alkyl, alkoxy, and halogen, and
optionally substituted cycloalkyl wherein the optionally substituted substituent, at each occurrence, is independently selected from the group consisting of alkyl, alkoxy, and halogen;
R⁹ is selected from the group consisting of optionally substituted alkyl, optionally substituted heterocyclyl, and optionally substituted cycloalkyl, wherein
   the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
   the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
   the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy; and
p is 1, 2, or 3,
provided that the compound is not:

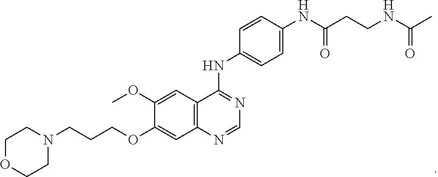 [SMILES: COc1cc2c(Nc3ccc(NC(=O)CCNC(C)=O)cc3)ncnc2cc1OCCCN1CCOCC1]

In some embodiments, R¹ is selected from the group consisting of H, haloalkyl, optionally substituted C₁-C₆ alkoxy wherein the optionally substituted substituent, at each occurrence, is independently, selected from the group consisting of alkoxy, acetamido, amine, haloalkyl, haloalkoxy, halogen, hydroxy, cyano, carboxylic acid, optionally substituted amido, and optionally substituted heterocyclyl, the substituent of the optionally substituted amido, at each occurrence, is independently, C₁-C₆ alkyl, the substituent of the optionally substituted heterocyclyl, at each occurrence, is independently, C₁-C₆ alkyl, amine, cyano, halogen, C₁-C₆ haloalkyl, C₁-C₆ alkoxy, C₁-C₆ haloalkoxy, hydroxy, and oxo, optionally substituted C₄-C₆ cycloalkyloxy wherein the optionally substituted substituent, at each occurrence, is independently, selected from the group consisting of independently, C₁-C₆ alkyl, amine, halogen, C₁-C₆ haloalkyl, C₁-C₆ alkoxy, C₁-C₆ haloalkoxy, hydroxy, and oxo, and optionally substituted C₁-C₆ alkyl wherein the optionally substituted substituent, at each occurrence, is independently, selected from the group consisting of alkoxy, amine, haloalkoxy, and hydroxy.

In some embodiments, R¹ is selected from the group consisting of H,

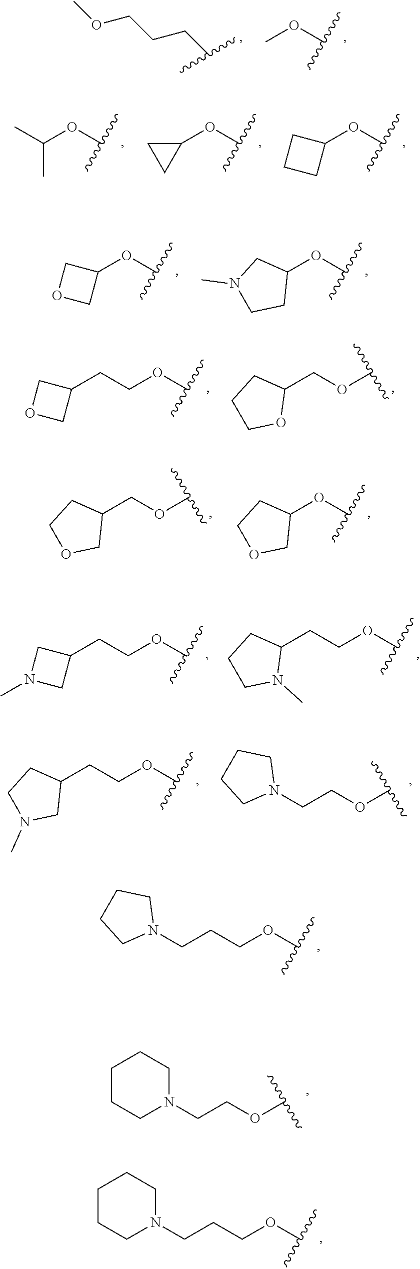

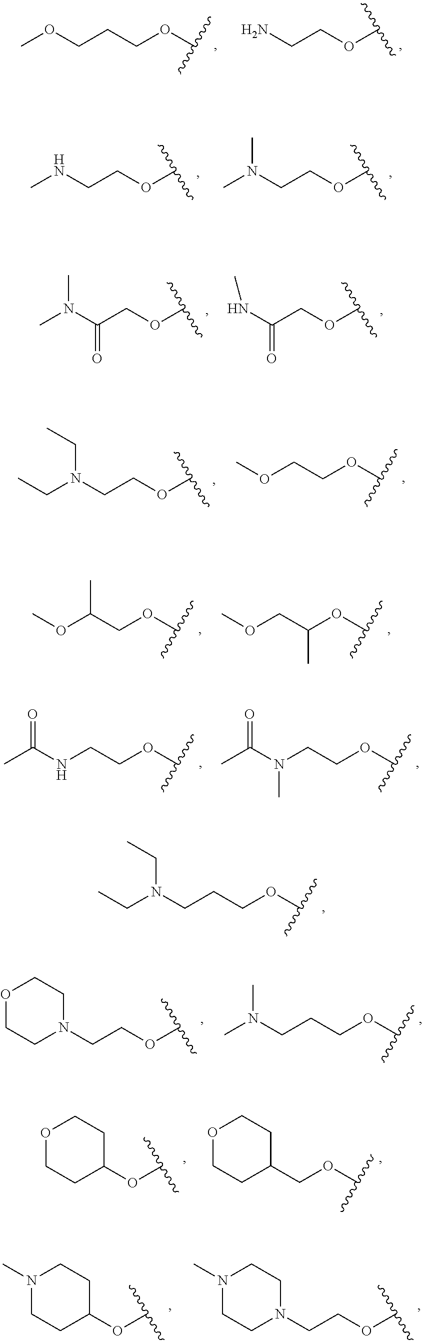

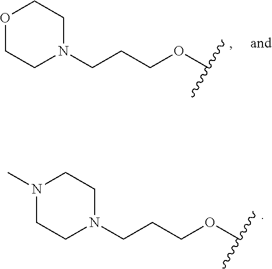 [SMILES: *OCCCN1CCOCC1 ; *OCCCN1CCN(C)CC1]

In some embodiments, R¹ is selected from the group consisting of H,

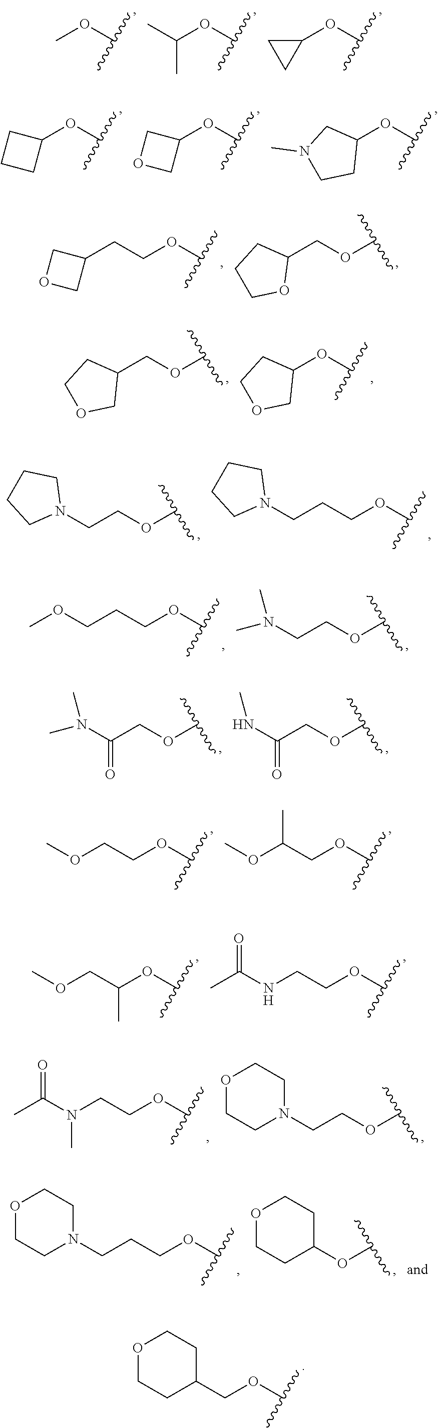

In some embodiments, R⁵ is selected from the group consisting of optionally substituted C₁-C₆ alkyl wherein the optionally substituted substituent, at each occurrence, is independently selected from the group consisting of alkoxy, acetamido, amine, haloalkyl, haloalkoxy, halogen, hydroxy, cyano, cycloalkyl, carboxylic acid, optionally substituted amido, and optionally substituted heterocyclyl, the substituent of the optionally substituted amido, at each occurrence, is independently C₁-C₆ alkyl, the substituent of the optionally substituted heterocyclyl, at each occurrence, is independently selected from the group consisting of C₁-C₆ alkyl, alkoxy, and halogen, optionally substituted C₁-C₆ heterocyclyl wherein the optionally substituted substituent, at each occurrence, is independently selected from the group consisting of alkyl, alkoxy, and halogen; optionally substituted C₁-C₆ cycloalkyl wherein the optionally substituted substituent, at each occurrence, is independently selected from the group consisting of alkyl, alkoxy, and halogen.

In some embodiments, R⁵ is selected from the group consisting of methyl, ethyl, CF₃, CHF₂, CH₂CF₃,

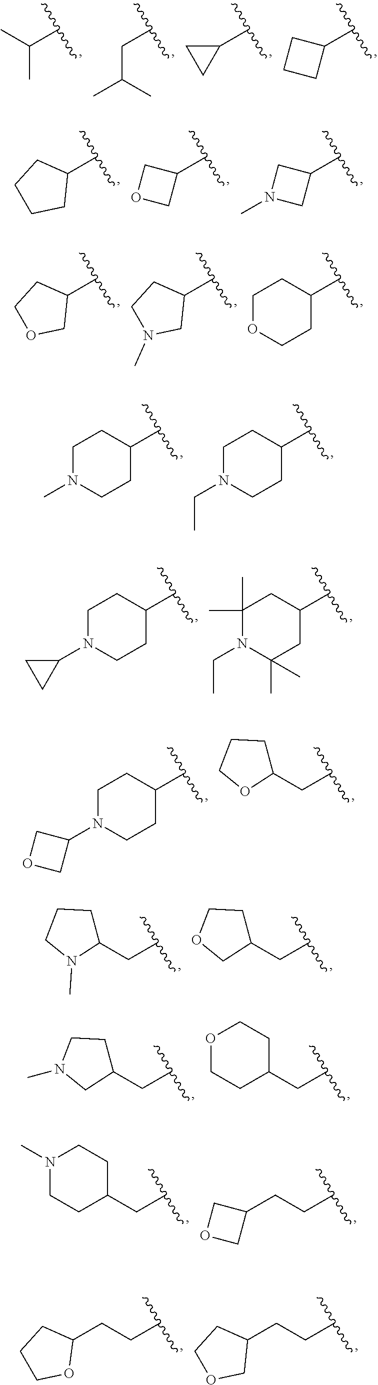

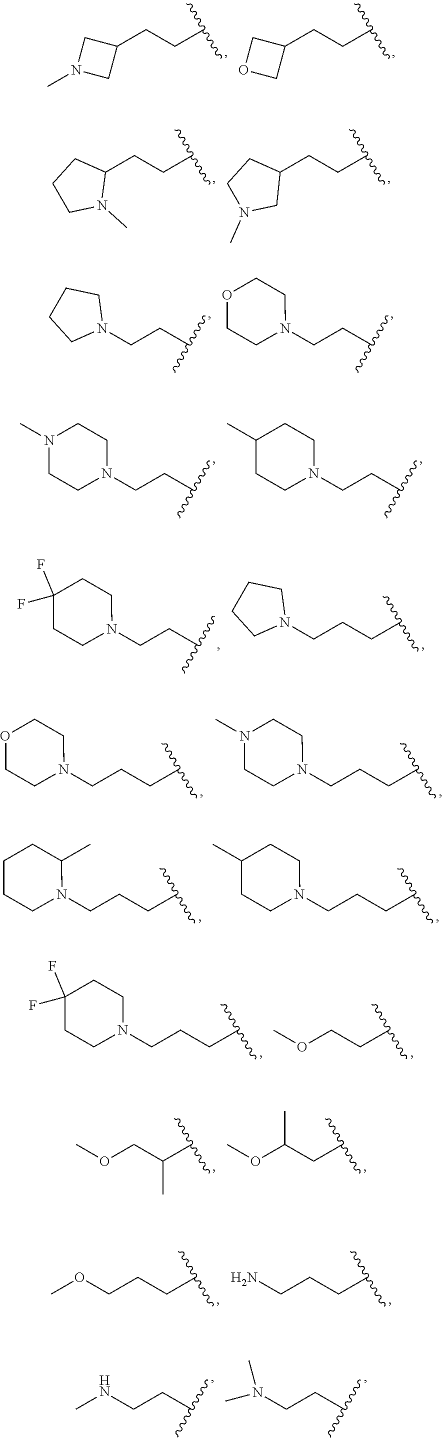

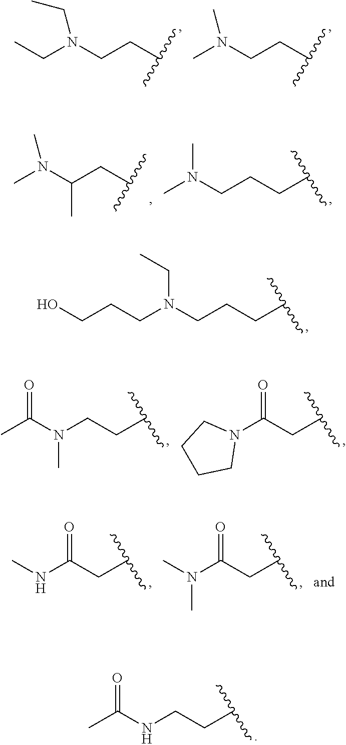

In some embodiments, R⁵ is selected from the group consisting of methyl,

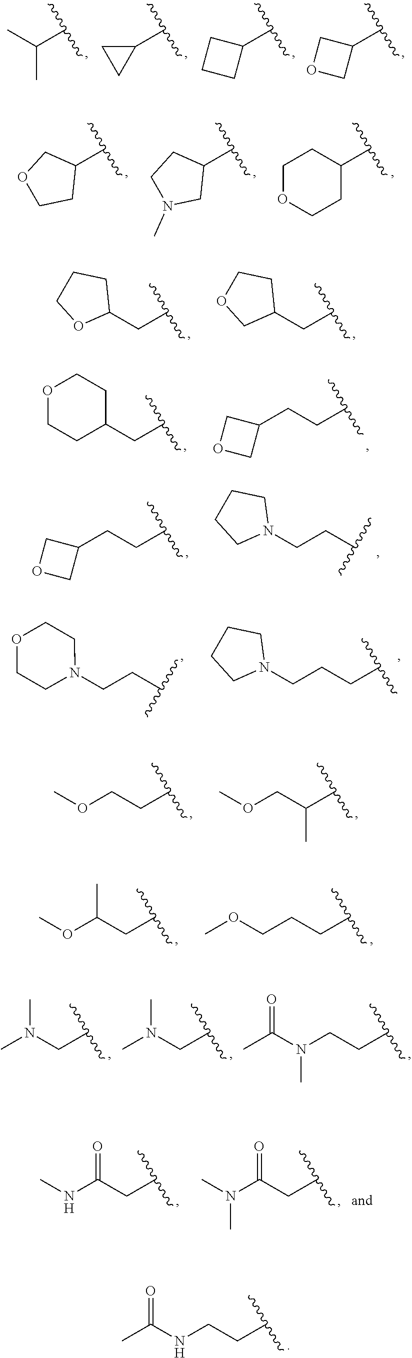

In some embodiments, R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

In some embodiments, R² is selected from the group consisting of H,

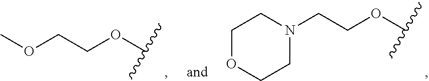

In some embodiments, R² is H.

In some embodiments, R¹ and R² taken together form a ring structure selected from the group consisting of

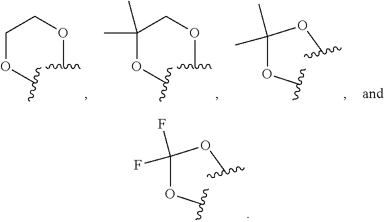

In some embodiments, R² and R⁵ taken together form a ring structure selected from the group consisting of consisting of

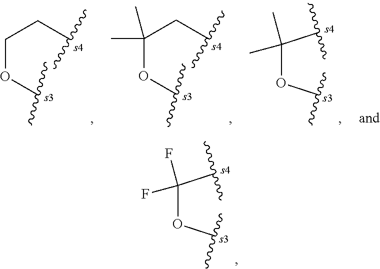

wherein s3 is the site covalently linked to the bicyclic ring structure and s4 is the site covalently linked to the oxygen atom.

In some embodiments, R³ is H, F, or alkoxy.

In some embodiments, R³ is F.

In some embodiments, R⁴ is selected from the group consisting of H, F, C₁-C₆ alkyl, and alkoxy.

In some embodiments, R⁴ is H.

In some embodiments, X¹ and X² are independently CH or N.

In some embodiments, X¹ is CH and X² is CH.

In some embodiments, X¹ is CH and X² is N.

In some embodiments, L is selected from the group consisting of direct bond and C₁-C₆ alkyl optionally substituted with (E¹¹)ₘ.

In some embodiments,

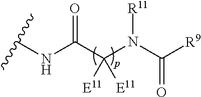

is selected from the group consisting

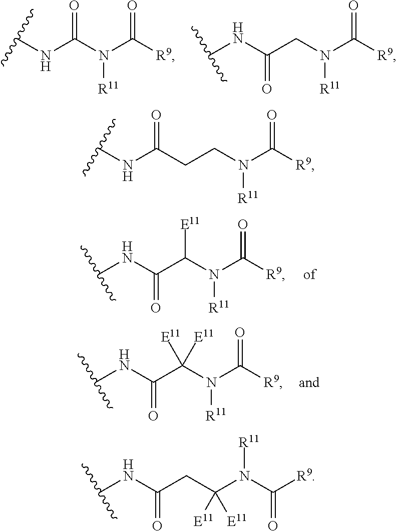

In some embodiments, E¹¹, at each occurrence, is independently, selected from the group consisting of H, C₁-C₆ alkyl, C₃-C₅ cycloalkyl, and halogen.

In some embodiments, E¹¹, at each occurrence, is independently selected from the group consisting of H, methyl, and fluoro.

In some embodiments,

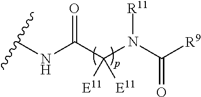

is selected from the group consisting

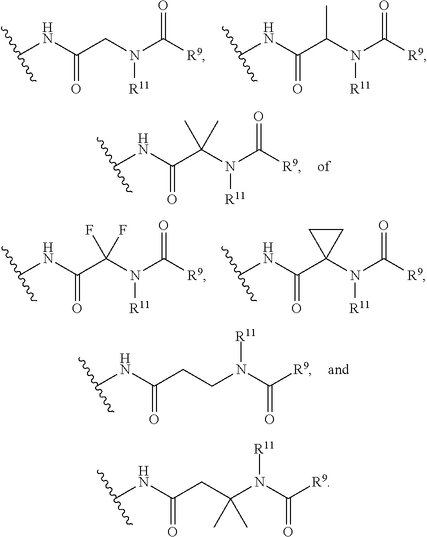

In some embodiments, R⁹ is C₁-C₆ alkyl.

In some embodiments, R⁹ is alkyl.

In some embodiments, R⁹ is selected from the group consisting of

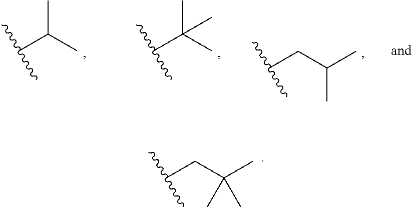

In some embodiments, R¹¹ is selected from the group consisting of H and alkyl.

In some embodiments, R¹¹ is selected from the group consisting of H and C₁-C₆ alkyl.

In some embodiments, R⁹ is C₁-C₆ alkyl and R¹¹ is selected from the group consisting of H and C₁-C₆ alkyl.

In some embodiments, R⁹ is selected from the group consisting of

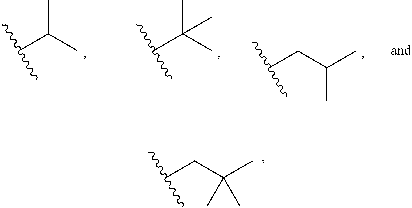

and R¹¹ is selected from the group consisting of H and methyl.

In some embodiments, R¹¹ taken together with R⁹ and the atoms to which R⁹ and R¹¹ are respectively attached form an optionally substituted ring structure having from 4 to 7 atoms in the ring structure wherein the optional substituent, at each occurrence, is independently selected from the group consisting of alkyl, halogen, haloalkyl, hydroxyl, alkoxy, and haloalkoxy.

In some embodiments, R¹¹ taken together with R9 and the atoms to which R9 and R¹¹ are respectively attached form an optionally substituted ring structure selected from the group

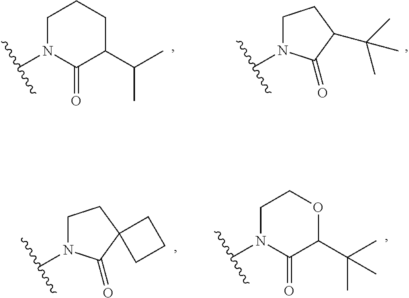 [SMILES: *N1CCCC(C(C)C)C1=O ; *N1CCC(C(C)(C)C)C1=O ; *N1CCC2(CCC2)C1=O ; *N1CCOC(C(C)(C)C)C1=O]

consisting of

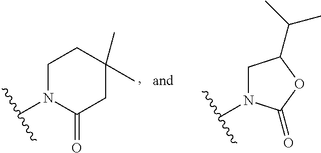

In one embodiment, described herein is a compound selected from:

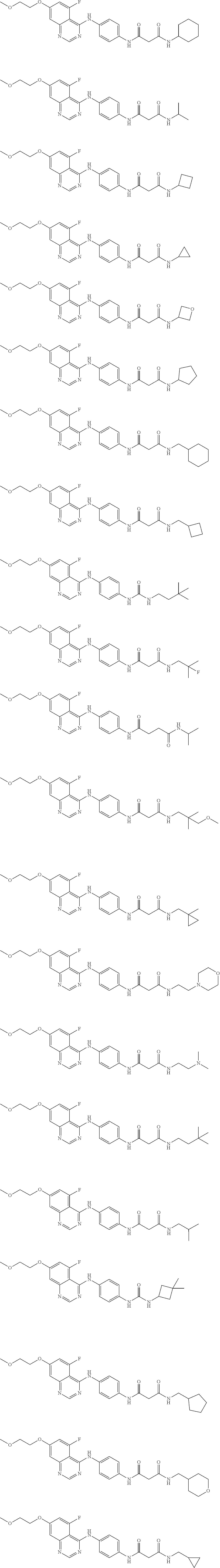

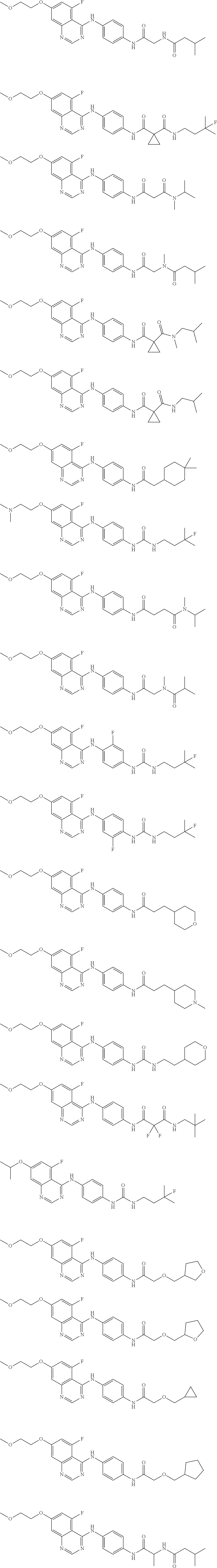

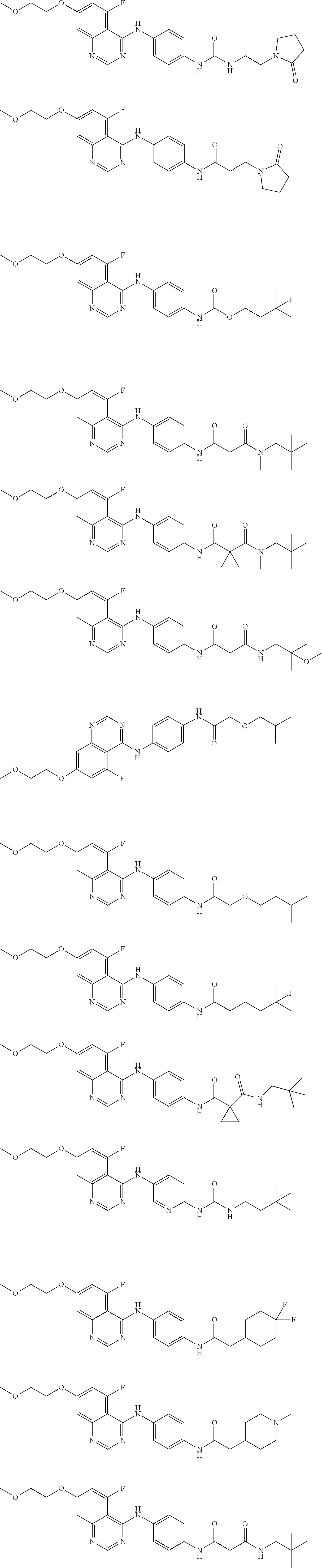

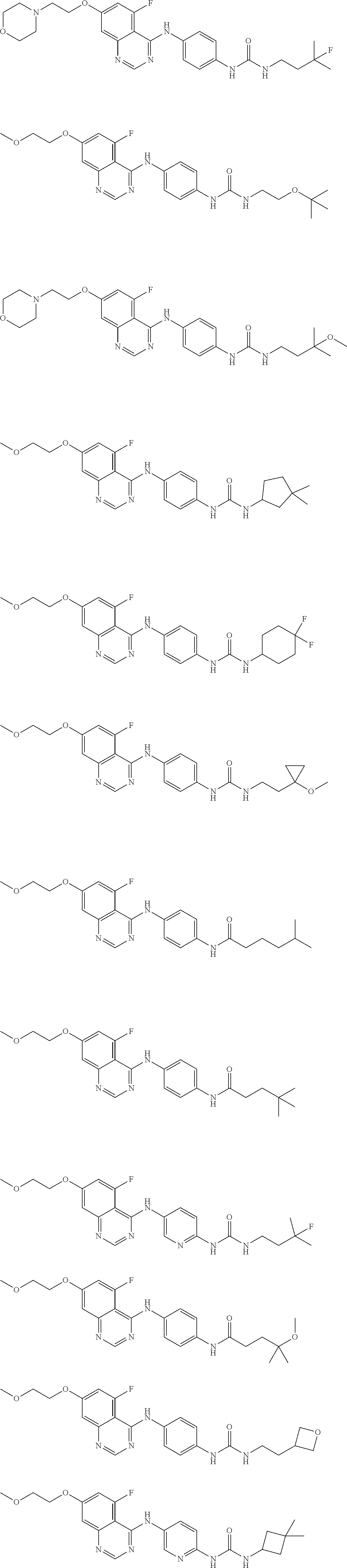

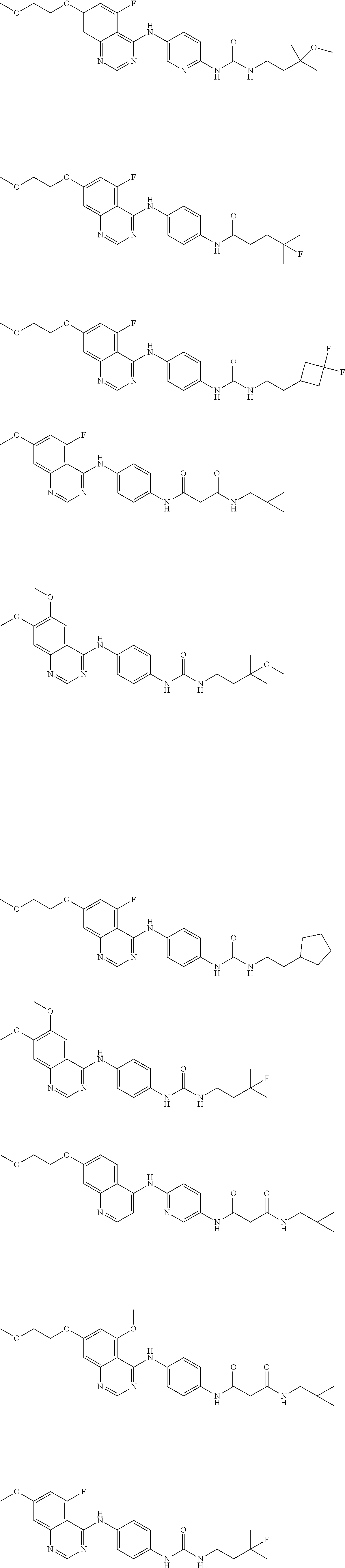

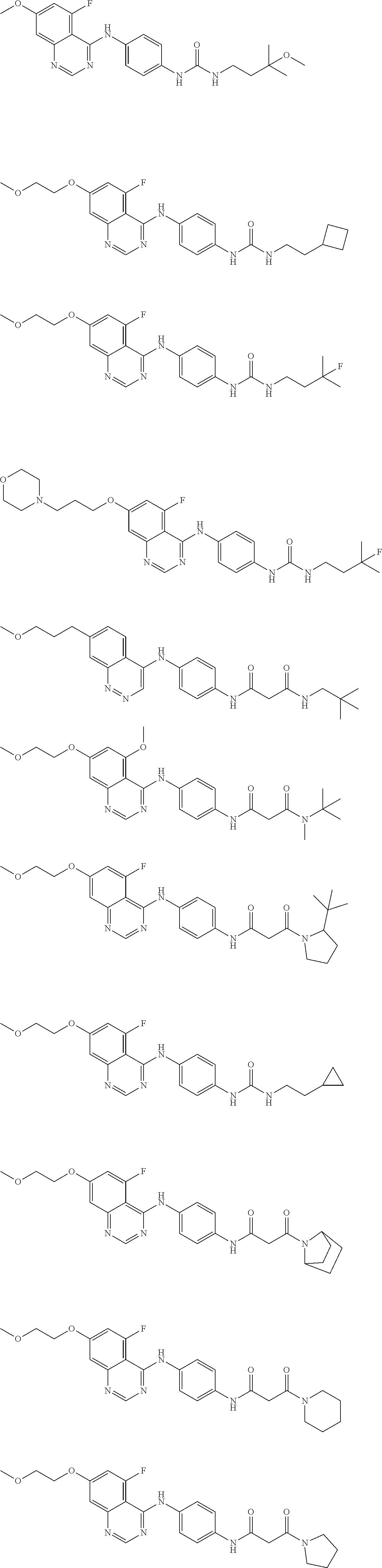

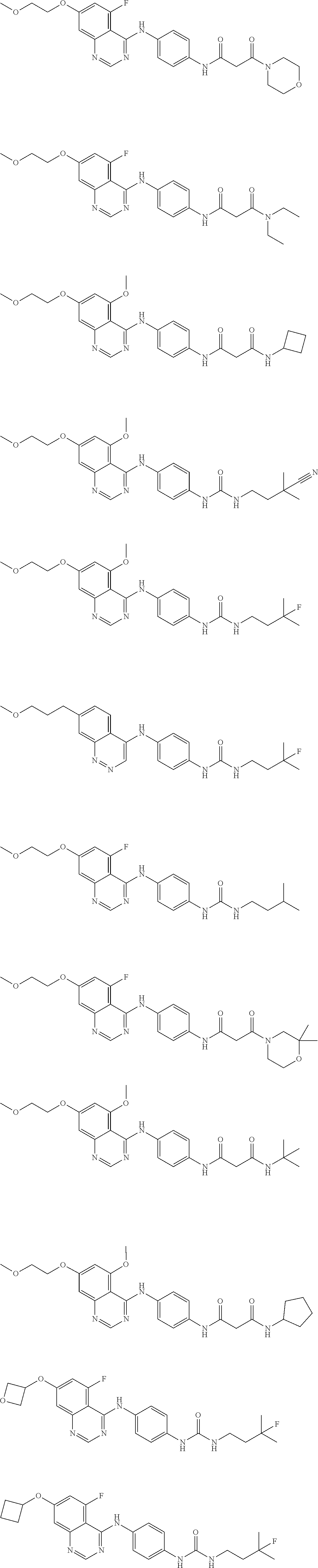

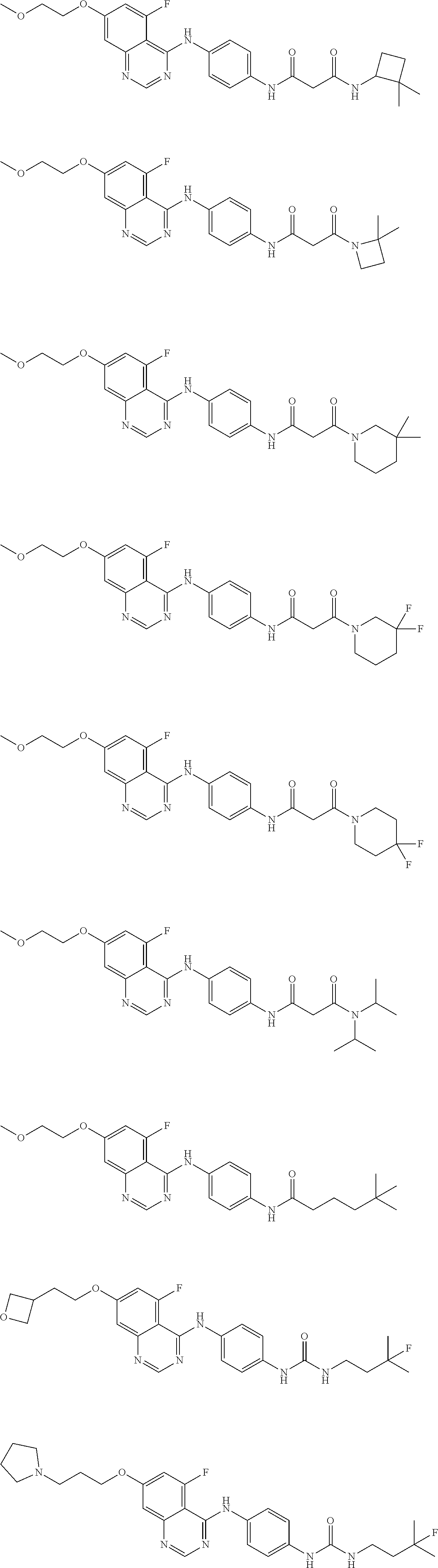

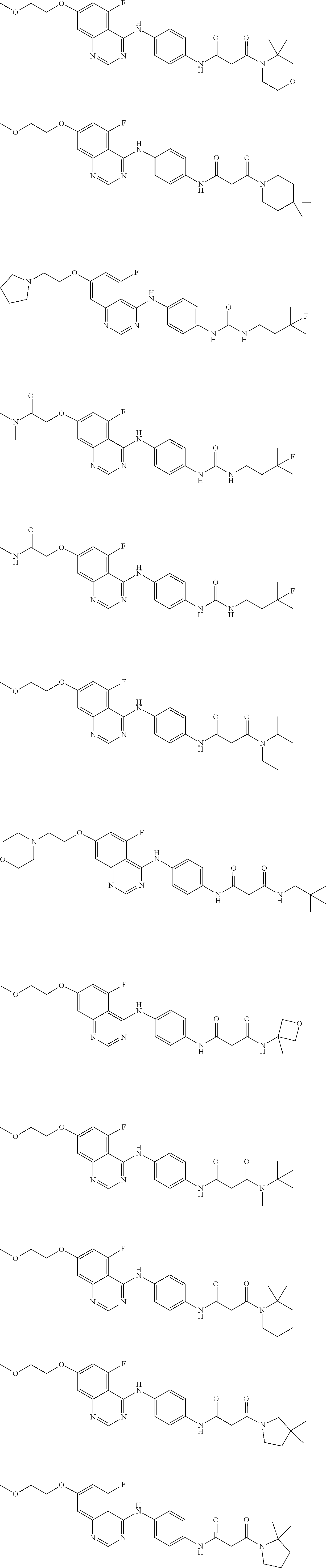

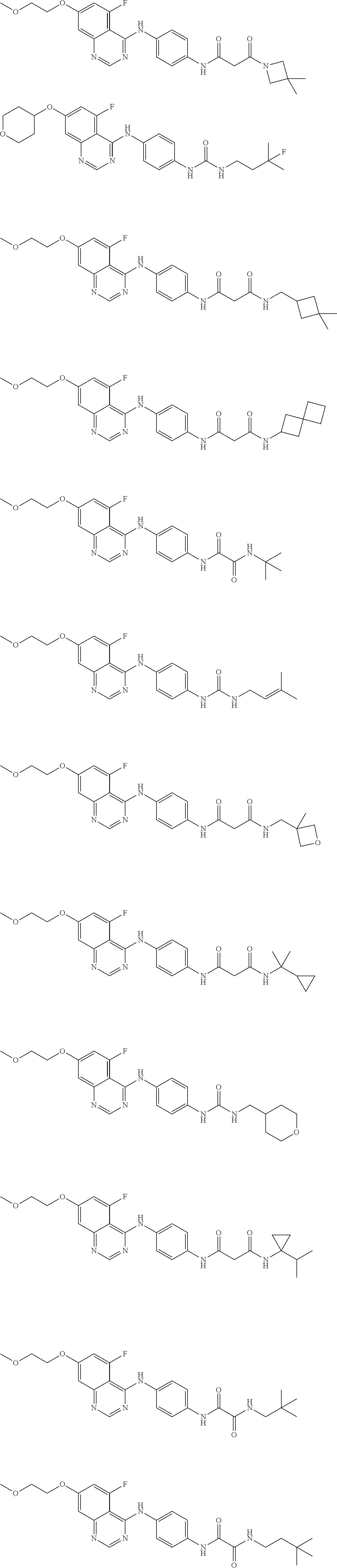

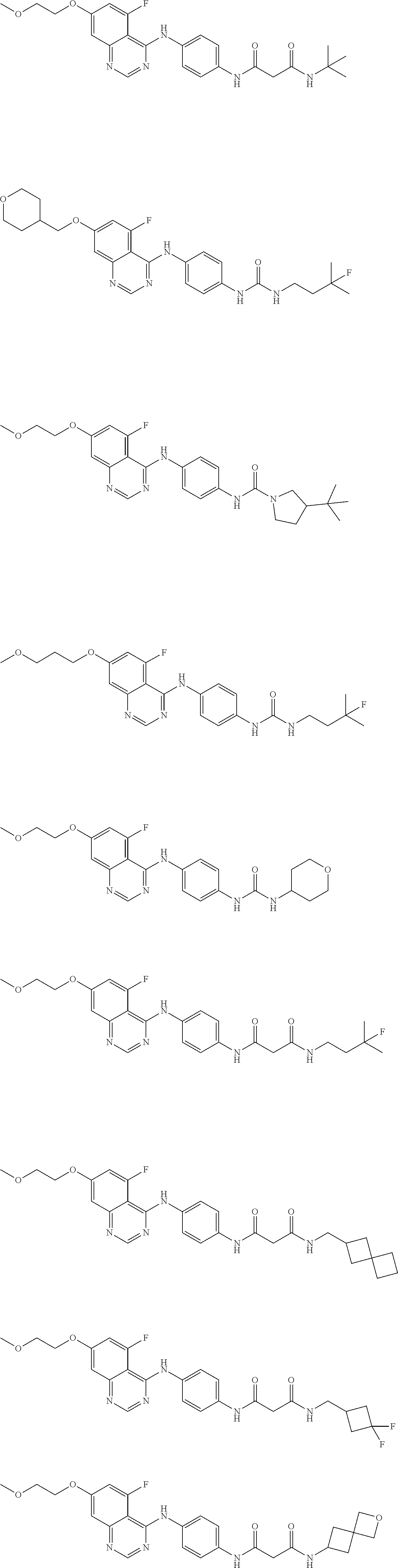

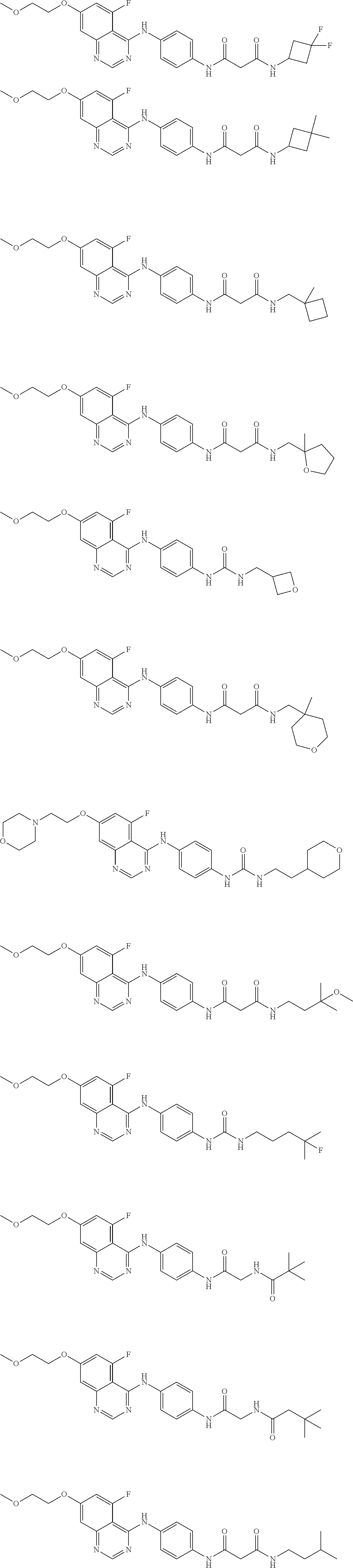

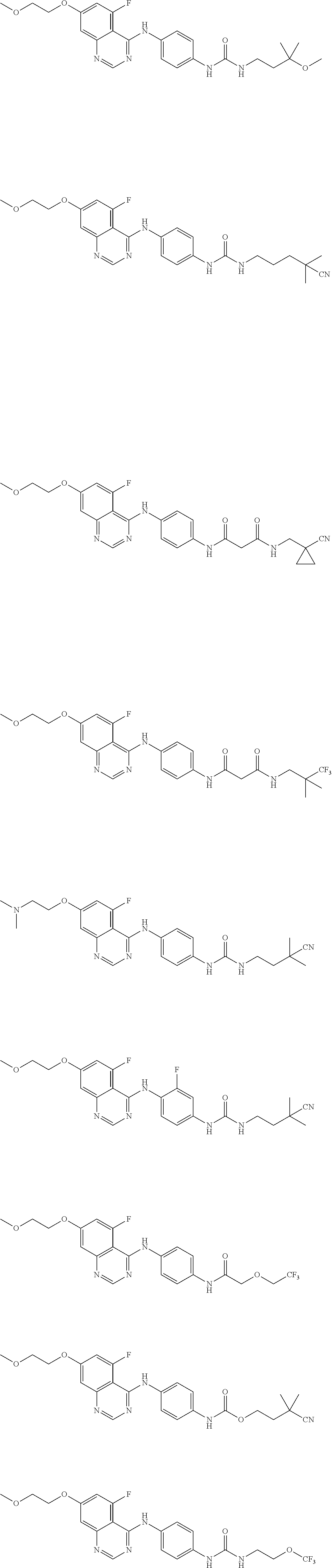

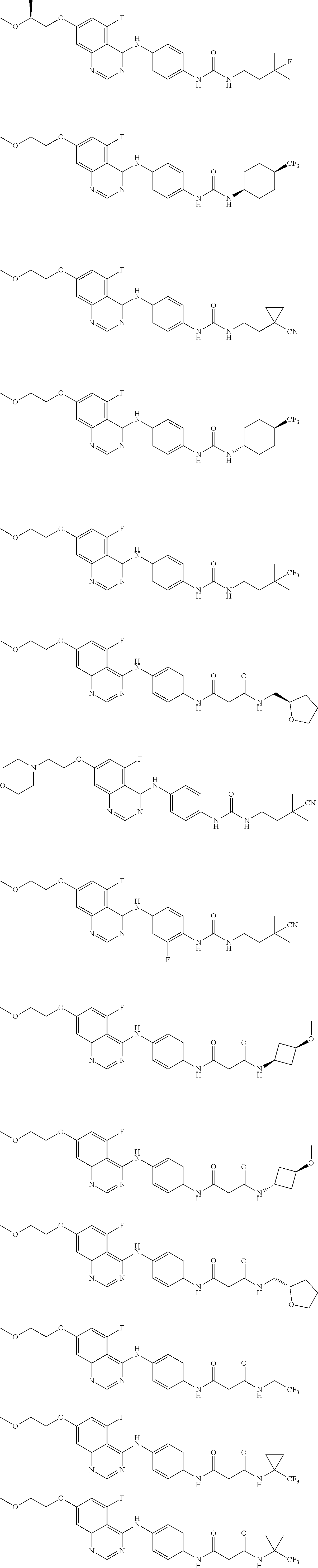

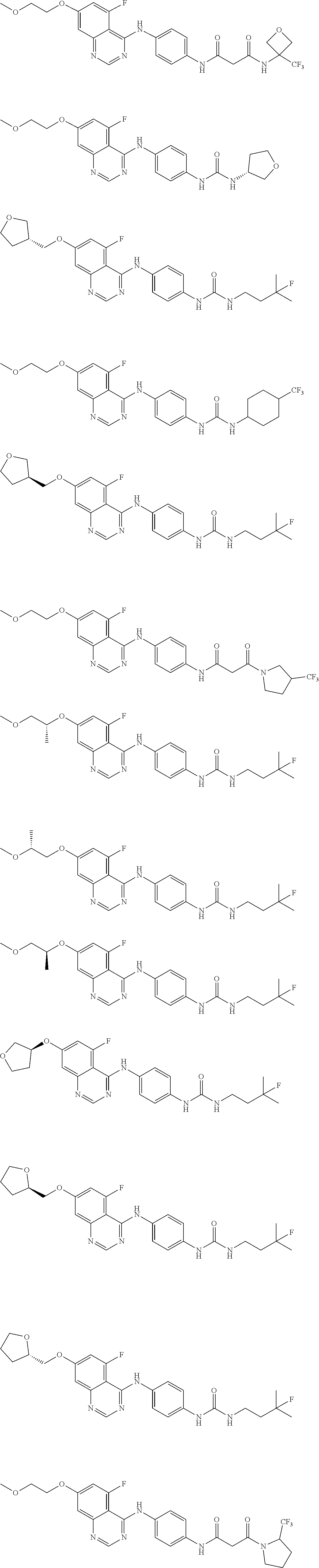

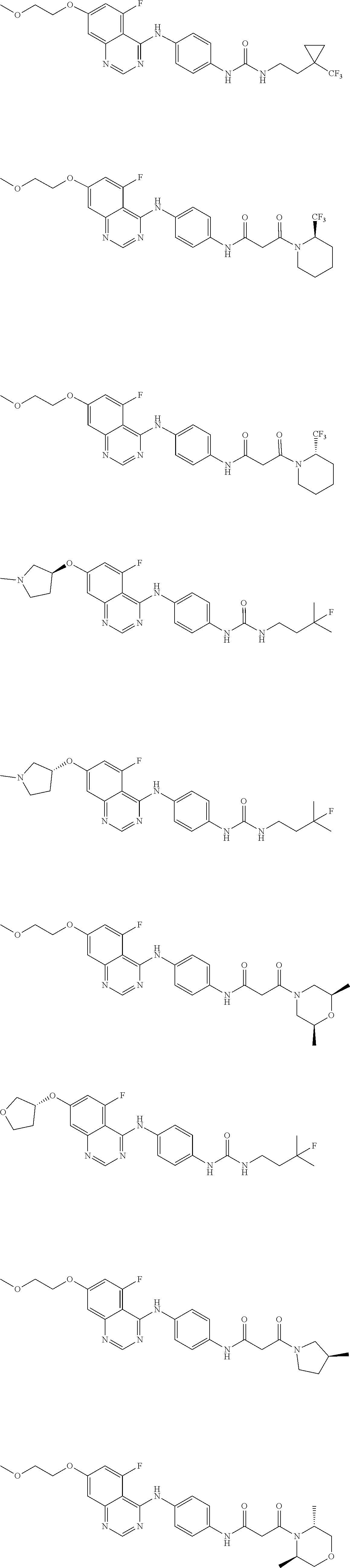

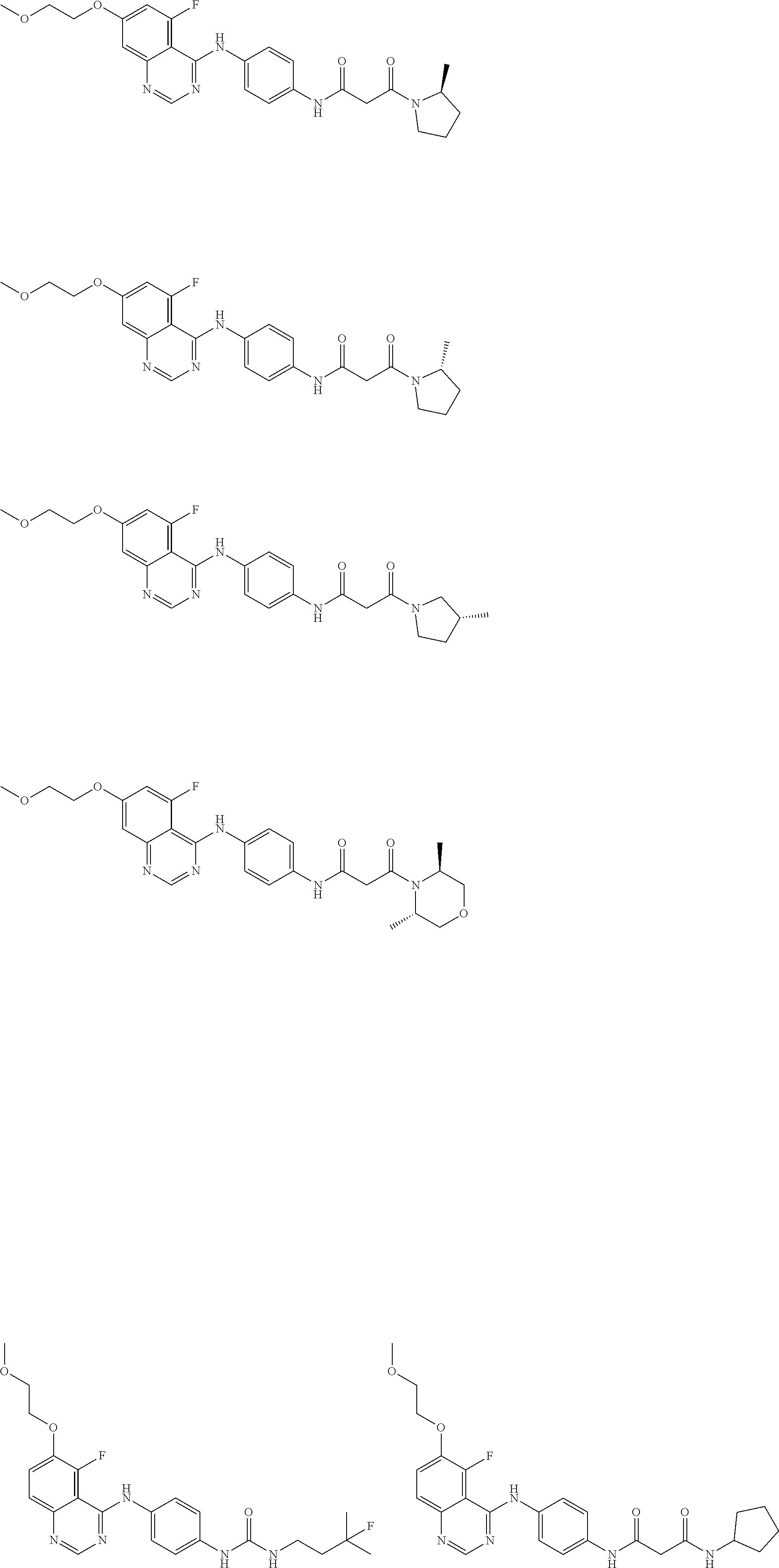

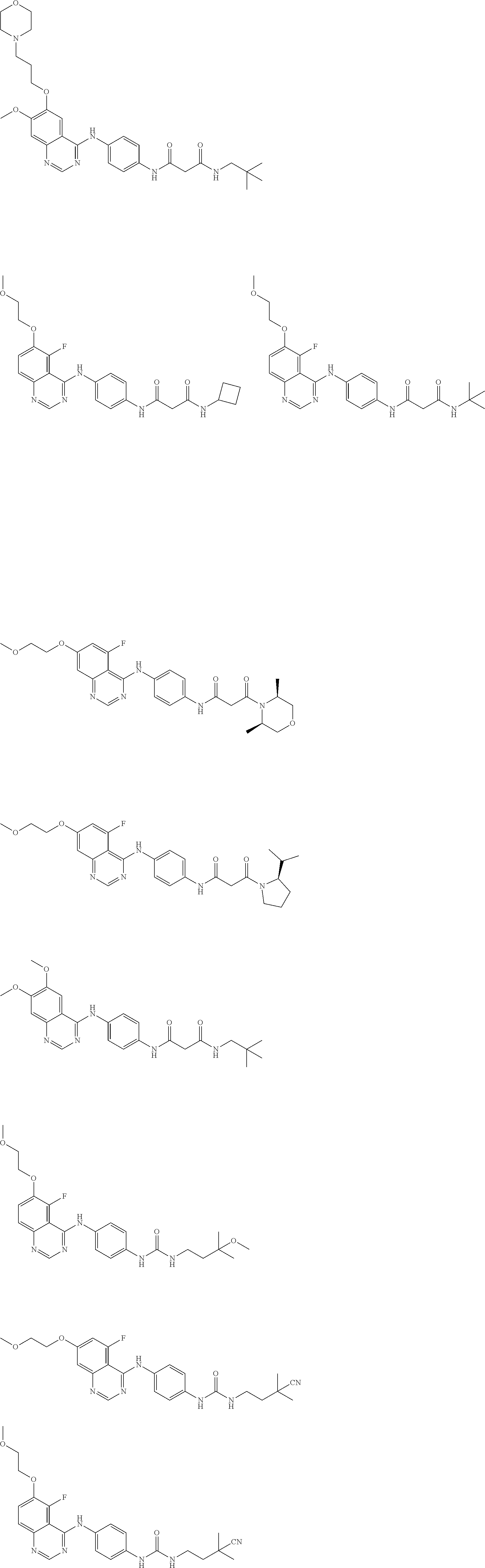

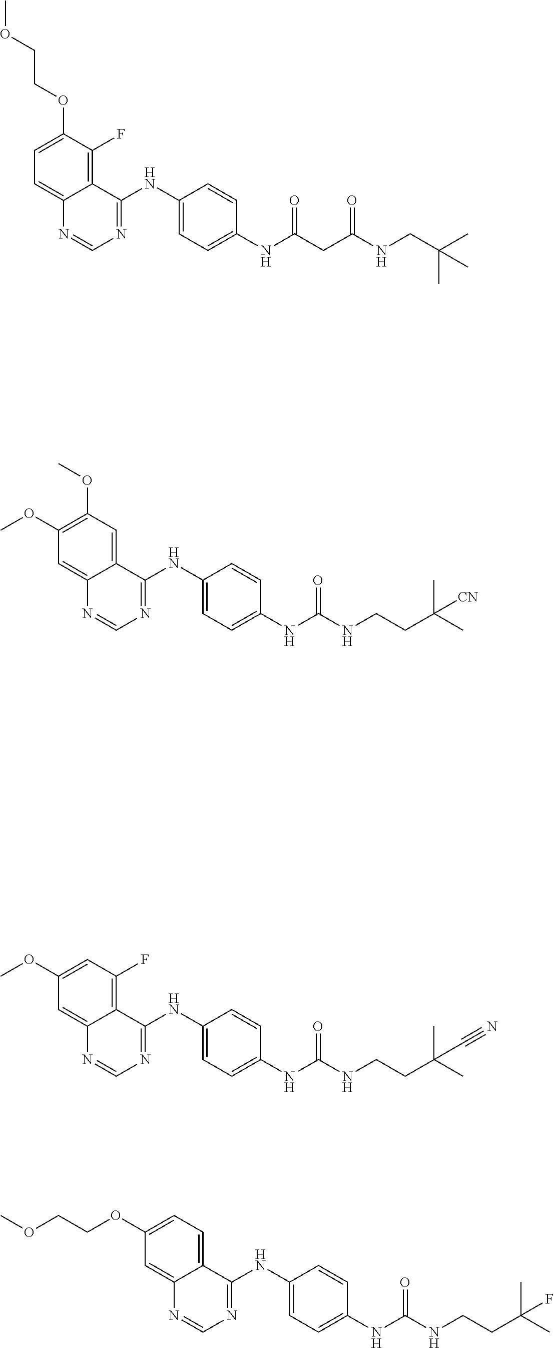

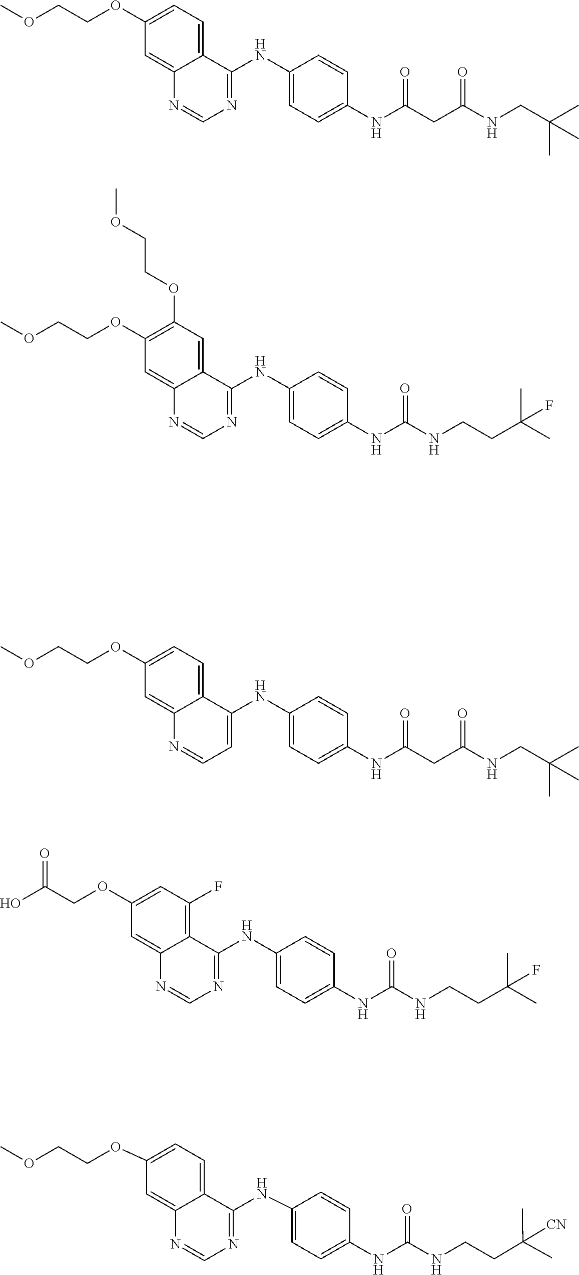

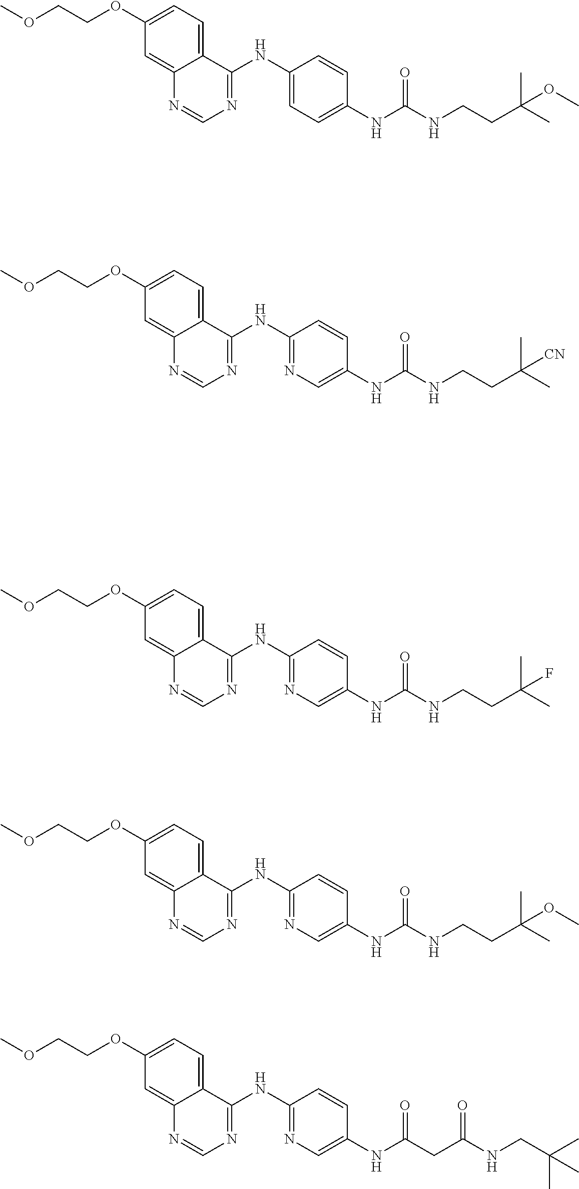

or pharmaceutically acceptable salts, enantiomers, stereoisomers, or tautomers thereof.

### Combination Therapy

In one embodiment, described herein relates to combination therapies that involve the compounds of the disclosure and one or more therapeutic agents. The combination therapies described herein can be used by themselves, or in further combination with one or more additional therapeutic agents (e.g., one or more additional therapeutic agents described below). For example, compounds of the disclosure can be administered together with a cancer targeted therapeutic agent, a cancer-targeted biological, an immune checkpoint inhibitor, or a chemotherapeutic agent. The therapeutic agents can be administered together with or sequentially with another therapeutic agent described herein in a combination therapy.

Combination therapy can be achieved by administering two or more therapeutic agents, each of which is formulated and administered separately. Alternatively, combination therapy can be achieved by administering two or more therapeutic agents in a single formulation.

Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, or 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

In some embodiments, the additional therapeutic agent may include, but are not limited to, cytotoxic agents, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarib, tipifarnib, 4-((5-((4-(3-chlorophenyl)-3-oxopiperazin-1-yl)methyl)-1H-imidazol-1-yl)methyl)benzonitrile hydrochloride, (R)-1-((1H-imidazol-5-yl)methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxafine, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

In some embodiments, the additional therapeutic agent may include, without limitation, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9 inhibitor, CBP/p300 inhibitor, EGFR inhibitor, endothelin receptor antagonist, RAF inhibitor, MEK (mitogen-activated protein kinase kinase) inhibitor, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ITK inhibitor, JAK1/JAK2/JAK3/TYK2 inhibitor, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

In some embodiments, the additional therapeutic agent is an immunomodulatory agent selected from the group consisting of CTLA4 inhibitors such as, but not limited to, ipilimumab and tremelimumab; PD1 inhibitors such as, but not limited to, pembrolizumab, and nivolumab; PDL1 inhibitors such as, but not limited to, atezolizumab (formerly MPDL3280A), durvalumab (formerly MEDI4736), avelumab, PDR001; 4 1BB or 4 1BB ligand inhibitors such as, but not limited to, urelumab and PF-05082566; OX40 ligand agonists such as, but not limited to, MEDI6469; GITR agents such as, but not limited to, TRX518; CD27 inhibitors such as, but not limited to, varlilumab; TNFRSF25 or TL1A inhibitors; CD40 agonists such as, but not limited to, CP-870893; HVEM or LIGHT or LTA or BTLA or CD160 inhibitors; LAG3 inhibitors such as, but not limited to, BMS-986016; TIM3 inhibitors; Siglecs inhibitors; ICOS or ICOS ligand agonists; B7 H3 inhibitors such as, but not limited to, MGA271; B7 H4 inhibitors; VISTA inhibitors; HHLA2 or TMIGD2 inhibitors; inhibitors of Butyrophilins, including BTNL2 inhibitors; CD244 or CD48 inhibitors; inhibitors of TIGIT and PVR family members; KIRs inhibitors such as, but not limited to, lirilumab; inhibitors of ILTs and LIRs; NKG2D and NKG2A inhibitors such as, but not limited to, IPH2201; inhibitors of MICA and MICB; CD244 inhibitors; CSF1R inhibitors such as, but not limited to, emactuzumab, cabiralizumab, pexidartinib, ARRY382, BLZ945; IDO inhibitors such as, but not limited to, INCB024360; thalidomide, lenalidomide, TGFβ inhibitors such as, but not limited to, galunisertib; adenosine or CD39 or CD73 inhibitors; CXCR4 or CXCL12 inhibitors such as, but not limited to, ulocuplumab and (3S,6S,9S,12R,17R,20S,23S,26S,29S,34aS)—N—((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide BKT140; phosphatidylserine inhibitors such as, but not limited to, bavituximab; SIRPA or CD47 inhibitors such as, but not limited to, CC-90002; VEGF inhibitors such as, but not limited to, bevacizumab; and neuropilin inhibitors such as, but not limited to, MNRP1685A.

In some embodiments, the additional therapeutic agent is a chemotherapeutic agent selected from the group consisting of chemotherapeutic agents including, but not limited to, anti-tubulin agents (paclitaxel, paclitaxel protein-bound particles for injectable suspension such as nab-paclitaxel, eribulin, docetaxel, ixabepilone, vincristine), vinorelbine, DNA-alkylating agents (including cisplatin, carboplatin, oxaliplatin, cyclophosphamide, ifosfamide, temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine and methotrexate.

In some embodiments, the additional therapeutic agent is selected from the group consisting of paclitaxel, paclitaxel protein-bound particles for injectable suspension, eribulin, docetaxel, ixabepilone, vincristine, vinorelbine, cisplatin, carboplatin, oxaliplatin, cyclophosphamide, ifosfamide, temozolomide, doxorubicin, pegylated liposomal doxorubicin, daunorubicin, idarubicin, epirubicin, 5-fluorouracil, capecitabine, cytarabine, decitabine, 5-azacytadine, gemcitabine, methotrexate, erlotinib, gefitinib, lapatinib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelasib, quizartinib, tamoxifen, fulvestrant, anastrozole, letrozole, exemestane, abiraterone acetate, enzalutamide, nilutamide, bicalutamide, flutamide, cyproterone acetate, prednisone, dexamethasone, irinotecan, camptothecin, topotecan, etoposide, etoposide phosphate, mitoxantrone, vorinostat, romidepsin, panobinostat, valproic acid, belinostat, DZNep 5-aza-2′-deoxycytidine, bortezomib, carfilzomib, thalidomide, lenalidomide, pomalidomide, trastuzumab, pertuzumab, cetuximab, panitumumab, ipilimumab, labrolizumab, nivolumab, MPDL3280A, bevacizumab, aflibercept, brentuximab vedotin, ado-trastuzumab emtansine, radiotherapy, and sipuleucel T.

In some embodiments, the additional therapeutic agent is a kinase inhibitor selected from the group consisting of erlotinib, gefitinib, lapatanib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelalisib, and quizartinib.

In some embodiments, the one or more additional therapeutic agents that may be administered in combination with a compound provided herein can be a MAPK pathway inhibitor. Such MAPK pathway inhibitors include, for example, MEK inhibitors, ERK inhibitors, and Ras inhibitors.

Exemplary MEK inhibitors include, but are not limited to, trametinib, selumetinib, cobimetinib, binimetinib, and pharmaceutically acceptable salts thereof. Exemplary ERK inhibitors include, but are not limited to, include, but are not limited to, ulixertinib, SCH772984, LY3214996, ravoxertinib, VX-11e, ASN-007, GDC-0994, MK-8353, ASTX-029, LTT462, KO-947, and pharmaceutically acceptable salts thereof. Exemplary Ras inhibitors include, but are not limited to, AMG-510, MRTX849, ARS-1620, ARS-3248, LY3499446, and pharmaceutically acceptable salts thereof.

In some embodiments, the additional therapeutic agent is an anti-PD1 therapeutic. Examples of anti-PD1 therapeutics that may be administered in combination with the compound of the disclosure or pharmaceutically acceptable salt thereof or a composition comprising the compound of the disclosure or pharmaceutically acceptable salt thereof described herein include, but are not limited to, nivolumab, pidilizumab, tislelizumab, AMP-224, AMP-514, and pembrolizumab.

In some embodiments, the additional therapeutic agent is selected from the group consisting of immunomodulatory agents including, but not limited to, anti-PD-L1 therapeutics including atezolizumab, durvalumab, BMS-936559, and avelumab, anti-TIM3 therapeutics including TSR-022 and MBG453, anti-LAG3 therapeutics including relatlimab, LAG525, and TSR-033, CD40 agonist therapeutics including SGN-40, CP-870,893 and RO7009789, anti-CD47 therapeutics including Hu5F9-G4, anti-CD20 therapeutics, anti-CD38 therapeutics, and other immunomodulatory therapeutics including thalidomide, lenalidomide, pomalidomide, prednisone, and dexamethasone. In some embodiments, the additional therapeutic agent is avelumab.

In some embodiments, the additional therapeutic agent is a chemotherapeutic agent selected from the group consisting of anti-tubulin agents (e.g., paclitaxel, paclitaxel protein-bound particles for injectable suspension, eribulin, abraxane, docetaxel, ixabepilone, taxiterem, vincristine or vinorelbine), LHRH antagonists including, but not limited to, leuprolide, goserelin, triptorelin, or histrelin, anti-androgen agents including, but not limited to, abiraterone, flutamide, bicalutamide, nilutamide, cyproterone acetate, enzalutamide, and apalutamide, anti-estrogen agents including, but not limited to tamoxifen, fulvestrant, anastrozole, letrozole, and exemestane, DNA-alkylating agents (including cisplatin, carboplatin, oxaliplatin, cyclophosphamide, ifosfamide, and temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine methotrexate, bortezomib, and carfilzomib.

In some embodiments, the additional therapeutic agent is selected from the group consisting of targeted therapeutics including kinase inhibitors erlotinib, gefitinib, lapatanib, everolimus, temsirolimus, abemaciclib, LEE011, palbociclib, crizotinib, cabozantinib, sunitinib, pazopanib, sorafenib, regorafenib, axitinib, dasatinib, imatinib, nilotinib, vemurafenib, dabrafenib, trametinib, cobimetinib, binimetinib, idelalisib, quizartinib, avapritinib, BLU-667, BLU-263, Loxo 292, larotrectinib, and quizartinib, anti-estrogen agents including, but not limited to, tamoxifen, fulvestrant, anastrozole, letrozole, and exemestane, anti-androgen agents including, but not limited to, abiraterone acetate, enzalutamide, nilutamide, bicalutamide, flutamide, cyproterone acetate, steroid agents including, but not limited to, prednisone and dexamethasone, PARP inhibitors including, but not limited to, neraparib, olaparib, and rucaparib, topoisomerase I inhibitors including, but not limited to, irinotecan, camptothecin, and topotecan, topoisomerase II inhibitors including, but not limited to, etoposide, etoposide phosphate, and mitoxantrone, Histone Deacetylase (HDAC) inhibitors including, but not limited to, vorinostat, romidepsin, panobinostat, valproic acid, and belinostat, DNA methylation inhibitors including, but not limited to, DZNep and 5-aza-2′-deoxycytidine, proteasome inhibitors including, but not limited to, bortezomib and carfilzomib, thalidomide, lenalidomide, pomalidomide, biological agents including, but not limited to, trastuzumab, ado-trastuzumab, pertuzumab, cetuximab, panitumumab, ipilimumab, tremelimumab, vaccines including, but not limited to, sipuleucel-T, and radiotherapy.

In some embodiments, the additional therapeutic agent is selected from the group consisting of an inhibitor of the TIE2 immunokinase including rebastinib or ARRY-614.

In some embodiments, the additional therapeutic agent is selected from the group consisting of an inhibitor of the TIE2 immunokinase including rebastinib or ARRY-614, and an anti-PD1 therapeutic.

In some embodiments, the additional therapeutic agent is selected from the group consisting of anti-angiogenic agents including AMG386, bevacizumab and aflibercept, and antibody-drug-conjugates (ADCs) including brentuximab vedotin, trastuzumab emtansine, and ADCs containing a payload such as a derivative of camptothecin, a pyrrolobenzodiazepine dimer (PBD), an indolinobenzodiazepine dimer (IGN), DM1, DM4, MMAE, or MMAF.

In some embodiments, the additional therapeutic agent is selected from a luteinizing hormone-releasing hormone (LHRH) analog, including goserelin and leuprolide.

In some embodiments, the additional therapeutic agent is selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C₁₁, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH2 acetate [C59H84N18Oi4—(C2H4O2)x where x=1 to 2.4] (SEQ ID NO: 3), goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, Gleevec, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, ipilumumab, vemurafenib, and mixtures thereof.

In some embodiments, the additional therapeutic agent is an HSP90 inhibitor (e.g., AT13387). In some embodiments, the additional therapeutic agent is cyclophosphamide. In some embodiments, the additional therapeutic agent is an AKT inhibitor (e.g., perifosine). In some embodiments, the additional therapeutic agent is a BCR-ABL inhibitor (e.g., nilotinib). In some embodiments, the additional therapeutic agent is an mTOR inhibitor (e.g., RAD001). In some embodiments, the additional therapeutic agent is an FGFR inhibitor (e.g., erdafitinib, K0947, or BGJ398). In some embodiments, the additional therapeutic agent is an anti-PDL1 therapeutic. In some embodiments, the additional therapeutic agent is a Bcl2 inhibitor (e.g., venetoclax). In some embodiments, the additional therapeutic agent is an autophagy inhibitor (e.g., hydroxychloroquine). In some embodiments, the additional therapeutic agent is a MET inhibitor.

### Pharmaceutical Compositions and Kits

In one embodiment, described herein is a pharmaceutical composition comprising compounds of the disclosure formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds of the disclosure, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules, and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax, or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Pharmaceutical compositions and compounds of the disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of the disclosure may be suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In one embodiment, provided are enteral pharmaceutical formulations including a compound of the disclosure, an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, provided herein are kits for use by a e.g., a consumer in need of treatment of cancer. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### Methods and Uses

Compounds described herein may act as KIT inhibitors and are therefore useful in the treatment of diseases and disorders in patients in need thereof, such as cancer. Exemplary diseases include, but are not limited to, gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, and non-small cell lung cancer. In some embodiments, the disease is gastrointestinal stromal tumors (GIST). In some embodiments, the disease is a KIT activated gastrointestinal stromal tumors (GIST). In some embodiments, the KIT activated gastrointestinal stromal tumors (GIST) has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the disease is melanoma. In some embodiments, the melanoma is a KIT activated melanoma. In some embodiments, the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the melanoma is selected from the group consisting of cutaneous melanoma and noncutaneous melanoma. In some embodiments, the cutaneous melanoma is selected from the group consisting of superficial spreading melanoma, nodular melanoma, acral-lentiginous melanoma, amelanotic melanoma, and desmoplastic melanoma. In some embodiments, noncutaneous melanoma is selected from ocular melanoma and mucosal melanoma.

In one embodiment, the use of the pharmaceutical composition may be used for preparing a medicament for the treatment of a disease selected from the group consisting of gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, and non-small cell lung cancer. In some embodiments, the disease is gastrointestinal stromal tumors (GIST). In some embodiments, the disease is a KIT activated gastrointestinal stromal tumors (GIST). In some embodiments, the KIT activated gastrointestinal stromal tumors (GIST) has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the disease is melanoma. In some embodiments, the melanoma is a KIT activated melanoma. In some embodiments, the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the melanoma is selected from the group consisting of cutaneous melanoma and noncutaneous melanoma. In some embodiments, the cutaneous melanoma is selected from the group consisting of superficial spreading melanoma, nodular melanoma, acral-lentiginous melanoma, amelanotic melanoma, and desmoplastic melanoma. In some embodiments, noncutaneous melanoma is selected from ocular melanoma and mucosal melanoma.

The compounds of the disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound provided herein may be administered orally, subcutaneously, topically, parenterally, by inhalation spray or rectally in dosage unit formulations containing conventional non-toxic pharmaceutically acceptable carriers, adjuvants, and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved.

### EXAMPLES

The compounds described herein can be prepared in a number of ways based on the teachings contained herein and disclosures of synthetic procedures in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

ChemDraw version 10 or 12 (CambridgeSoft Corporation, Cambridge, MA) was used to name the structures of intermediates and exemplified compounds.

The following abbreviation are used in this disclosure and have the following definitions: “ADP” is adenosine diphosphate, “Aq” is aqueous, “ATP” is adenosine triphosphate, “Ar” is argon gas, “Boc” is t-butylcarbonate, “BSA” is bovine serum albumin, “BTFFH” is 1-(fluoro(pyrrolidin-1-yl)methylene)pyrrolidin-1-ium hexafluorophosphate(V), “conc.” is concentrated, “Cs₂CO₃” is cesium carbonate, “CuCN” is copper(I) cyanide, “CuI” is copper(I) iodide, “DBU” is 1,8-diazabicyclo[5.4.0]undec-7-ene, “DCM” is dichloromethane, “DIAD” is diisopropyl azodicarboxylate, “DIEA” is N,N-diisopropylethylamine, “DMA” is N,N-dimethylacetamide, “DMAP” is 4-(dimethylamino)pyridine, “DMF” is N,N-dimethylformamide, “DMEM” is Dulbecco's Modified Eagle Media, “DMSO” is dimethylsulfoxide, “DPPA” is diphenylphosphoryl azide, “EDC” is 1-ethyl-3-(3-dimethylaminopropyl)carbodiimide, “ESI” is electrospray ionization, “Et₃N” is triethylamine, “Et₂O” is diethylether, “EtOAc” is ethyl acetate, “EtOH” is ethanol, “GST” is glutathione S-transferase, “h” is hour, “HATU” is hexafluorophosphate azabenzotriazole tetramethyl uronium, “H₂” is hydrogen gas, “HCl” is hydrochloric acid, “Hex” is hexane, “H₂SO₄” is sulfuric acid, “HOBt” is hydroxybenzotriazole “ICso” is half maximal inhibitory concentration, “K₂CO₃” is potassium carbonate, “KI” is potassium iodide, “K₃PO₄” is potassium phosphate, “LAH” is lithium aluminum hydride, “LiOH” is lithium hydroxide, “mCPBA” is meta-chloroperoxybenzoic acid, “CH₃CN” is acetonitrile, “MeOH” is methanol, “MgSO₄” is magnesium sulfate, “MHz” is megahertz, “min” is minute or minutes, “MS” is mass spectrometry, “MTBE” is methyl tert-butyl ether, “NADH” is nicotinamide adenine dinucleotide, “NaH” is sodium hydride, “NaHCO₃” is sodium bicarbonate, “NaOH” is sodium hydroxide, “NaOMe” is sodium methoxide, “Na₂SO₄” is sodium sulfate, “NH₄Cl” is ammonium chloride, “NH₄OH” is ammonium hydroxide, “NMR” is nuclear magnetic resonance, “OTs” is O-tosylate, “PBS” is phosphate buffered saline, “Pd” is palladium, “Pd—C” is palladium on carbon, “Ph₃P” is triphenylphosphine, “prep-HPLC” is preparative high performance liquid chromatography, “PyAOP” is ((3H-[1,2,3]triazolo[4,5-b]pyridin-3-yl)oxy)tri(pyrrolidin-1-yl)phosphonium hexafluorophosphate(V), “rt” is room temperature which is also known as “ambient temp,” which will be understood to consist of a range of normal laboratory temperatures ranging from 15-25° C., “SFC” is supercritical fluid chromatography, “SM” is starting material, “T3P” is n-propanephosphonic acid anhydride, “TBAF” is tertabutyl ammonium fluoride, “TCFH” is chloro-N,N,N′,N′-tetramethylformamidinium hexafluorophosphate, “TEA” is triethylamine, “TFA” is trifluoroacetic acid, “Tf₂O” is trifluoromethanesulfonic anhydride, “THF” is tetrahydrofuran, “TrocCl” is 2,2,2-trichloroethyl chloroformate, and “XtalFluor-E” is diethylaminodifluorosulfiniun tetrafluoroborate.

### General Chemistry

Exemplary compounds described herein are available by the general synthetic methods illustrated in the Schemes below, Intermediate preparations, and the accompanying Examples.

### Synthetic Schemes

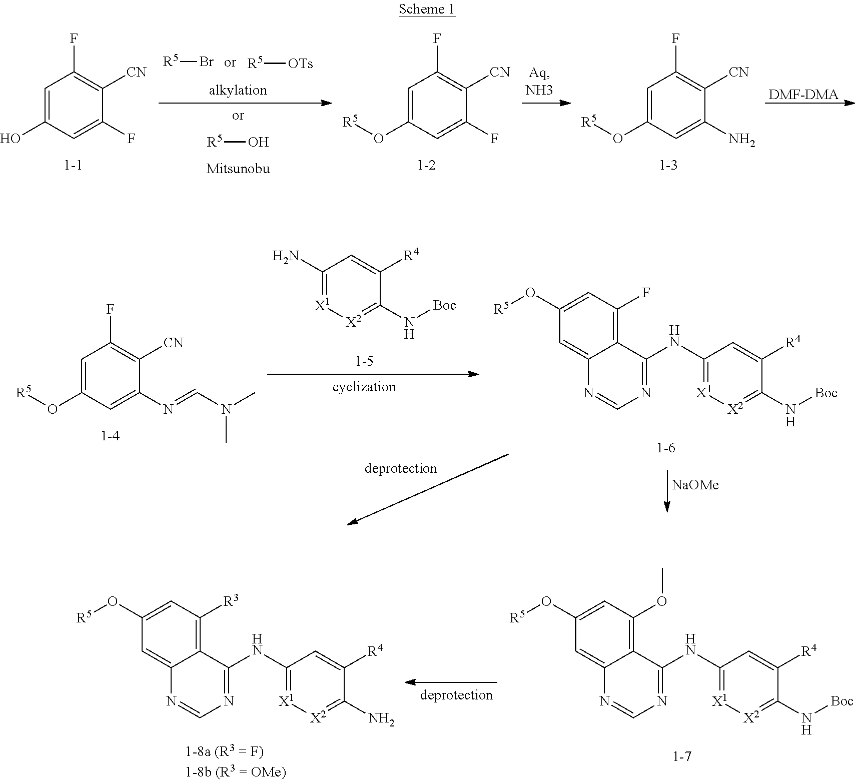

Scheme 1 illustrates an exemplary preparation of quinazoline intermediates 1-8a, and 1-8b. Alkylation of 1-1 (2,6-difluoro-4-hydroxybenzonitrile) with R⁵—Br, and R⁵—OTs (commercially available or synthesized by those skilled in the art) in the presence of base (e.g., K₂CO₃, Cs₂CO₃ or NaH) and a polar aprotic solvent (DMSO, DMF, THF, or the like), at temperatures between ambient and 150° C. gives ethers 1-2. Alternatively, compounds 1-1 react with alcohols (R⁵—OH) under standard Mitsunobu conditions (conducted for example in the presence of Ph₃P and DIAD) to provide the ethers 1-2. Ethers 1-2 react with Aq. NH₃ to obtain anilines 1-3 by an aromatic substitution reaction. Anilines 1-3 react with DMF-DMA under elevated temperature to form formamidines 1-4. Cyclization between 1-4 and anilines/aminopyridines 1-5 (commercially available or synthesized by those skilled in the art) in acetic acid at elevated temperature obtains quinazolines 1-6. Intermediates 1-6 react with NaOMe in dry aprotic solvents such as THF at elevated temperature to obtain 1-7. Finally, boc-deprotection of 1-6 and 1-7 under acidic conditions such as HCl and TFA in an aprotic solvent such as DCM forms 1-8a (R³═F) and 1-8b (R³═OMe) respectively.

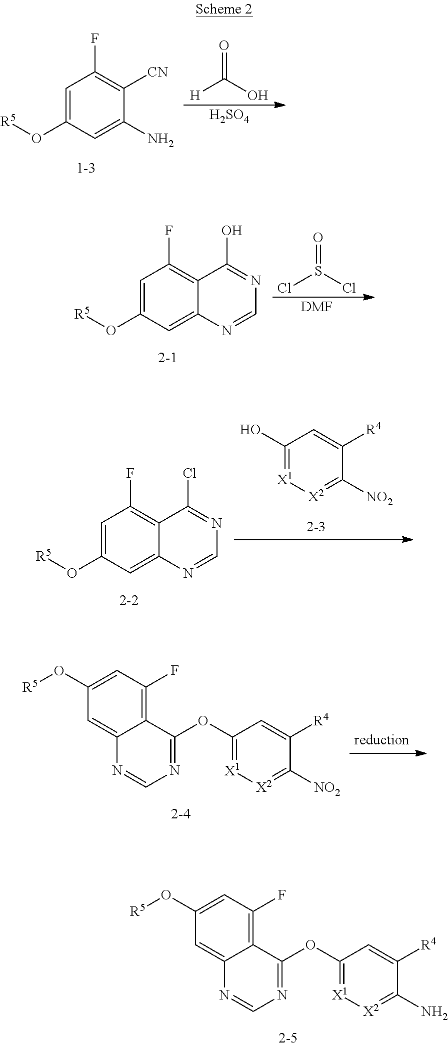

Scheme 2 illustrates an exemplary preparation of quinazoline intermediates 2-5. Cyclization of compounds 1-3 (see scheme 1) with formic acid under acidic conditions such as H₂SO₄ at elevated temperature affords quinazolinols 2-1. Quinazolinols 2-1 react with thionyl chloride in the presence of catalyst DMF to afford chlorides 2-2. The aromatic nucleophilic substitution reaction of chlorides 2-2 with nucleophiles 2-3 in the presence of catalyst DMAP, and in the presence of a base such as NaH, Et₃N, Cs₂CO₃ in a polar solvent such as DMSO, DMF, DMA, DCM, THF at 0° C. to elevated temperature affords 2-4. Nitro reduction of 2-4 in the presence of a hydrogenation catalyst, such as palladium or nickel, or mild reducing conditions such as zinc or iron and NH₄Cl provides the corresponding anilines 2-5.

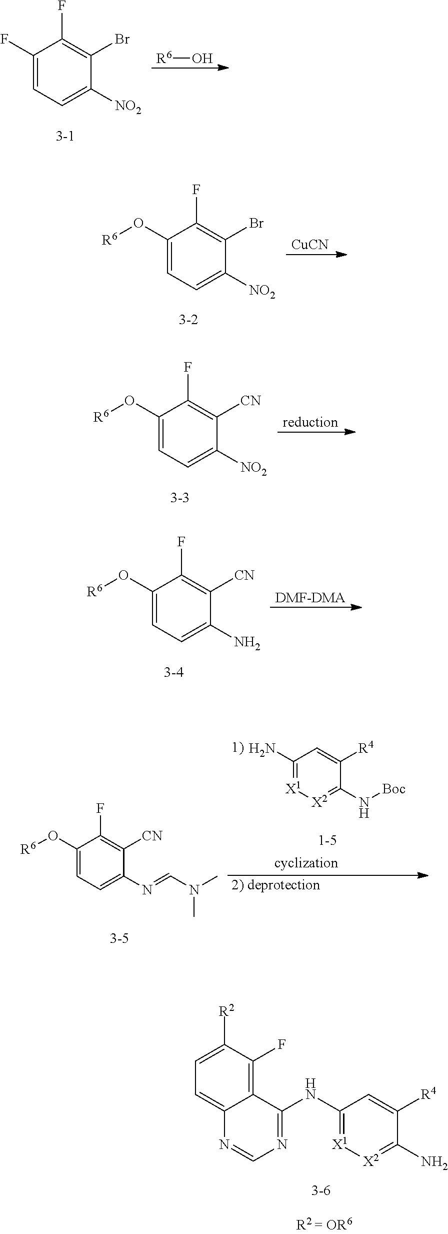

Scheme 3 illustrates an exemplary preparation of quinazoline intermediates 3-6. Base-promoted nucleophilic substitution of 3-1 (2-bromo-3,4-difluoro-1-nitrobenzene) with R⁶—OH (commercially available or synthesized by those skilled in the art) in a polar aprotic solvent (DCM, DMSO, DMF, THF or the like) at temperatures between ambient and 70° C. provides ethers 3-2 which can be purified by SFC purification, crystallization, or chromatography. Cyanation of aryl bromide 3-2 with CuCN at elevated temperature in polar solvents such as DMA, DMF or DMSO affords 3-3. Nitro reduction of 3-3 in the presence of a hydrogenation catalyst, such as Pd or nickel, or mild reducing conditions such as SnCl₂, zinc or iron and NH₄Cl provides the corresponding anilines 3-4. In a similar manner in scheme 1, anilines 3-4 react with DMF-DMA under elevated temperature to form formamidines 3-5. Cyclization between 3-5 and anilines/aminopyridines 1-5 (commercially available or synthesized by those skilled in the art) in acetic acid at elevated temperature obtains boc-quinazolines which can be deprotected under acidic conditions such as HCl and TFA in an aprotic solvent such as DCM to obtain 3-6 (R²═OR⁶)

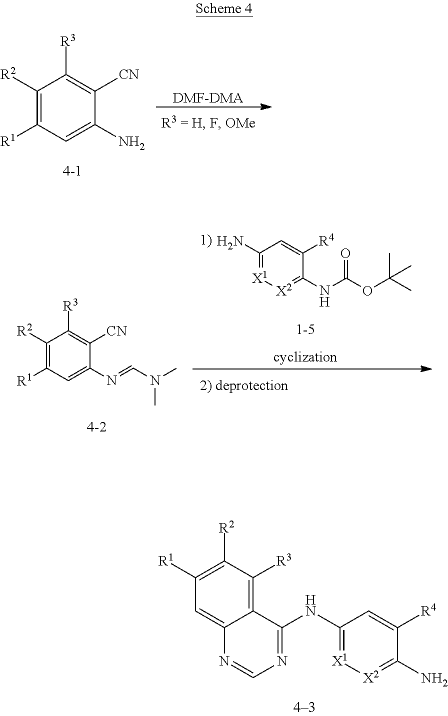

Scheme 4 illustrates an exemplary preparation of quinazoline intermediates 4-3. In a similar manner in scheme 1, anilines 4-1 (commercially available or synthesized by those skilled in the art) react with DMF-DMA under elevated temperature to form formamidines 4-2. Cyclization between 4-2 and anilines/aminopyridines 1-5 (commercially available or synthesized by those skilled in the art) in acetic acid at elevated temperature obtains boc-quinazolines which can be deprotected under acidic conditions such as HCl and TFA in an aprotic solvent such as DCM to obtain 4-3.

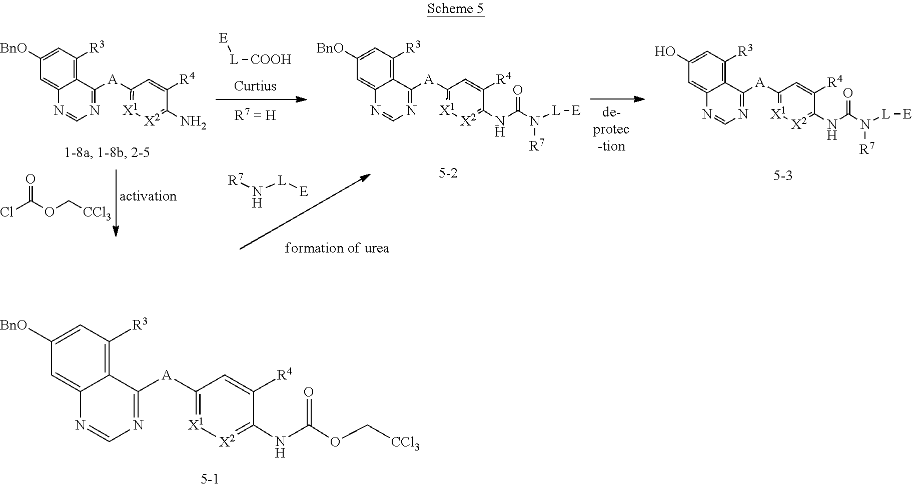

Scheme 5 illustrates an exemplary preparation of intermediates 5-3. Activation of 1-8a, 1-8b, and 2-5 with 2,2,2-trichloroethyl chloroformate (or isopropenyl chloroformate) under Schotten-Baumann conditions (for example in a mixture of saturated aqueous NaHCO₃ and EtOAc) affords 5-1 which is further reacted with amines NHR⁷-L-E (commercially available or synthesized by those skilled in the art) to furnish urea 5-2. Alternatively, urea 5-2 can be prepared by reaction of 1-8a, 1-8b, and 2-5 with carboxylic acids E-L-COOH (commercially available or synthesized by those skilled in the art) under Curtius rearrangement (in situ generation of isocyanate) conditions (DPPA and a base [Et₃N] in a suitable solvent such as 1,4-dioxane at elevated temperature). Finally, Pd—C catalyzed deprotection of 5-2 under hydrogen obtains intermediates 5-3.

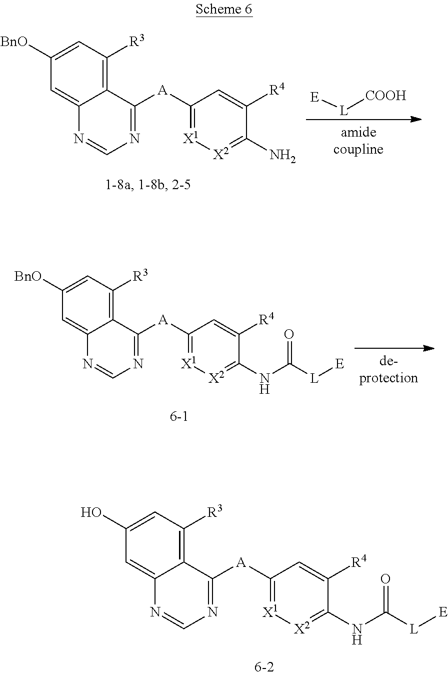

Scheme 6 illustrates an exemplary preparation of intermediates 6-2. Amides 6-1 can be prepared by amide coupling reaction of 1-8a, 1-8b, and 2-5 with acids E-L-COOH (commercially available or synthesized by those skilled in the art) in the presence of coupling reagents such as T3P, TCFH, HATU, EDC, and XtalFluor-E. In a similar manner as scheme 4, Pd—C catalyzed deprotection of 6-1 under hydrogen obtains intermediates 6-2.

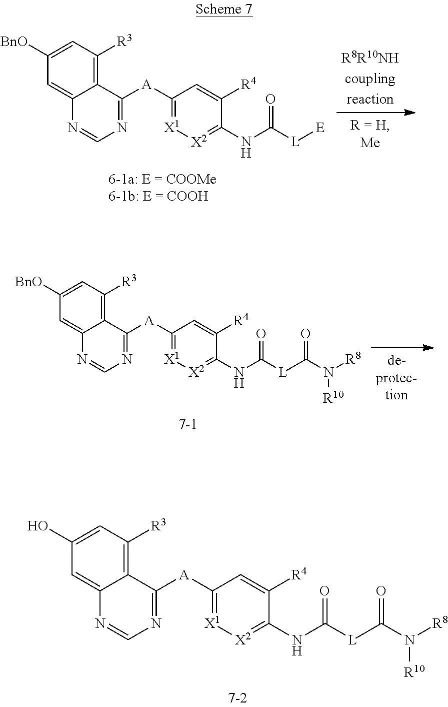

Scheme 7 illustrates an exemplary preparation of intermediates 7-2. Intermediates 6-1a react with amines NHR⁸R¹⁰ (commercially available or synthesized by those skilled in the art) in a suitable solvent such as 1,4-dioxane or THF at elevated temperature to furnish 7-1. Alternatively, amide coupling reaction of 6-1b with amines NR⁸R¹⁰H (commercially available or synthesized by those skilled in the art) in the presence of coupling reagents such as T3P, TCFH, HATU, and EDC, and XtalFluor-E to afford 7-1. In a similar manner as scheme 4, Pd—C catalyzed deprotection of 7-1 under hydrogen obtains intermediates 7-2.

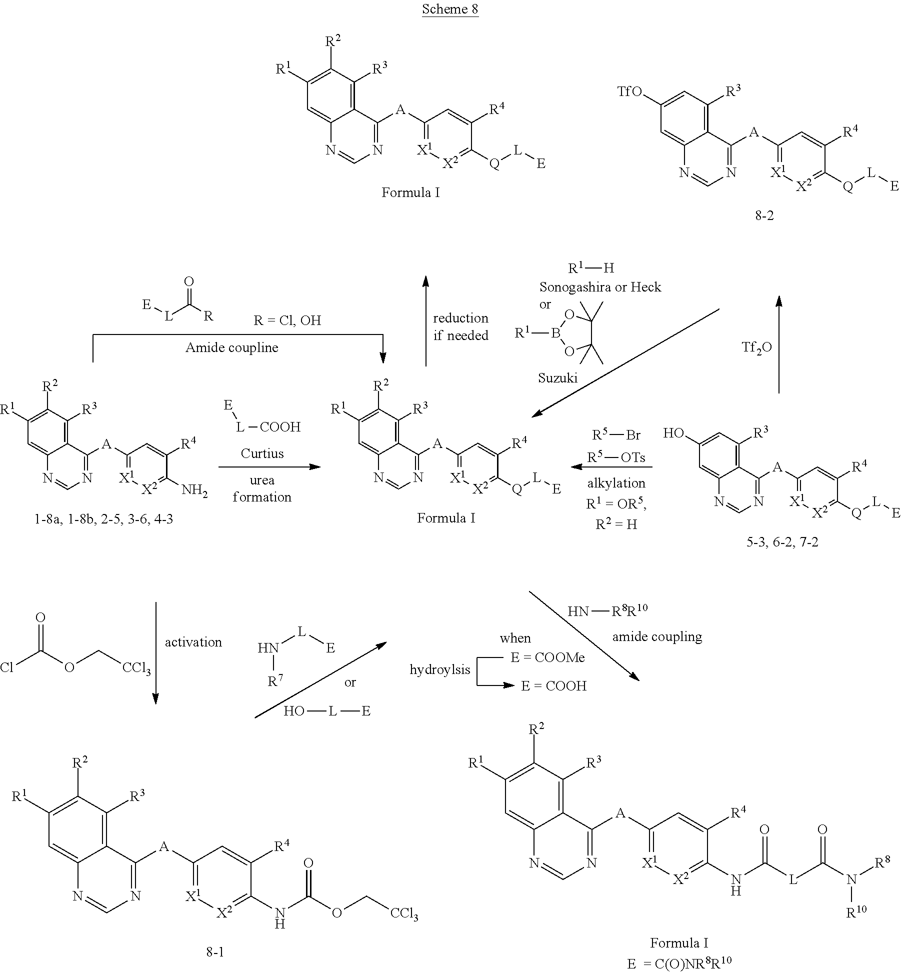

Scheme 8 illustrates an exemplary preparation of compounds of Formula I. Activation of 1-8a, 1-8b, 2-5, 3-6, and 4-3 with 2,2,2-trichloroethyl chloroformate (or isopropenyl chloroformate) under Schotten-Baumann conditions (for example in a mixture of saturated aqueous NaHCO₃ and EtOAc) affords 8-1 which is further reacted with amines HNR⁷-L-E or alcohols HO-L-E (commercially available or synthesized by those skilled in the art) to furnish Formula I as a urea. Alternatively, Formula I as a urea can be prepared by reaction of 1-8a, 1-8b, 2-5, 3-6, and 4-3 with carboxylic acids E-L-COOH (commercially available or synthesized by those skilled in the art) under Curtius rearrangement (in situ generation of isocyanate) conditions (DPPA and a base [Et₃N] in a suitable solvent such as 1,4-dioxane at elevated temperature). Formula I as an amide can be prepared by amide coupling reaction of 1-8a, 1-8b, 2-5, 3-6, and 4-3 with acids E-L-COOH (commercially available or synthesized by those skilled in the art) in the presence of coupling reagents such as T3P, TCFH, HATU, EDC, and XtalFluor-E or with acyl chlorides E-L-COCl (commercially available or synthesized by those skilled in the art) in the presence of base such as Et₃N, and DIEA. Activation of 5-3, 6-2, and 7-2 with Tf₂O, in a presence of base such as Et₃N, DIEA, pyridine, or DBU as a base in an organic solvent such as CH₃CN. DCM, or EtOAc afford intermediates 8-2. Sonogashira or Heck coupling reaction of 8-2 following by appropriate reduction to furnish C-C linked Formula I. Alternatively, C-C linked Formula I can be prepared from 8-2 with boronates or boronic acid under Suzuki conditions, followed by appropriate reduction if needed. In another embodiment, alkylation of 5-3, 6-2, and 7-2 with R⁵—Br, and R⁵—OTs (commercially available or synthesized by those skilled in the art) in the presence of base (e.g., K₂CO₃, Cs₂CO₃ or NaH) and a polar aprotic solvent (DMSO, DMF, THF or the like), at temperatures between ambient and 150° C. gives compounds of Formula I (R¹═ORs, R²═H). Finally, compounds of Formula I which contains an ester functionality can be hydrolyzed under basic conditions such as LiOH or NaOH in a mixture of water/THF or 1,4-dioxane to afford the corresponding acids. In a similar manner as scheme 6, Formula I as an ester reacts with amines NR⁸R¹⁰H (commercially available or synthesized by those skilled in the art) in a suitable solvent such as 1,4-dioxane, THF at elevated temperature to furnish Formula I (E=C(O)NR⁸R¹⁰). Alternatively, amide coupling reaction of the corresponding acids with amines NR⁸R¹⁰H (commercially available or synthesized by those skilled in the art) in the presence of coupling reagents such as T3P, TCFH, HATU, EDC, and XtalFluor-E to afford Formula I (E=C(O)NR⁸R¹⁰).

### Preparation of Intermediates.

Using the synthetic procedures and methods described herein and methods known to those skilled in the art, the following compounds were made:

General Method A: Alkylation with Alkyl Halide

### Intermediate A1: 2,6-Difluoro-4-(2-methoxyethoxy)benzonitrile

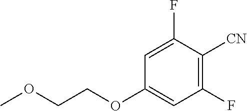

A suspension of 2,6-difluoro-4-hydroxybenzonitrile (50 g, 322 mmol) and 1-bromo-2-methoxyethane (50 g, 360 mmol) in DMF (300 mL) was treated with K₂CO₃ (100 g, 724 mmol). The resulting reaction mixture was heated at 80° C. for 5 h. The reaction mixture diluted with water (700 mL) extracted with dichloromethane (3×). The combined organic layers were washed with brine, dried over anhydrous Na₂SO₄, filtered, and concentrated under reduced pressure. The crude product was recrystallized from hexane and MTBE to provide 2,6-difluoro-4-(2-methoxyethoxy)benzonitrile (50 g, 73%) as white solid. ¹H NMR (500 MHz, DMSO-d₆): δ 6.38 (s, 1H), 6.15 (m, 1H), 4.06 (m, 2H), 3.63 (m, 2H), 3.29 (s, 3H); MS (ESI) m/z: 214.0 (M+H⁺).

Using the General Method A above, the following Intermediates of Table A were prepared.

TABLE A

| Ex. | | | Yield | ¹H NMR (400 or 500 | MS (m/z: |
|---|---|---|---|---|---|
| No | SM | Product | (%) | MHz, DMSO-d₆): δ | M + H⁺) |
| A2 | 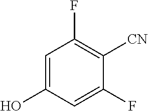 | 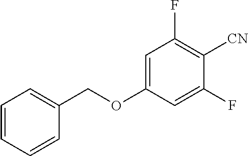 [SMILES: N#Cc1c(F)cc(OCc2ccccc2)cc1F] | 89 | 7.37-7.47 (m, 5H), 7.19 (d, J = 10.4 Hz, 2H), 5.23 (s, 2H). | No Data |
| | 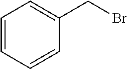 | | | | |
| A3 | 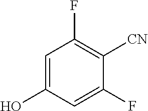 | 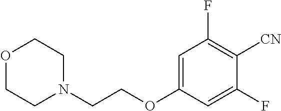 [SMILES: N#Cc1c(F)cc(OCCN2CCOCC2)cc1F] | 27 | 7.13 (d, J = 21.2 Hz, 2H), 4.23 (t, J = 5.6 Hz, 2H), 3.55 (m, 4H), 2.69 (t, J = 5.6 Hz, 2H), 2.45 (m, 4H). | 268.9 |
| | 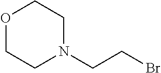 | | | | |
| A4 | 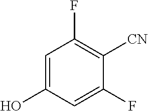 | 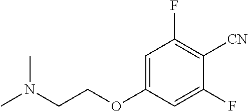 [SMILES: CN(C)CCOc1cc(F)c(C#N)c(F)c1] | 55 | 7.11 (d, J = 10.4 Hz, 2H), 4.19 (t, J = 5.6 Hz, 2H), 2.64 (t, J = 5.6 Hz, 2H), 2.20 (s, 6H). | 226.9 |
| | 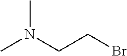 | | | | |
| A5 | 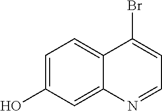 | 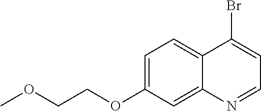 [SMILES: COCCOc1ccc2c(Br)ccnc2c1] | crude | 8.64 (d, J = 4.7 Hz, 1H), 8.05 (dd, J = 9.3 Hz, 1H), 7.77 (d, J = 4.7 Hz, 1H), 7.48 (d, J = 2.6 Hz, 1H), 7.43 (d, J = 9.0 Hz, 1H), 4.31 (m, 2H), 3.75 (m, 2H), 3.34 (s, 3H) | 282.0 284.0 |
| | 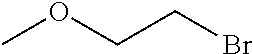 | | | | |

General Method B: Substitution with NH₄OH

### Intermediate B1:2-Amino-6-fluoro-4-(2-methoxyethoxy)benzonitrile

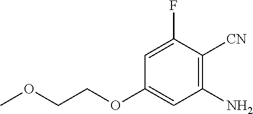

A suspension of 2,6-difluoro-4-(2-methoxyethoxy)benzonitrile (50 g, 235 mmol) in isopropanol (25 mL) was treated with ammonium hydroxide (100 mL, 856 mmol). The resulting reaction mixture heated at 70° C. for 10 days. The reaction mixture cooled to rt and stirred. Crystallized solid filtered, washed with cold water, dried under high vacuum to provide, 2-amino-6-fluoro-4-(2-methoxyethoxy)benzonitrile (45 g, 91%) as a white solid. MS (ESI) m/z: 211.0 (M+H⁺).

Using the General Method B above, the following Intermediates of Table B were prepared.

TABLE B

| Ex. | | | Yield | ¹H NMR (400 or 500 MHz, DMSO-d₆): | MS (m/z: |
|---|---|---|---|---|---|
| No | SM | Product | (%) | δ | M + H⁺) |
| B2 | A2 | 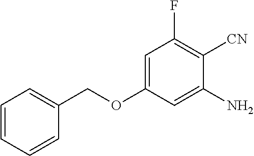 [SMILES: N#Cc1c(N)cc(OCc2ccccc2)cc1F] | 90 | 7.34-7.47 (m, 5H), 6.40 (br s, 2H), 6.25 (d, J = 12.0 Hz, 1H), 6.20 (br s, 1H), 5.07 (s, 2H), | 241.1 |
| B3 | A3 | 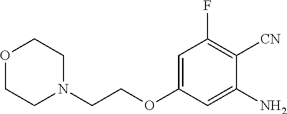 [SMILES: N#Cc1c(N)cc(OCCN2CCOCC2)cc1F] | 72 | 6.36 (br s, 2H), 6.18 (dd, J = 11.6, 2.0 Hz, 1H), 6.11 (br s, 1H), 4.04 (t, J = 5.6 HZ, 2H), 3.55 (m, 4H), 2.64 (t, J = 5.6 Hz, 2H), 2.43 (m, 4H). | 266.1 |
| B4 | A4 | 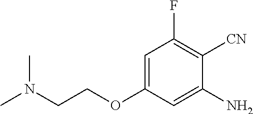 [SMILES: CN(C)CCOc1cc(N)c(C#N)c(F)c1] | 66 | 6.37 (br s, 2H), 6.18 (dd, J = 12.0, 2.0 Hz, 1H), 6.12 (br s, 1H), 4.05 (m, 2H), 2.72 (m, 2H), 2.28 (s, 6H). | 224.0 |

### Intermediate B5: 6-amino-2-fluoro-3-(2-methoxyethoxy)benzonitrile

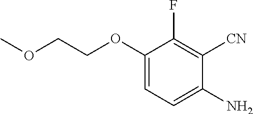

A stirred solution of 2-bromo-3,4-difluoro-1-nitrobenzene (5.0 g, 22.5 mmol) in DCM (100 mL) was treated with tetra butyl ammonium hydrogen sulphate (0.07 g, 0.22 mmol). 2-Methoxyethan-1-ol (1.75 g, 22.5 mmol) was added at rt and then 1.0 M NaOH solution (30 mL) was added dropwise at the same temperature. This reaction mixture was further stirred at rt for 3 h. After complete consumption of starting material, the reaction mass was diluted with water (100 mL) and aqueous layer was extracted with DCM (2×). The combined organic extracts were dried over anhydrous Na₂SO₄, filtered, and concentrated under reduced pressure. The crude material was purified by silica gel column chromatography (15 to 20% EtOAc/hexanes) to afford 2-bromo-3-fluoro-4-(2-methoxyethoxy)-1-nitrobenzene (3.4 g, 55%) as an off white solid. ¹H NMR (400 MHz, DMSO-d₆): δ 8.01 (dd, J=9.2, 1.6 Hz, 1H), 7.43 (d, J=8.8 Hz, 1H), 4.35 (m, 2H), 3.71 (m, 2H), 3.31 (s, 3H).

A stirred solution of 2-bromo-3-fluoro-4-(2-methoxyethoxy)-1-nitrobenzene (3.4 g, 11.5 mmol) in DMF (75 mL) was treated with copper(I) cyanide (2.1 g, 23.5 mmol) at rt. This suspension was further heated at 130° C. for 2 h. After complete consumption of starting material, the reaction mass was cooled to rt and then poured into the ice water (˜75 mL) slowly (Solids were precipitated during this addition process). The solid was filtered, washed with water, and dried under high vacuum to obtained 2-fluoro-3-(2-methoxyethoxy)-6-nitrobenzonitrile (2.2 g, 80%) as a yellow solid. ¹H NMR (400 MHz, DMSO-d₆): δ 8.29 (d, J=9.2 Hz, 1H), 7.73 (d, J=8.4 Hz, 1H), 4.42 (m, 2H), 3.72 (m, 2H), 3.31 (s, 3H).

A stirred solution of 2-fluoro-3-(2-methoxyethoxy)-6-nitrobenzonitrile (2.2 g, 8.9 mmol), in water (30 mL) was treated with sodium dithionite (3.2 g, 18.5 mmol) at rt. This suspension was further heated at 100° C. for 16 h. After consumption of starting material, the reaction was diluted with water (10 mL) and aqueous layer was extracted with 10% MeOH/DCM (2×). The organic extracts were dried over anhydrous Na₂SO₄, filtered, and concentrated under reduced pressure. The crude material was purified by silica gel column chromatography to afford 6-amino-2-fluoro-3-(2-methoxyethoxy)benzonitrile (0.5 g, 25%) as a yellow solid. ¹H NMR (400 MHz, DMSO-d₆): δ 7.25 (t, J=9.6 Hz, 1H), 6.52 (dd, J=9.2, 1.6 Hz, 1H), 5.98 (br s, 2H), 4.03 (m, 2H), 3.58 (m, 2H), 3.29 (s, 3H); MS (ESI) m/z: 211.1 (M+H⁺).

Intermediate B6: 4-(3-((4-chloro-7-methoxyquinazolin-6-yl)oxy)propyl)morpholine

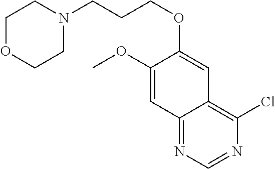 [SMILES: COc1cc2ncnc(Cl)c2cc1OCCCN1CCOCC1]

A solution of 7-methoxy-6-(3-morpholinopropoxy)quinazolin-4-ol (1.0 g, 3.1 mmol) CHCl₃ (25 mL) was treated with DMF (1-2 drops). Oxalyl chloride (excess) was added into the solution drop by drop. The mixture was refluxed overnight. The mixture was concentrated under reduced pressure to dryness. The residue was treated with CHCl₃ (10 mL) and evaporated to dryness. The residue was triturated with DCM (25 mL) and filtered to obtain 4-(3-((4-chloro-7-methoxyquinazolin-6-yl)oxy)propyl)morpholine (1.0 g, 57%) as an off white solid. MS (ESI) m/z: 320.2 (M+H⁺).

### General Method C: Formation of Formamidines and Cyclization

### Intermediate C1: tert-Butyl (4-((5-fluoro-7-(2-methoxyethoxy)quinazolin-4-yl)amino)phenyl)carbamate

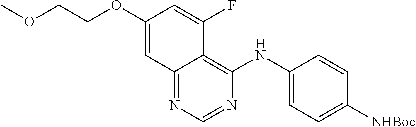 [SMILES: COCCOc1cc(F)c2c(Nc3ccc(NC(=O)OC(C)(C)C)cc3)ncnc2c1]

A suspension of 2-amino-6-fluoro-4-(2-methoxyethoxy)benzonitrile (13 g, 62 mmol) and 1,1-dimethoxy-N,N-dimethylmethanamine (30 g, 250 mmol) was heated at 70° C. for 2 h. The reaction mixture was cooled to rt and then the solution was concentrated to provide colorless viscous intermediate. The residue was treated with tert-butyl (4-aminophenyl)carbamate (13 g, 62 mmol) and acetic acid (100 mL). The resulting reaction mixture was heated at 70° C. for 2 h. The reaction mixture cooled to rt and the reaction mixture was concentrated under reduced pressure. The residue was treated with water (100 mL) and stirred at rt. Solids were filtered, washed with water, and dried under high vacuum to provide tert-butyl (4-((5-fluoro-7-(2-methoxyethoxy)quinazolin-4-yl)amino)phenyl)carbamate (18.6 g, 70%) as a brown solid. MS (ESI) m/z: 429.2 (M+H⁺).

Using the General Method C above, the following Intermediates of Table C were prepared.

TABLE C

| | | | | | MS |
|---|---|---|---|---|---|
| | | | | | (m/z: |
| Ex. | | | Yield | ¹H NMR (400 or 500 MHz, | M + |
| No | SM | Product | (%) | DMSO-d₆): δ | H⁺) |
| C2 | 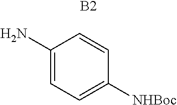 | 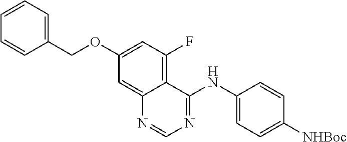 [SMILES: CC(C)(C)OC(=O)Nc1ccc(Nc2ncnc3cc(OCc4ccccc4)cc(F)c23)cc1] | 78 | 9.31 (s, 1H), 8.89 (d, J = 12.4 Hz, 1H), 8.42 (s, 1H), 7.55 (d, J = 8.8 Hz, 2H), 7.50 (d, J = 7.2 Hz, 2H), 7.34-7.45 (m, 5H), 7.18 (dd, J = 14.0, 2.4 Hz, 1H), 7.13 (d, J = 2.0 Hz, 1H), 5.29 (s, 2H), 1.48 (s, 9H). | 461.3 |
| C3 | 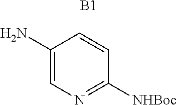 | 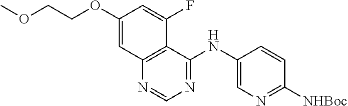 [SMILES: COCCOc1cc(F)c2c(Nc3ccc(NC(=O)OC(C)(C)C)nc3)ncnc2c1] | 89 | No Data | 430.2 |
| C4 | 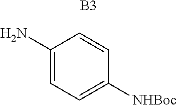 | 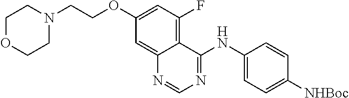 [SMILES: CC(C)(C)OC(=O)Nc1ccc(Nc2ncnc3cc(OCCN4CCOCC4)cc(F)c23)cc1] | 50 | 9.33 (s, 1H), 8.90 (d, J = 12.4 Hz, 1H), 8.42 (s, 1H), 7.54 (d, J = 8.8 Hz, 2H), 7.44 (d, J = 8.8 Hz, 2H), 7.11 (dd, J = 14.0, 2.4 Hz, 1H), 7.05 (d, J = 2.0 Hz, 1H), 4.27 (t, J = 5.2 Hz, | 484.0 |
| | | | | 2H), 3.59 (m, 4H), 2.74 (t, | |
| | | | | J = 5.2 Hz, 2H), 2.49 (m, | |
| | | | | 4H). 1.48 (s, 9H). | |
| C5 | 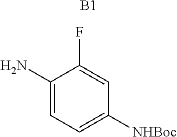 | 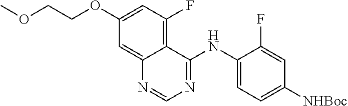 [SMILES: COCCOc1cc(F)c2c(Nc3ccc(NC(=O)OC(C)(C)C)cc3F)ncnc2c1] | 62 | 9.60 (s, 1H), 8.87 (d, J = 12.0 Hz, 1H), 8.38 (s, 1H), 7.54 (t, J = 8.8 Hz, 1H), 7.46 (d, J = 12.8 Hz, 1H), 7.22 (d, J = 8.4 Hz, 1H), 7.13 (dd, J = 13.6, 2.0 Hz, 1H), 7.06 (d, J = 2.0 Hz, 1H), 4.28 (m, 2H), 3.71 (m, 2H), 3.32 (s, 3H), 1.49 | 447.3 |
| | | | | (s, 9H). | |
| C6 | 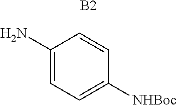 | 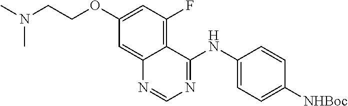 [SMILES: CN(C)CCOc1cc(F)c2c(Nc3ccc(NC(=O)OC(C)(C)C)cc3)ncnc2c1] | 57 | 9.34 (s, 1H), 8.89 (d, J = 12.4 Hz, 1H), 8.42 (s, 1H), 7.54 (t, J = 8.8 Hz, 2H), 7.44 (d, J = 8.8 Hz, 2H), 7.11 (dd, J = 12.0, 2.4 Hz, 1H), 7.06 (m, 1H), 4.22 (t, J = 5.6 Hz, 2H), 2.67 (t, J = | 442.3 |
| | | | | 5.6 Hz, 2H), 2.23 (s, 6H), | |
| | | | | 1.48 (s, 9H). | |
| C7 | 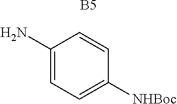 [SMILES: "B5" ; CC(C)(C)OC(=O)Nc1ccc(N)cc1] | 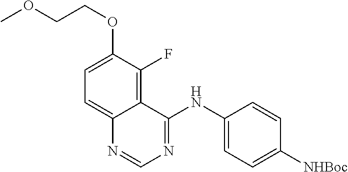 [SMILES: COCCOc1ccc2ncnc(Nc3ccc(NC(=O)OC(C)(C)C)cc3)c2c1F] | 50 | 9.32 (s, 1H), 8.97 (d, J = 13.2 Hz, 1H), 8.40 (s, 1H), 7.83 (t, J = 9.2 Hz, 1H), 7.62 (d, J = 8.8 Hz, 2H), 7.60 (s, 1H), 7.44 (d, J = 8.4 Hz, 2H), 4.32 (m, 2H), 3.71 (m, 2H), 3.31 (s, 3H), 1.47 (s, 9H). | 429.1 |
| C8 | 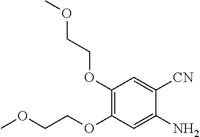 [SMILES: COCCOc1cc(N)c(C#N)cc1OCCOC] | 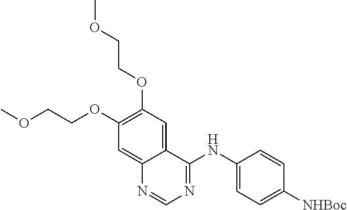 [SMILES: COCCOc1cc2ncnc(Nc3ccc(NC(=O)OC(C)(C)C)cc3)c2cc1OCCOC] | 44 | 9.60 (s, 1H), 8.30 (br s, 1H), 7.77 (s, 1H), 7.68 (s, 1H), 7.61 (d, J = 8.0 Hz, 2H), 7.34 (d, J = 8.4 Hz, 2H), 7.01 (s, 1H), 4.21 (m, 2H), 4.18 (m, 2H), 3.70 (m, 4H), 3.31 (s, 6H), 1.49 (s, 9H). | 485.1 |
| | 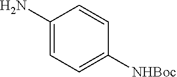 [SMILES: CC(C)(C)OC(=O)Nc1ccc(N)cc1] | | | | |
| C9 | 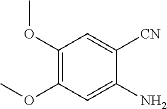 | 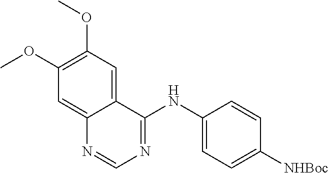 [SMILES: COc1cc2ncnc(Nc3ccc(NC(=O)OC(C)(C)C)cc3)c2cc1OC] | 36 | 9.42 (br s, 1H), 9.30 (br s, 1H), 8.38 (s, 1H), 7.81 (s, 1H), 7.61 (d, J = 8.8 Hz, 2H), 7.44 (d, J = 8.8 Hz, 2H), 7.15 (s, 1H), 3.93 (s, 3H), 3.91 (s, 3H), 1.48 (s, 9H). | 397.3 |
| | 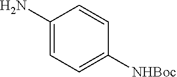 [SMILES: CC(C)(C)OC(=O)Nc1ccc(N)cc1] | | | | |
| C10 | 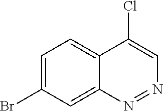 [SMILES: Clc1cnnc2cc(Br)ccc12] | 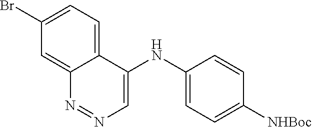 [SMILES: CC(C)(C)OC(=O)Nc1ccc(Nc2cnnc3cc(Br)ccc23)cc1] | crude | No Data | 415.0 417.0 |
| | 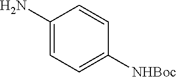 [SMILES: CC(C)(C)OC(=O)Nc1ccc(N)cc1] | | | | |
| C11 | 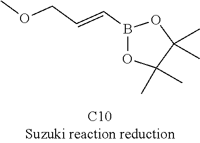 | 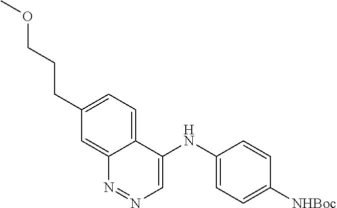 [SMILES: COCCCc1ccc2c(Nc3ccc(NC(=O)OC(C)(C)C)cc3)cnnc2c1] | 60 | No Data | 409.2 |
| C12 | 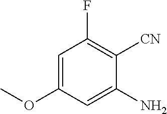 | 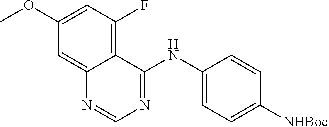 [SMILES: COc1cc(F)c2c(Nc3ccc(NC(=O)OC(C)(C)C)cc3)ncnc2c1] | 50 | 9.31 (br s, 1H), 8.88 (d, J = 12.4 Hz, 1H), 8.42 (s, 1H), 7.54 (br m, 2H), 7.44 (br m, 2H), 7.09 (d, J = 13.6 Hz, 1H), 7.02 (d, J = 1.6 Hz, 1H), 3.91 (s, 3H), 1.47 (s, 9H). | 385.1 |
| | 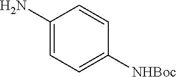 [SMILES: CC(C)(C)OC(=O)Nc1ccc(N)cc1] | | | | |
| C13 | 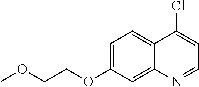 [SMILES: COCCOc1ccc2c(Cl)ccnc2c1] | 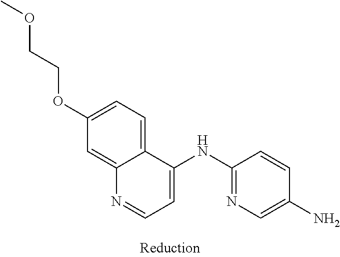 [SMILES: COCCOc1ccc2c(Nc3ccc(N)cn3)ccnc2c1] | 77 | 8.94 (s, 1H), 8.38 (m, 1H), 8.28 (m, 1H), 7.77 (d, J = 2.6 Hz, 1H), 7.65 (d, J = 5.4 Hz, 1H), 7.25 (m, 1H), 7.16 (m, 1H), 7.06 (m, 2H), 5.06 (s, 2H), 4.21-4.32 (m, 2H), 3.74 (m, 2H), 3.35 (s, 3H). | 311.2 |
| | 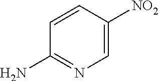 | | | | |
| C14 | 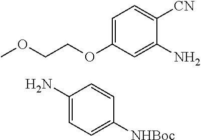 | 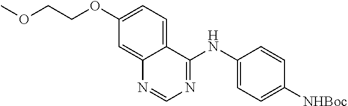 [SMILES: COCCOc1ccc2c(Nc3ccc(NC(=O)OC(C)(C)C)cc3)ncnc2c1] | 40 | 9.57 (s, 1H), 9.31 (br s, 1H), 8.45 (s, 1H), 8.42 (d, J = 9.2 Hz, 1H), 7.65 (d, J = 9.2 Hz, 2H), 7.44 (d, J = 8.8 Hz, 2H), 7.22 (dd, J = 9.2, 2.4 Hz, 1H), 7.15 (d, J = 2.8 Hz, 1H), 4.26 (m, 2H), 3.72 (m, 2H), 1.48 (s, 9H), 3Hs are under solvent | 411.5 |
| C15 | 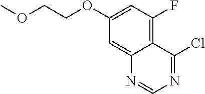 [SMILES: COCCOc1cc(F)c2c(Cl)ncnc2c1] | 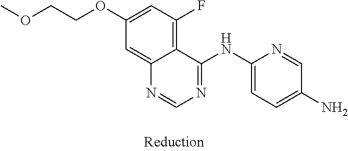 [SMILES: COCCOc1cc(F)c2c(Nc3ccc(N)cn3)ncnc2c1] | 50 | No Data | 330.2 |
| | 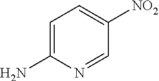 | | | | |

General Method D: Substitution with NaOMe

### Intermediate D1: tert-butyl (4-((5-methoxy-7-(2-methoxyethoxy)quinazolin-4-yl)amnino)phenyl)carbamate

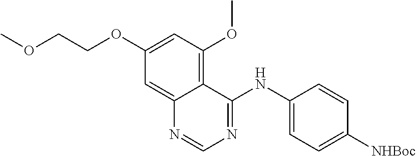 [SMILES: COCCOc1cc(OC)c2c(Nc3ccc(NC(=O)OC(C)(C)C)cc3)ncnc2c1]

A stirred solution of tert-butyl (4-((5-fluoro-7-(2-methoxyethoxy)quinazolin-4-yl)amino)phenyl)carbamate (5.0 g, 11.7 mmol) in dry THF (100 mL) was treated with NaOMe (25% in MeOH, 5.0 mL, 23.4 mmol) at rt. The reaction mixture was heated at 50° C. for 20 h. The reaction mixture was cooled to rt and then poured into the ice water (500 mL) slowly. The precipitated solids were filtered, washed thoroughly with water, and dried under high vacuum. The solids were further purified on silica gel column chromatography (6-8% MeOH/DCM) to afford tert-butyl (4-((5-methoxy-7-(2-methoxyethoxy)quinazolin-4-yl)amino)phenyl)carbamate (3.0 g, 58%) as an off white solid. ¹H NMR (500 MHz, DMSO-d₆): δ 9.67 (s, 1H), 9.29 (br s, 1H), 8.36 (s, 1H), 7.64 (d, J=8.8 Hz, 2H), 7.44 (d, J=8.4 Hz, 2H), 6.76 (d, J=2.0 Hz, 1H), 6.69 (d, J=2.4 Hz, 1H), 4.23 (m, 2H), 4.07 (s, 3H), 3.71 (m, 2H), 3.30 (s, 3H), 1.48 (s, 9H); MS (ESI) m/z: 441.1 (M+H⁺).

### General Method E: Deprotection

### Intermediate E1: N1-(5-Fluoro-7-(2-methoxyethoxy)quinazolin-4-yl)benzene-1,4-diamine hydrochloride

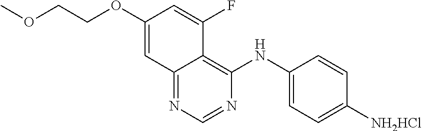

A suspension of tert-butyl (4-((5-fluoro-7-(2-methoxyethoxy)quinazolin-4-yl)amino)phenyl)carbamate (12.0 g, 28 mmol) in dioxane (24 mL), 4.0 M HCl in 1,4-dioxane (24 Ml, 96 mmol) was stirred at rt for 6 h. MTBE was added and the solution was stirred at rt. Solids were filtered, washed with MTBE and acetonitrile to provide N1-(5-fluoro-7-(2-methoxyethoxy)quinazolin-4-yl)benzene-1,4-diamine hydrochloride (9.4 g, 92%) as gray colored solid. ¹H NMR (500 MHz, DMSO-d₆): δ 10.4 (br s, 1H), 8.75 (s, 1H), 7.56 (br d, J=8.8 Hz, 2H), 7.46 (dd, J=14.4, 2.0 Hz, 1H), 7.27 (br d, J=8.4 Hz, 2H), 7.20 (d, J=1.6 Hz, 1H), 4.34 (m, 2H), 3.73 (m, 2H), 3.40-4.10 (br s, 3H), 3.33 (s, 3H); MS (ESI) m/z: 329.2 (M+H⁺).

Using the General Methods D, and E above, the following Intermediates of Table D were prepared.

TABLE D

| | | | | | MS |
|---|---|---|---|---|---|
| | | | | | (m/z: |
| Ex. | | | Yield | ¹H NMR (400 or 500 MHz, | M + |
| No | SM | Product | (%) | DMSO-d₆): δ | H⁺) |
| E2 | C2 | 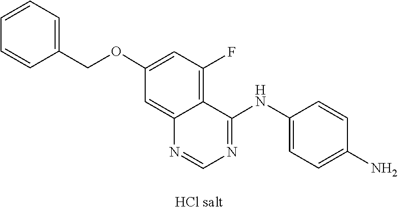 [SMILES: Nc1ccc(Nc2ncnc3cc(OCc4ccccc4)cc(F)c23)cc1] | 98 | 10.6 (br s, 1H), 8.81 (s, 1H), 7.64 (d, J = 8.8 Hz, 2H), 7.57 (d, J = 2.4 Hz, 1H), 7.53 (m, 3H), 7.49 (d, J = 8.8 Hz, 2H), 7.40-7.46 (m, 5H), 5.36 (s, 2H). | 361.1 |
| E3 | D1 | 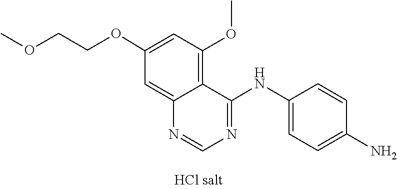 [SMILES: COCCOc1cc(OC)c2c(Nc3ccc(N)cc3)ncnc2c1] | 93 | 10.7 (s, 1H), 8.70 (s, 1H), 7.51 (d, J = 8.4 Hz, 2H), 7.20 (d, J = 8.0 Hz, 2H), 6.95 (br s, 1H), 6.91 (br s, 1H), 4.31 (m, 2H), 4.12 (s, 3H), 3.74 (m, 2H), 3.34 (s, 3H). (NH2 protons are not visible) | 341.0 |
| E4 | C3 | 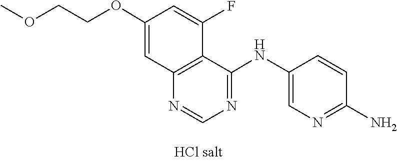 [SMILES: COCCOc1cc(F)c2c(Nc3ccc(N)nc3)ncnc2c1] | 99 | No Data | 330.2 |
| E5 | C4 | 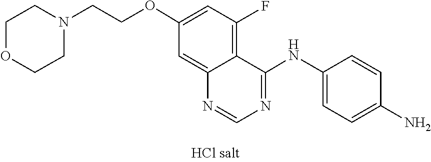 [SMILES: Nc1ccc(Nc2ncnc3cc(OCCN4CCOCC4)cc(F)c23)cc1] | 98 | 10.5 (br s, 1H), 8.80 (s, 1H), 7.63 (d, J = 8.8 Hz, 2H), 7.52 (dd, J = 13.6, 2.0 Hz, 1H), 7.42 (d, J = 8.4 Hz, 2H), 7.36 (d, J = 2.0 Hz, 1H), 4.70 (t, J = 4.0 HZ, 2H), 3.94 (m, 4H), 3.63 (t, J = 4.4 Hz, 2H), 3.50 (m, 2H), 3.36 (m, 2H). NH₂ protons are missing | 384.0 |
| E6 | C5 | 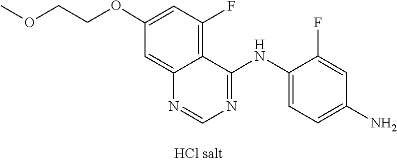 [SMILES: COCCOc1cc(F)c2c(Nc3ccc(N)cc3F)ncnc2c1] | 96 | 10.3 (br s, 1H), 8.78 (s, 1H), 7.47 (dd, J = 13.6, 2.0 Hz, 1H), 7.23 (t, J = 8.4 Hz, 1H), 7.20 (d, J = 2.0 Hz, 1H), 6.65 (m, 2H), 4.34 (m, 2H), 3.73 (m, 2H), 3.32 (s, 3H). NH₂ protons are missing | 347.4 |
| E7 | C6 | 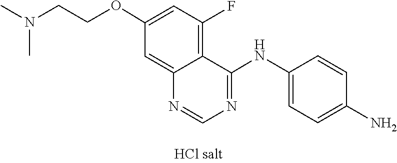 [SMILES: CN(C)CCOc1cc(F)c2c(Nc3ccc(N)cc3)ncnc2c1] | 98 | 10.9 (br s, 1H), 10.4 (br s, 1H), 8.77 (s, 1H), 7.61 (d, J = 8.4 Hz, 2H), 7.49 (d, J = 13.6, 1H), 7.34 (m, 3H), 4.61 (m, 2H), 3.59 (m, 2H), 2.85 (s, 6H). NH₂ protons are missing | 342.3 |
| E8 | C7 | 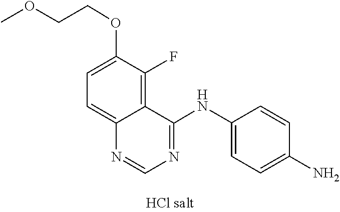 [SMILES: COCCOc1ccc2ncnc(Nc3ccc(N)cc3)c2c1F] | 98 | 10.9 (s, 1H), 8.79 (s, 1H), 8.11 (t, J = 9.2 Hz, 1H), 7.81 (dd, J = 10.0, 1.6 Hz, 1H), 7.79 (d, J = 8.8 Hz, 2H), 7.44 (d, J = 8.8 Hz, 2H), 4.41 (m, 2H), 3.68 (m, 2H), 3.39 (s, 3H). NH₂ protons are missing | 329.3 |
| E9 | C8 | 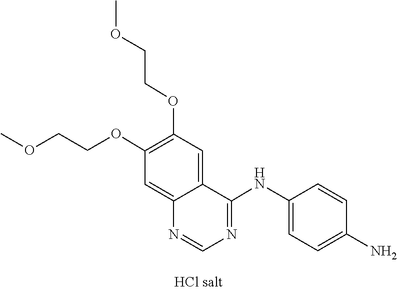 [SMILES: COCCOc1cc2ncnc(Nc3ccc(N)cc3)c2cc1OCCOC] | 99 | 10.2 (s, 1H), 8.56 (br s, 1H), 8.37 (s, 1H), 8.25 (s, 1H), 7.54 (d, J = 8.4 Hz, 2H), 7.43 (s, 1H), 7.21 (d, J = 7.6 Hz, 2H), 4.38 (m, 2H), 4.31 (m, 2H), 3.75 (m, 4H), 3.34 (s, 6H). ). NH₂ protons are missing | 385.3 |
| E10 | C9 | 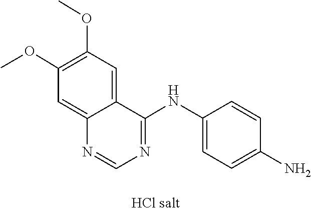 [SMILES: COc1cc2ncnc(Nc3ccc(N)cc3)c2cc1OC] | 98 | 11.5 (s, 1H), 8.81 (d, J = 2.4 Hz 1H), 8.33 (t, J = 6.4 Hz, 1H), 7.72 (dd, J = 8.8, 2.8 Hz, 2H), 7.34 (m, 3H), 4.01 (s, 3H), 3.99 (s, 3H). NH₂ protons are missing | 297.3 |
| E11 | C11 | 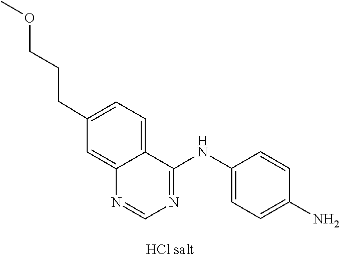 [SMILES: COCCCc1ccc2c(Nc3ccc(N)cc3)ncnc2c1] | 83 | 11.70 (s, 1H), 8.71 (d, J = 8.7 Hz, 1H), 8.40 (s, 1H), 7.80 (d, J = 8.3 Hz, 2H), 7.37 (d, J = 8.0 Hz, 2H), 7.03 (s, 1H), 4.32 (s, 2H), 3.38 (m, 2H), 3.27 (s, 3H), 2.92 (t, J = 7.7 Hz, 2H), 1.88-1.97 (m, 2H). | 309.2 |
| E12 | C12 | 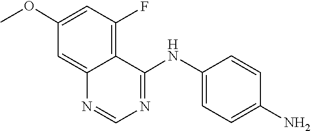 [SMILES: COc1cc(F)c2c(Nc3ccc(N)cc3)ncnc2c1] | 95 | 10.50 (br s, 1H), 8.79 (s, 1H), 7.61 (d, J = 8.2 Hz, 2H), 7.45 (dd, J = 13.6, 2.4 Hz, 1H), 7.40 (d, J = 8.4 Hz, 2H), 7.28 (d, J = 1.6 Hz, 1H), 3.98 (s, 3H). NH₂ are missing. | 284.9 |
| E13 | C14 | 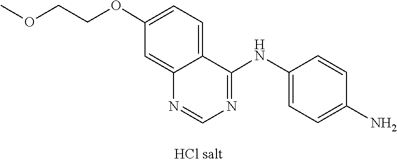 [SMILES: COCCOc1ccc2c(Nc3ccc(N)cc3)ncnc2c1] | 95 | 11.64 (s, 1H), 8.85 (t, J = 4.4 Hz, 2H), 7.71 (d, J = 8.8 Hz, 2H), 7.51 (dd, J = 9.2, 2.4 Hz, 1H), 7.36 (d, J = 2.4 Hz, 1H), 7.34 (s, 1H), 7.31 (s, 1H), 4.33 (t, J = 4.0 Hz, 2H), 3.75 (t, J = 4.4 Hz, 2H), 3.33 (s, 3H). 2Hs are missing | 311.0 |
| E14 | C15 | 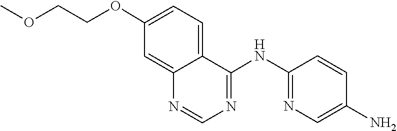 [SMILES: COCCOc1ccc2c(Nc3ccc(N)cn3)ncnc2c1] | 59 | 9.84 (s, 1H), 8.49 (d, J = 9.2 Hz, 1H), 8.46 (s, 1H), 7.85 (d, J = 8.8 Hz, 1H), 7.73 (d, J = 2.4 Hz, 1H), 7.15 (m, 2H), 7.04 (dd, J = 8.8, 2.8 Hz, 1H), 5.18 (br s, 2H), 4.25 (m, 2H), 3.71 (m, 2H). 3Hs are under solvents. | 311.9 |

General Method F: Activation of Amine with TrocCl

### Example F1: 2,2,2-trichloroethyl (4-((5-fluoro-7-(2-methoxyethoxy)quinazolin-4-yl)amino)phenyl)carbamate

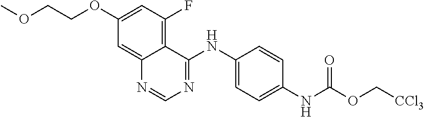

A suspension of N1-(5-fluoro-7-(2-methoxyethoxy)quinazolin-4-yl)benzene-1,4-diamine hydrochloride (5.0 g, 14 mmol) in DCM (10 mL) was treated with TEA (4.0 g, 40 mmol). The reaction mixture was stirred at rt and TrocCl (5.0 g, 24 mmol) was added. The reaction mixture stirred at rt for 2 h and water (100 mL) was added. The organic layer was washed with brine, dried over anhydrous Na₂SO₄, filtered, and concentrated under reduced pressure. The residue was treated with MTBE (60 mL) and the solids were filtered to provide, 2,2,2-trichloroethyl(4-((5-fluoro-7-(2-methoxyethoxy)quinazolin-4-yl)amino)phenyl)carbamate (5.8 g, 84%) as a light greenish solid. MS (ESI) m/z: 503.0 (M+H⁺) and 505.0.

Using the General Method F above, the following Intermediates of Table E were prepared.

TABLE E

| | | | | | MS |
|---|---|---|---|---|---|
| | | | | | (m/z: |
| Ex. | | | Yield | ¹H NMR (400 or 500 MHz, | M + |
| No | SM | Product | (%) | DMSO-d₆): δ | H⁺) |
| F2 | E4 | 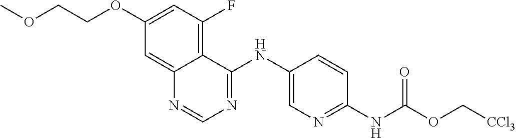 | crude | No Data | 504.0 506.0 |
| F3 | E5 | 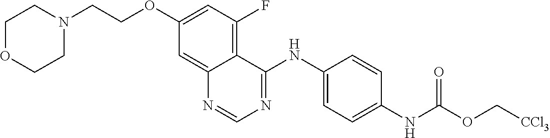 | 61 | 10.12 (br s, 1H), 8.95 (d, J = 12.4 Hz, 1H), 8.43 (s, 1H), 7.63 (d, J = 8.8 Hz, 2H), 7.50 (d, J = 8.4 Hz, 2H), 7.13 (dd, J = 14.0, 2.4 Hz, 1H), 7.06 (d, J = 2.0 Hz, 1H), 4.95 (s, 2H), 4.27 (t, J = 5.6 Hz, 2H), 3.58 (m, 4H), 2.74 (t, J = 5.6 Hz, 2H), 2.49 (m, 4H). | 557.9 559.9 |
| F4 | E6 | 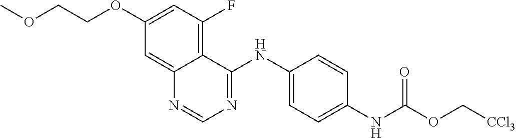 | 82 | 10.4 (s, 1H), 8.93 (d, J = 12.0 Hz, 1H), 8.40 (s, 1H), 7.63 (t, J = 8.4 Hz, 1H), 7.50 (d, J = 12.0 Hz, 1H), 7.32 (d, J = 8.8 Hz, 1H), 7.14 (dd, J = 13.6, 2.0 Hz, 1H), 7.07 (d, J = 2.4 Hz, 1H), 4.97 (s, 2H), 4.28 (m, 2H), 3.71 (m, 2H), 3.31 (s, 3H). | 521.1 523.1 |
| F5 | E7 | 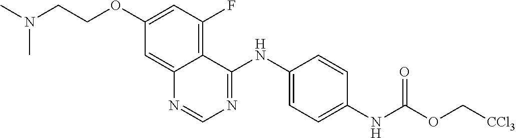 | 20 | 10.1 (br s, 1H), 8.95 (d, J = 12.4 Hz, 1H), 8.43 (s, 1H), 7.62 (d, J = 8.8 Hz, 2H), 7.50 (d, J = 8.4 Hz, 2H), 7.11 (d, J = 13.6 Hz, 1H), 7.05 (br s, 1H), 4.95 (s, 2H), 4.23 (m, 2H), 2.60 (m, 2H), 2.20 (s, 6H). | 516.2 518.2 |
| F6 | E9 | 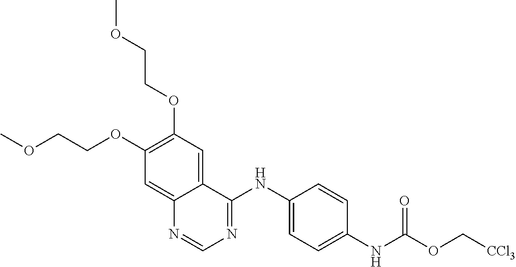 | 37 | 10.5 (br s, 1H), 8.03 (s, 1H), 7.83 (s, 1H), 7.73 (d, J = 8.0 Hz, 2H), 7.65 (s, 1H), 7.51 (d, J = 8.4 Hz, 2H), 7.19 (m, 1H), 4.98 (s, 2H), 4.23 (m, 2H), 4.20 (m, 2H), 3.72 (m, 4H), 3.32 (s, 6H). | 560.8 |
| F7 | E3 | 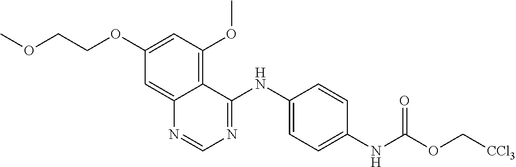 | 25 | 10.1 (br s, 1H), 9.80 (br s, 1H), 8.34 (s, 1H), 7.62 (d, J = 7.6 Hz, 2H), 7.50 (d, J = 7.2 Hz, 2H), 6.77 (d, J = 2.0 Hz, 1H), 6.71 (d, J = 2.4 Hz, 1H), 4.98 (s, 2H), 4.23 (m, 2H), 4.07 (s, 3H), 3.70 (m, 2H), 3.31 (s, 3H). | 516.8 |
| F8 | E12 | 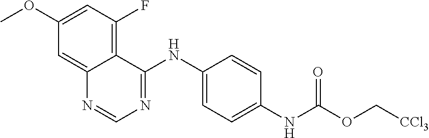 | 31 | 10.45 (br s, 1H), 10.32 (s, 1H), 8.76 (s, 1H), 7.58 (m, 2H), 7.49 (m, 2H), 7.43 (br d, J = 13.2 Hz, 1H), 7.17 (br s, 1H), 4.96 (s, 2H), 3.98 (s, 3H). | 460.8 |
| F9 | E10 | 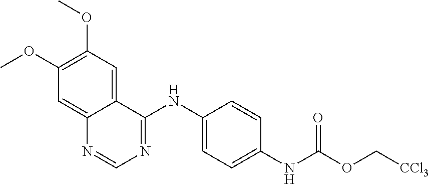 | 35 | 11.3 (br s, 1H), 10.3 (br s, 1H), 8.80 (s, 1H), 8.27 (s, 1H), 7.61 (m, 4H), 7.33 (s, 1H), 5.02 (s, 2H), 4.01 (s, 3H), 3.99 (s, 3H). | 471.1 |
| F10 | E8 | 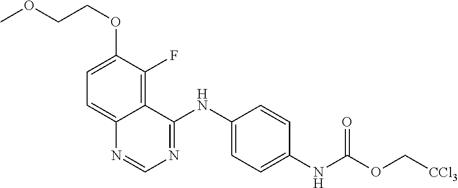 | 40 | 10.7 (s, 1H), 9.04 (d, J = 13.2 Hz, 1H), 8.41 (s, 1H), 7.86 (m, 1H), 7.70 (m, 2H), 7.62 (m, 1H), 7.50 (m, 2H), 4.97 (s, 2H), 4.34 (t, J = 4.4 Hz, 2H), 3.71 (t, J = 4.4 Hz, 2H), 3.32 (s, 3H). | 505.0 |
| F11 | E14 | 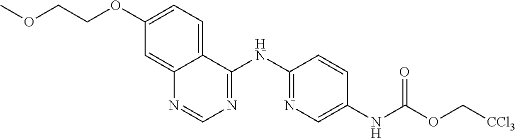 | crude | No Data | 486.0 |

### General Method G: Coupling Reaction

### Example 1: 1-(4-((5-fluoro-7-(2-methoxyethoxy)quinazolin-4-yl)amino)phenyl)-3-(4,4,4-trifluoro-3,3-dimethylbutyl)urea

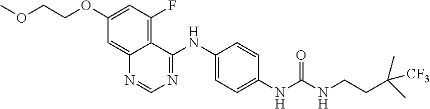 [SMILES: COCCOc1cc(F)c2c(Nc3ccc(NC(=O)NCCC(C)(C)C(F)(F)F)cc3)ncnc2c1]

A suspension of 2,2,2-trichloroethyl(4-((5-fluoro-7-(2-methoxyethoxy)quinazolin-4-yl)amino)phenyl)carbamate (F1, 0.30 g, 0.60 mmol) in CH₃CN (10 mL) was treated with followed TEA (0.150 g, 0.0015 mmol). 4,4,4-Trifluoro-3,3-dimethylbutan-1-amine hydrochloride (0.10 g, 0.52 mmol) was added and the reaction mixture was stirred at 60° C. for 14 h. The reaction mixture cooled to rt and concentrated under reduced pressure. The residue was dissolved in DCM (10 mL). The solution was filtered through a pad of silica gel and washed with EtOAc (20 mL). The filtrate was concentrated under reduced pressure and crystallized from CH₃CN to provide 1-(4-((5-fluoro-7-(2-methoxyethoxy)quinazolin-4-yl)amino)phenyl)-3-(4,4,4-trifluoro-3,3-dimethylbutyl)urea (0.14 g, 46%) as a white solid. ¹H NMR (500 MHz, DMSO-d₆): δ 8.86 (d, J=12.6 Hz, 1H), 8.50 (s, 1H), 8.40 (s, 1H), 7.50 (d, J=8.7 Hz, 2H), 7.38 (d, J=8.7 Hz, 2H), 7.11 (dd, J=13.8, 2.4 Hz, 1H), 7.03 (d, J=2.4 Hz, 1H), 6.13 (t, J=5.8 Hz, 1H), 4.27 (t, J=4.3 Hz, 2H), 3.70 (t, J=4.3 Hz, 2H), 3.33 (s, 3H), 3.19 (m, 2H), 1.62 (m, 2H), 1.12 (s, 6H); MS (ESI) m/z: 510.2 (M+H⁺).

### General Method H: Curtius Reaction

### Example 2: 1-(3-fluoro-3-methylbutyl)-3-(4-((5-fluoro-7-(2-methoxyethoxy)quinazolin-4-yl)amino)phenyl)urea

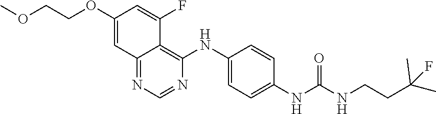 [SMILES: COCCOc1cc(F)c2c(Nc3ccc(NC(=O)NCCC(C)(C)F)cc3)ncnc2c1]

A suspension of 5-fluoro-5-methylhexanoic acid (0.25 g, 1.7 mmol) in 1,4-dioxane (10 mL) was treated with TEA (0.5 mL). The reaction mixture was cooled to 0° C. under an ice-bath and diphenylphosphoryl azide (0.50 g, 1.8 mmol) was added under the same conditions. The reaction mixture slowly warmed to rt and then stirred at rt for 30 min. N1-(5-fluoro-7-(2-methoxyethoxy)quinazolin-4-yl)benzene-1,4-diamine hydrochloride (E1, 0.30 g, 0.82 mmol) followed by TEA (0.5 mL) and stirred at 100° C. for 2 h. The reaction mixture was cooled to rt and a precipitate formed. The precipitated solids were filtered off and washed with acetonitrile. The filtrate was concentrated under reduced pressure and partitioned between DCM (30 mL) and water (20 mL). The organic layer was dried over anhydrous Na₂SO₄, filtered, and concentrated under reduced pressure to obtain dark residue. The resulting solid was dissolved in DCM (10 mL) and filtered through a pad of silica gel. The filtrate was treated with CH₃CN and the solid was filtered to provide, 1-(3-fluoro-3-methylbutyl)-3-(4-((5-fluoro-7-(2-methoxyethoxy)quinazolin-4-yl)amino)phenyl)urea (0.14 g, 37%) as white solids. ¹H NMR (500 MHz, DMSO-d₆): δ 8.86 (d, J=12.6 Hz, 1H), 8.50 (s, 1H), 8.40 (s, 1H), 7.50 (d, J=8.6 Hz, 2H), 7.38 (d, J=8.6 Hz, 2H), 7.11 (dd, J=13.8, 2.4 Hz, 1H), 7.03 (d, J=2.3 Hz, 1H), 6.07 (t, J=5.7 Hz, 1H), 4.27 (t, J=4.3 Hz, 2H), 3.70 (t, J=4.2 Hz, 2H), 3.33 (s, 3H), 3.21 (m, 2H), 1.78 (m, 2H); 1.36 (s, 3H), 1.32 (s, 3H); MS (ESI) m/z: 460.2 (M+H⁺).

General Method I: Amide Formation with HATU

### Example 3: N-(4-((5-fluoro-7-(2-methoxyethoxy)quinazolin-4-yl)amino)phenyl)-4,4-dimethylpentanamide

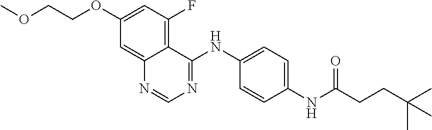 [SMILES: COCCOc1cc(F)c2c(Nc3ccc(NC(=O)CCC(C)(C)C)cc3)ncnc2c1]

A suspension of N1-(5-fluoro-7-(2-methoxyethoxy)quinazolin-4-yl)benzene-1,4-diamine hydrochloride (E1, 0.20 g, 0.55 mmol) in CH₃CN (10 mL) was treated with HATU (0.25 g, 0.66 mmol), DIEA (0.25 g, 1.9 mmol) and 4,4-dimethylpentanoic acid (0.10 g, 0.77 mmol). The reaction mixture was stirred at 70° C. for 30 min. The reaction mixture was cooled to rt and the resulting solids were filtered, washed with CH₃CN to provide N-(4-((5-fluoro-7-(2-methoxyethoxy)quinazolin-4-yl)amino)phenyl)-4,4-dimethylpentanamide (0.15 g, 62%) as a white solid. ¹H NMR (500 MHz, DMSO-d₆): δ 9.90 (s, 1H), 8.92 (d, J=12.4 Hz, 1H), 8.42 (s, 1H), 7.58 (s, 4H), 7.12 (dd, J=13.8, 2.4 Hz, 1H), 7.04 (s, 1H), 4.27 (t, J=4.2 Hz, 2H), 3.70 (t, J=4.2 Hz, 2H), 3.32 (s, 3H), 2.27 (t, J=8.2 Hz, 2H), 1.51 (t, J=8.3 Hz, 2H), 0.90 (s, 9H); MS (ESI) m/z: 441.2 (M+H⁺).

General Method J: Amide Formation with Acyl Chloride

### Example G1: methyl 3-((4-((5-fluoro-7-(2-methoxyethoxy)quinazolin-4-yl)amino)phenyl)amino)-3-oxopropanoate

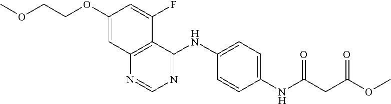

A suspension of N1-(5-fluoro-7-(2-methoxyethoxy)quinazolin-4-yl)benzene-1,4-diamine hydrochloride (E1, 0.60 g, 1.6 mmol) in DCM (10 mL) was treated with TEA (0.17 g, 1.6 mmol) at rt. Methyl 3-chloro-3-oxopropanoate (0.60 g, 4.4 mmol) was slowly added into the reaction mixture and the resulting reaction mixture stirred at rt for 1 h. The mixture was concentrated and water was added. The solution was stirred and the solids were filtered, washed with water, and dried under high vacuum to provide methyl 3-((4-((5-fluoro-7-(2-methoxyethoxy)quinazolin-4-yl)amino)phenyl)amino)-3-oxopropanoate (0.48 g, 68%) as an off-white solid. ¹H NMR (500 MHz, DMSO-d₆): δ 10.22 (s, 1H), 8.97 (d, J=12.3 Hz, 1H), 8.46 (s, 1H), 7.63 (d, J=9.2 Hz, 2H), 7.59 (d, J=9.2 Hz, 2H), 7.15 (dd, J=13.9, 2.4 Hz, 1H), 7.06 (d, J=2.4 Hz, 1H), 4.28 (m, 2H), 3.71 (m, 2H), 3.67 (s, 3H), 3.49 (s, 2H), 3.33 (s, 3H). MS (ESI) m/z: 429.0 (M+H⁺).

Using the General Methods G, H, I and J above, the following Intermediates of Table F were prepared.

TABLE F

| Ex. No | SM | | Product | Method |
|---|---|---|---|---|
| G2 | 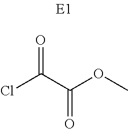 | | 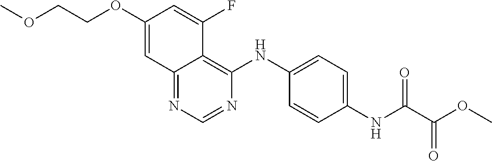 | J |
| G3 | 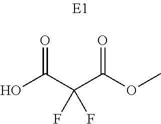 | | 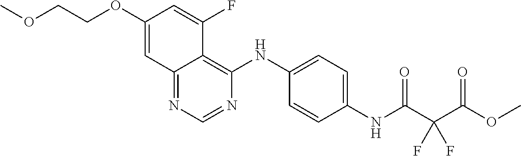 | I |
| G4 | 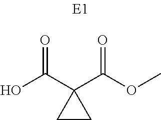 | | 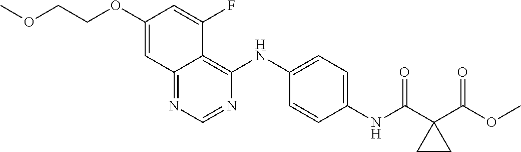 | I |
| G5 | 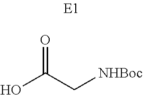 | | 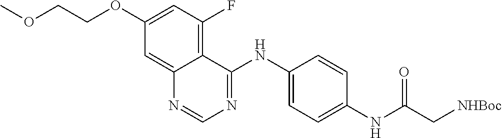 [SMILES: COCCOc1cc(F)c2c(Nc3ccc(NC(=O)CNC(=O)OC(C)(C)C)cc3)ncnc2c1] | I |
| G6 | 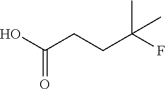 | | 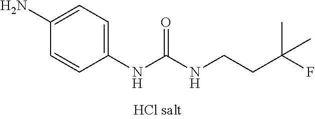 [SMILES: CC(C)(F)CCNC(=O)Nc1ccc(N)cc1] | H & E |
| | 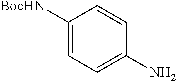 | | | |
| G7 | 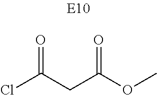 | | 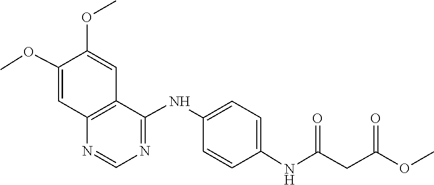 | J |
| G8 | 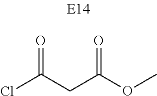 | | 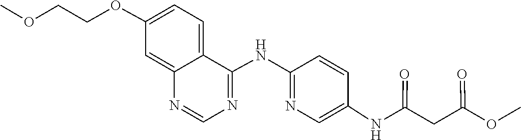 | J |
| | Ex. | | ¹H NMR (400 or 500 | MS (m/z: |
| | No | Yield (%) | MHz, DMSO-d₆): δ | M + H⁺) |
| | G2 | 85 | 10.82 (s, 1H), 8.99 (d, J = 12.1 Hz, 1H), 8.45 (s, 1H), 7.71 (d, J = 8.8 Hz, 2H), 7.65 | 415.2 |
| | | | (d, J = 8.8 Hz, 2H), 7.14 (dd, J = 13.9, 2.4 Hz, 1H), 7.05 (d, J = 2.4 Hz, 1H), 4.28 (m, | |
| | | | 2H), 3.81 (s, 3H), 3.64 (m, 2H), 3.31 (s, 3H). | |
| | G3 | 66 | No Data | 451.2 |
| | G4 | 58 | 10.35 (s, 1H), 8.96 (d, J = 12.3 Hz, 1H), 8.45 (s, 1H), 7.58-7.68 (m, 4H), 7.15 | 465.2 |
| | | | (dd, J = 13.9, 2.4 Hz, 1H), 7.06 (d, J = 2.4 Hz, 1H), 4.29 (m, 2H), 3.71 (m, 2H), | |
| | | | 3.70 (s, 3H), 3.34 (s, 3H), 1.37-1.48 (m, 4H). | |
| | G5 | 50 | 9.93 (s, 1H), 8.95 (d, J = 12.4 Hz, 1H), 8.44 (s, 1H), 7.62 (d, J = 9.2 Hz, 2H), 7.57 | 486.5 |
| | | | (d, J = 8.8 Hz, 2H), 7.11 (dd, J = 14.0, 2.4 Hz, 1H), 7.05 (d, J = 2.0 Hz, 1H), 4.28 (m, | |
| | | | 2H), 3.71 (m, 4H), 3.32 (s, 3H), 1.44 (s, 9H). | |
| | G6 | 90 | No Data | 340.2 |
| | G7 | 61 | No Data | 397.2 |
| | G8 | 50 | 10.50 (s, 1H), 9.60 (br s, 1H), 8.67 (d, J = 2.4 Hz, 1H), 8.60 (d, J = 7.2 Hz, 2H), | 411.9 |
| | | | 8.26 (br s, 1H), 7.99 (dd, J = 6.0, 2.8 Hz, 1H), 7.24 (m, 2H), 4.28 (m, 2H), 3.72 | |
| | | | (m, 2H), 3.64 (s, 3H), 3.53 (s, 2H), 3.33 (s, 3H). | |

### General Method K: Hydrolysis

### Intermediate H1: 3-((4-((5-fluoro-7-(2-methoxyethoxy)quinazolin-4-yl)amino)phenyl)amino)-3-oxopropanoic acid

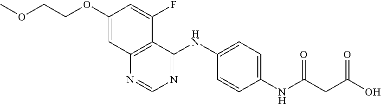 [SMILES: COCCOc1cc(F)c2c(Nc3ccc(NC(=O)CC(=O)O)cc3)ncnc2c1]

A suspension of methyl 3-((4-((5-fluoro-7-(2-methoxyethoxy)quinazolin-4-yl)amino)phenyl)amino)-3-oxopropanoate (G1, 1.0 g, 2.3 mmol), lithium hydroxide hydrate (2.5 g, 60 mmol) in water (60 mL) was stirred at 50° C. for 5 h and then at rt for 2 days. The resulting solids were filtered, washed with water, and dried under high vacuum to provide 3-((4-((5-fluoro-7-(2-methoxyethoxy)quinazolin-4-yl)amino)phenyl)amino)-3-oxopropanoic acid (0.62 g, 64%) as a white solid. MS (ESI) m/z: 415.2 (M+HJ).

Using the General Method E, and K above, the following Intermediates of Table G were prepared.

TABLE G

| | | | | ¹H NMR (400 or 500 MHz, | |
|---|---|---|---|---|---|
| Ex. No | SM | Product | Yield (%) | DMSO-d₆): δ | MS (m/z: M + H⁺) |
| H2 | G2 | 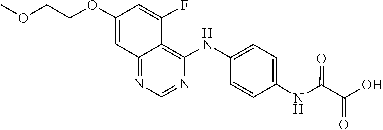 [SMILES: COCCOc1cc(F)c2c(Nc3ccc(NC(=O)C(=O)O)cc3)ncnc2c1] | 76 | 10.73 (s, 1H), 9.11 (br s, 1H), 8.47 (s, 1H), 7.72 (d, J = 8.8 Hz, 2H), 7.65 (d, J = 8.9 Hz, 2H), 7.16 (dd, J = 13.9, 2.4 Hz, 1H), 7.05 (d, J = 2.4 Hz, 1H), 4.27 (m, 2H), 3.69 (m, 2H), 3.31 (s, 3H). acid H missing | 415.2 |
| H3 | G3 | 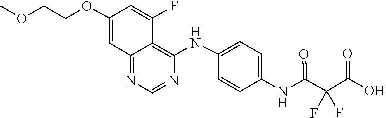 [SMILES: COCCOc1cc(F)c2c(Nc3ccc(NC(=O)C(F)(F)C(=O)O)cc3)ncnc2c1] | 56 | 11.05 (s, 1H), 10.55 (br s, 1H), 8.80 (s, 1H), 7.71 (d, J = 8.8 Hz, 2H), 7.62 (d, J = 8.9 Hz, 2H), 7.51 (dd, J = 14.0, 5.0 Hz, 1H), 7.01 (d, J = 2.4 Hz, 1H), 4.30 (m, 2H), 3.71 (m, 2H), 3.31 (s, 3H). acid H missing | 451.2 |
| H4 | G4 | 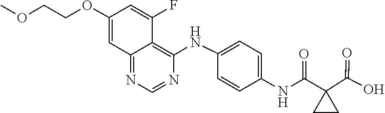 [SMILES: COCCOc1cc(F)c2c(Nc3ccc(NC(=O)C4(C(=O)O)CC4)cc3)ncnc2c1] | 86 | 13.15 (s, 1H), 10.64 (s, 1H), 9.35 (s, 1H), 8.54 (s, 1H), 7.57- 7.66 (m, 4H), 7.23 (dd, J = 13.8, 2.4 Hz, 1H), 7.08 (d, J = 2.4 Hz, 1H), 4.30 (m, 2H), 3.72 (m, 2H), 3.34 (s, 3H), 1.45 (s, 4H). | 441.2 |
| H5 | G5 | 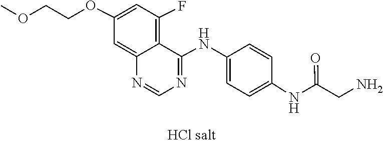 [SMILES: COCCOc1cc(F)c2c(Nc3ccc(NC(=O)CN)cc3)ncnc2c1] | 99 | 8.72 (s, 1H), 8.24 (m, 3H), 7.70 (d, J = 9.2 Hz, 2H), 7.57 (d, J = 8.8 Hz, 2H), 7.42 (dd, J = 13.6, 2.0 Hz, 1H), 7.21 (d, J = 2.0 Hz, 1H), 4.33 (m, 2H), 3.81 (m, 2H), 3.71 (m, 2H), 3.31 (s, 3H). NH₂ protons are missing | 386.4 |
| H6 | G8 | 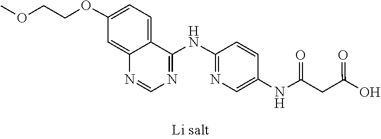 [SMILES: COCCOc1ccc2c(Nc3ccc(NC(=O)CC(=O)O)cn3)ncnc2c1] | 60 | No Data | 398.0 |

### Preparation of 1-(3-fluoro-3-methylbutyl)-3-(4-((5-fluoro-7-hydroxyquinazolin-4-yl)amino)phenyl)urea

| | | | | ¹H NMR (400 or 500 MHz, | MS (m/z: |
|---|---|---|---|---|---|
| Ex. No | SM | Product | Yield (%) | DMSO-d₆): δ | M + H⁺) |
| I1 | 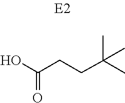 | 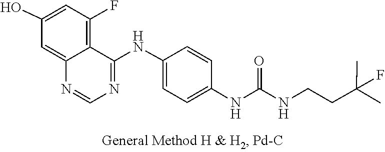 [SMILES: CC(C)(F)CCNC(=O)Nc1ccc(Nc2ncnc3cc(O)cc(F)c23)cc1] | 65 | 10.9 (br s, 1H), 8.77 (d, J = 12.8 Hz, 1H), 8.50 (s, 1H), 8.34 (s, 1H), 7.56 (d, J = 9.2 Hz, 2H), 7.37 (d, J = 8.8 Hz, 2H), 6.89 (dd, J = 13.6, 2.0 Hz, 1H), 6.85 (d, J = 2.4 Hz, 1H), 6.07 (t, J = 5.6 Hz, 1H), 3.23 (m, 2H), 1.79 (m, 2H), 1.35 (d, J = 21.6 Hz, 6H). | 402.3 |

### Preparation of Intermediate J1: 1-(4-((7-bromocinnolin-4-yl)amino)phenyl)-3-(3-fluoro-3-methylbutyl)urea

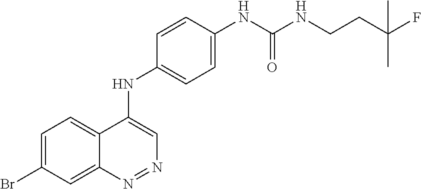 [SMILES: CC(C)(F)CCNC(=O)Nc1ccc(Nc2cnnc3cc(Br)ccc23)cc1]

A solution of 1-(4-aminophenyl)-3-(3-fluoro-3-methylbutyl)urea hydrochloride (G6, 0.27 g, 0.99 mmol) in toluene (8 mL) was treated with DIEA (0.32 g, 2.5 mmol). 7-Bromo-4-chlorocinnoline (0.20, 0.82 mmol) was added and then stirred 15 min. n-BuOH (2 mL) was added and then the reaction mixture was heated at 90° C. for 3 h. The reaction mixture was cooled to rt and then concentrated under reduced pressure. The residue was purified by silica gel column chromatography (0-15% (5% NH₄OH/MeOH)/DCM) to obtain 1-(4-((7-bromocinnolin-4-yl)amino)phenyl)-3-(3-fluoro-3-methylbutyl)urea (0.24 g, 64%). MS (ESI) m/z: 116.0 (M+H⁺) 448.0.

General Method L: Amide Coupling with HATU

### Example 4: N1-(4-((5-fluoro-7-(2-methoxyethoxy)quinazolin-4-yl)amino)phenyl)-N3-neopentylmalonamide

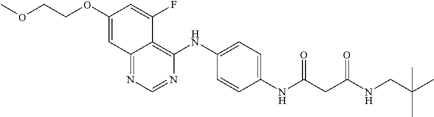 [SMILES: COCCOc1cc(F)c2c(Nc3ccc(NC(=O)CC(=O)NCC(C)(C)C)cc3)ncnc2c1]

A solution of 3-((4-((5-fluoro-7-(2-methoxyethoxy)quinazolin-4-yl)amino)phenyl)amino)-3-oxopropanoic acid (H1, 0.10 g, 0.025 mmol) in DMF (1 mL) was treated with DIEA (0.10 g, 0.076 mmol). 2,2-Dimethylpropan-1-amine (0.03 g, 0.033 mmol) and HATU (0.013 g, 0.033 mmol) were added and the reaction mixture was stirred at 50° C. for 2 h. The mixture was cooled to rt, diluted with water (1 mL), and stirred to precipitate. The resulting solids were filtered, washed with water, dried under high vacuum. The solids were triturated with CH₃CN (3 mL) and stirred at rt. The resulting solids were filtered and dried under high vacuum oven at 80° C. to obtain N1-(4-((5-fluoro-7-(2-methoxyethoxy)quinazolin-4-yl)amino)phenyl)-N3-neopentylmalonamide (0.10 g, 80%). ¹H NMR (500 MHz, DMSO-d₆): δ 10.14 (s, 1H), 8.98 (d, J=12.3 Hz, 1H), 8.45 (s, 1H), 8.00 (t, J=6.3 Hz, 1H), 7.56-7.67 (m, 4H), 7.15 (dd, J=13.8, 2.4 Hz, 1H), 7.06 (d, J=2.5 Hz, 1H), 4.26-4.32 (m, 2H), 3.69-3.75 (m, 2H), 3.33 (s, 3H), 3.31 (s, 2H), 2.94 (d, J=6.3 Hz, 2H), 0.87 (s, 9H); MS (ESI) m/z: 484.2 (M+H⁺).

### General Method M: Amide Coupling Under Heating

### Example 5: N¹-(4-((5-fluoro-7-(2-methoxyethoxy)quinazolin-4-yl)amino)phenyl)-N³-(2-methoxy-2-methylpropyl)malonamide

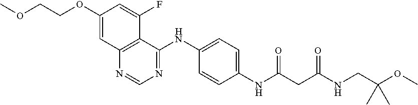

A suspension of methyl 3-((4-((5-fluoro-7-(2-methoxyethoxy)quinazolin-4-yl)amino)phenyl)amino)-3-oxopropanoate (G1, 0.40 g, 0.93 mmol), 2-methoxy-2-methylpropan-1-amine (0.25 g, 2.4 mol) in CH₃CN (6 mL) was stirred at 90° C. for 13 h. The reaction mixture was cooled to rt and then the solution was concentrated under reduced pressure. The residue was dissolved in DCM (30 mL), passed through a pad of silica gel, washed with EtOAc (100 mL) and acetone (100 mL). The fraction from acetone was contained pure compound. The fraction was concentrated under reduced pressure. The residue was recrystallized in CH₃CN to provide N1-(4-((5-fluoro-7-(2-methoxyethoxy)quinazolin-4-yl)amino)phenyl)-N3-(2-methoxy-2-methylpropyl)malonamide (0.12 g, 24%) as a pale yellow solid. ¹H NMR (500 MHz, DMSO-d₆): δ 10.12 (s, 1H), 8.97 (d, J=12.3 Hz, 1H), 8.45 (s, 1H), 7.96 (t, J=6.0 Hz, 1H), 7.62 (m, 4H), 7.15 (dd, J=13.8, 2.4 Hz, 1H), 7.06 (d, J=2.4 Hz, 1H), 4.29 (m, 2H), 3.72 (m, 2H), 3.34 (s, 3H), 3.30 (s, 2H), 3.17 (d, J=5.9 Hz, 2H), 3.13 (s, 3H), 1.09 (s, 6H); MS (ESI) m/z: 500.2 (M+H⁺).

General Method N: Alkylation with Alkyl Bromide

### Example 6: 1-(3-fluoro-3-methylbutyl)-3-(4-((5-fluoro-7-isopropoxyquinazolin-4-yl)amino)phenyl)urea

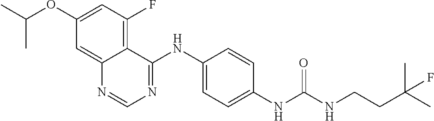 [SMILES: CC(C)Oc1cc(F)c2c(Nc3ccc(NC(=O)NCCC(C)(C)F)cc3)ncnc2c1]

A stirred solution of 1-(3-fluoro-3-methylbutyl)-3-(4-((5-fluoro-7-hydroxyquinazolin-4-yl)amino)phenyl)urea (I1, 0.20 g, 0.50 mmol) in CH₃CN (4 mL), was treated with Cs₂CO₃ (0.49 g, 1.49 mmol), KI (0.008 g, 0.05 mmol) and 2-bromopropane (0.12 g, 0.10 mmol) at rt. This suspension was further heated at 90° C. for 16 h. The reaction mixture was quenched with ice water (10 mL) and then the solution was extracted with 10% MeOH in DCM (3×). The organic extracts were dried over anhydrous Na₂SO₄, filtered, and concentrated under reduced pressure. The crude was purified on silica gel column chromatography (6-8% MeOH/DCM) to afford 1-(3-fluoro-3-methylbutyl)-3-(4-((5-fluoro-7-isopropoxyquinazolin-4-yl)amino)phenyl)urea (0.055 g, 25%) as an off white solid. ¹H NMR (500 MHz, DMSO-d₆): δ 8.84 (d, J=12.4 Hz, 1H), 8.51 (s, 1H), 8.40 (s, 1H), 7.51 (d, J=9.2 Hz, 2H), 7.38 (d, J=8.8 Hz, 2H), 7.05 (dd, J=14.0, 2.4 Hz, 1H), 7.00 (d, J=2.4 Hz, 1H), 6.08 (t, J=5.6 Hz, 1H), 4.85 (m, 1H), 3.20 (m, 2H), 1.79 (dt, J=20.0, 7.6 Hz, 2H), 1.35 (d, J=21.6 Hz, 6H), 1.33 (d, J=6.0 Hz, 6H); MS (ESI) m/z: 444.3 (M+H⁺).

General Method O: Alkylation with Alkyl Benzenesulfonate

### Example 7: (S)-1-(3-fluoro-3-methylbutyl)-3-(4-((5-fluoro-7-(2-methoxypropoxy)quinazolin-4-yl)amino)phenyl)urea

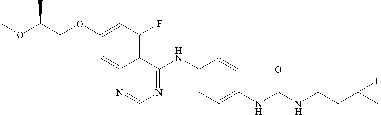 [SMILES: CO[C@@H](C)COc1cc(F)c2c(Nc3ccc(NC(=O)NCCC(C)(C)F)cc3)ncnc2c1]

A stirred solution of 1-(3-fluoro-3-methylbutyl)-3-(4-((5-fluoro-7-hydroxyquinazolin-4-yl)amino)phenyl)urea (I1, 0.25 g, 0.62 mmol) in CH₃CN (4 mL) was treated with Cs₂CO₃ (0.6 g, 1.87 mmol), KI (0.001 g, 0.062 mmol) and (S)-2-methoxypropyl 4-methylbenzenesulfonate (0.23 g, 0.93 mmol) at rt. The reaction mixture was further heated at 90° C. for 16 h. The reaction mixture was quenched with ice water (10 mL) and then the solution was extracted with 10% MeOH in DCM (3×). The organic extracts were dried over anhydrous Na₂SO₄, filtered, and concentrated under reduced pressure. The crude was purified on silica gel column chromatography (6-8% MeOH/DCM) to afford desired compound of (S)-1-(3-fluoro-3-methylbutyl)-3-(4-((5-fluoro-7-(2-methoxypropoxy)quinazolin-4-yl)amino)phenyl)urea (0.021 g, 7%) as a yellow solid. ¹H NMR (500 MHz, DMSO-d₆): δ 8.88 (d, J=12.8 Hz, 1H), 8.51 (s, 1H), 8.41 (s, 1H), 7.51 (d, J=9.2 Hz, 2H), 7.38 (d, J=8.8 Hz, 2H), 7.12 (dd, J=13.6, 2.0 Hz, 1H), 7.03 (d, J=2.0 Hz, 1H), 6.08 (t, J=5.6 Hz, 1H), 4.10 (m, 2H), 3.71 (m, 1H), 3.21 (m, 2H), 1.81 (m, 1H), 1.76 (m, 1H), 1.35 (d, J=21.6 Hz, 6H), 1.20 (d, J=6.4 Hz, 3H). 3Hs are under solvents; MS (ESI) m/z: 474.4 (M+H⁺).

### General Method P: Suzuki Reaction and Reduction

### Example 190: 1-(3-fluoro-3-methylbutyl)-3-(4-((7-(3-methoxypropyl)cinnolin-4-yl)amino)phenyl)urea

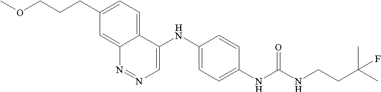 [SMILES: COCCCc1ccc2c(Nc3ccc(NC(=O)NCCC(C)(C)F)cc3)cnnc2c1]

A mixture of (E)-2-(3-methoxyprop-1-en-1-yl)-4,4,5,5-tetramethyl-1,3,2-dioxaborolane (0.16 g, 0.79 mmol) and 1-(4-((7-bromocinnolin-4-yl)amino)phenyl)-3-(3-fluoro-3-methylbutyl)urea (J1, 0.12 g, 0.26 mmol) in 1,4-dioxane/water (3 mL, 2:1) was treated with K₂CO₃ (0.11 g, 0.79 mmol). The mixture was degassed with Ar and treated with PdCl₂(dppf)-CH₂Cl₂ adduct (11 mg, 0.013 mmol). The mixture was heated at 80° C. for 1 h, and then cooled to rt. The mixture was diluted with water (10 mL) and extracted with 10% MeOH/DCM (3×). The organic phases washed with brine (40 mL) and dried over anhydrous Na₂SO₄, filtered, and concentrated under reduced pressure to obtain the crude, (E)-1-(3-fluoro-3-methylbutyl)-3-(4-((7-(3-methoxyprop-1-en-1-yl)cinnolin-4-yl)amino)phenyl)urea. MS (ESI) m/z 438.2 (M+H⁺).

A solution of (E)-1-(3-fluoro-3-methylbutyl)-3-(4-((7-(3-methoxyprop-1-en-1-yl)cinnolin-4-yl)amino)phenyl)urea (135 mg, 0.31 mmol) in MeOH (8 mL) was treated with 10% Pd/C (33 mg). The mixture was stirred under a H₂ atmosphere (balloon) (flushed 3×) and stirred at rt for 8 h. The mixture was filtered through a pad of celite and rinsed well with MeOH (15 mL). The filtrates concentrated and the residue was purified by reverse phase column chromatography (20-60% CH₃CN/H₂O (0.1% formic acid)). Combined pure fractions were neutralized with solid K₂C₃. The solution was extracted with 10% MeOH/DCM (2×) and the organic layers were washed with brine. The organic layer was dried over anhydrous Na₂SO₄, filtered, and concentrated under reduced pressure to obtain 1-(3-fluoro-3-methylbutyl)-3-(4-((7-(3-methoxypropyl)cinnolin-4-yl)amino)phenyl)urea (0.013 g, 9%). ¹H NMR (500 MHz, DMSO-d₆): δ 9.05 (s, 1H), 8.57 (m, 2H), 8.26 (d, J=8.7 Hz, 1H), 7.88 (s, 1H), 7.53 (d, J=8.6 Hz, 1H), 7.43 (d, J=8.6 Hz, 2H), 7.19 (d, J=8.5 Hz, 2H), 6.07 (t, J=5.8 Hz, 1H), 3.31 (t, J=6.4 Hz, 2H), 3.18 (d, J=12.9 Hz, 3H), 3.03-3.17 (m, 2H), 2.80 (t, J=7.7 Hz, 2H), 1.87 (m, 2H), 1.74 (m, 2H), 1.29 (d, J=21.6 Hz, 6H); MS (ESI) m/z: 440.2 (M+H⁺).

Using the General Methods L, M, N, and O above, the following Intermediates of Table H were prepared.

TABLE H

| | | | | | | MS |
|---|---|---|---|---|---|---|
| | | | | | | (m/z: |
| Ex. | | | | Yield | ¹H NMR (400 or 500 | M + |
| No | SM | Product | Method | (%) | MHz, DMSO-d₆): δ | H⁺) |
| 8 | 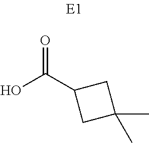 | 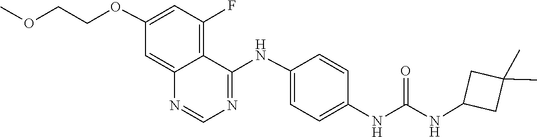 | H | 32 | 8.86 (d, J = 12.5 Hz, 1H), 8.40 (s, 1H), 8.28 (s, 1H), 7.49 (d, J = 8.5 Hz, 2H), 7.35 (d, J = 8.6 Hz, 2H), 7.11 (dd, J = 13.8, 2.4 Hz, 1H), 7.03 (s, 1H), 6.33 (d, J = 7.9 Hz, 1H), 4.27 (t, J = 4.2 Hz, 2H), 4.12 (m, 1H), | 454.2 |
| | | | | | 3.70 (t, J = 4.2 Hz, 2H), | |
| | | | | | 2.07 (t, J = 9.2 Hz, 2H), | |
| | | | | | 1.64 (t, J = 9.6 Hz, 2H), | |
| | | | | | 1.20 (s, 3H), 1.00 (s, | |
| | | | | | 3H). 3Hs are under | |
| | | | | | solvent | |
| 9 | 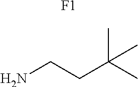 | 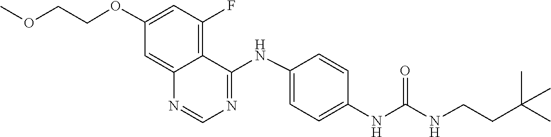 [SMILES: COCCOc1cc(F)c2c(Nc3ccc(NC(=O)NCCC(C)(C)C)cc3)ncnc2c1] | G | 24 | 8.86 (d, J = 12.6 Hz, 1H), 8.40 (s, 2H), 7.49 (d, J = 8.6 Hz, 2H), 7.37 (d, J = 8.7 Hz, 2H), 7.11 (dd, J = 13.8, 2.4 Hz, 1H), 7.02 (d, J = 2.4 Hz, 1H), 5.99 (t, J = 5.6 Hz, 1H), 4.27 (t, J = 4.3 Hz, 2H), 3.70 (t, J = 4.2 Hz, | 456.2 |
| | | | | | 2H), 3.32 (s, 3H), 3.07- | |
| | | | | | 3.11 (m, 2H), 1.33-1.36 | |
| | | | | | (m, 2H), 0.90 (s, 9H). | |
| 10 | 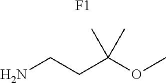 | 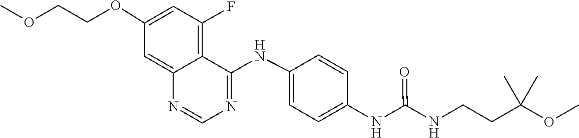 | G | 18 | 8.86 (d, J = 12.6 Hz, 1H), 8.47 (s, 1H), 8.40 (s, 1H), 7.49 (d, J = 8.5 Hz, 2H), 7.37 (d, J = 8.5 Hz, 2H), 7.11 (d, J = 13.8 Hz, 1H), 7.02 (s, 1H), 6.03 (t, J = 5.6 Hz, 1H), 4.27 (d, J = 4.8 Hz, 2H), 3.70 (m, 2H), 3.31 | 472.2 |
| | | | | | (s, 3H), 3.13 (m, 5H), | |
| | | | | | 1.60 (m, 2H), 1.10 | |
| | | | | | (s, 6H). | |
| 11 | 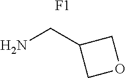 | 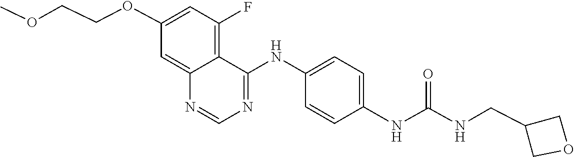 [SMILES: COCCOc1cc(F)c2c(Nc3ccc(NC(=O)NCC4COC4)cc3)ncnc2c1] | G | 12 | 8.86 (d, J = 12.5 Hz, 1H), 8.40 (s, 1H), 8.28 (s, 1H), 7.49 (d, J = 8.5 Hz, 2H), 7.35 (d, J = 8.6 Hz, 2H), 7.11 (dd, J = 13.8, 2.4 Hz, 1H), 7.03 (s, 1H), 6.33 (d, J = 7.9 Hz, 1H), 4.27 (t, J = 4.2 Hz, 2H), 4.12 (m, 1H), 3.70 (t, J = 4.2 Hz, 2H), | 442.2 |
| | | | | | 2.07 (t, J = 9.2 Hz, 2H), | |
| | | | | | 1.64 (t, J = 9.6 Hz, 2H), | |
| | | | | | 1.20 (s, 3H), 1.00 (s, | |
| | | | | | 3H). 3Hs are under | |
| | | | | | solvent | |
| 12 | 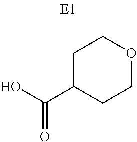 | 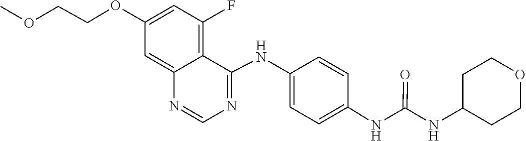 [SMILES: COCCOc1cc(F)c2c(Nc3ccc(NC(=O)NC4CCOCC4)cc3)ncnc2c1] | H | 33 | 8.86 (d, J = 12.5 Hz, 1H), 8.40 (s, 1H), 8.34 (s, 1H), 7.50 (d, J = 8.6 Hz, 2H), 7.37 (d, J = 8.6 Hz, 2H), 7.11 (dd, J = 13.8, 2.4 Hz, 1H), 7.03 (d, J = 2.3 Hz, 1H), 6.16 (d, J = 7.6 Hz, 1H), 4.27 (t, J = 4.2 Hz, 2H), 3.80 | 456.2 |
| | | | | | (m, 2H), 3.70 (m, | |
| | | | | | 3H), 3.37 (m, 2H), 3.33 | |
| | | | | | (s, 3H), 1.79 (m, 2H), | |
| | | | | | 1.37 (m, 2H). | |
| 13 | 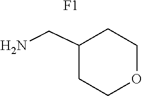 | 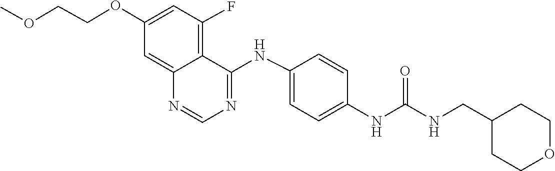 [SMILES: COCCOc1cc(F)c2c(Nc3ccc(NC(=O)NCC4CCOCC4)cc3)ncnc2c1] | G | 56 | 8.86 (d, J = 12.6 Hz, 1H), 8.40 (d, J = 3.9 Hz, 2H), 7.50 (d, J = 8.7 Hz, 2H), 7.37 (d, J = 8.7 Hz, 2H), 7.11 (dd, J = 13.8, 2.4 Hz, 1H), 7.03 (d, J = 2.4 Hz, 1H), 6.18 (t, J = 5.9 Hz, 1H), 4.27 (t, J = 4.3 Hz, 2H), 3.84 (dd, J = 11.3, 4.1 Hz, 2H), 3.70 (t, J = 4.3 Hz, 2H), | 470.2 |
| | | | | | 3.32 (s, 3H), 3.26 (m, | |
| | | | | | 2H), 2.99 (t, J = 6.3 Hz, | |
| | | | | | 2H), 1.66 (m, 1H), 1.51- | |
| | | | | | 1.63 (m, 2H), 1.17 (m, | |
| | | | | | 2H). | |
| 14 | 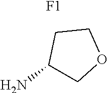 | 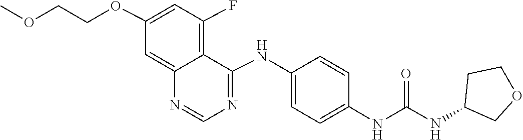 [SMILES: COCCOc1cc(F)c2c(Nc3ccc(NC(=O)N[C@@H]4CCOC4)cc3)ncnc2c1] | G | 45 | 8.87 (d, J = 12.5 Hz, 1H), 8.40 (s, 1H), 8.34 (s, 1H), 7.51 (d, J = 8.5 Hz, 2H), 7.37 (d, J = 8.4 Hz, 2H), 7.11 (d, J = 13.8 Hz, 1H), 7.03 (s, 1H), 6.39 (d, J = 6.8 Hz, 1H), 4.27 (m, 2H), 4.20 (m, 1H), 3.72 (m, 5H), | 442.2 |
| | | | | | 3.48 (dd, J = 8.9, 3.5 Hz, | |
| | | | | | 1H), 3.32 (s, 3H), 2.10 | |
| | | | | | (m, 1H), 1.70 (m, 1H). | |
| 15 | 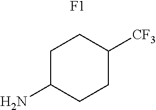 | 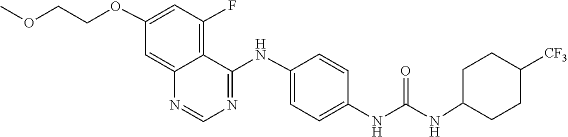 [SMILES: COCCOc1cc(F)c2c(Nc3ccc(NC(=O)NC4CCC(C(F)(F)F)CC4)cc3)ncnc2c1] | G | 51 | 8.86 (d, J = 12.5 Hz, 1H), 8.40 (s, 1H), 8.31 (s, 1H), 7.51 (d, J = 8.3 Hz, 2H), 7.37 (d, J = 8.2 Hz, 2H), 7.11 (d, J = 13.7 Hz, 1H), 7.03 (s, 1H), 6.41 (d, J = 7.4 Hz, 1H), 4.27 (m, 2 H), 3.87 (m, 1 H), 3.70 (m, 2H), | 522.2 |
| | | | | | 3.32 (s, 3H), 2.49 (br s, | |
| | | | | | 1H), 1.97-1.88 (m, 1H), | |
| | | | | | 1.72 (t, J = 13.1 Hz, | |
| | | | | | 3H), 1.57-1.45 (m, 3H), | |
| | | | | | 1.30-1.66 (m, 1H). | |
| 16 | 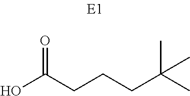 | 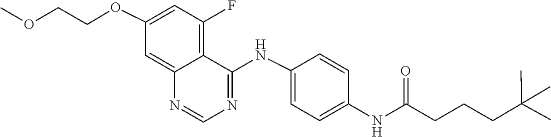 [SMILES: COCCOc1cc(F)c2c(Nc3ccc(NC(=O)CCCC(C)(C)C)cc3)ncnc2c1] | I | 34 | 9.87 (s, 1H), 8.92 (d, J = 12.4 Hz, 1H), 8.43 (s, 1H), 7.58 (s, 4H), 7.12 (dd, J = 13.8, 2.4 Hz, 1H), 7.04 (d, J = 2.4 Hz, 1H), 4.27 (t, J = 4.3 Hz, 2H), 3.70 (t, J = 4.2 Hz, 2H), 3.32 (s, 3H), 2.24 (m, 2H), 1.55 (m, 2H), | 455.2 |
| | | | | | 1.18 (m, 2H), 0.87 (s, | |
| | | | | | 9H). | |
| 17 | 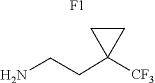 | 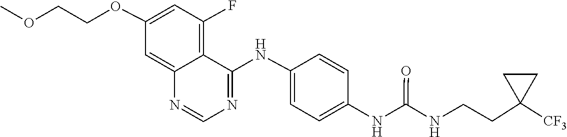 [SMILES: COCCOc1cc(F)c2c(Nc3ccc(NC(=O)NCCC4(C(F)(F)F)CC4)cc3)ncnc2c1] | G | 5 | 8.86 (d, J = 12.5 Hz, 1H), 8.52 (s, 1H), 8.40 (s, 1H), 7.50 (d, J = 8.7 Hz, 2H), 7.37 (d, J = 8.8 Hz, 2H), 7.11 (dd, J = 13.8, 2.4 Hz, 1H), 7.03 (d, J = 2.4 Hz, 1H), 6.12 (t, J = 5.8 Hz, 1H), 4.27 (t, J = 4.3 Hz, 2H), 3.70 | 508.2 |
| | | | | | (t, J = 4.3 Hz, 2H), 3.32 | |
| | | | | | (s, 3H), 3.19 (t, J = 4.3 | |
| | | | | | Hz, 2H), 1.72 (t, J = 7.8 | |
| | | | | | Hz, 2H), 0.91 (t, J = 5.6 | |
| | | | | | Hz, 2H), 0.77 (t, J = 5.6 | |
| | | | | | Hz, 2H). | |
| 18 | 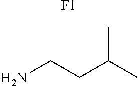 | 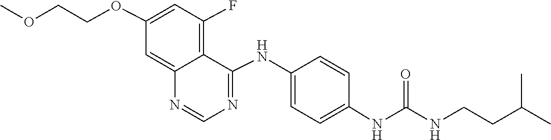 [SMILES: COCCOc1cc(F)c2c(Nc3ccc(NC(=O)NCCC(C)C)cc3)ncnc2c1] | G | 58 | 8.87 (d, J = 12.6 Hz, 1H), 8.42 (t, J = 9.8 Hz, 2H), 7.51 (d, J = 8.6 Hz, 2H), 7.39 (d, J = 8.6 Hz, 2H), 7.13 (dd, J = 13.8, 2.4 Hz, 1H), 7.04 (d, J = 2.3 Hz, 1H), 6.07 (t, J = 5.6 Hz, 1H), 4.29 (t, J = 4.3 Hz, 2H), 3.72 (t, J = | 442.2 |
| | | | | | 4.2 Hz, 2H), 3.33 (s, | |
| | | | | | 3H), 3.11 (q, J = 6.7 Hz, | |
| | | | | | 2H), 1.62 (m, 1H), 1.34 | |
| | | | | | (q, J = 7.1 Hz, 2H), 0.91 | |
| | | | | | (d, J = 6.6 Hz, 6H). | |
| 19 | 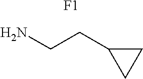 | 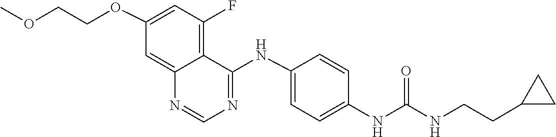 [SMILES: COCCOc1cc(F)c2c(Nc3ccc(NC(=O)NCCC4CC4)cc3)ncnc2c1] | G | 24 | 8.80 (d, J = 12.6 Hz, 1H), 8.35-8.39 (m, 2H), 7.42 (dd, J = 16.9, 8.6 Hz, 2H), 7.32 (d, J = 8.7 Hz, 2H), 7.03-7.08 (m, 1H), 6.97 (m, 1H), 6.04 (t, J = 5.6 Hz, 1H), 4.21 (t, J = 4.3 Hz, 2H), 3.66 (t, J = 4.3 Hz, 2H), 3.33 | 440.2 |
| | | | | | (s, 3H), 3.09 (q, J = 6.6 | |
| | | | | | Hz, 2H), 1.28 (q, J = 7.0 | |
| | | | | | Hz, 2H), 0.63 (m, 1H), | |
| | | | | | 0.35 (m, 2H), 0.012 (m, | |
| | | | | | 2H). | |
| 20 | 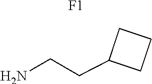 | 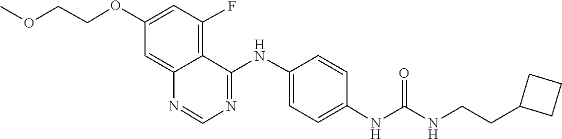 | G | 36 | 8.87 (d, J = 12.6 Hz, 1H), 8.41 (d, J = 10.7 Hz, 2H), 7.51 (d, J = 8.6 Hz, 2H), 7.39 (d, J = 8.6 Hz, 2H), 7.13 (dd, J = 13.8, 2.4 Hz, 1H), 7.04 (d, J = 2.3 Hz, 1H), 6.03 (t, J = 5.6 Hz, 1H), 4.29 (t, J = 4.3 Hz, 2H), 3.73 | 454.2 |
| | | | | | (m, 2H), 3.34 (s, 3H), | |
| | | | | | 3.01 (q, J = 6.6 Hz, 2H), | |
| | | | | | 2.30 (m, 1H), 2.00-2.08 | |
| | | | | | (m, 2H), 1.75-1.89 (m, | |
| | | | | | 2H), 1.52-1.66 (m, 4H). | |
| 21 | 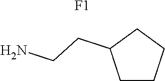 | 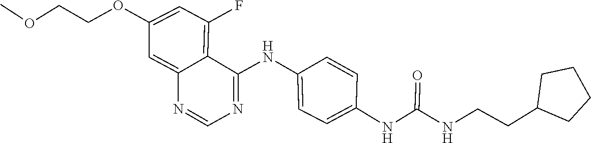 [SMILES: COCCOc1cc(F)c2c(Nc3ccc(NC(=O)NCCC4CCCC4)cc3)ncnc2c1] | G | 26 | 8.87 (d, J = 12.6 Hz, 1H), 8.42 (t, J = 7.2 Hz, 2H), 7.51 (t, J = 8.6 Hz, 2H), 7.39 (d, J = 8.6 Hz, 2H), 7.12 (m, 1H), 7.05 (m, 1H), 6.09 (t, J = 5.6 Hz, 1H), 4.28 (t, J = 4.3 Hz, 2H), 3.70 (m, 2H), 3.34 (s, 3H), 3.10 (q, J = | 468.2 |
| | | | | | 6.7 Hz, 2H), 1.70-1.80 | |
| | | | | | (m, 3H), 1.53-1.58 (m, | |
| | | | | | 2H), 1.41-1.53 (m, 4H), | |
| | | | | | 1.00-1.10 (m, 2H). | |
| 22 | 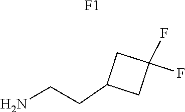 | 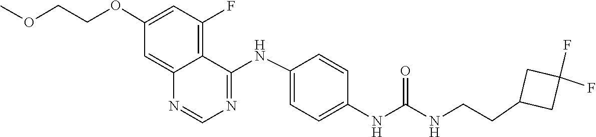 [SMILES: COCCOc1cc(F)c2c(Nc3ccc(NC(=O)NCCC4CC(F)(F)C4)cc3)ncnc2c1] | G | 11 | 8.89 (t, J = 12.5 Hz, 1H), 8.42 (m, 2H), 7.52 (d, J = 8.6 Hz, 2H), 7.39 (d, J = 8.6 Hz, 2H), 7.13 (d, J = 13.8 Hz, 1H), 7.03 (s, 1H), 6.12 (t, J = 5.7 Hz, 1H), 4.29 (m, 2H), 3.70 (m, 2H), 3.34 (s, 3H), 3.07 (q, J = 6.4 | 490.2 |
| | | | | | Hz, 2H), 2.68 (m, 2H), | |
| | | | | | 2.25 (m, 2H), 2.11 (br | |
| | | | | | m, 1H), 1.64 (q, J = 7.1 | |
| | | | | | Hz, 2H). | |
| 23 | 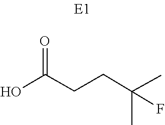 | 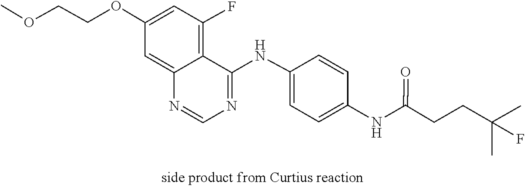 [SMILES: COCCOc1cc(F)c2c(Nc3ccc(NC(=O)CCC(C)(C)F)cc3)ncnc2c1] | H | 2 | 9.99 (s, 1H), 8.94 (d, J = 12.4 Hz, 1H), 8.45 (s, 1H); 7.60 (m, 4H), 7.14 (dd, J = 13.8, 2.4 Hz, 1H), 7.06 (d, J = 2.4 Hz, 1H), 4.29 (t, J = 4.3 Hz, 2H), 3.72 (t, J = 4.3 Hz, 2H), 3.34 (s, 3H), 2.42 (m, 2H), 1.94 (m, 2H), 1.37 (s, 3 H), 1.33 (s, 3H). | 445.2 |
| 24 | 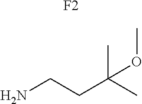 | 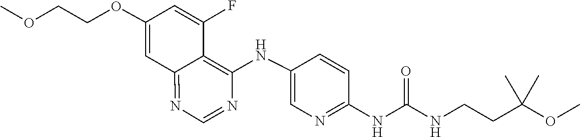 | G | 3 | 9.20 (s, 1H), 9.03 (d, J = 11.4 Hz, 1H), 8.43 (s, 2H), 8.04 (s, 1H), 7.90 (dd, J = 8.8, 2.7 Hz, 1H), 7.39 (t, J = 8.9 Hz, 1H), 7.16 (dd, J = 13.6, 2.4 Hz, 1H), 7.07 (d, J = 2.4 Hz, 1H), 4.29 (t, J = 4.3 Hz, 2H), 3.72 (t, J = 4.2 Hz, 2H), 3.35 (s, | 473.2 |
| | | | | | 3H), 3.21 (m, 2H), 3.13 | |
| | | | | | (s, 3H), 1.67 (t, J = 7.6 | |
| | | | | | Hz, 2H), 1.14 (s, 6H). | |
| 25 | 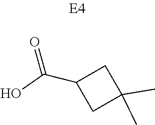 | 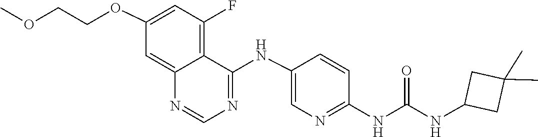 | H | 30 | 9.08 (s, 1H), 9.02 (d, J = 11.3 Hz, 1H), 8.84 (s, 1H), 8.43 (s, 1H), 8.01 (s, 1H), 7.90 (dd, J = 8.9, 2.6 Hz, 1H), 7.46 (d, J = 8.9 Hz, 1H), 7.17 (dd, J = 13.7, 2.4 Hz, 1H), 7.07 (d, J = 2.4 Hz, 1H), 4.29 (m, 2H), 4.20 | 455.2 |
| | | | | | (m, 1H), 3.71 (m, 2H), | |
| | | | | | 2.13 (m, 2H), 1.69 (m, | |
| | | | | | 2H), 1.14 (s, 3H), 1.12 | |
| | | | | | (s, 3H). | |
| 26 | 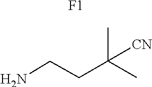 | 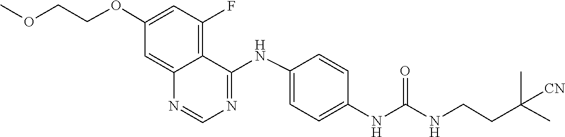 [SMILES: COCCOc1cc(F)c2c(Nc3ccc(NC(=O)NCCC(C)(C)C#N)cc3)ncnc2c1] | G | 26 | 8.93 (d, J = 11.0 Hz, 1H), 8.56 (s, 1H), 8.43 (m, 1H), 7.50 (d, J = 8.5 Hz, 2H), 7.40 (d, J = 8.5 Hz, 2H), 7.13 (dd, J = 13.0, 2.5 Hz, 1H), 7.04 (d, J = 3.0 Hz, 1H), 6.21 (t, J = 6.0 Hz, 1H), 4.28 (m, 2H), 3.72 (m, 2H), | 467.2 |
| | | | | | 3.30 (s, 3H), 3.25 (m, | |
| | | | | | 2H), 1.73 (m, 2H), 1.35 | |
| | | | | | (s, 6H). | |
| 27 | 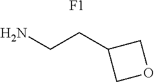 | 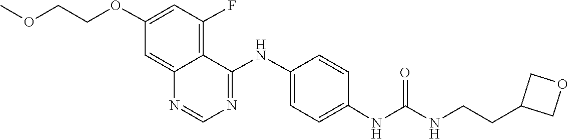 [SMILES: COCCOc1cc(F)c2c(Nc3ccc(NC(=O)NCCC4COC4)cc3)ncnc2c1] | G | 66 | 8.85 (d, J = 13.0 Hz, 1H), 8.38 (s, 1H), 8.10 (s, 1H), 7.46 (s, 4H), 7.08 (dd, J = 13.5, 2.5 Hz, 1H), 6.99 (d, J = 2.5 Hz, 1H), 4.69 (t, J = 5.0 Hz, 1H), 4.23 (m, 2H), 3.66 (m, 2H), 3.51 (m, 1H), 1.74 (m, 2H), 1.80 (m, 2H), 3.30 (s, 3H), | 456.2 |
| | | | | | 3.12 (m, 1H), 1.24 (m, | |
| | | | | | 1H), 1.15 (m, 1H), 1.17 | |
| | | | | | (m, 1H). | |
| 28 | 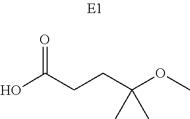 | 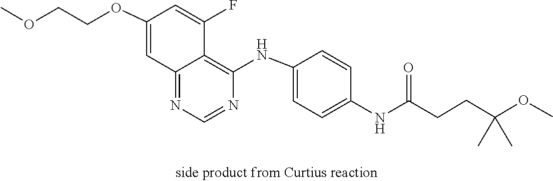 | H | 2 | 9.96 (s, 1H), 8.96 (d, J = 12.3 Hz, 1H), 8.44 (s, 1H), 7.59 (s, 4H), 7.15 (dd, J = 13.8, 2.4 Hz, 1H), 7.06 (d, J = 2.4 Hz, 1H), 4.30 (m, 2H), 3.70 (m, 2H), 3.33 (s, 3H), 3.10 (s, 3H), 2.30 (m, 2H), 1.75 (m, 2H), 1.12 (s, 6H). | 457.2 |
| 29 | 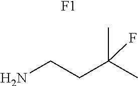 | 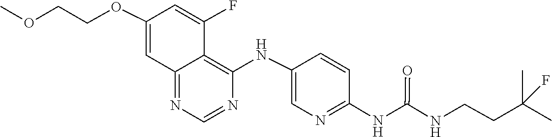 [SMILES: COCCOc1cc(F)c2c(Nc3ccc(NC(=O)NCCC(C)(C)F)nc3)ncnc2c1] | G | 13 | 9.26 (s, 1H), 9.05 (d, J = 11.3 Hz, 1H), 8.45 (m, 2H), 8.11 (s, 1H), 7.91 (dd, J = 9.0, 2.7 Hz, 1H), 7.38 (d, J = 8.9 Hz, 1H), 7.16 (dd, J = 13.7, 2.4 Hz, 1H), 7.07 (d, J = 2.4 Hz, 1H), 4.30 (m, 2H), 3.72 (m, 2H), 3.33 | 461.2 |
| | | | | | (s, 3H), 3.30 (m, | |
| | | | | | 2H), 1.85 (m, 2H), 1.35 | |
| | | | | | (d, J = 21.5 Hz, 6H). | |
| 30 | 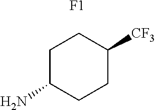 | 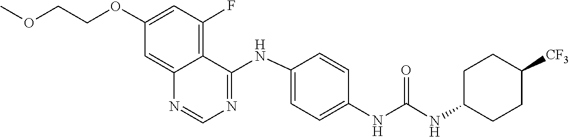 [SMILES: COCCOc1cc(F)c2c(Nc3ccc(NC(=O)N[C@H]4CC[C@H](C(F)(F)F)CC4)cc3)ncnc2c1] | G | 22 | 8.87 (d, J = 12.8 Hz, 1H), 8.41 (s, 1H), 8.31 (s, 1H), 7.51 (d, J = 8.8 Hz, 1H), 7.36 (d, J = 8.8 Hz, 2H), 7.12 (dd, J = 13.6, 2.0 Hz, 1H), 7.03 (d, J = 1.6 Hz, 1H), 6.06 (d, J = 7.6 Hz, 1H), 4.28 (m, 2H), 3.71 (m, 2H), | 522.4 |
| | | | | | 3.41 (m, 1H), 3.32 (s, | |
| | | | | | 3H), 2.27 (m, 1H), 1.97 | |
| | | | | | (m, 2H), 1.88 (m, 2H), | |
| | | | | | 1.30-1.39 (m, 2H), 1.16- | |
| | | | | | 1.25 (m, 2H). | |
| 31 | 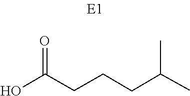 | 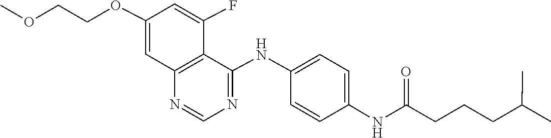 [SMILES: COCCOc1cc(F)c2c(Nc3ccc(NC(=O)CCCC(C)C)cc3)ncnc2c1] | 1 (EDC + HOBT) | 51 | 9.91 (s, 1H), 8.96 (d, J = 12.3 Hz, 1H), 8.84 (s, 1H), 7.60 (s, 4H), 7.15 (dd, J = 13.8, 2.5 Hz, 1H), 7.06 (d, J = 2.4 Hz, 1H), 4.29 (m, 2H), 3.72 (m, 2H), 3.33 (s, 3H), 2.29 (t, J = 7.5 Hz, 2H), 1.61 (m, 2H), 1.55 (m, | 441.2 |
| | | | | | 1H), 1.20 (m, 2H), 0.88 | |
| | | | | | (d, J = 6.6 Hz, 6H). | |
| 32 | 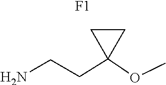 | 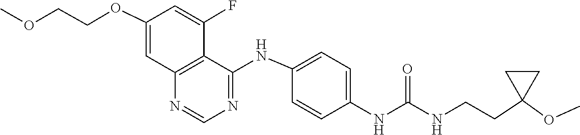 | G | 14 | 8.88 (d, J = 12.4 Hz, 1H), 8.54 (s, 1H), 8.40 (s, 1H), 7.50 (d, J = 8.8 Hz, 2H), 7.38 (d, J = 8.8 Hz, 2H), 7.12 (dd, J = 13.6, 2.0 Hz, 1H), 7.03 (d, J = 2.0 Hz, 1H), 6.07 (t, J = 5.6 Hz, 1H), 4.27 (m, 2H), 3.70 (m, 2H), | 470.4 |
| | | | | | 3.32 (s, 3H), 3.22 (m, | |
| | | | | | 2H), 3.18 (s, 3H), 1.70 | |
| | | | | | (t, J = 7.2 Hz, 2H), 0.66 | |
| | | | | | (m, 2H), 0.42 (m, 2H). | |
| 33 | 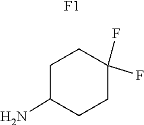 | 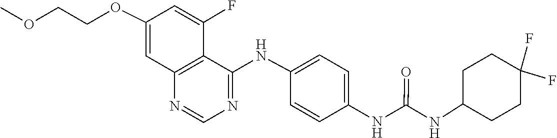 [SMILES: COCCOc1cc(F)c2c(Nc3ccc(NC(=O)NC4CCC(F)(F)CC4)cc3)ncnc2c1] | G | 6 | 8.87 (d, J = 12.4 Hz, 1H), 8.41 (s, 1H), 8.32 (s, 1H), 7.51 (d, J = 8.8 Hz, 2H), 7.37 (d, J = 8.8 Hz, 2H), 7.12 (dd, J = 14.0, 2.4 Hz, 1H), 7.03 (d, J = 2.0 Hz, 1H), 6.24 (d, J = 7.6 Hz, 1H), 4.28 (m, 2H), 3.71 (m, 2H), | 490.2 |
| | | | | | 3.66 (m, 1H), 3.32 (s, | |
| | | | | | 3H), 1.93-2.01 (m, 3H), | |
| | | | | | 1.85-1.93 (m, 3H), 1.44- | |
| | | | | | 1.54 (m, 2H). | |
| 34 | 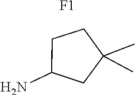 | 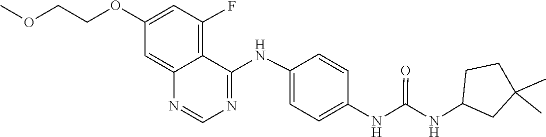 | G | 24 | 8.88 (d, J = 12.8 Hz, 1H), 8.41 (s, 1H), 8.27 (s, 1H), 7.50 (d, J = 8.8 Hz, 2H), 7.36 (d, J = 8.8 Hz, 2H), 7.12 (dd, J = 13.6, 2.4 Hz, 1H), 7.03 (d, J = 2.0 Hz, 1H), 6.15 (d, J = 7.6 Hz, 2H), 4.28 (m, 2H), 4.08 (m, 1H), | 468.3 |
| | | | | | 3.71 (m, 2H), 3.32 (s, | |
| | | | | | 3H), 1.97-2.04 (m, 1H), | |
| | | | | | 1.75-1.80 (m, 1H), 1.41- | |
| | | | | | 1.53 (m, 2H), 1.34-1.39 | |
| | | | | | (m, 1H), 1.18-1.23 (m, | |
| | | | | | 1H), 1.06 (s, 3H), 0.98 | |
| | | | | | (s, 3H). | |
| 35 | 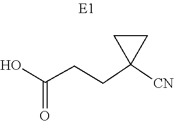 | 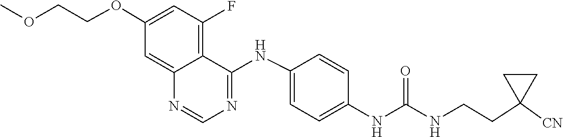 [SMILES: COCCOc1cc(F)c2c(Nc3ccc(NC(=O)NCCC4(C#N)CC4)cc3)ncnc2c1] | H | 12 | 8.87 (d, J = 12.4 Hz, 1H), 8.54 (s, 1H), 8.41 (s, 1H), 7.51 (d, J = 8.8 Hz, 2H), 7.39 (d, J = 8.8 Hz, 2H), 7.12 (dd, J = 14.0, 2.4 Hz, 1H), 7.03 (d, J = 2.0 Hz, 1H), 6.21 (t, J = 5.6 Hz, 1H), 4.28 (m, 2H), 3.71 (m, 2H), | 465.3 |
| | | | | | 3.32 (s, 3H), 3.25-3.31 | |
| | | | | | (m, 2H), 1.63 (t, J = 7.2 | |
| | | | | | Hz, 2H), 1.19 (m, 2H), | |
| | | | | | 0.92 (m, 2H). | |
| 36 | 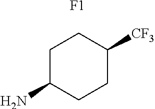 | 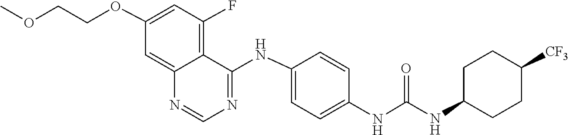 [SMILES: COCCOc1cc(F)c2c(Nc3ccc(NC(=O)N[C@H]4CC[C@@H](C(F)(F)F)CC4)cc3)ncnc2c1] | G | 27 | 8.88 (d, J = 12.4 Hz, 1H), 8.41 (s, 1H), 8.33 (s, 1H), 7.52 (d, J = 8.8 Hz, 2H), 7.38 (d, J = 8.8 Hz, 2H), 7.12 (dd, J = 14.0, 2.0 Hz, 1H), 7.03 (d, J = 2.0 Hz, 1H), 6.42 (d, J = 7.2 Hz, 1H), 4.28 (m, 2H), 3.88 (m, 1H), | 522.3 |
| | | | | | 3.71 (m, 2H), 3.32 (s, | |
| | | | | | 3H), 2.32 (m, 1H), 1.70- | |
| | | | | | 1.76 (m, 4H), 1.55-1.61 | |
| | | | | | (m, 2H), 1.42-1.51 (m, | |
| | | | | | 2H). | |
| 37 | 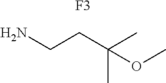 | 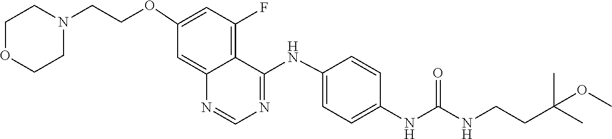 [SMILES: COC(C)(C)CCNC(=O)Nc1ccc(Nc2ncnc3cc(OCCN4CCOCC4)cc(F)c23)cc1] | G | 46 | 8.86 (br d, J = 10.4 Hz, 1H), 8.51 (s, 1H), 8.41 (s, 1H), 7.50 (d, J = 8.8 Hz, 2H), 7.38 (d, J = 8.8 Hz, 2H), 7.11 (dd, J = 13.6, 2.0 Hz, 1H), 7.04 (d, J = 2.0 Hz, 1H), 6.07 (t, J = 5.2 Hz, 1H), 4.27 (t, J = 5.6 Hz, 2H), 3.58 | 527.4 |
| | | | | | (m, 4H), 3.11 (s, 3H), | |
| | | | | | 3.09-3.14 (m, 2H), 2.74 | |
| | | | | | (t, J = 5.2 Hz, 2H), 2.50 | |
| | | | | | (br m, 4H), 1.61 (m, | |
| | | | | | 2H), 1.12 (s, 6H). | |
| 38 | 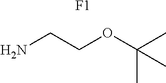 | 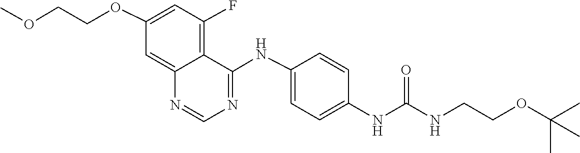 [SMILES: COCCOc1cc(F)c2c(Nc3ccc(NC(=O)NCCOC(C)(C)C)cc3)ncnc2c1] | G | 64 | 8.87 (d, J = 12.4 Hz, 1H), 8.60 (s, 1H), 8.41 (s, 1H), 7.51 (d, J = 8.8 Hz, 2H), 7.38 (d, J = 8.8 Hz, 2H), 7.12 (dd, J = 13.6, 2.4 Hz, 1H), 7.04 (d, J = 2.0 Hz, 1H), 6.07 (t, J = 5.6 Hz, 1H), 4.28 (m, 2H), 3.71 (m, 2H), 3.35 (m, 2H), 3.32 (s, | 472.4 |
| | | | | | 3H), 3.18 (m, 2H), 1.16 | |
| | | | | | (s, 9H). | |
| 39 | 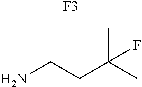 | 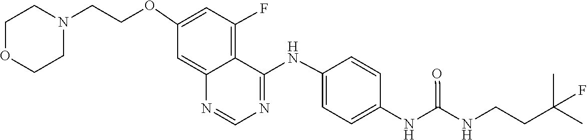 [SMILES: CC(C)(F)CCNC(=O)Nc1ccc(Nc2ncnc3cc(OCCN4CCOCC4)cc(F)c23)cc1] | G | 21 | 8.86 (d, J = 12.4 Hz, 1H), 8.51 (s, 1H), 8.41 (s, 1H), 7.51 (d, J = 8.8 Hz, 2H), 7.38 (d, J = 8.8 Hz, 2H), 7.11 (dd, J = 13.6, 2.0 Hz, 1H), 7.04 (d, J = 2.0 Hz, 1H), 6.08 (t, J = 5.6 Hz, 1H), 4.26 (t, J = 5.6 Hz, 2H), 3.58 | 515.5 |
| | | | | | (br m, 4H), 3.31 (br m, | |
| | | | | | 4H), 3.18-3.23 (m, 2H), | |
| | | | | | 2.74 (t, J = 5.6 Hz, 2H), | |
| | | | | | 1.74-1.83 (m, 2H), 1.34 | |
| | | | | | (d, J = 21.6 Hz, 6H). | |
| 40 | 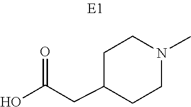 | 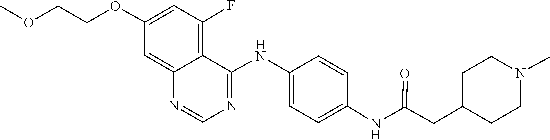 | I | 48 | 9.91 (s, 1H), 8.95 (d, J = 12.3 Hz, 1H), 8.44 (s, 1H), 7.60 (s, 4H), 7.14 (dd, J = 13.8, 2.4 Hz, 1H), 7.05 (d, J = 2.4 Hz, 1H), 4.29 (m, 2H), 3.72 (m, 2H), 3.33 (s, 3H), 2.73 (dd, J = 11.8, 3.4 Hz, 2H), 2.23 (d, J = 7.1 | 468.2 |
| | | | | | Hz, 2H), 2.13 (s, 3H), | |
| | | | | | 1.83 (td, J = 11.6, 2.4 | |
| | | | | | Hz, 2H), 1.70 (m, | |
| | | | | | 1H), 1.64 (m, 2H), 1.23 | |
| | | | | | (qd, J = 12.0, 3.8 Hz, | |
| | | | | | 2H). | |
| 41 | 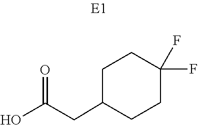 [SMILES: O=C(O)CC1CCC(F)(F)CC1] | 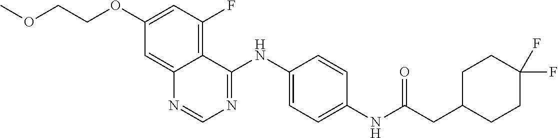 [SMILES: COCCOc1cc(F)c2c(Nc3ccc(NC(=O)CC4CCC(F)(F)CC4)cc3)ncnc2c1] | I | 61 | 9.94 (s, 1H), 8.95 (d, J = 12.3 Hz, 1H), 8.44 (s, 1H), 7.59 (m, 4H), 7.14 (dd, J = 13.8, 2.4 Hz, 1H), 7.06 (d, J = 2.7 Hz, 1H), 4.29 (m, 2H), 3.72 (m, 2H), 3.33 (s, 3H), 2.28 (d, J = 7.1 Hz, 2H), 1.95 (m, 4H), 1.80 (m, | 489.2 |
| | | | | | 3H), 1.27 (m, 2H). | |
| 42 | 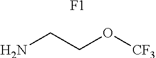 | 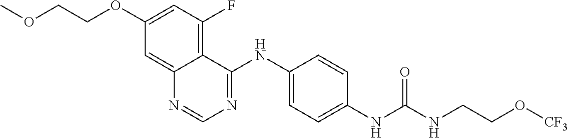 [SMILES: COCCOc1cc(F)c2c(Nc3ccc(NC(=O)NCCOC(F)(F)F)cc3)ncnc2c1] | G | 8 | 8484.3.88 (d, J = 12.4 Hz, 1H), 8.62 (s, 1H), 8.41 (s, 1H), 7.52 (d, J = 8.8 Hz, 2H), 7.39 (d, J = 8.8 Hz, 2H), 7.12 (dd, J = 14.0, 2.4 Hz, 1H), 7.04 (d, J = 2.0 Hz, 1H), 6.36 (t, J = 5.6 Hz, 1H), 4.28 (m, 2H), 4.12 (t, J = | |
| | | | | | 5.2 Hz, 2H), 3.71 (m, | |
| | | | | | 2H), 3.42 (q, J = 5.2 Hz, | |
| | | | | | 2H), 3.32 (s, 3H). | |
| 43 | 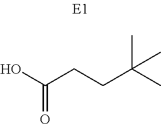 | 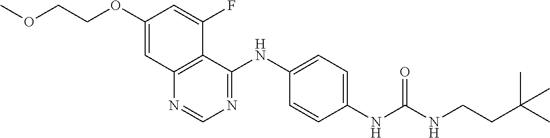 [SMILES: COCCOc1cc(F)c2c(Nc3ccc(NC(=O)NCCC(C)(C)C)cc3)ncnc2c1] | H | 10 | 9.17 (s, 1H), 9.02 (d, J = 11.2 Hz, 1H), 8.41 (d, J = 2.4 Hz, 2H), 7.96 (br m, 1H), 7.89 (dd, J = 8.8 Hz, 2.4 Hz, 1H), 7.38 (d, J = 9.2 Hz, 1H), 7.15 (dd, J = 13.6, 2.4 Hz, 1H), 7.06 (d, J = 2.0 Hz, 1H), 4.28 (m, 2H), 3.71 | 457.4 |
| | | | | | (m, 2H), 3.32 (s, 3H), | |
| | | | | | 3.19 (m, 2H), 1.41 (m, | |
| | | | | | 2H), 0.92 (s, 9H). | |
| 44 | 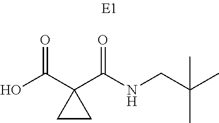 | 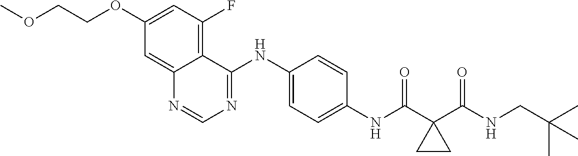 [SMILES: COCCOc1cc(F)c2c(Nc3ccc(NC(=O)C4(C(=O)NCC(C)(C)C)CC4)cc3)ncnc2c1] | I | 57 | 10.47 (s, 1H), 9.05 (d, J = 12.2 Hz, 1H), 8.48 (s, 1H), 8.02 (t, J = 6.3 Hz, 1H), 7.63 (m, 2H), 7.58 (m, 2H), 7.17 (dd, J = 13.8, 2.5 Hz, 1H), 7.06 (d, J = 2.4 Hz, 1H), 4.26-4.33 (m, 2H), 3.69- 3.75 (m, 2H), 3.33 (s, 3H), 2.97 (s, 2H), 1.33- | 510.2 |
| | | | | | 1.49 (m, 4H), 0.85 (s, | |
| | | | | | 9H). | |
| 45 | 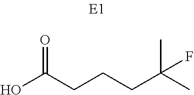 | 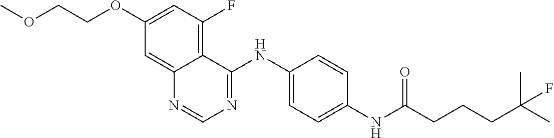 [SMILES: COCCOc1cc(F)c2c(Nc3ccc(NC(=O)CCCC(C)(C)F)cc3)ncnc2c1] | I (EDC + HOBT) | 60 | 9.91 (s, 1H), 8.93 (d, J = 12.0 Hz, 1H), 8.43 (s, 1H), 7.59 (m, 4H), 7.13 (dd, J = 14.0, 2.2 Hz, 1H), 7.05 (d, J = 2.0 Hz, 1H), 4.28 (m, 2H), 3.71 (m, 2H), 3.32 (s, 3H), 2.31 (m, 2H), 1.58-1.68 (m, 4H), 1.32 (d, J = | 459.4 |
| | | | | | 21.2 Hz, 6H) | |
| 46 | 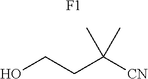 | 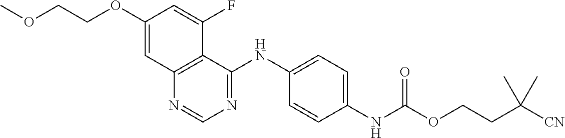 [SMILES: COCCOc1cc(F)c2c(Nc3ccc(NC(=O)OCCC(C)(C)C#N)cc3)ncnc2c1] | G | 9 | 9.66 (s, 1H), 8.93 (d, J = 12.4 Hz, 1H), 8.42 (s, 1H), 7.57 (d, J = 8.8 Hz, 2H), 7.46 (d, J = 8.4 Hz, 2H), 7.13 (dd, J = 14.0 Hz, 2.4 Hz, 1H), 7.04 (d, J = 2.4 Hz, 1H), 4.22- 4.30 (m, 4H), 3.71 (m, 2H), 3.32 (s, 3H), 1.94 | 468.3 |
| | | | | | (t, J = 6.8 Hz, 2H), 1.38 | |
| | | | | | (s, 6H). | |
| 47 | 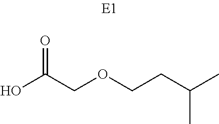 | 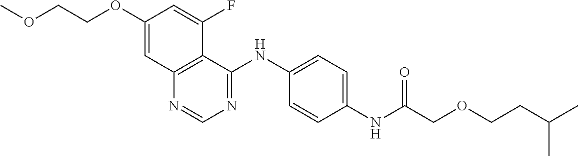 [SMILES: COCCOc1cc(F)c2c(Nc3ccc(NC(=O)COCCC(C)C)cc3)ncnc2c1] | I | 63 | 9.69 (s, 1H), 8.98 (d, J = 12.3 Hz, 1H), 8.46 (d, J = 1.3 Hz, 1H), 7.60-7.68 (m, 4H), 7.16 (dd, J = 13.9, 2.5 Hz, 1H), 7.06 (d, J = 2.4 Hz, 1H), 4.27-4.32 (m, 2H), 4.02 (d, J = 16.4 Hz, 2H), 3.69-3.75 (m, 2H), 3.55 (t, J = 6.7 Hz, 2H), 3.34 | 457.2 |
| | | | | | (s, 3H), 1.72 (m, 1H), | |
| | | | | | 1.50 (m, 2H), 0.91 (d, | |
| | | | | | J = 6.7 Hz, 6H). | |
| 48 | 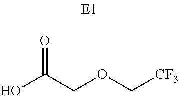 | 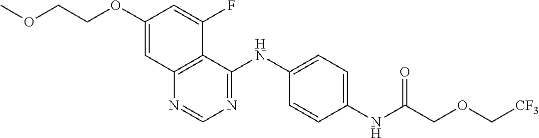 [SMILES: COCCOc1cc(F)c2c(Nc3ccc(NC(=O)COCC(F)(F)F)cc3)ncnc2c1] | I | 75 | 9.98 (s, 1H), 9.57 (s, 1H), 8.59 (s, 1H), 7.66 (d, J = 8.0 Hz, 2H), 7.60 (d, J = 8.0 Hz, 2H), 7.28 (dd, J = 13.8, 2.4 Hz, 1H), 7.06 (d, J = 2.4 Hz, 1H), 4.33 (m, 2H), 4.31 (s, 2H), 4.22- (q, J = 4.0 Hz, 2H), 3.73 (m, 2H), | 469.2 |
| | | | | | 3.34 (s, 3H). | |
| 49 | 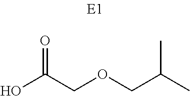 | 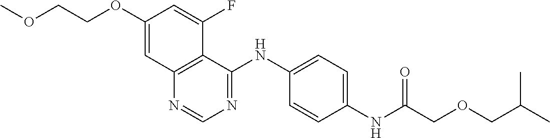 [SMILES: COCCOc1cc(F)c2c(Nc3ccc(NC(=O)COCC(C)C)cc3)ncnc2c1] | I | 74 | 9.68 (s, 1H), 8.98 (d, J = 12.3 Hz, 1H), 8.46 (s, 1H), 7.59-7.68 (m, 4H), 7.15 (dd, J = 13.9, 2.4 Hz, 1H), 7.06 (d, J = 2.4 Hz, 1H), 4.28 (m, 2H), 4.05 (s, 2H), 3.72 (m, 2H), 3.34 (s, 3H), 3.30 (m, 2H), 1.91 (m, 1H), 0.92 (d, J = 6.7 Hz, 6H). | 443.2 |
| 50 | 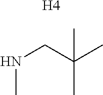 | 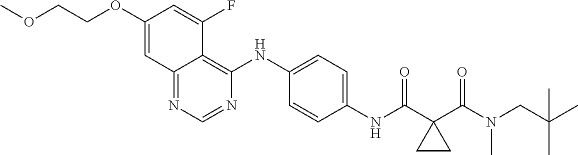 [SMILES: COCCOc1cc(F)c2c(Nc3ccc(NC(=O)C4(C(=O)N(C)CC(C)(C)C)CC4)cc3)ncnc2c1] | L | 72 | 9.35 (s, 1H), 8.96 (d, J = 12.2 Hz, 1H), 8.45 (s, 1H), 7.54-7.66 (m, 4H), 7.15 (dd, J = 13.8, 2.4 Hz, 1H), 7.06 (d, J = 2.4 Hz, 1H), 4.29 (m, 2H), 3.71 (m, 2H), 3.33 (s, 3H), 3.21 (s, 2H), 3.10 (s, 3H), 1.45 (m, 2H), 1.21 (m, 2H), 0.91 (s, | 524.2 |
| | | | | | 9H). | |
| 51 | 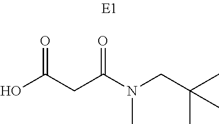 | 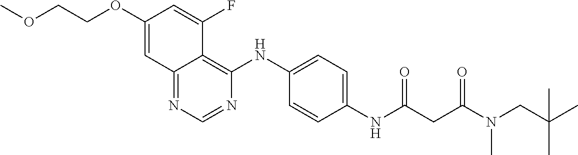 [SMILES: COCCOc1cc(F)c2c(Nc3ccc(NC(=O)CC(=O)N(C)CC(C)(C)C)cc3)ncnc2c1] | I | 46 | 10.13 (d, J = 2.6 Hz, 1H), 8.96 (d, J = 12.3 Hz, 1H), 8.45 (s, 1H), 7.56-7.67 (m, 4H), 7.15 (dd, J = 13.8, 2.5 Hz, 1H), 7.06 (d, J = 2.4 Hz, 1H), 4.29 (m, 2H), 3.71 (m, 2H), 3.53 (d, J = 2.7 Hz, 2H), 3.34 (s, 3H), 3.32 (s, 2H), 3.10 (s, | 498.2 |
| | | | | | 3H), 0.91 (s, 9H). | |
| 52 | 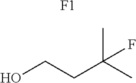 | 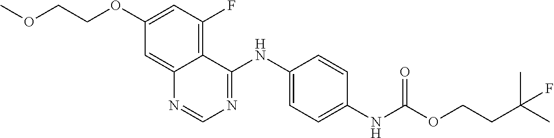 [SMILES: COCCOc1cc(F)c2c(Nc3ccc(NC(=O)OCCC(C)(C)F)cc3)ncnc2c1] | G | 8 | 9.64 (s, 1H), 9.02 (br d, J = 12.0 Hz, 1H), 8.44 (s, 1H), 7.56 (d, J = 9.2 Hz, 2H), 7.46 (d, J = 8.8 Hz, 2H), 7.14 (dd, J = 12.0, 2.4 Hz, 1H), 7.04 (d, J = 2.4 Hz, 1H), 4.28 (m, 2H), 4.21 (t, J = 6.8 Hz, 2H), 3.71 (m, 2H), | 461.3 |
| | | | | | 3.32 (s, 3H), 2.02 (t, J = | |
| | | | | | 7.2 Hz, 1H), 1.97 (t, J = | |
| | | | | | 6.8 Hz, 1H), 1.38 (d, J = | |
| | | | | | 21.6 Hz, 6H). | |
| 53 | 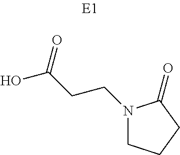 [SMILES: O=C(O)CCN1CCCC1=O] | 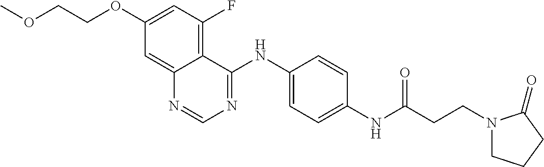 [SMILES: COCCOc1cc(F)c2c(Nc3ccc(NC(=O)CCN4CCCC4=O)cc3)ncnc2c1] | I | 55 | 11.02 (br s, 1H), 9.08 (d, J = 15.2 Hz, 1H), 8.92 (s, 1H), 8.12 (d, J = 8.8 Hz, 2H), 8.02 (d, J = 8.8 Hz, 2H), 7.40 (d, J = 2.0 Hz, 1H), 7.03 (dd, J = 14.0, 2.0 Hz, 1H), 4.27 (m, 2H), 3.84 (t, J = 6.8 Hz, 2H), 3.72 (m, 2H), 3.34 (s, 3H), 3.30 (t, J = 7.2 Hz, 2H), 2.83 (t, J = | 468.3 |
| | | | | | 6.8 Hz, 2H), 2.21 (t, J = | |
| | | | | | 8.4 Hz, 2H), 1.62-1.70 | |
| | | | | | (m, 2H). In Pyridine-d5. | |
| 54 | 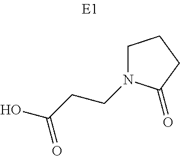 [SMILES: O=C(O)CCN1CCCC1=O] | 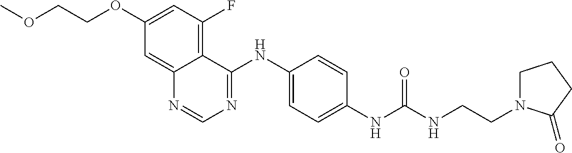 [SMILES: COCCOc1cc(F)c2c(Nc3ccc(NC(=O)NCCN4CCCC4=O)cc3)ncnc2c1] | H | 19 | 8.87 (d, J = 12.4 Hz, 1H), 8.54 (s, 1H), 8.41 (s, 1H), 7.50 (d, J = 8.8 Hz, 2H), 7.38 (d, J = 8.8 Hz, 2H), 7.12 (dd, J = 14.0, 2.4 Hz, 1H), 7.03 (d, J = 2.4 Hz, 1H), 6.09 (br t, J = 4.8 Hz, 1H), 4.27 (m, 2H), 3.71 (m, 2H), 3.39 (t, J = 7.2 Hz, | 483.4 |
| | | | | | 2H), 3.32 (s, 3H), 3.22- | |
| | | | | | 3.30 (m, 4H), 2.20 (t, | |
| | | | | | J = 8.0 Hz, 2H), 1.87- | |
| | | | | | 1.97 (m, 2H). | |
| 55 | 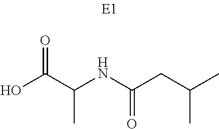 | 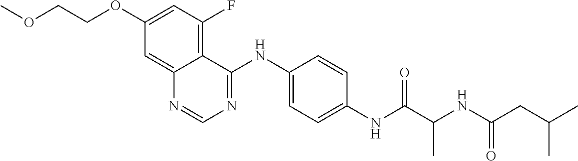 [SMILES: COCCOc1cc(F)c2c(Nc3ccc(NC(=O)C(C)NC(=O)CC(C)C)cc3)ncnc2c1] | I | 36 | 9.70 (s, 1H), 8.76 (br d, J = 13.2 Hz, 1H), 8.45 (s, 1H), 7.78 (d, J = 6.8 Hz, 1H), 7.65 (d, J = 9.2 Hz, 2H), 7.57 (d, J = 8.8 Hz, 2H), 7.08 (m, 2H), 4.46 (m, 1H), 4.30 (m, 2H), 3.73 (m, 2H), 3.35 (s, 3H), 1.98-2.06 (m, 3H), 1.32 (d, J = 7.2 Hz, 3H), 0.91 (d, J = 6.4 Hz, | 484.4 |
| | | | | | 6H). at high Temp. | |
| 56 | 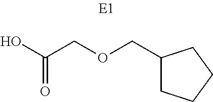 [SMILES: O=C(O)COCC1CCCC1] | 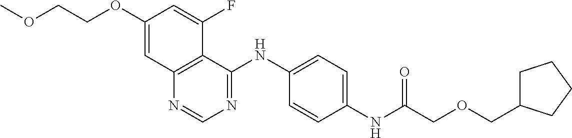 [SMILES: COCCOc1cc(F)c2c(Nc3ccc(NC(=O)COCC4CCCC4)cc3)ncnc2c1] | I | 64 | 9.61 (s, 1H), 8.89 (d, J = 12.3 Hz, 1H), 8.38 (s, 1H), 7.56 (m, 4H), 7.07 (dd, J = 13.9, 2.4 Hz, 1H), 6.98 (d, J = 2.4 Hz, 1H), 4.21 (m, 2H), 3.97 (s, 2H), 3.64 (m, 2H), 3.31 (dd, J = 10.9, 7.1 | 469.2 |
| | | | | | Hz, 2H), 3.26 (s, 3H), | |
| | | | | | 2.13 (m, 1H), 1.64 (m, | |
| | | | | | 2H), 1.38-1.55 (m, 4H), | |
| | | | | | 1.19 (m, 2H). | |
| 57 | 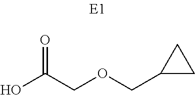 | 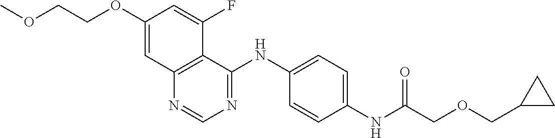 [SMILES: COCCOc1cc(F)c2c(Nc3ccc(NC(=O)COCC4CC4)cc3)ncnc2c1] | I | 62 | 9.47 (s, 1H), 8.72 (d, J = 12.3 Hz, 1H), 8.21 (s, 1H), 7.42 (d, J = 9.1 Hz, 2H), 7.38 (d, J = 9.1 Hz, 2H), 6.90 (dd, J = 13.8, 2.5 Hz, 1H), 6.82 (d, J = 2.4 Hz, 1H), 4.04 (m, 2H), 3.83 (s, 2H), 3.48 (m, 2H), 3.15 (d, J = 6.9 | 441.2 |
| | | | | | Hz, 2H), 3.09 (s, 3H), | |
| | | | | | 0.85 (m, 1H), 0.27 (m, | |
| | | | | | 2H). | |
| 58 | 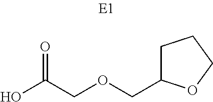 [SMILES: O=C(O)COCC1CCCO1] | 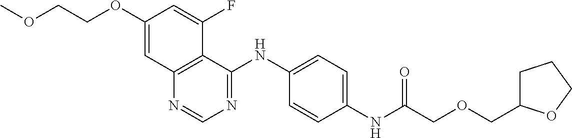 [SMILES: COCCOc1cc(F)c2c(Nc3ccc(NC(=O)COCC4CCCO4)cc3)ncnc2c1] | I | 63 | 9.63 (s, 1H), 8.89 (d, J = 12.3 Hz, 1H), 8.38 (s, 1H), 7.57 (s, 4H), 7.07 (dd, J = 13.9, 2.4 Hz, 1H), 6.99 (d, J = 2.4 Hz, 1H), 4.24-4.19 (m, 2H), 4.03 (s, 2H), 4.02- 3.94 (m, 1H), 3.72 (dt, J = 8.2, 6.7 Hz, 1H), | 471.2 |
| | | | | | 3.67-3.59 (m, 3H), 3.53 | |
| | | | | | (dd, J = 10.6, 3.7 Hz, | |
| | | | | | 1H), 3.43 (dd, J = 10.6, | |
| | | | | | 6.6 Hz, 1H), 3.30 (s, | |
| | | | | | 3H), 1.86 (m, 1H), 1.76 | |
| | | | | | (m, 2H), 1.51 (m, 1H). | |
| 59 | 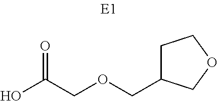 [SMILES: O=C(O)COCC1CCOC1] | 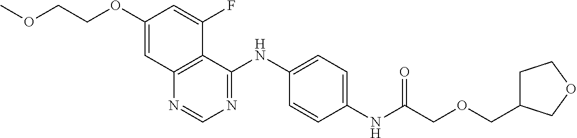 [SMILES: COCCOc1cc(F)c2c(Nc3ccc(NC(=O)COCC4CCOC4)cc3)ncnc2c1] | I | 38 | 9.73 (s, 1H), 8.96 (d, J = 12.4 Hz, 1H), 8.45 (s, 1H), 7.64 (s, 4H), 7.15 (dd, J = 13.8, 2.5 Hz, 1H), 7.06 (d, J = 2.4 Hz, 1H), 4.29 (m, 2H), 4.07 (s, 2H), 3.63 (m, 4H), 3.62 (m, 1H), 3.47-3.57 (m, 2H), 3.44 (m, 1H), | 471.2 |
| | | | | | 3.34 (s, 3H), 2.57 (m, | |
| | | | | | 1H), 1.96 (m, 1H), 1.58 | |
| | | | | | (m, 1H). | |
| 60 | 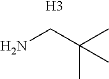 | 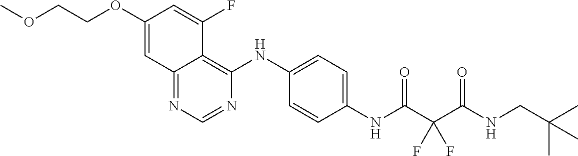 [SMILES: COCCOc1cc(F)c2c(Nc3ccc(NC(=O)C(F)(F)C(=O)NCC(C)(C)C)cc3)ncnc2c1] | L | 5 | 10.81 (s, 1H), 9.03 (d, J = 12.1 Hz, 1H), 8.96 (t, J = 6.6 Hz, 1H), 8.48 (s, 1H), 7.71 (m, 4H), 7.17 (dd, J = 13.8, 2.4 Hz, 1H), 7.08 (d, J = 2.4 Hz, 1H), 4.30 (m, 2H), 3.73 (m, 2H), 3.34 (s, 3H), 3.01 (m, 2H), 0.89 (d, J = 11.1 Hz, 9H). | 520.2 |
| 61 | 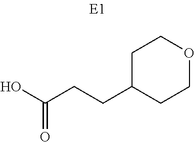 [SMILES: O=C(O)CCC1CCOCC1] | 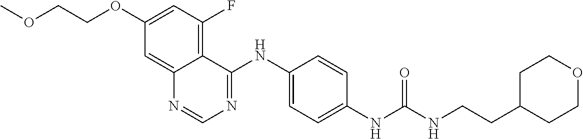 [SMILES: COCCOc1cc(F)c2c(Nc3ccc(NC(=O)NCCC4CCOCC4)cc3)ncnc2c1] | H | 19 | 8.86 (d, J = 12.4 Hz, 1H), 8.41 (s, 1H), 8.39 (s, 1H), 7.50 (d, J = 8.8 Hz, 2H), 7.38 (d, J = 8.8 Hz, 2H), 7.12 (dd, J = 13.6, 2.0 Hz, 1H), 7.03 (d, J = 2.0 Hz, 1H), 6.08 (br t, J = 5.2 Hz, 1H), 4.28 (m, 2H), 3.83 (dd, | 484.4 |
| | | | | | J = 10.8, 3.2 Hz, 2H), | |
| | | | | | 3.71 (m, 2H), 3.32 (s, | |
| | | | | | 3H), 3.27 (m, 2H), 3.13 | |
| | | | | | (q, J = 6.4 Hz, 2H), | |
| | | | | | 1.57-1.63 (m, 2H), 1.49- | |
| | | | | | 1.56 (m, 1H), 1.38 (q, | |
| | | | | | J = 6.8 Hz, 2H), 1.11- | |
| | | | | | 1.21 (m, 2H). | |
| 62 | 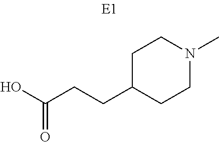 | 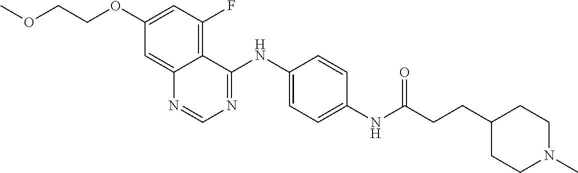 | I | 63 | 8.40 (s, 1H), 7.64 (AB d, J = 8.8 Hz, 2H), 7.60 (AB d, J = 8.8 Hz, 2H), 7,04 (s, 1H), 7.01 (m, 1H), 4.29 (m, 2H), 3.81 (m, 2H), 3.44 (s, 3H), 3.42 (m, 2H), 2.91 (m, 2H), 2.80 (s, 3H), 2.44 (m, 2H), 2.03 (m, 2H), 1.72 (q, J = 7.2 Hz, 2H), 1.57-1.70 (m, 1H), 1.45 | 482.2 |
| | | | | | (m, 2H) in MeOD. two | |
| | | | | | NHs are missing | |
| 63 | 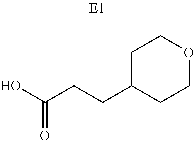 [SMILES: O=C(O)CCC1CCOCC1] | 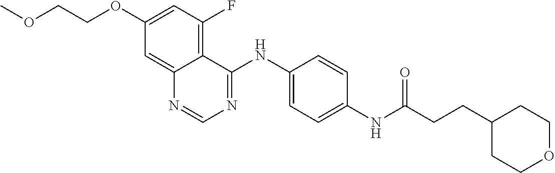 [SMILES: COCCOc1cc(F)c2c(Nc3ccc(NC(=O)CCC4CCOCC4)cc3)ncnc2c1] | I | 55 | 8.41 (s, 1H), 7.64 (AB d, J = 9.6 Hz, 2H), 7.61 (AB d, J = 9.6 Hz, 2H), 7,04 (s, 1H), 7.01 (m, 1H), 4.30 (m, 2H), 3.95 (dd, J = 10.8, 3.63 Hz, 2H) 3.82 (m, 2H), 3.45 (s, 3H), 3.41 (m, 2H), 2.44 (m, 2H), 1.68 (m, 4H), 1.56-1.68 (m, 1H), 1.33 (m, 2H) in MeOD. | 469.4 |
| | | | | | two NHs are missing | |
| 64 | 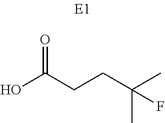 | 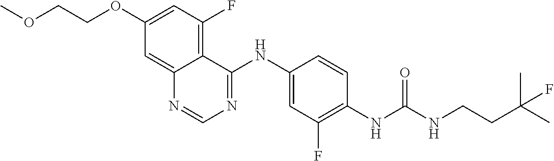 [SMILES: COCCOc1cc(F)c2c(Nc3ccc(NC(=O)NCCC(C)(C)F)c(F)c3)ncnc2c1] | H | 6 | 8.98 (d, J = 12.0 Hz, 1H), 8.48 (s, 1H), 8.30 (s, 1H), 8.04 (t, J = 9.2 Hz, 1H), 7.74 (dd, J = 13.6, 2.0 Hz, 1H), 7.34 (d, J = 8.8 Hz, 1H), 7.16 (dd, J = 13.6, 2.0 Hz, 1H), 7.06 (d, J = 2.0 Hz, 1H), 6.53 (t, J = 5.6 Hz, 1H), 4.29 (m, 2H), 3.71 (m, 2H), 3.32 (s, 3H), 3.22 (dd, J = 14.8, 6.0 | 478.3 |
| | | | | | Hz, 2H), 1.75-1.83 (m, | |
| | | | | | 2H), 1.35 (d, J = 21.6 | |
| | | | | | Hz, 6H). | |
| 65 | 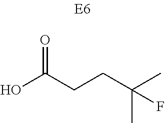 | 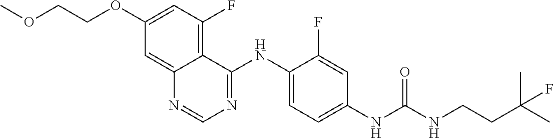 [SMILES: COCCOc1cc(F)c2c(Nc3ccc(NC(=O)NCCC(C)(C)F)cc3F)ncnc2c1] | H | 11 | 8.84 (d, J = 12.0 Hz, 1H), 8.78 (s, 1H), 8.38 (s, 1H), 7.55 (dd, J = 13.2, 2.0 Hz, 1H), 7.49 (t, J = 8.8 Hz, 1H), 7.13 (dd, J = 14.0, 2.4 Hz, 1H), 7.05 (br m, 2H), 6.19 (t, J = 5.6 Hz, 1H), 4.28 (m, 2H), 3.71 (m, | 478.3 |
| | | | | | 2H), 3.32 (s, 3H), 3.21 | |
| | | | | | (m, 2H), 1.80 (dt, J = | |
| | | | | | 20.0, 8.0 Hz, 2H), 1.35 | |
| | | | | | (d, J = 21.6 Hz, 6H). | |
| 66 | 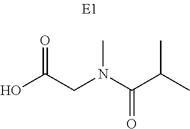 | 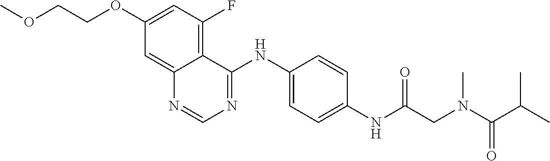 [SMILES: COCCOc1cc(F)c2c(Nc3ccc(NC(=O)CN(C)C(=O)C(C)C)cc3)ncnc2c1] | I | 39 | 9.70-9.90 (br s, 1H), 8.76 (d, J = 13.2 Hz, 1H), 8.46 (s, 1H), 7.66 (d, J = 8.8 Hz, 2H), 7.57 (d, J = 8.8 Hz, 2H), 7.11 (m, 1H), 7.06 (s, 1H), 4.31 (m, 2H), 4.03-4.30 (br m, 2H), 3.73 (m, 2H), 3.35 (s, 3H), 3.07 (s, 3H), 2.85-3.00 (br m, | 470.4 |
| | | | | | 1H), 1.05 (d, J = 6.4 Hz, | |
| | | | | | 6H). at high Temp. | |
| 67 | 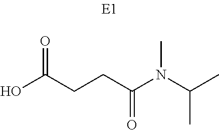 | 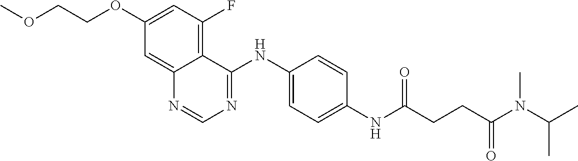 [SMILES: COCCOc1cc(F)c2c(Nc3ccc(NC(=O)CCC(=O)N(C)C(C)C)cc3)ncnc2c1] | I | 27 | 9.72 (s, 1H), 8.74 (d, J = 13.2 Hz, 1H), 8.45 (s, 1H), 7.63 (d, J = 8.4 Hz, 2H), 7.57 (d, J = 8.8 Hz, 2H), 7.01-7.09 (m, 2H), 4.66 (br m, 1H), 4.31 (m, 2H), 3.73 (m, 2H), 3.35 (s, 3H), 2.71-2.85 (br m, 3H), 2.57-2.63 (m, 4H), 0.96-1.20 (br m, 6H). at high Temp. | 484.4 |
| 68 | 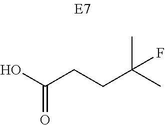 | 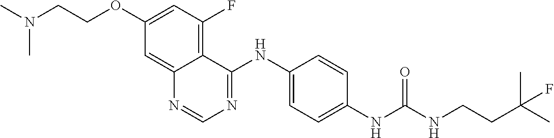 [SMILES: CN(C)CCOc1cc(F)c2c(Nc3ccc(NC(=O)NCCC(C)(C)F)cc3)ncnc2c1] | H | 6 | 8.86 (d, J = 12.4 Hz, 1H), 8.51 (s, 1H), 8.41 (s, 1H), 7.51 (d, J = 8.8 Hz, 2H), 7.38 (d, J = 8.8 Hz, 2H), 7.12 (dd, J = 13.6, 2.0 Hz, 1H), 7.05 (d, J = 2.0 Hz, 1H), 6.08 (br t, J = 5.6 Hz, 1H), 4.25 (m, 2H), 3.20 (m, | 473.5 |
| | | | | | 2H), 2.76 (br m, 2H), | |
| | | | | | 2.29 (s, 6H), 1.79 (dt, | |
| | | | | | J = 20, 7.6 Hz, 2H), 1.35 | |
| | | | | | (d, J = 21.6 Hz, 6H). | |
| 69 | 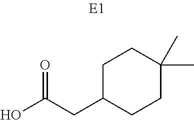 | 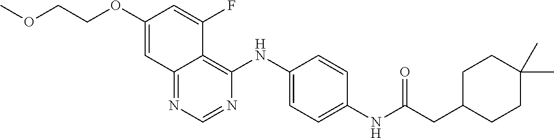 | I | 40 | 9.87 (s, 1H), 8.94 (d, J = 12.4 Hz, 1H), 8.44 (s, 1H), 7.60 (s, 4H), 7.14 (dd, J = 13.8, 2.4 Hz, 1H), 7.06 (d, J = 2.4 Hz, 1H), 4.29 (m, 2H), 3.72 (m, 2H), 3.35 (s, 3H), 2.22 (d, J = 7.2 Hz, 2H), 1.54 (m, 2H), 1.36 (m, | 481.2 |
| | | | | | 2H), 1.18 (m, 5H), 0.87 | |
| | | | | | (s, 3H), 0.90 (s, 3H). | |
| 70 | 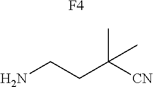 | 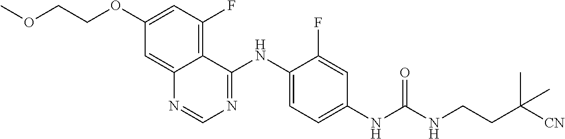 [SMILES: COCCOc1cc(F)c2c(Nc3ccc(NC(=O)NCCC(C)(C)C#N)cc3F)ncnc2c1] | G | 21 | 9.40 (br s, 1H), 9.37 (s, 1H), 8.84 (d, J = 12.0 Hz, 1H), 8.38 (s, 1H), 7.74 (br d, J = 11.7 Hz, 1H), 7.49 (t, J = 8.8 Hz, 1H), 7.39 (br d, J = 8.4 Hz, 1H), 7.13 (dd, J = 13.7, 2.3 Hz, 1H), 7.05 (d, J = 2.1 Hz, 1H), 4.28 | 485.4 |
| | | | | | (m, 2H), 3.71 (m, 2H), | |
| | | | | | 3.44 (t, J = 6.9 Hz, 2H), | |
| | | | | | 3.32 (s, 3H), 1.80 (t, J = | |
| | | | | | 6.8 Hz, 2H), 1.17 (s, 6H). | |
| 71 | 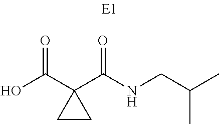 | 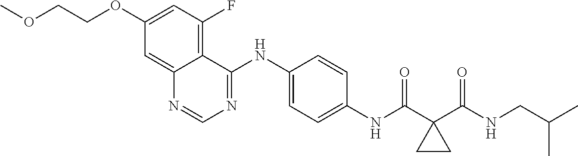 [SMILES: COCCOc1cc(F)c2c(Nc3ccc(NC(=O)C4(C(=O)NCC(C)C)CC4)cc3)ncnc2c1] | I | 42 | 10.47 (br s, 1H), 8.77 (d, J = 13.6 Hz, 1H), 8.46 (s, 1H), 7.79 (br s, 1H), 7.66 (d, J = 8.8 Hz, 2H), 7.56 (d, J = 9.2 Hz, 2H), 7.08 (m, 2H), 4.31 (m, 2H), 3.73 (m, 2H), 3.35 (s, 3H), 2.98 (m, 2H), 1.80 (m, 1H), 1.43 (m, 2H), 1.38 (m, 2H), 0.86 | 496.4 |
| | | | | | (d, J = 6.8 Hz, 6H). at | |
| | | | | | high Temp. | |
| 72 | 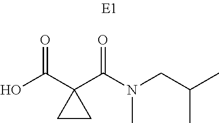 | 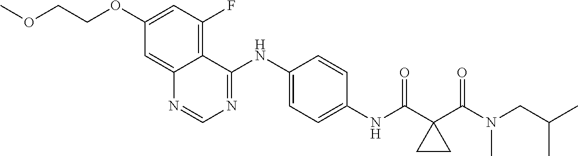 [SMILES: COCCOc1cc(F)c2c(Nc3ccc(NC(=O)C4(C(=O)N(C)CC(C)C)CC4)cc3)ncnc2c1] | I | 34 | 9.18 (br s, 1H), 8.76 (d, J = 13.2 Hz, 1H), 8.45 (s, 1H), 7.63 (d, J = 8.8 Hz, 2H), 7.56 (d, J = 9.2 Hz, 2H), 7.08 (m, 2H), 4.31 (m, 2H), 3.73 (m, 2H), 3.35 (s, 3H), 3.19 (d, J = 7.2 Hz, 2H), 2.99 (br s, 3H), 1.98 (m, 1H), 1.41 (m, 2H), 1.20 (m, | 510.5 |
| | | | | | 2H), 0.85 (d, J = 6.8 Hz, | |
| | | | | | 6H). at high Temp. | |
| 73 | 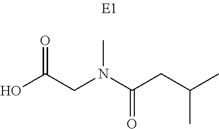 | 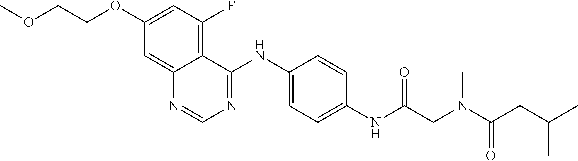 [SMILES: COCCOc1cc(F)c2c(Nc3ccc(NC(=O)CN(C)C(=O)CC(C)C)cc3)ncnc2c1] | I | 34 | 9.87 (br s, 0.3H), 9.72 (br s, 0.7H), 8.77 (d, J = 13.6 Hz, 1H), 8.46 (s, 1H), 7.66 (br d, J = 7.6 Hz, 2H), 7.57 (br d, J = 8.4 Hz, 2H), 7.08 (m, 2H), 4.31 (m, 2H), 4.12 (br m, 2H), 3.73 (m, 2H), 3.35 (s, 3H), 2.88 (br s, 3H), 2.17-2.25 (br m, 2H), 2.06 (m, 1H), | 485.5 |
| | | | | | 0.94 (br d, J = 6.8 Hz, | |
| | | | | | 6H). at high Temp. | |
| 74 | 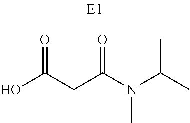 | 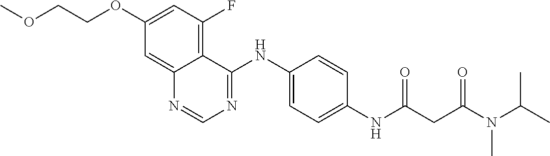 [SMILES: COCCOc1cc(F)c2c(Nc3ccc(NC(=O)CC(=O)N(C)C(C)C)cc3)ncnc2c1] | I (EDC + HOBT) | 27 | 10.12 (s, 1H), 8.95 (d, J = 12.3 Hz, 1H), 8.44 (s, 1H), 7.62 (d, 8.8 Hz, 2H), 7.58 (d, J = 9.2 Hz, 2H), 7.14 (dd, J = 16.0, 2.0 Hz, 1H), 7.05 (d, J = 2.0 Hz, 1H), 4.64 (m, 0.5H, rotamer 1), 4.28 (m, 2H), 4.14 (m, 0.5H, rotamer 2), 3.72 (m, | 470.4 |
| | | | | | 2H), 3.51 (s, 1H, | |
| | | | | | rotamer 1), 3.45 (s, 1H, | |
| | | | | | rotamer 2), 2.82 (s, | |
| | | | | | 1.5H, rotamer 1), 2.69 | |
| | | | | | (s, 1.5H, rotamer 2), | |
| | | | | | 2.40-2.45 (m, 3H), 1.15 | |
| | | | | | (d, J = 6.2 Hz, 3H, | |
| | | | | | rotamer 1), 1.04 (d, J = | |
| | | | | | 6.2 Hz, 3H, rotamer 2). | |
| | | | | | 1:1 rotamers. | |
| 75 | 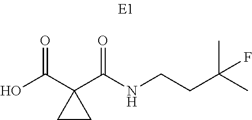 | 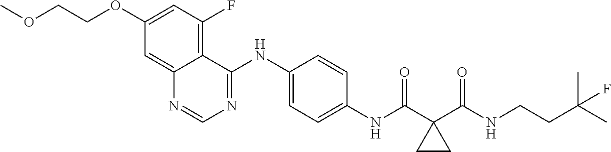 [SMILES: COCCOc1cc(F)c2c(Nc3ccc(NC(=O)C4(C(=O)NCCC(C)(C)F)CC4)cc3)ncnc2c1] | I | 14 | 10.72 (s, 1H), 8.97 (d, J = 12.0 Hz, 1H), 8.45 (s, 1H), 7.93 (br t, J = 5.6 Hz, 1H), 7.63 (d, J = 9.2 Hz, 2H), 7.58 (d, J = 8.8 Hz, 2H), 7.14 (dd, J = 14.0, 2.0 Hz, 1H), 7.05 (d, J = 2.0 Hz, 1H), 4.28 (m, 2H), 3.71 (m, 2H), 3.32 (s, 3H), 3.23 (m, | 528.5 |
| | | | | | 2H), 1.78 (dt, J = 20.0, | |
| | | | | | 7.2 Hz, 2H), 1.38 (m, | |
| | | | | | 2H), 1.34 (m, 2H), 1.32 | |
| | | | | | (d, J = 24.0 Hz, 6H). | |
| 76 | 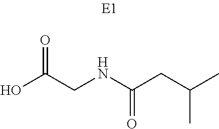 | 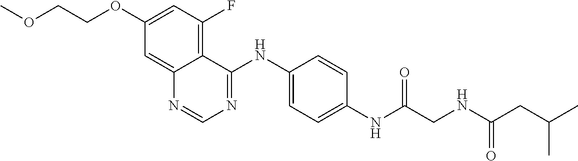 [SMILES: COCCOc1cc(F)c2c(Nc3ccc(NC(=O)CNC(=O)CC(C)C)cc3)ncnc2c1] | I | 31 | 9.99 (s, 1H), 8.97 (d, J = 12.0 Hz, 1H), 8.44 (s, 1H), 8.14 (br t, J = 6.0 Hz, 1H), 7.62 (d, J = 9.2 Hz, 2H), 7.57 (d, J = 9.2 Hz, 2H), 7.14 (dd, J = 14.0, 2.0 Hz, 1H), 7.05 (d, J = 2.0 Hz, 1H), 4.28 (m, 2H), 3.87 (d, J = 5.6 Hz, 2H), 3.71 (m, 2H), | 470.5 |
| | | | | | 3.32 (s, 3H), 2.03 (m, | |
| | | | | | 2H), 1.98 (m, 1H), 0.90 | |
| | | | | | (d, J = 6.4 Hz, 6H). | |
| 77 | 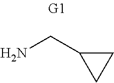 | 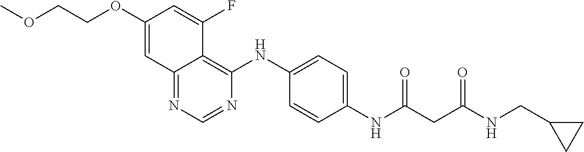 [SMILES: COCCOc1cc(F)c2c(Nc3ccc(NC(=O)CC(=O)NCC4CC4)cc3)ncnc2c1] | M | 55 | 10.13 (s, 1H), 8.95 (d, J = 12.4 Hz, 1H), 8.44 (s, 1H), 8.15 (t, J = 5.7 Hz, 1H), 7.60 (m, 4H), 7.14 (dd, J = 13.8, 2.5 Hz, 1H), 7.05 (d, J = 2.4 Hz, 1H), 4.28 (m, 2H), 3.71 (m, 2H), 3.32 (m, 3H), 3.26 (s, 2H), 2.99 (t, J = 6.2 Hz, 2H), 0.91 (m, | 468.2 |
| | | | | | 1H), 0.42 (m, 2H), 0.18 | |
| | | | | | (m, 2H). | |
| 78 | 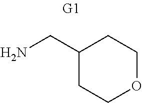 | 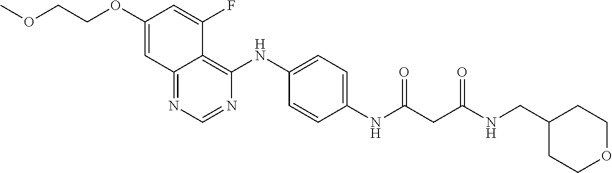 [SMILES: COCCOc1cc(F)c2c(Nc3ccc(NC(=O)CC(=O)NCC4CCOCC4)cc3)ncnc2c1] | M | 67 | δ 10.13 (s, 1H), 8.96 (d, J = 12.3 Hz, 1H), 8.45 (s, 1H), 8.10 (t, J = 5.9 Hz, 1H), 7.62 (m, 4H), 7.15 (dd, J = 13.8, 2.4 Hz, 1H), 7.06 (d, J = 2.4 Hz, 1H), 4.29 (m, 2H), 3.85 (ddd, J = 11.4, 4.5, 1.9 Hz, 2H), 3.72 (m, 2H), 3.34 (s, 3H), 3.27 (m, 4H), 3.01 (t, J = 6.3 | 512.2 |
| | | | | | Hz, 2H), 1.66 (dqt, J = | |
| | | | | | 10.7, 6.7, 3.5 Hz, 1H), | |
| | | | | | 1.58 (ddd, J = 13.0, 4.1, | |
| | | | | | 1.9 Hz, 2H), 1.17 (qd, | |
| | | | | | J = 12.0, 4.5 Hz, 2H). | |
| 79 | 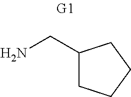 | 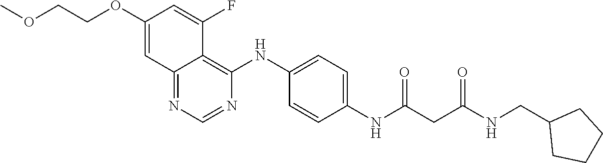 [SMILES: COCCOc1cc(F)c2c(Nc3ccc(NC(=O)CC(=O)NCC4CCCC4)cc3)ncnc2c1] | M | 16 | 10.12 (s, 1H), 8.97 (d, J = 12.2 Hz, 1H), 8.45 (s, 1H), 8.07 (t, J = 5.7 Hz, 1H), 7.62 (m, 4H), 7.15 (dd, J = 13.8, 2.4 Hz, 1H), 7.06 (d, J = 2.4 Hz, 1H), 4.29 (m, 2H), 3.72 (m, 2H), 3.34 (s, 3H), 3.25 (s, 2H), 3.04 (dd, J = 7.2, 5.7 Hz, 2H), 2.00 (m, 1H), 1.67 (m, 2H), | 496.2 |
| | | | | | 1.57 (m, 2H), 1.50 (m, | |
| | | | | | 2H), 1.20 (m, 2H). | |
| 80 | 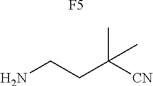 | 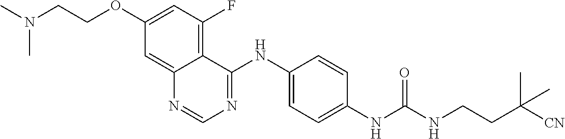 [SMILES: CN(C)CCOc1cc(F)c2c(Nc3ccc(NC(=O)NCCC(C)(C)C#N)cc3)ncnc2c1] | G | 8 | 9.33 (br s, 1H), 9.12 (br s, 1H), 8.87 (d, J = 12.8 Hz, 1H), 8.41 (s, 1H), 7.64 (d, J = 9.2 Hz, 2H), 7.50 (d, J = 9.2 Hz, 2H), 7.10 (dd, J = 14.0, 2.4 Hz, 1H), 7.04 (d, J = 2.0 Hz, 1H), 4.22 (t, J = 6.0 Hz, 2H), 3.43 (t, J = 7.2 Hz, 2H), 2.66 (m, 2H), | 480.5 |
| | | | | | 2.23 (s, 6H), 1.79 (t, J = | |
| | | | | | 7.2 Hz, 2H), 1.16 (s, | |
| | | | | | 6H). | |
| 81 | 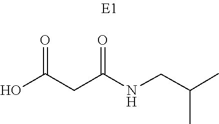 | 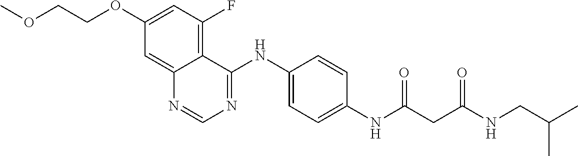 [SMILES: COCCOc1cc(F)c2c(Nc3ccc(NC(=O)CC(=O)NCC(C)C)cc3)ncnc2c1] | I | 31 | 10.1 (s, 1H), 8.95 (d, J = 12.4 Hz, 1H), 8.44 (s, 1H), 8.04 (br t, J = 5.6 Hz, 1H), 7.63 (d, J = 8.8 Hz, 2H), 7.58 (d, J = 9.2 Hz, 2H), 7.14 (dd, J = 14.0, 2.4 Hz, 1H), 7.05 (d, J = 2.4 Hz, 1H), 4.28 (m, 2H), 3.71 (m, 2H), 3.32 (s, 3H), 3.26 (s, | 470.5 |
| | | | | | 2H), 2.92 (t, J = 6.0 Hz, | |
| | | | | | 2H), 1.70 (m, 1H), 0.86 | |
| | | | | | (d, J = 6.8 Hz, 6H). | |
| 82 | 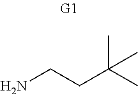 | 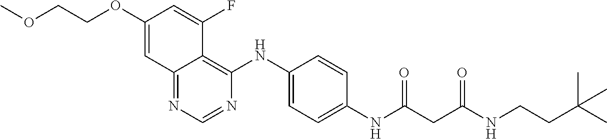 [SMILES: COCCOc1cc(F)c2c(Nc3ccc(NC(=O)CC(=O)NCCC(C)(C)C)cc3)ncnc2c1] | M | 79 | 10.12 (s, 1H), 8.96 (d, J = 12.4 Hz, 1H), 8.45 (s, 1H), 8.01 (t, J = 5.5 Hz, 1H), 7.61 (m, 4H), 7.14 (dd, J = 13.8, 2.4 Hz, 1H), 7.06 (d, J = 2.4 Hz, 1H), 4.29 (m, 2H), 3.72 (m, 2H), 3.33 (s, 3H), 3.23 (s, 2H), 3.11 (m, | 498.2 |
| | | | | | 2H), 1.37 (m, 2H), 0.90 | |
| | | | | | (s, 9H). | |
| 83 | 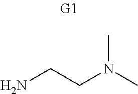 | 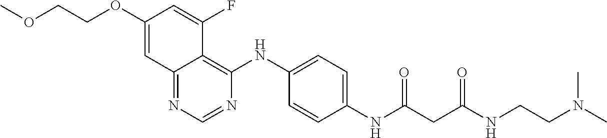 | M | 82 | 10.12 (s, 1H), 8.96 (d, J = 12.4 Hz, 1H), 8.45 (d, J = 1.1 Hz, 1H), 8.02 (t, J = 5.6 Hz, 1H), 7.61 (m, 4H), 7.14 (dt, J = 13.8, 2.0 Hz, 1H), 7.05 (m, 1H), 4.29 (m, 2H), 3.72 (m 2H), 3.34 (m, 3H), 3.27 (s, 2H), 3.19 | 485.2 |
| | | | | | (q, J = 6.3 Hz, 2H), 2.31 | |
| | | | | | (t, J = 6.6 Hz, 2H), 2.16 | |
| | | | | | (s, 6H). | |
| 84 | 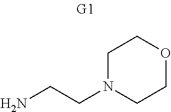 | 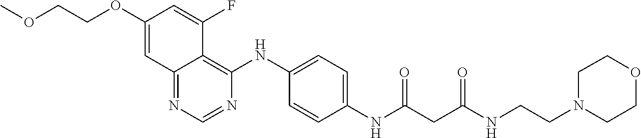 [SMILES: COCCOc1cc(F)c2c(Nc3ccc(NC(=O)CC(=O)NCCN4CCOCC4)cc3)ncnc2c1] | M | 34 | 10.13 (s, 1H), 8.96 (d, J = 12.3 Hz, 1H), 8.45 (s, 1H), 8.04 (t, J = 5.6 Hz, 1H), 7.62 (m, 4H), 7.15 (dd, J = 13.7, 2.5 Hz, 1H), 7.06 (d, J = 2.4 Hz, 1H), 4.29 (m, 2H), 3.72 (m, 2H), 3.57 (t, J = 4.6 Hz, 4H), 3.33 (s, 3H), | 527.2 |
| | | | | | 3.26 (s, 2H), 3.22 (m, | |
| | | | | | 2H), 2.38 (m, 6H). | |
| 85 | 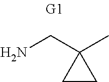 | 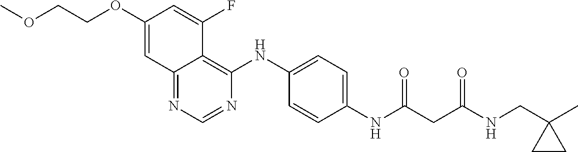 | M | 51 | 10.12 (s, 1H), 8.96 (d, J = 12.3 Hz, 1H), 8.44 (s, 1H), 8.08 (t, J = 5.9 Hz, 1H), 7.60 (m, 4H), 7.14 (dd, J = 13.7, 2.4 Hz, 1H), 7.05 (d, J = 2.4 Hz, 1H), 4.28 (m, 2H), 3.71 (m, 2H), 3.33 (s, 3H), 3.28 (s, 2H), 3.01 (d, J = 5.8 Hz, 2H), 1.03 (s, | 482.2 |
| | | | | | 3H), 0.40 (t, J = 2.9 Hz, | |
| | | | | | 2H), 0.23 (t, J = 2.9 Hz, | |
| | | | | | 2H). | |
| 86 | 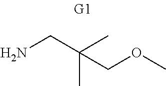 | 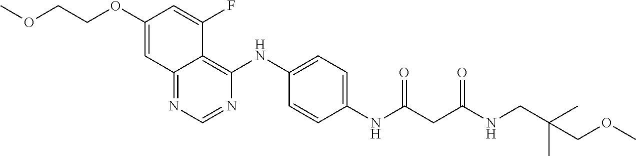 | M | 36 | 10.13 (s, 1H), 8.96 (d, J = 12.3 Hz, 1H), 8.45 (s, 1H), 7.91 (t, J = 6.2 Hz, 1H), 7.61 (m, 4H), 7.15 (dd, J = 13.8, 2.4 Hz, 1H), 7.06 (d, J = 2.4 Hz, 1H), 4.29 (m, 2H), 3.72 (m, 2H), 3.33 (s, 3H), 3.31 (s, 2H), 3.26 (s, 3H), 3.06 (s, 2H), 3.02 | 514.2 |
| | | | | | (d, J = 6.3 Hz, 2H), 0.84 | |
| | | | | | (s, 6H). | |
| 87 | 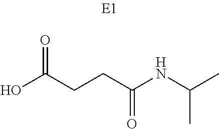 | 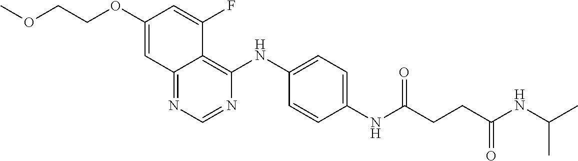 [SMILES: COCCOc1cc(F)c2c(Nc3ccc(NC(=O)CCC(=O)NC(C)C)cc3)ncnc2c1] | I | 9 | 9.95 (s, 1H), 8.93 (d, J = 12.4 Hz, 1H), 8.43 (s, 1H), 7.73 (d, J = 7.6 Hz, 1H), 7.60 (d, J = 9.6 Hz, 2H), 7.57 (d, J = 10.2 Hz, 2H), 7.14 (dd, J = 14.0, 2.4 Hz, 1H), 7.05 (d, J = 2.0 Hz, 1H), 4.28 (m, 2H), 3.82 (m, 1H), 3.72 (m, 2H), 3.31 (s, | 470.2 |
| | | | | | 3H), 2.36 (m, 2H), 1.03 | |
| | | | | | (d, J = 6.8 Hz, 6H). 2Hs | |
| | | | | | are under solvent. | |
| 88 | 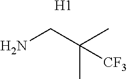 | 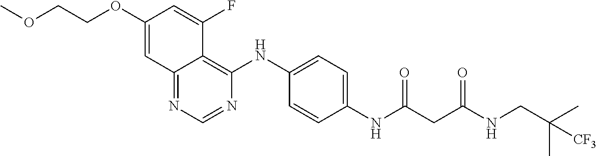 [SMILES: COCCOc1cc(F)c2c(Nc3ccc(NC(=O)CC(=O)NCC(C)(C)C(F)(F)F)cc3)ncnc2c1] | L | 97 | 10.11 (s, 1H), 8.96 (d, J = 12.3 Hz, 1H), 8.43 (s, 1H), 8.19 (t, J = 6.5 Hz, 1H), 7.65-7.53 (m, 4H), 7.13 (dd, J = 13.8, 2.4 Hz, 1H), 7.03 (d, J = 2.4 Hz, 1H), 4.30-4.23 (m, 2H), 3.73-3.66 (m, 2H), 3.31 (s, 3H), 3.28 (s, 2H), 3.26 (s, 2H), | 538.2 |
| | | | | | 1.06 (s, 6H). | |
| 89 | 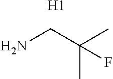 | 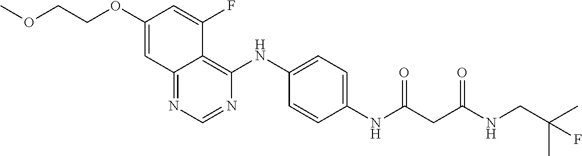 [SMILES: COCCOc1cc(F)c2c(Nc3ccc(NC(=O)CC(=O)NCC(C)(C)F)cc3)ncnc2c1] | L | 100 | 10.12 (s, 1H), 9.08 (d, J = 12.0 Hz, 1H), 8.46 (s, 1H), 8.25 (t, J = 6.3 Hz, 1H), 7.64-7.46 (m, 4H), 7.15 (dd, J = 13.8, 2.4 Hz, 1H), 7.03 (d, J = 2.4 Hz, 1H), 4.31-4.24 (m, 2H), 3.73-3.66 (m, 2H), 3.32 (s, 3H), 3.28 (s, 2H), 3.26 (s, 2H), | 488.2 |
| | | | | | 1.31 (s, 3H), 1.26 (s, | |
| | | | | | 3H). | |
| 90 | 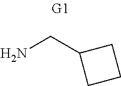 | 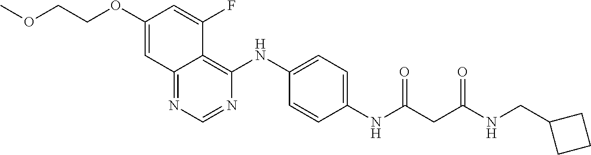 [SMILES: COCCOc1cc(F)c2c(Nc3ccc(NC(=O)CC(=O)NCC4CCC4)cc3)ncnc2c1] | M | 95 | 10.09 (s, 1H), 8.94 (d, J = 12.4 Hz, 1H), 8.43 (s, 1H), 8.02 (t, J = 5.7 Hz, 1H), 7.64-7.53 (m, 4H), 7.12 (dd, J = 13.9, 2.5 Hz, 1H), 7.03 (d, J = 2.4 Hz, 1H), 4.30-4.23 (m, 2H), 3.73-3.66 (m, 2H), 3.31 (s, 3H), 3.23 (s, 2H), 3.11 (dd, J = | 482.2 |
| | | | | | 7.0, 5.7 Hz, 2H), 2.39 | |
| | | | | | (dt, J = 14.9, 7.5 Hz, | |
| | | | | | 1H), 2.01-1.89 (m, | |
| | | | | | 2H), 1.78 (ddd, J = 10.3, | |
| | | | | | 7.6, 5.2 Hz, 2H), 1.66 | |
| | | | | | (td, J = 9.4, 7.2 Hz, 2H). | |
| 91 | 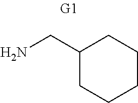 | 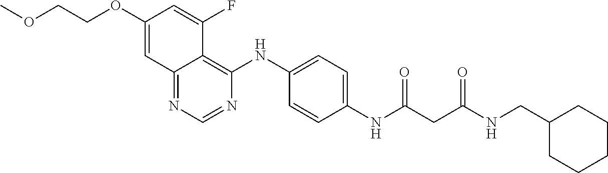 [SMILES: COCCOc1cc(F)c2c(Nc3ccc(NC(=O)CC(=O)NCC4CCCCC4)cc3)ncnc2c1] | M | 76 | 10.14 (s, 1H), 8.96 (s, 1H), 8.45 (s, 1H), 8.05 (t, J = 5.9 Hz, 1H), 7.61 (m, 4H), 7.14 (dd, J = 13.8, 2.4 Hz, 1H), 7.06 (d, J = 2.4 Hz, 1H), 4.29 (m, 2H), 3.72 (m, 2H), 3.33 (s, 3H), 3.26 (s, 2H), 2.95 (t, J = 6.3 Hz, 2H), 1.68 (m, 5H), 1.41 (m, 1H), 1.16 (m, 3H), | 510.2 |
| | | | | | 0.89 (m, 2H) | |
| 92 | 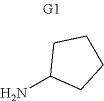 | 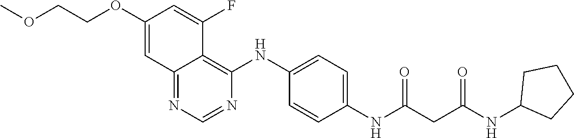 [SMILES: COCCOc1cc(F)c2c(Nc3ccc(NC(=O)CC(=O)NC4CCCC4)cc3)ncnc2c1] | M | 76 | 10.11 (s, 1H), 8.96 (s, 1H), 8.45 (s, 1H), 8.06 (d, J = 7.3 Hz, 1H), 7.61 (m, 4H), 7.15 (dd, J = 13.8, 2.5 Hz, 1H), 7.06 (d, J = 2.4 Hz, 1H), 4.29 (m, 2H), 4.02 (q, J = 6.8 Hz, 1H), 3.72 (m, 2H), 3.33 (s, 3H), 3.22 (s, | 482.2 |
| | | | | | 2H), 1.82 (m, 2H), 1.65 | |
| | | | | | (m, 2H), 1.52 (m, 2H), | |
| | | | | | 1.41 (m, 2H). | |
| 93 | 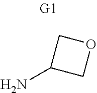 | 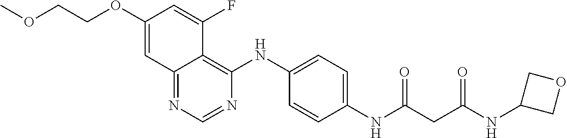 [SMILES: COCCOc1cc(F)c2c(Nc3ccc(NC(=O)CC(=O)NC4COC4)cc3)ncnc2c1] | M | 50 | 10.12 (s, 1H), 8.96 (d, J = 12.3 Hz, 1H), 8.87 (d, J = 6.7 Hz, 1H), 8.45 (s, 1H), 7.62 (m, 4H), 7.15 (dd, J = 13.8, 2.4 Hz, 1H), 7.06 (d, J = 2.4 Hz, 1H), 4.82 (m, 1H), 4.74 (dd, J = 7.5, 6.1 Hz, 2H), 4.45 (t, J = 6.2 Hz, | 470.2 |
| | | | | | 2H), 4.29 (m, 2H), 3.72 | |
| | | | | | (m, 2H), 3.33 (s, 3H), | |
| | | | | | 3.28 (s, 2H). | |
| 94 | 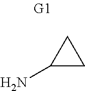 | 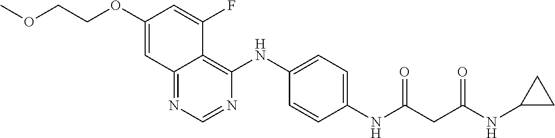 [SMILES: COCCOc1cc(F)c2c(Nc3ccc(NC(=O)CC(=O)NC4CC4)cc3)ncnc2c1] | M | 59 | 10.11 (s, 1H), 8.96 (d, J = 12.4 Hz, 1H), 8.45 (s, 1H), 8.16 (d, J = 4.2 Hz, 1H), 7.61 (m, 4H), 7.15 (dd, J = 13.8, 2.4 Hz, 1H), 7.06 (d, J = 2.4 Hz, 1H), 4.29 (m, 2H), 3.72 (m, 2H), 3.34 (s, 3H), 3.19 (s, 2H), 2.67 (m, | 454.2 |
| | | | | | 1H), 0.64 (td, J = 6.9, | |
| | | | | | 4.7 Hz, 2H), 0.43 (m, | |
| | | | | | 2H). | |
| 95 | 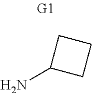 | 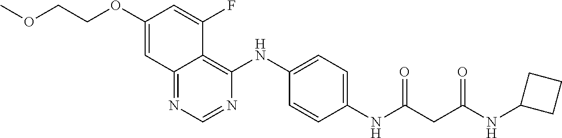 [SMILES: COCCOc1cc(F)c2c(Nc3ccc(NC(=O)CC(=O)NC4CCC4)cc3)ncnc2c1] | M | 66 | δ 10.11 (s, 1H), 8.96 (d, J = 12.3 Hz, 1H), 8.45 (s, 1H), 8.33 (d, J = 7.7 Hz, 1H), 7.61 (m, 4H), 7.15 (dd, J = 13.8, 2.5 Hz, 1H), 7.06 (d, J = 2.4 Hz, 1H), 4.29 (m, 2H), 4.21 (q, J = 8.1 Hz, 1H), | 468.2 |
| | | | | | 3.72 (m, 2H), 3.33 (s, | |
| | | | | | 3H), 3.20 (s, 2H), 2.17 | |
| | | | | | (m, 2H), 1.91 (m, 2H), | |
| | | | | | 1.63 (m, 2H). | |
| 96 | 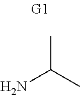 | 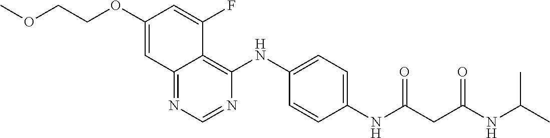 [SMILES: COCCOc1cc(F)c2c(Nc3ccc(NC(=O)CC(=O)NC(C)C)cc3)ncnc2c1] | M | 86 | 10.11 (s, 1H), 8.96 (d, J = 12.4 Hz, 1H), 8.45 (s, 1H), 7.96 (d, J = 7.6 Hz, 1H), 7.61 (m, 4H), 7.15 (dd, J = 13.8, 2.4 Hz, 1H), 7.06 (d, J = 2.4 Hz, 1H), 4.29 (m, 2H), 3.86 (dq, J = 13.5, 6.7 Hz, 1H), 3.72 (m, 2H), 3.33 | 456.2 |
| | | | | | (s, 3H), 3.21 (s, 2H), | |
| | | | | | 1.09 (d, J = 6.6 Hz, 6H). | |
| 97 | 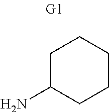 | 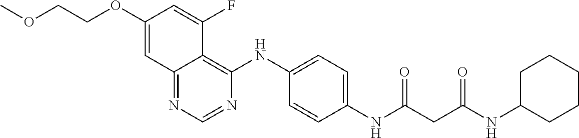 [SMILES: COCCOc1cc(F)c2c(Nc3ccc(NC(=O)CC(=O)NC4CCCCC4)cc3)ncnc2c1] | M | 77 | 10.11 (s, 1H), 8.96 (d, J = 12.3 Hz, 1H), 8.45 (s, 1H), 7.97 (d, J = 7.8 Hz, 1H), 7.61 (m, 4H), 7.15 (dd, J = 13.8, 2.4 Hz, 1H), 7.06 (d, J = 2.4 Hz, 1H), 4.29 (m, 2H), 3.72 (m, 2H), 3.56 (m, 1H), 3.33 (s, 3H), 3.23 (s, | 496.2 |
| | | | | | 2H), 1.76 (m, 2H), 1.69 | |
| | | | | | (m, 2H), 1.56 (m, 1H), | |
| | | | | | 1.22 (m, 5H). | |
| 98 | 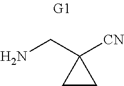 | 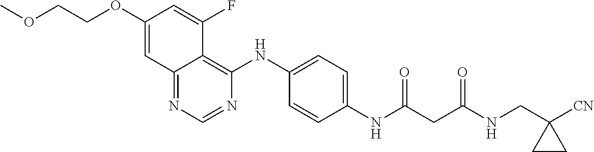 [SMILES: COCCOc1cc(F)c2c(Nc3ccc(NC(=O)CC(=O)NCC4(C#N)CC4)cc3)ncnc2c1] | L | 71 | 10.11 (s, 1H), 8.94 (d, J = 12.3 Hz, 1H), 8.49 (t, J = 6.1 Hz, 1H), 8.43 (s, 1H), 7.65-7.53 (m, 4H), 7.12 (dd, J = 13.9, 2.4 Hz, 1H), 7.03 (d, J = 2.5 Hz, 1H), 4.30-4.23 (m, 2H), 3.73-3.66 (m, 2H), 3.31 (s, 3H), 3.29 (s, 2H), 3.28-3.25 (m, | 493.2 |
| | | | | | 2H), 1.17 (q, J = 4.6 Hz, | |
| | | | | | 2H), 1.07-1.00 (m, | |
| | | | | | 2H). | |
| 99 | 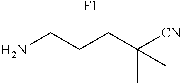 | 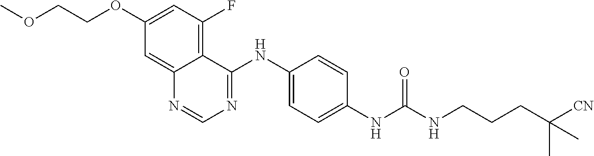 [SMILES: COCCOc1cc(F)c2c(Nc3ccc(NC(=O)NCCCC(C)(C)C#N)cc3)ncnc2c1] | G | 67 | 8.87 (d, J = 12.4 Hz, 1H), 8.44 (s, 1H), 8.41 (s, 1H), 7.51 (d, J = 8.8 Hz, 2H), 7.39 (d, J = 9.2 Hz, 2H), 7.12 (dd, J = 13.6, 2.0 Hz, 1H), 7.03 (d, J = 2.0 Hz, 1H), 6.19 (br t, J = 5.6 Hz, 1H), 4.28 (m, 2H), 3.71 (m, 2H), 3.12 (br m, 2H), | 481.2 |
| | | | | | 1.54-1.60 (m, 4H), 1.30 | |
| | | | | | (s, 6H). 3Hs are under | |
| | | | | | solvents | |
| 100 | 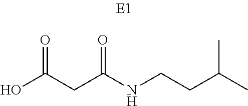 | 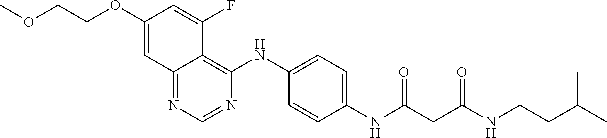 [SMILES: COCCOc1cc(F)c2c(Nc3ccc(NC(=O)CC(=O)NCCC(C)C)cc3)ncnc2c1] | I | 26 | 10.1 (s, 1H), 8.96 (d, J = 12.4 Hz, 1H), 8.44 (s, 1H), 8.01 (br t, J = 5.2 Hz, 1H), 7.63 (d, J = 9.2 Hz, 2H), 7.58 (d, J = 9.2 Hz, 2H), 7.14 (dd, J = 14.0, 2.4 Hz, 1H), 7.05 (d, J = 2.4 Hz, 1H), 4.28 (m, 2H), 3.71 (m, 2H), | 484.2 |
| | | | | | 3.22 (s, 2H), 3.10 (q, J = | |
| | | | | | 6.8 Hz, 2H), 1.59 (m, | |
| | | | | | 1H), 1.32 (q, J = 7.2 Hz, | |
| | | | | | 2H), 0.87 (d, J = 6.8 Hz, | |
| | | | | | 6H). 3Hs are under | |
| | | | | | solvents. | |
| 101 | 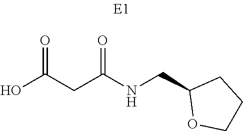 [SMILES: O=C(O)CC(=O)NC[C@H]1CCCO1] | 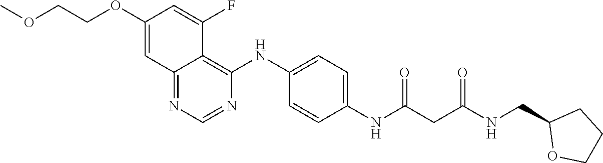 [SMILES: COCCOc1cc(F)c2c(Nc3ccc(NC(=O)CC(=O)NC[C@H]4CCCO4)cc3)ncnc2c1] | I | 9 | 10.1 (s, 1H), 8.99 (br d, J = 12.0 Hz, 1H), 8.45 (s, 1H), 8.13 (br t, J = 5.6 Hz, 1H), 7.63 (d, J = 8.8 Hz, 2H), 7.58 (d, J = 8.8 Hz, 2H), 7.14 (dd, J = 13.6, 2.0 Hz, 1H), 7.05 (d, J = 2.4 Hz, 1H), 4.28 (m, 2H), 3.85 (m, 1H), 3.77 (m, 1H), 3.71 (m, 2H), 3.63 (m, 1H), | 498.1 |
| | | | | | 3.28 (s, 2H), 3.11-3.22 | |
| | | | | | (m, 2H), 1.78-1.92 (m, | |
| | | | | | 3H), 1.50-1.56 (m, 1H). | |
| | | | | | 3Hs are under solvents | |
| 102 | 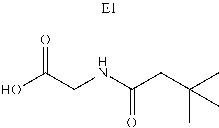 | 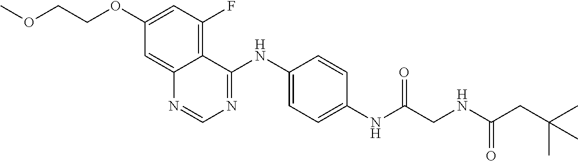 [SMILES: COCCOc1cc(F)c2c(Nc3ccc(NC(=O)CNC(=O)CC(C)(C)C)cc3)ncnc2c1] | I | 9 | 9.98 (s, 1H), 8.95 (d, J = 12.4 Hz, 1H), 8.44 (s, 1H), 8.06 (br t, J = 6.4 Hz, 1H), 7.62 (d, J = 8.8 Hz, 2H), 7.57 (d, J = 8.8 Hz, 2H), 7.14 (br d, J = 13.6 Hz, 1H), 7.05 (s, 1H), 4.28 (br m, 2H), 3.86 (d, J = 5.6 Hz, 2H), 3.71 (br m, 2H), 2.05 (s, | 484.3 |
| | | | | | 2H), 0.98 (s, 9H). 3Hs | |
| | | | | | are under solvents. | |
| 103 | 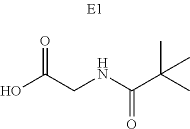 | 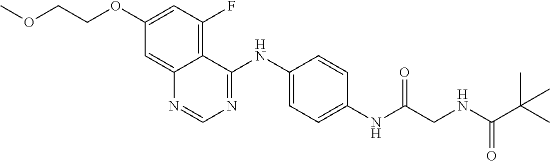 [SMILES: COCCOc1cc(F)c2c(Nc3ccc(NC(=O)CNC(=O)C(C)(C)C)cc3)ncnc2c1] | I | 8 | 9.95 (s, 1H), 8.95 (d, J = 12.4 Hz, 1H), 8.44 (s, 1H), 7.76 (br t, J = 6.0 Hz, 1H), 7.62 (d, J = 9.2 Hz, 2H), 7.57 (d, J = 9.2 Hz, 2H), 7.14 (dd, J = 13.6, 1.6 Hz, 1H), 7.05 (s, 1H), 4.28 (m, 2H), 3.84 (d, J = 6.0 Hz, 2H), 3.71 (m, 2H), 1.13 (s, 9H). 3Hs are under | 470.2 |
| 104 | 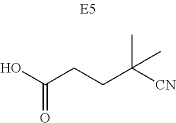 | 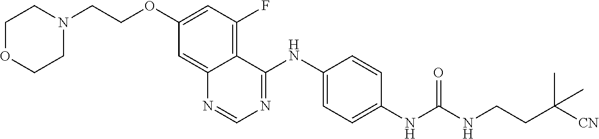 [SMILES: CC(C)(C#N)CCNC(=O)Nc1ccc(Nc2ncnc3cc(OCCN4CCOCC4)cc(F)c23)cc1] | H | 33 | 8.38 (s, 1H), 7.55 (d, J = 8.8 Hz, 2H), 7.41 (d, J = 8.4 Hz, 2H), 6.97-7.20 (m, 2H), 4.31 (t, J = 5.2 Hz, 2H), 4.09 (m, 4H), 3.41 (m, 2H), 2.88 (t, J = 5.2 Hz, 2H), 2.62 (m, 4H), 1.82 (m, 2H), 1.41 (s, 6H) in MeOD. 3NHs | 522.5 |
| | | | | | are missing | |
| 105 | 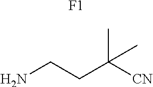 | 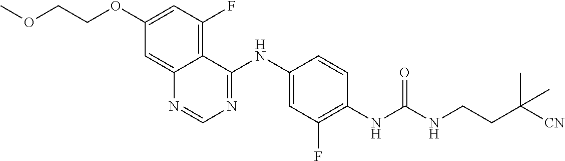 [SMILES: COCCOc1cc(F)c2c(Nc3ccc(NC(=O)NCCC(C)(C)C#N)c(F)c3)ncnc2c1] | G | 19 | 8.84 (d, J = 12.4 Hz, 1H), 8.79 (s, 1H), 8.38 (s, 1H), 7.55 (dd, J = 13.2, 2.0 Hz, 1H), 7.50 (t, J = 8.8 Hz, 1H), 7.13 (dd, J = 13.6, 2.0 Hz, 1H), 7.07 (m, 2H), 6.30 (t, J = 5.6 Hz, 1H), 4.28 (m, 2H), 3.71 (m, 2H), 3.25 (m, 2H), 1.73 (m, | 485.4 |
| | | | | | 2H), 1.34 (s, 6H); 3Hs | |
| | | | | | are under solvents. | |
| 106 | 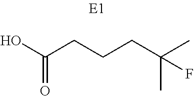 | 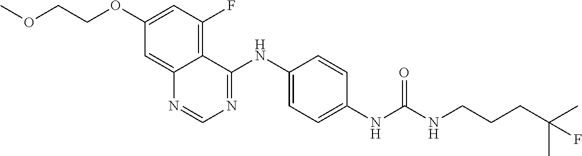 [SMILES: COCCOc1cc(F)c2c(Nc3ccc(NC(=O)NCCCC(C)(C)F)cc3)ncnc2c1] | H | 3 | 8.87 (d, J = 12.4 Hz, 1H), 8.44 (s, 1H), 8.41 (s, 1H), 7.50 (d, J = 8.8 Hz, 2H), 7.38 (d, J = 8.8 Hz, 2H), 7.12 (dd, J = 13.6, 2.4 Hz, 1H), 7.03 (d, J = 2.4 Hz, 1H), 6.15 (t, J = 5.6 Hz, 1H), 4.28 (m, 2H), 3.71 (m, 2H), 3.09 (m, 2H), 1.48-1.64 | 474.4 |
| | | | | | (m, 4H), 1.31 (d, J = | |
| | | | | | 21.1 Hz, 6H); 3Hs are | |
| | | | | | under solvents. | |
| 107 | 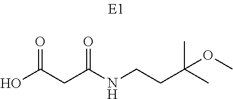 | 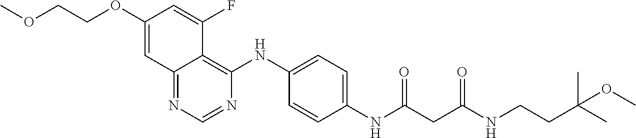 | I | 5 | 10.1 (s, 1H), 8.95 (d, J = 12.4 Hz, 1H), 8.44 (s, 1H), 8.02 (br t, J = 4.8 Hz, 1H), 7.63 (d, J = 9.2 Hz, 2H), 7.58 (d, J = 9.2 Hz, 2H), 7.14 (dd, J = 14.0, 2.0 Hz, 1H), 7.05 (d, J = 2.0 Hz, 1H), 4.28 (m, 2H), 3.71 (m, 2H), | 514.5 |
| | | | | | 3.22 (s, 2H), 3.12 (m, | |
| | | | | | 2H), 3.08 (s, 3H), 1.60 | |
| | | | | | (m, 2H), 1.10 (s, 6H). | |
| | | | | | 3Hs are under solvents. | |
| 108 | 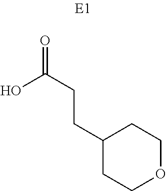 [SMILES: O=C(O)CCC1CCOCC1] | 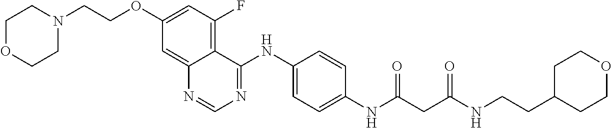 [SMILES: O=C(CC(=O)Nc1ccc(Nc2ncnc3cc(OCCN4CCOCC4)cc(F)c23)cc1)NCCC1CCOCC1] | H | 29 | 8.86 (d, J = 12.4 Hz, 1H), 8.40 (s, 2H), 7.50 (d, J = 8.8 Hz, 2H), 7.38 (d, J = 8.8 Hz, 2H), 7.11 (dd, J = 14.0, 2.4 Hz, 1H), 7.04 (d, J = 2.0 Hz, 1H), 6.09 (t, J = 5.2 Hz, 1H), 4.26 (t, J = 5.6 Hz, 2H), 3.83 (dd, J = 11.2, 3.2 Hz, 2H), 3.58 (m, 4H), 3.30 (m, 4H), 3.25 (m, 2H), 3.12 (q, J = 6.8 | 539.4 |
| | | | | | Hz, 2H), 2.74 (t, J = 5.6 | |
| | | | | | Hz, 2H), 1.59 (br d, J = | |
| | | | | | 13.2 Hz, 2H), 1.52 (m, | |
| | | | | | 1H), 1.37 (q, J = 6.8 Hz, | |
| | | | | | 2H), 1.11-1.21 (m, 2H). | |
| 109 | 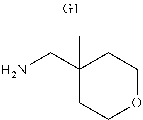 | 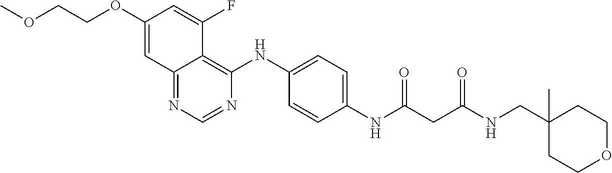 [SMILES: COCCOc1cc(F)c2c(Nc3ccc(NC(=O)CC(=O)NCC4(C)CCOCC4)cc3)ncnc2c1] | M | 6 | 10.1 (s, 1H), 8.97 (d, J = 12.0 Hz, 1H), 8.44 (s, 1H), 8.02 (t, J = 6.0 Hz, 1H), 7.65 (d, J = 10.8 Hz, 2H), 7.60 (d, J = 10.4 Hz, 2H), 7.14 (dd, J = 13.6, 2.0 Hz, 1H), 7.05 (d, J = 2.0 Hz, 1H), 4.28 (m, 2H), 3.71 (m, 2H), 3.58-3.65 (m, 2H), 3.46-3.52 (m, 2H), 3.30 | 526.5 |
| | | | | | (s, 2H), 3.06 (d, J = 6.0 | |
| | | | | | Hz, 2H), 1.39-1.45 (m, | |
| | | | | | 2H), 1.21 (m, 2H), 0.93 | |
| | | | | | (s, 3H), 3Hs are under | |
| | | | | | solvents. | |
| 110 | 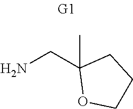 | 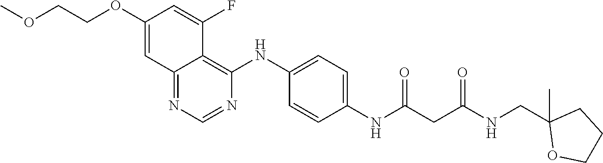 [SMILES: COCCOc1cc(F)c2c(Nc3ccc(NC(=O)CC(=O)NCC4(C)CCCO4)cc3)ncnc2c1] | M | 10 | 10.1 (s, 1H), 8.96 (d, J = 12.4 Hz, 1H), 8.44 (s, 1H), 8.03 (t, J = 6.0 Hz, 1H), 7.63 (d, J = 9.2 Hz, 2H), 7.58 (d, J = 9.2 Hz, 2H), 7.14 (dd, J = 13.6, 2.4 Hz, 1H), 7.05 (d, J = 2.4 Hz, 1H), 4.28 (m, 2H), 3.73 (m, 4H), 3.28 (s, 2H), 3.16 (d, J = 6.4 Hz, 2H), 1.75-1.89 (m, | 512.4 |
| | | | | | 3H), 1.51 (m, 1H), 1.14 | |
| | | | | | (m, 3H). 3Hs are under | |
| | | | | | solvents. | |
| 111 | 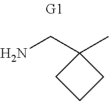 | 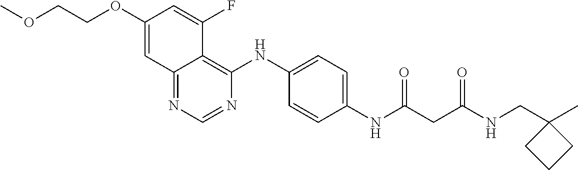 | M | 15 | 10.1 (s, 1H), 8.96 (d, J = 12.4 Hz, 1H), 8.44 (s, 1H), 8.03 (t, J = 5.6 Hz, 1H), 7.63 (d, J = 9.2 Hz, 2H), 7.58 (d, J = 8.8 Hz, 2H), 7.14 (dd, J = 13.6, 2.0 Hz, 1H), 7.05 (d, J = 2.0 Hz, 1H), 4.28 (m, 2H), 3.71 (m, 2H), 3.30 (s, 2H), 3.10 (d, J = 6.0 Hz, 2H), 1.73-1.89 (m, | 496.3 |
| | | | | | 4H), 1.58 (m, 2H), 1.07 | |
| | | | | | (s, 3H). 3Hs are under | |
| | | | | | solvents. | |
| 112 | 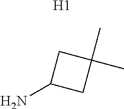 | 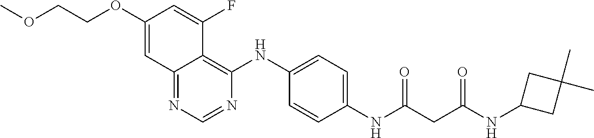 | L | 97 | 10.09 (s, 1H), 8.94 (d, J = 12.3 Hz, 1H), 8.43 (s, 1H), 8.26 (d, J = 7.6 Hz, 1H), 7.61 (d, J = 8.8 Hz, 2H), 7.57 (d, J = 8.8 Hz, 2H), 7.12 (dd, J = 13.9, 2.4 Hz, 1H), 7.04 (d, J = 2.4 Hz, 1H), 4.27 (m, 2H), 4.18 (m, 1H), 3.70 | 496.2 |
| | | | | | (m, 2H), 3.18 (s, 3H), | |
| | | | | | 2.04 (m, 2H), 1.69 (m, | |
| | | | | | 2H), 1.23 (m, 2H), 1.10 | |
| | | | | | (s, 3H), 0.98 (s, 3H). | |
| 113 | 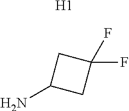 | 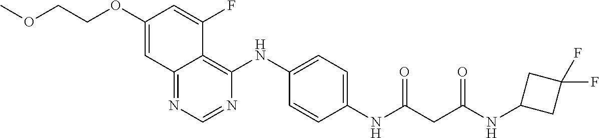 [SMILES: COCCOc1cc(F)c2c(Nc3ccc(NC(=O)CC(=O)NC4CC(F)(F)C4)cc3)ncnc2c1] | L | 95 | 10.11 (s, 1H), 8.94 (d, J = 12.3 Hz, 1H), 8.54 (dd, J = 21.7, 7.0 Hz, 1H), 8.43 (s, 1H), 7.62 (d, J = 8.8 Hz, 2H), 7.56 (d, J = 8.8 Hz, 2H), 7.12 (dd, J = 13.9, 2.4 Hz, 1H), 7.04 (d, J = 2.5 Hz, 1H), 4.27 (m, 2H), 4.05 | 504.2 |
| | | | | | (m, 1H), 3.69 (m, 2H), | |
| | | | | | 3.31 (s, 3H), 3.23 (s, | |
| | | | | | 2H), 2.89 (m, 2H), 2.55 | |
| | | | | | (m, 2H). | |
| 114 | 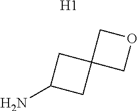 | 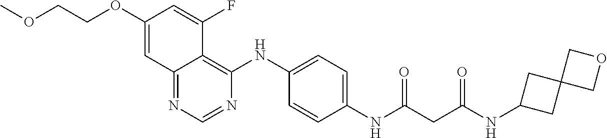 [SMILES: COCCOc1cc(F)c2c(Nc3ccc(NC(=O)CC(=O)NC4CC5(COC5)C4)cc3)ncnc2c1] | L | 80 | 10.08 (s, 1H), 8.94 (d, J = 12.3 Hz, 1H), 8.43 (s, 1H), 8.27 (d, J = 7.3 Hz, 1H), 7.62 (d, J = 8.8 Hz, 2H), 7.56 (d, J = 8.8 Hz, 2H), 7.12 (dd, J = 13.8, 2.4 Hz, 1H), 7.04 (d, J = 2.4 Hz, 1H), 4.57 (s, 2H), 4.47 (s, 2H), 4.26 | 510.2 |
| | | | | | (m, 2H), 3.99 (h, J = 8.0 | |
| | | | | | Hz, 1H), 3.70 (m, 2H), | |
| | | | | | 3.31 (s, 3H), 3.17 (s, | |
| | | | | | 2H), 2.52 (m, 2H), 2.05 | |
| | | | | | (m, 2H). | |
| 115 | 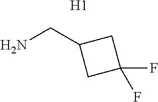 | 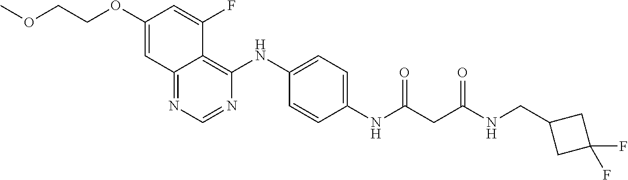 [SMILES: COCCOc1cc(F)c2c(Nc3ccc(NC(=O)CC(=O)NCC4CC(F)(F)C4)cc3)ncnc2c1] | L | 74 | 10.11 (s, 1H), 9.03 (d, J = 12.3 Hz, 1H), 8.45 (s, 1H), 8.20 (d, J = 6.1 Hz, 1H), 7.62 (d, J = 8.8 Hz, 2H), 7.57 (d, J = 8.8 Hz, 2H), 7.14 (dd, J = 13.8, 2.4 Hz, 1H), 7.04 (d, J = 2.4 Hz, 1H), 4.27 (dd, J = 5.5, 3.2 Hz, 2H), 3.73- 3.66 (m, 2H), 3.26- 3.22 (m, 5H), 2.57- 2.54 (m, 2H), 2.33- | 518.2 |
| | | | | | 2.23 (m, 2H). 3Hs are | |
| | | | | | under solvents | |
| 116 | 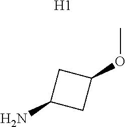 | 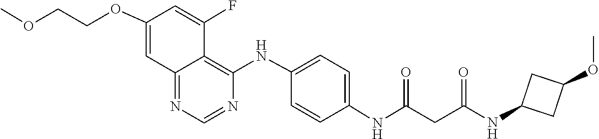 [SMILES: COCCOc1cc(F)c2c(Nc3ccc(NC(=O)CC(=O)N[C@H]4C[C@@H](OC)C4)cc3)ncnc2c1] | L | 72 | 10.08 (s, 1H), 8.97 (d, J = 12.2 Hz, 1H), 8.43 (s, 1H), 8.31 (d, J = 7.6 Hz, 1H), 7.68-7.55 (m, 4H), 7.13 (dd, J = 13.9, 2.5 Hz, 1H), 7.03 (d, J = 2.5 Hz, 1H), 4.30-4.23 (m, 2H), 3.87-3.73 (m, 1H), 3.73-3.66 (m, | 498.2 |
| | | | | | 2H), 3.61-3.49 (m, | |
| | | | | | 1H), 3.31 (s, 3H), 3.18 | |
| | | | | | (s, 2H), 3.11 (s, 3H), | |
| | | | | | 2.59-2.48 (m, 2H), | |
| | | | | | 1.79-1.67 (m, 2H). | |
| 117 | 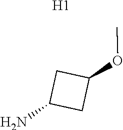 | 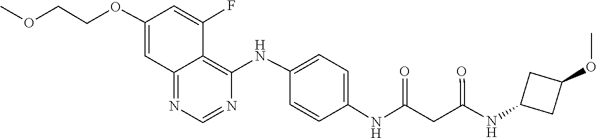 [SMILES: COCCOc1cc(F)c2c(Nc3ccc(NC(=O)CC(=O)N[C@H]4C[C@H](OC)C4)cc3)ncnc2c1] | L | 65 | 10.08 (s, 1H), 8.94 (s, 1H), 8.41 (m, 2H), 7.63- 7.54 (m, 4H), 7.61- 7.56 (m, 1H), 7.12 (s, 1H), 4.26 (m, 2H), 4.17 (m, 1H), 3.94 (m, 1H), 3.69 (s, 2H), 3.31 (s, 3H), 3.12 (m, 1H), 2.86 (m, 1H), 2.70 (m, 1H), | 498.2 |
| | | | | | 2.49 (d, J = 3.3 Hz, 2H), | |
| | | | | | 2.18 (m, 1H), 2.09 (m, | |
| | | | | | 1H), 1.23 (m, 2H). | |
| 118 | 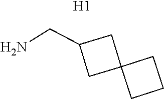 | 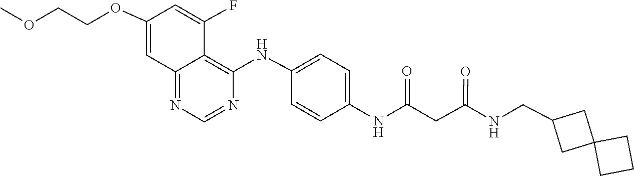 [SMILES: COCCOc1cc(F)c2c(Nc3ccc(NC(=O)CC(=O)NCC4CC5(CCC5)C4)cc3)ncnc2c1] | L | 71 | 10.09 (s, 1H), 8.94 (d, J = 12.3 Hz, 1H), 8.43 (s, 1H), 8.00 (t, J = 5.7 Hz, 1H), 7.63 (d, J = 8.8 Hz, 2H), 7.56 (d, J = 8.8 Hz, 2H), 7.13 (dd, J = 13.9, 2.4 Hz, 1H), 7.04 (d, J = 2.4 Hz, 1H), 4.27 (m, 2H), 3.70 (m, 2H), 3.31 (s, 3H), 3.22 (s, 2H), 3.07 (dd, J = 7.1, 5.7 Hz, | 522.2 |
| | | | | | 2H), 2.21 (p, J = 7.5 Hz, | |
| | | | | | 1H), 1.82-2.04 (m, 6H), | |
| | | | | | 1.68-1.79 (m, 2H), 1.59- | |
| | | | | | 1.68 (m, 2H). | |
| 119 | 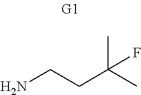 | 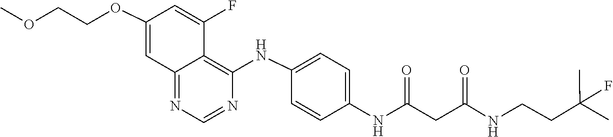 [SMILES: COCCOc1cc(F)c2c(Nc3ccc(NC(=O)CC(=O)NCCC(C)(C)F)cc3)ncnc2c1] | M | 9 | 10.11 (s, 1H), 8.96 (d, J = 12.4 Hz, 1H), 8.44 (s, 1H), 8.08 (br m, 1H), 7.62 (d, J = 8.4 Hz, 2H), 7.58 (d, J = 8.4 Hz, 2H), 7.14 (d, J = 14.0 Hz, 1H), 7.05 (s, 1H), 4.28 (m, 2H), 3.71 (m, 2H), 3.22 (s, 2H), 3.18 (m, | 502.3 |
| | | | | | 2H), 1.73-1.82 (m, 2H), | |
| | | | | | 1.32 (d, J = Hz, 6H). | |
| | | | | | 3Hs are under solvents | |
| 120 | 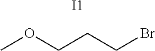 | 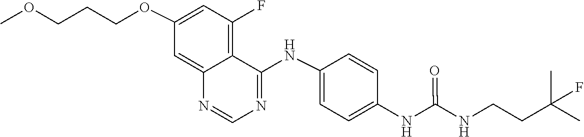 [SMILES: COCCCOc1cc(F)c2c(Nc3ccc(NC(=O)NCCC(C)(C)F)cc3)ncnc2c1] | N | 13 | 8.86 (d, J = 12.4 Hz, 1H), 8.54 (s, 1H), 8.41 (s, 1H), 7.51 (d, J = 8.8 Hz, 2H), 7.38 (d, J = 8.8 Hz, 2H), 7.10 (d, J = 14.0 Hz, 1H), 7.01 (s, 1H), 6.11 (br m, 1H), 4.19 (m, 2H), 3.49 (m, 2H), 3.26 (s, 3H), 3.19 | 474.4 |
| | | | | | (m, 2H), 2.00 (m, 2H), | |
| | | | | | 1.78 (dt, J = 19.6, 7.6 | |
| | | | | | Hz, 2H), 1.34 (d, J = | |
| | | | | | 22.0 Hz, 6H). | |
| 121 | 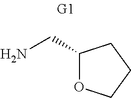 | 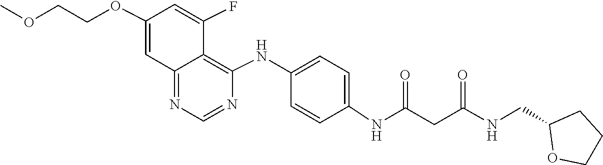 [SMILES: COCCOc1cc(F)c2c(Nc3ccc(NC(=O)CC(=O)NC[C@@H]4CCCO4)cc3)ncnc2c1] | M | 13 | 10.10 (s, 1H), 8.95 (d, J = 12.4 Hz, 1H), 8.44 (s, 1H), 8.13 (t, J = 5.6 Hz, 1H), 7.63 (d, J = 9.2 Hz, 2H), 7.57 (d, J = 9.2 Hz, 2H), 7.14 (dd, J = 13.6, 2.4 Hz, 1H), 7.05 (d, J = 2.4 Hz, 1H), 4.28 (m, 2H), 3.82 (m, 1H), 3.76 (m, 1H), 3.71 (m, 2H), 3.62 (m, 1H), 3.27 (s, | 498.3 |
| | | | | | 2H), 3.11-3.21 (m, 2H), | |
| | | | | | 1.75-1.92 (m, 3H), 1.48- | |
| | | | | | 1.56 (m, 1H). 3Hs are | |
| | | | | | under solvents | |
| 122 | 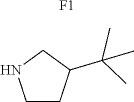 | 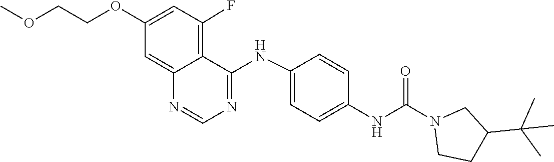 [SMILES: COCCOc1cc(F)c2c(Nc3ccc(NC(=O)N4CCC(C(C)(C)C)C4)cc3)ncnc2c1] | G | 41 | 8.87 (d, J = 12.8 Hz, 1H), 8.42 (s, 1H), 8.11 (s, 1H), 7.52 (d, J = 9.2 Hz, 2H), 7.49 (d, J = 9.2 Hz, 2H), 7.12 (dd, J = 13.6, 2.0 Hz, 1H), 7.04 (d, J = 2.0 Hz, 1H), 4.28 (m, 2H), 3.71 (m, 2H), 3.57 (m, 1H), 3.47 (m, 1H), 3.24 (m, 1H), 3.06 | 482.4 |
| | | | | | (m, 1H), 2.05 (m, 1H), | |
| | | | | | 1.84 (m, 1H), 1.61 (m, | |
| | | | | | 1H), 0.92 (s, 9H), 3Hs | |
| | | | | | are under solvents | |
| 123 | 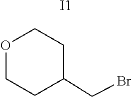 | 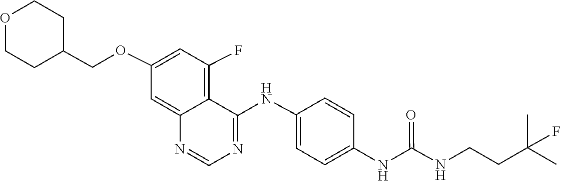 [SMILES: CC(C)(F)CCNC(=O)Nc1ccc(Nc2ncnc3cc(OCC4CCOCC4)cc(F)c23)cc1] | N | 20 | 8.85 (d, J = 12.8 Hz, 1H), 8.51 (s, 1H), 8.41 (s, 1H), 7.51 (d, J = 8.8 Hz, 2H), 7.38 (d, J = 8.8 Hz, 2H), 7.10 (dd, J = 14.0, 2.4 Hz, 1H), 7.02 (d, J = 2.4 Hz, 1H), 6.08 (t, J = 5.2 Hz, 1H), 4.02 (d, J = 6.4 Hz, 2H), 3.90 (m, 2H), 3.36 (m, 2H), 3.20 (m, 2H), 2.06 (m, 1H), 1.79 (dt, J = 20.0, | 500.4 |
| | | | | | 7.6 Hz, 2H), 1.70 (m, | |
| | | | | | 2H), 1.37 (m, 2H), 1.35 | |
| | | | | | (d, J = 22.0 Hz, 6H). | |
| 124 | 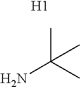 | 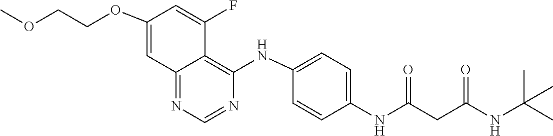 [SMILES: COCCOc1cc(F)c2c(Nc3ccc(NC(=O)CC(=O)NC(C)(C)C)cc3)ncnc2c1] | L (Xtal- Fluor-E) | 63 | 10.07 (s, 1H), 8.95 (d, J = 12.4 Hz, 1H), 8.44 (s, 1H), 7.71 (s, 1H), 7.61 (m, 4H), 7.14 (dd, J = 13.9, 2.4 Hz, 1H), 7.05 (m, 1H), 4.28 (m, 2H), 3.71 (m, 2H), 3.32 (s, 3H), 3.21 (s, 2H), 1.28 (s, 9H). | 470.2 |
| 125 | 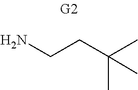 | 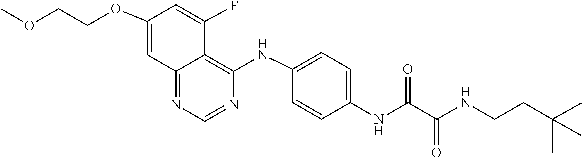 [SMILES: COCCOc1cc(F)c2c(Nc3ccc(NC(=O)C(=O)NCCC(C)(C)C)cc3)ncnc2c1] | M | 91 | 10.60 (s, 1H), 8.97 (m, 1H), 8.92 (m, 1H), 8.44 (s, 1H), 7.80 (d, J = 8.8 Hz, 2H), 7.65 (d, J = 8.8 Hz, 2H), 7.13 (dd, J = 13.9, 2.4 Hz, 1H), 7.04 (d, J = 2.4 Hz, 1H), 4.27 (m, 2H), 3.70 (m, 2H), 3.31 (s, 3H), 3.20 (m, 2H), 1.41 (m, 2H), 0.90 | 484.2 |
| | | | | | (s, 9H). | |
| 126 | 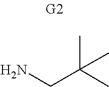 | 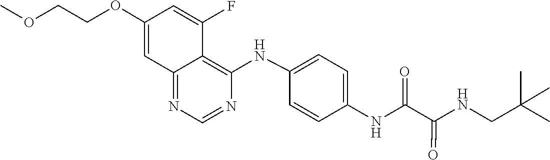 [SMILES: COCCOc1cc(F)c2c(Nc3ccc(NC(=O)C(=O)NCC(C)(C)C)cc3)ncnc2c1] | M | 64 | 10.66 (s, 1H), 8.98 (d, J = 12.2 Hz, 1H), 8.78 (t, J = 6.8 Hz, 1H), 8.45 (s, 1H), 7.85-7.76 (m, 2H), 7.67-7.65 (m, 2H), 7.14 (dd, J = 13.9, 2.4 Hz, 1H), 7.05 (d, J = 2.4 Hz, 1H), 4.27 (m, 2H), 3.70 (m, 2H), 3.34 (s, 3H), 3.03 (d, J = 6.8 Hz, 2H), 0.87 (s, 9H). | 470.2 |
| 127 | 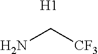 | 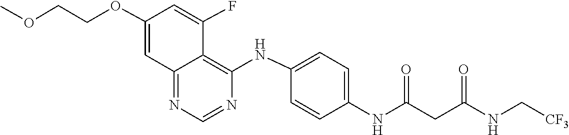 [SMILES: COCCOc1cc(F)c2c(Nc3ccc(NC(=O)CC(=O)NCC(F)(F)F)cc3)ncnc2c1] | L (Xtal- Fluor-E) | 65 | 10.15 (s, 1H), 8.97 (d, J = 12.3 Hz, 1H), 8.75 (t, J = 6.3 Hz, 1H), 8.45 (s, 1H), 7.72 (d, J = 8.8 Hz, 2H), 7.68 (d, J = 8.8 Hz, 2H), 7.15 (d, J = 13.8, 2.4 Hz, 1H), 7.06 (d, J = 2.4 Hz, 1H), 4.29 (m, 2H), 3.97 (m, 2H), 3.72 | 496.2 |
| | | | | | (m, 2H), 3.36 (s, 2H), | |
| | | | | | 3.33 (s, 3H). | |
| 128 | 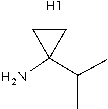 | 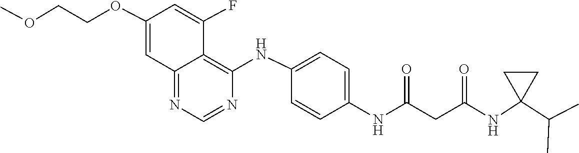 [SMILES: COCCOc1cc(F)c2c(Nc3ccc(NC(=O)CC(=O)NC4(C(C)C)CC4)cc3)ncnc2c1] | L | 63 | 10.08 (s, 1H), 8.96 (d, J = 12.4 Hz, 1H), 8.45 (s, 1H), 8.14 (s, 1H), 7.61 (m, 4H), 7.14 (dd, J = 13.8, 2.4 Hz, 1H), 7.06 (d, J = 2.4 Hz, 1H), 4.29 (m, 2H), 3.72 (m, 2H), 3.33 (s, 3H), 3.19 (s, 2H), 1.46 (m, 1H), 0.89 | 496.2 |
| | | | | | (d, J = 6.8 Hz, 6H), 0.61 | |
| | | | | | (m, 4H). | |
| 129 | 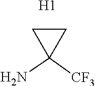 | 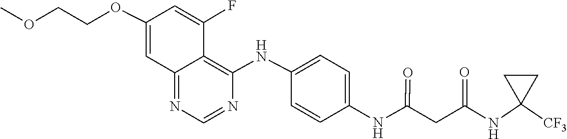 [SMILES: COCCOc1cc(F)c2c(Nc3ccc(NC(=O)CC(=O)NC4(C(F)(F)F)CC4)cc3)ncnc2c1] | L | 52 | 10.11 (s, 1H), 8.97 (d, J = 12.3 Hz, 1H), 8.86 (s, 1H), 8.45 (s, 1H), 7.62 (m, 4H), 7.15 (dd, J = 13.8, 2.4 Hz, 1H), 7.06 (d, J = 2.4 Hz, 1H), 4.29 (m, 2H), 3.72 (m, 2H), 3.33 (s, 3H), 3.27 (s, 2H), 1.25 (m, 2H), 1.06 | 522.2 |
| | | | | | (m, 2H). | |
| 130 | 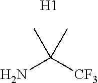 | 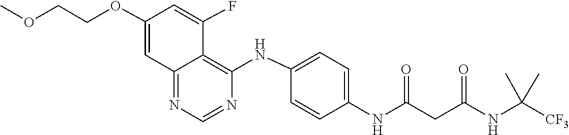 [SMILES: COCCOc1cc(F)c2c(Nc3ccc(NC(=O)CC(=O)NC(C)(C)C(F)(F)F)cc3)ncnc2c1] | L | 63 | 10.09 (s, 1H), 8.97 (d, J = 12.4 Hz, 1H), 8.45 (s, 1H), 8.17 (s, 1H), 7.62 (m, 4H), 7.15 (dd, J = 13.7, 2.4 Hz, 1H), 7.06 (d, J = 2.4 Hz, 1H), 4.29 (m, 2H), 3.72 (m, 2H), 3.33 (s, 3H), 3.31 (s, | 524.2 |
| | | | | | 2H), 1.52 (s, 6H). | |
| 131 | 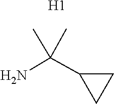 | 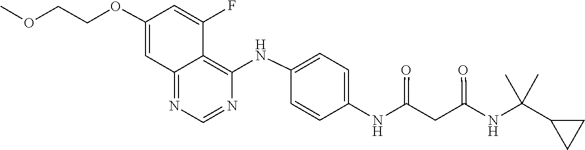 [SMILES: COCCOc1cc(F)c2c(Nc3ccc(NC(=O)CC(=O)NC(C)(C)C4CC4)cc3)ncnc2c1] | L | 57 | 10.08 (s, 1H), 8.95 (d, J = 12.4 Hz, 1H), 8.44 (s, 1H), 7.61 (m, 5H), 7.14 (dd, J = 13.7, 2.4 Hz, 1H), 7.05 (d, J = 2.4 Hz, 1H), 4.28 (m, 2H), 3.71 (m, 2H), 3.33 (s, 3H), 3.23 (s, 2H), 1.24 (m, 1H), 1.18 (m, 6H), 0.32 (m, 4H). | 496.2 |
| 132 | 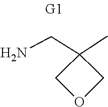 | 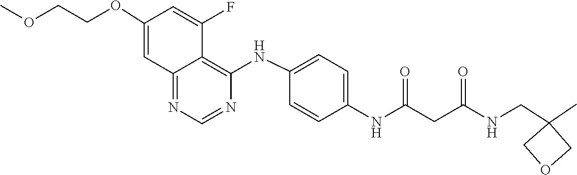 | M | 5 | 10.11 (s, 1H), 8.95 (d, J = 12.4 Hz, 1H), 8.45 (s, 1H), 8.24 (t, J = 6.0 Hz, 1H), 7.63 (d, J = 9.2 Hz, 2H), 7.58 (d, J = 9.2 Hz, 2H), 7.13 (dd, J = 14.0, 2.4 Hz, 1H), 7.05 (d, J = 2.4 Hz, 1H), 4.38 (d, J = 5.6 Hz, 2H), 4.28 (m, 2H), 4.18 (d, J = 5.6 Hz, 2H), 3.70 (t, J = 4.4 Hz, | 498.4 |
| | | | | | 2H), 3.30 (s, 3H), 3.29 | |
| | | | | | (s, 2H), 1.23 (s, 3H). | |
| 133 | 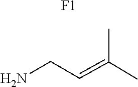 | 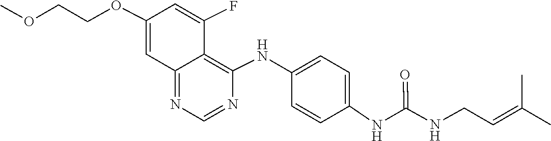 [SMILES: COCCOc1cc(F)c2c(Nc3ccc(NC(=O)NCC=C(C)C)cc3)ncnc2c1] | G | 19 | 8.86 (d, J = 12.4 Hz, 1H), 8.41 (s, 1H), 8.39 (s, 1H), 7.50 (d, J = 8.8 Hz, 2H), 7.37 (d, J = 8.8 Hz, 2H), 7.12 (dd, J = 14.0, 2.4 Hz, 1H), 7.03 (d, J = 1.6 Hz, 1H), 6.07 (t, J = 5.2 Hz, 1H), 5.20 (t, J = 6.4 Hz, 1H), 4.28 | 440.0 |
| | | | | | (m, 2H), 3.71 (m, 2H), | |
| | | | | | 3.67 (m, 2H), 3.33 (s, | |
| | | | | | 3H), 1.69 (s, 3H), 1.65 | |
| | | | | | (s, 3H). | |
| 134 | 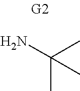 | 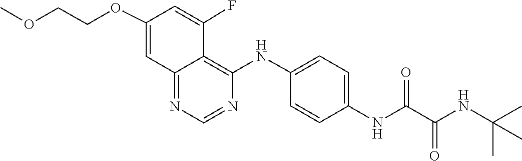 [SMILES: COCCOc1cc(F)c2c(Nc3ccc(NC(=O)C(=O)NC(C)(C)C)cc3)ncnc2c1] | M | 22 | 10.64 (s, 1H), 8.97 (d, J = 12.2 Hz, 1H), 8.44 (s, 1H), 8.04 (s, 1H), 7.78 (d, J = 8.6 Hz, 2H), 7.65 (d, J = 8.6 Hz, 2H), 7.13 (dd, J = 13.7, 2.4 Hz, 1H), 7.04 (d, J = 2.4 Hz, 1H), 4.27 (t, J = 4.5 Hz, 2H), 3.70 (t, J = 4.4 Hz, 2H), 3.27 (s, 3H), 1.36 (s, 9H). | 456.2 |
| 135 | 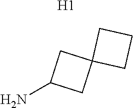 | 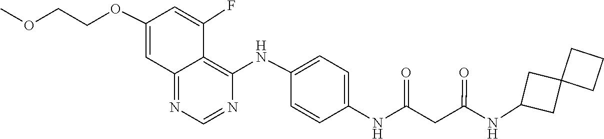 [SMILES: COCCOc1cc(F)c2c(Nc3ccc(NC(=O)CC(=O)NC4CC5(CCC5)C4)cc3)ncnc2c1] | L | 81 | 10.08 (s, 1H), 9.00 (m, 1H), 8.44 (s, 1H), 8.24 (d, J = 7.5 Hz, 1H), 7.58 (m, 4H), 7.13 (d, J = 14.5 Hz, 1H), 7.03 (d, J = 2.3 Hz, 1H), 4.27 (m, 2H), 4.03 (m, 1H), 3.70 (m, 2H), 3.17 (s, 2H), 2.25 (m, 2H), 1.98 (m, | 508.2 |
| | | | | | 2H), 1.72-1.90 (m, 6H). | |
| | | | | | 3Hs are under solvent | |
| 136 | 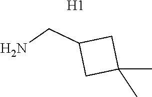 | 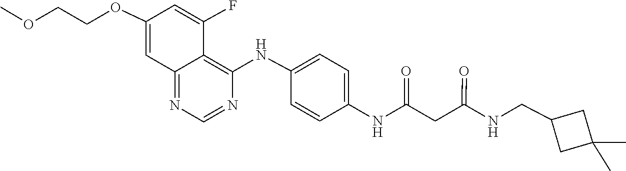 | L | 76 | 10.10 (s, 1H), 9.01 (d, J = 12.2 Hz, 1H), 8.44 (s, 1H), 8.00 (t, J = 5.8 Hz, 1H), 7.59 (m, 4H), 7.14 (dd, J = 13.8, 2.5 Hz, 1H), 7.04 (d, J = 2.5 Hz, 1H), 4.27 (m, 2H), 3.70 (m, 2H), 3.31 (s, 3H), 3.23 (s, 2H), 3.08 (m, 2H), 2.32 (m, 1H), 1.73 (m, 2H), 1.47 (m, 2H), | 510.2 |
| | | | | | 1.04 (s, 3H), 1.02 (s, | |
| | | | | | 3H). | |
| 137 | 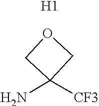 | 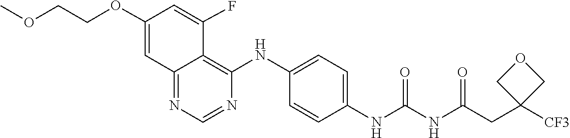 [SMILES: COCCOc1cc(F)c2c(Nc3ccc(NC(=O)NC(=O)CC4(C(F)(F)F)COC4)cc3)ncnc2c1] | L | 48 | 10.16 (s, 1H), 9.19 (s, 1H), 8.97 (d, J = 2.4 Hz, 1H), 8.45 (s, 1H), 7.62 (m, 4H), 7.15 (dd, J = 13.8, 2.5 Hz, 1H), 7.06 (d, J = 2.5 Hz, 1H), 4.74 (m, 4H), 4.29 (m, 2H), 3.72 (m, 3H), 3.36 (s, 2H), 3.33 (s, 3H). | 538.2 |
| 138 | 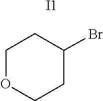 | 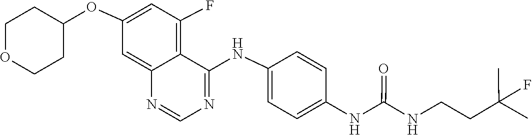 [SMILES: CC(C)(F)CCNC(=O)Nc1ccc(Nc2ncnc3cc(OC4CCOCC4)cc(F)c23)cc1] | N | 2 | 8.85 (d, J = 12.4 Hz, 1H), 8.51 (s, 1H), 8.40 (s, 1H), 7.51 (d, J = 8.8 Hz, 2H), 7.38 (d, J = 8.8 Hz, 2H), 7.12 (dd, J = 16.8, 2.4 Hz, 1H), 7.10 (s, 1H), 6.08 (t, J = 5.6 Hz, 1H), 4.85 (m, 1H), 3.84-3.90 (m, 2H), 3.51- | 486.3 |
| | | | | | 3.57 (m, 2H), 3.18-3.23 | |
| | | | | | (m, 2H), 2.00-2.08 (m, | |
| | | | | | 2H), 1.76-1.84 (m, 2H), | |
| | | | | | 1.59-1.68 (m, 2H), 1.35 | |
| | | | | | (d, J = 21.6 Hz, 6H). | |
| 139 | 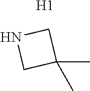 | 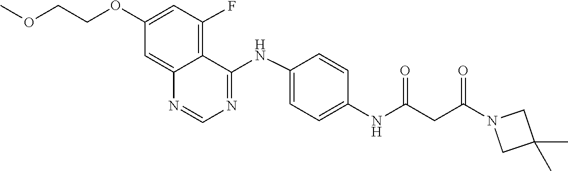 | L | 58 | 10.17 (s, 1H), 8.98 (d, J = 12.3 Hz, 1H), 8.45 (s, 1H), 7.63 (d, J = 8.7 Hz, 2H), 7.59 (d, J = 8.8 Hz, 2H), 7.15 (dd, J = 13.8, 2.4 Hz, 1H), 7.06 (d, J = 2.4 Hz, 1H), 4.29 (dd, J = 5.6, 3.3 Hz, 2H), 3.88 (s, 2H), 3.72 (dd, J = 5.5, 3.3 Hz, 2H), 3.57 (s, 2H), 3.34 (s, | 482.2 |
| | | | | | 3H), 3.23 (s, 2H), 1.25 | |
| | | | | | (s, 6H). | |
| 140 | 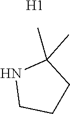 | 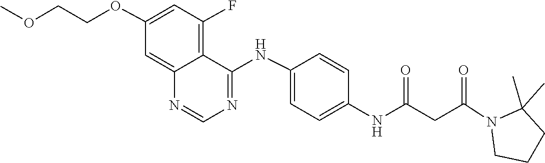 [SMILES: COCCOc1cc(F)c2c(Nc3ccc(NC(=O)CC(=O)N4CCCC4(C)C)cc3)ncnc2c1] | L | 62 | 10.13 (s, 1H), 8.96 (d, J = 12.4 Hz, 1H), 8.45 (s, 1H), 7.61 (q, J = 9.1 Hz, 4H), 7.15 (dd, J = 13.8, 2.5 Hz, 1H), 7.06 (d, J = 2.4 Hz, 1H), 4.29 (m, 2H), 3.72 (m, 2H), 3.54 (t, J = 6.6 Hz, 2H), 3.38 (s, 2H), 3.34 (s, 3H), 1.72-1.86 (m, 4H), 1.39 (s, 6H). | 496.2 |
| 141 | 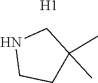 | 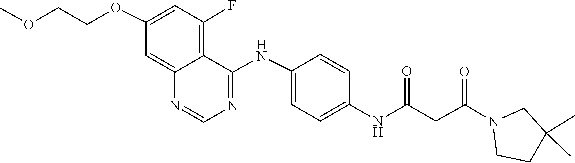 | L | 43 | 10.17 (d, J = 11.8 Hz, 1H), 8.96 (d, J = 12.3 Hz, 1H), 8.45 (s, 1H), 7.63 (d, J = 8.9 Hz, 2H), 7.59 (dd, J = 9.1, 1.9 Hz, 2H), 7.15 (dd, J = 13.8, 2.4 Hz, 1H), 7.06 (d, J = 2.4 Hz, 1H), 4.29 (m, 2H), 3.72 (m, 2H), 3.60 (m, 1H), 3.41 (m, 3H), 3.34 (s, 3H), 3.27 (s, | 496.2 |
| | | | | | 1H), 3.10 (s, 1H), 1.72 | |
| | | | | | (t, J = 7.2 Hz, 1H), 1.64 | |
| | | | | | (t, J = 7.2 Hz, 1H), 1.07 | |
| | | | | | (s, 3H), 1.05 (s, 3H). | |
| 142 | 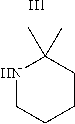 | 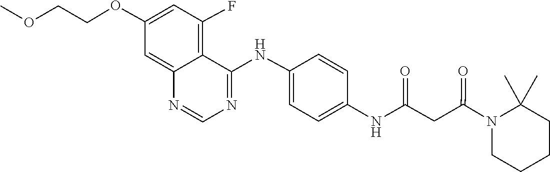 [SMILES: COCCOc1cc(F)c2c(Nc3ccc(NC(=O)CC(=O)N4CCCCC4(C)C)cc3)ncnc2c1] | L | 30 | 9.98 (s, 1H), 8.89 (d, J = 12.4 Hz, 1H), 8.37 (s, 1H), 7.53 (q, J = 8.9 Hz, 4H), 7.07 (dd, J = 13.8, 2.4 Hz, 1H), 6.98 (d, J = 2.4 Hz, 1H), 4.22 (dd, J = 5.5, 3.3 Hz, 2H), 3.65 (dd, J = 5.6, 3.2 Hz, 2H), 3.37 (s, 2H), 3.29 (m, 2H), 3.28 (s, 3H), 1.55 (m, 2H), 1.44-1.52 | 510.2 |
| | | | | | (m, 4H), 1.34 (s, 6H). | |
| 143 | 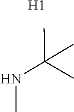 | 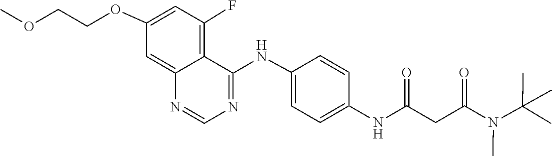 [SMILES: COCCOc1cc(F)c2c(Nc3ccc(NC(=O)CC(=O)N(C)C(C)(C)C)cc3)ncnc2c1] | L | 54 | 10.07 (s, 1H), 8.96 (d, J = 12.4 Hz, 1H), 8.45 (s, 1H), 7.61 (q, J = 9.0 Hz, 4H), 7.15 (dd, J = 13.9, 2.4 Hz, 1H), 7.06 (d, J = 2.4 Hz, 1H), 4.29 (dd, J = 5.8, 3.1 Hz, 2H), 3.75- 3.69 (m, 2H), 3.47 (s, 2H), 3.34 (s, 3H), 2.91 (s, 3H), 1.38 (s, 9H). | 484.2 |
| 144 | 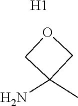 | 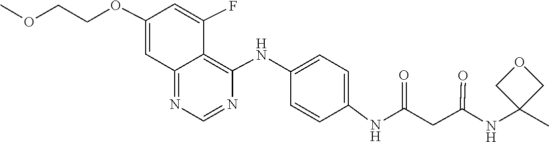 | L | 50 | 10.12 (s, 1H), 8.97 (d, J = 12.4 Hz, 1H), 8.59 (s, 1H), 8.45 (s, 1H), 7.62 (m, 4H), 7.15 (dd, J = 13.8, 2.4 Hz, 1H), 7.06 (d, J = 2.4 Hz, 1H), 4.61 (d, J = 6.1 Hz, 2H), 4.32 (d, J = 6.1 Hz, 2H), 4.29 (m, 2H), 3.72 (m, 2H), | 484.2 |
| | | | | | 3.33 (s, 3H), 3.26 (s, | |
| | | | | | 2H), 1.53 (s, 3H). | |
| 145 | 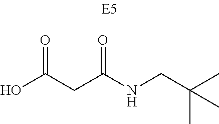 | 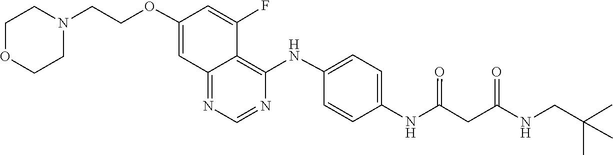 [SMILES: CC(C)(C)CNC(=O)CC(=O)Nc1ccc(Nc2ncnc3cc(OCCN4CCOCC4)cc(F)c23)cc1] | I (T3P) | 7 | 10.1 (s, 1H), 8.95 (d, J = 12.0 Hz, 1H), 8.44 (s, 1H), 7.97 (t, J = 6.0 Hz, 1H), 7.63 (d, J = 9.2 Hz, 2H), 7.57 (d, J = 8.8 Hz, 2H), 7.13 (dd, J = 14.0, 2.4 Hz, 1H), 7.06 (d, J = 2.4 Hz, 1H), 4.27 (t, J = 2.0 Hz, 2H), 3.58 (m, 4H), 3.30 (s, 2H), 2.93 | 539.4 |
| | | | | | (d, J = 6.4 Hz, 2H), 2.74 | |
| | | | | | (t, J = 5.6 Hz, 2H), 0.86 | |
| | | | | | (s, 9H). 4Hs are under | |
| | | | | | solvents | |
| 146 | 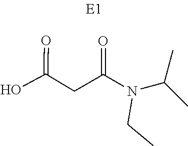 | 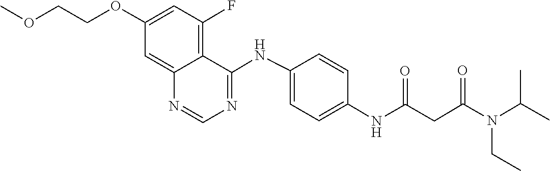 [SMILES: CCN(C(=O)CC(=O)Nc1ccc(Nc2ncnc3cc(OCCOC)cc(F)c23)cc1)C(C)C] | I | 25 | 10.06 (d, J = 4.9 Hz, 1H), 8.90 (d, J = 12.3 Hz, 1H), 8.38 (s, 1H), 7.54 (m, 4H), 7.07 (dd, J = 13.8, 2.4 Hz, 1H), 6.98 (d, J = 2.4 Hz, 1H), 4.42 (m, 0.5H), 4.21 (m, 2H), 4.04 (m, 0.5H), 3.64 (m, 2H), 3.43 (s, 1H), 3.37 (s, 1H), 3.12 (q, J = 6.9 Hz, 2H), 1.10 | 484.2 |
| | | | | | (m, 4.5H), 1.04 (d, J = | |
| | | | | | 6.6 Hz, 3H), 0.98 (t, J = | |
| | | | | | 7.2 Hz, 1.5H), 3Hs are | |
| | | | | | under solvents, 1:1 | |
| | | | | | rotamers | |
| 147 | 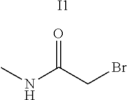 | 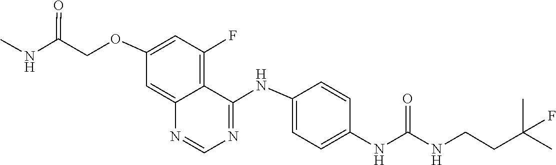 [SMILES: CNC(=O)COc1cc(F)c2c(Nc3ccc(NC(=O)NCCC(C)(C)F)cc3)ncnc2c1] | N | 7 | 8.89 (d, J = 12.8 Hz, 1H), 8.51 (s, 1H), 8.42 (s, 1H), 8.15 (q, J = 4.4 Hz, 1H), 7.51 (d, J = 8.8 Hz, 2H), 7.39 (d, J = 8.8 Hz, 2H), 7.14 (dd, J = 13.6, 2.0 Hz, 1H), 6.99 (d, J = 2.0 Hz, 1H), 6.08 (t, J = 7.2 Hz, 1H), 4.67 (s, 2H), 3.21 (m 2H), 2.67 (d, J = 4.8 Hz, 3H), | 473.3 |
| | | | | | 1.79 (m, 2H), 1.35 (d, J = | |
| | | | | | 21.6 Hz, 6H). | |
| 148 | 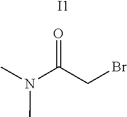 | 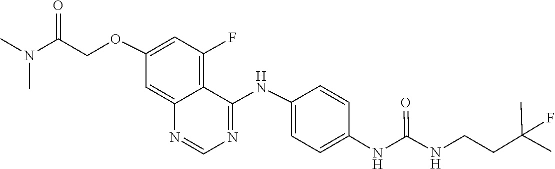 [SMILES: CN(C)C(=O)COc1cc(F)c2c(Nc3ccc(NC(=O)NCCC(C)(C)F)cc3)ncnc2c1] | N | 26 | 8.87 (d, J = 12.4 Hz, 1H), 8.51 (s, 1H), 8.40 (s, 1H), 7.51 (d, J = 8.8 Hz, 2H), 7.38 (d, J = 8.8 Hz, 2H), 7.13 (dd, J = 14.0, 2.4 Hz, 1H), 6.97 (d, J = 2.4 Hz, 1H), 6.08 (t, J = 5.6 Hz, 1H), 5.04 (s, 2H), 3.20 (m, 2H), 3.02 (s, 3H), 2.86 (s, 3H), 1.79 (m, 2H), 1.35 | 487.3 |
| | | | | | (d, J = 21.6 Hz, 6H). | |
| 149 | 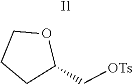 | 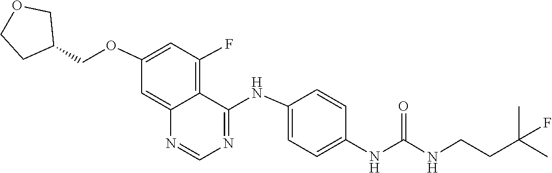 [SMILES: CC(C)(F)CCNC(=O)Nc1ccc(Nc2ncnc3cc(OC[C@@H]4CCOC4)cc(F)c23)cc1] | O | 9 | 8.86 (d, J = 12.4 Hz, 1H), 8.51 (s, 1H), 8.41 (s, 1H), 7.51 (d, J = 8.8 Hz, 2H), 7.38 (d, J = 8.8 Hz, 2H), 7.10 (dd, J = 14.0, 2.4 Hz, 1H), 7.04 (d, J = 2.0 Hz, 1H), 6.08 (d, J = 7.2 Hz, 1H), 4.05-4.15 (m, 2H), 3.76- 3.83 (m, 2H), 3.67 (m, 1H), 3.57 (m, 1H), 3.21 | 486.3 |
| | | | | | (m, 2H), 2.71 (m, 1H), | |
| | | | | | 2.04 (m, 1H), 1.75-1.83 | |
| | | | | | (m, 2H), 1.66-1.72 (m, | |
| | | | | | 1H), 1.35 (d, J = 21.6 | |
| | | | | | Hz, 6H). | |
| 150 | 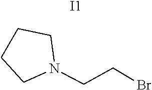 | 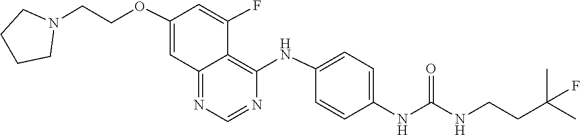 [SMILES: CC(C)(F)CCNC(=O)Nc1ccc(Nc2ncnc3cc(OCCN4CCCC4)cc(F)c23)cc1] | N | 8 | 8.87 (d, J = 12.8 Hz, 1H), 8.59 (s, 1H), 8.40 (s, 1H), 7.50 (d, J = 8.8 Hz, 2H), 7.38 (d, J = 8.8 Hz, 2H), 7.11 (dd, J = 14.0, 2.4 Hz, 1H), 7.03 (d, J = 2.0 Hz, 1H), 6.16 (t, J = 5.6 Hz, 1H), 4.23 (t, J = 6.0 Hz, 2H), 3.19 | 499.3 |
| | | | | | (m, 2H), 2.83 (t, J = 5.6 | |
| | | | | | Hz, 2H), 1.78 (m, 2H), | |
| | | | | | 1.68 (m, 4H), 1.35 (d, | |
| | | | | | J = 21.6 Hz, 6H). 4Ha | |
| | | | | | are under solvents | |
| 151 | 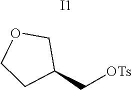 | 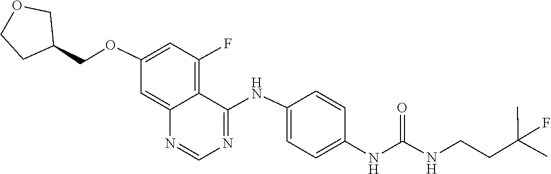 [SMILES: CC(C)(F)CCNC(=O)Nc1ccc(Nc2ncnc3cc(OC[C@H]4CCOC4)cc(F)c23)cc1] | O | 18 | 8.87 (d, J = 12.4 Hz, 1H), 8.51 (s, 1H), 8.41 (s, 1H), 7.51 (d, J = 9.2 Hz, 2H), 7.38 (d, J = 9.2 Hz, 2H), 7.10 (dd, J = 14.0, 2.4 Hz, 1H), 7.04 (d, J = 2.4 Hz, 1H), 6.08 (t, J = 5.6 Hz, 1H), 4.05- 4.12 (m, 2H), 3.76-3.83 (m, 2H), 3.64-3.70 (m, 1H), 3.51-3.62 (m, 2H), | 486.3 |
| | | | | | 3.20 (m, 2H), 2.00-2.09 | |
| | | | | | (m, 1H), 1.78-1.89 (m, | |
| | | | | | 2H), 1.66-1.78 (m, 1H), | |
| | | | | | 1.35 (d, J = 21.6 Hz, | |
| | | | | | 6H). | |
| 152 | 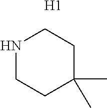 | 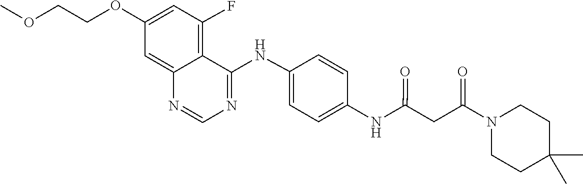 | L | 64 | 10.13 (s, 1H), 8.96 (d, J = 12.4 Hz, 1H), 8.45 (s, 1H), 7.61 (m, 4H), 7.14 (dd, J = 13.8, 2.4 Hz, 1H), 7.06 (d, J = 2.4 Hz, 1H), 4.29 (m, 2H), 3.72 (m, 2H), 3.51 (s, 2H), 3.46 (m, 4H), 3.33 (s, 3H), 1.36 (m, 2H), 1.27 (m, 2H), 0.96 (s, 6H). | 510.2 |
| 153 | 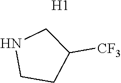 | 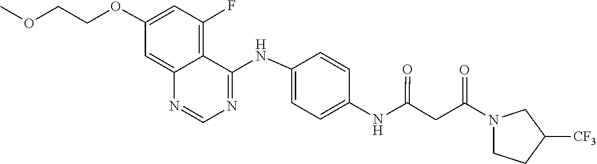 [SMILES: COCCOc1cc(F)c2c(Nc3ccc(NC(=O)CC(=O)N4CCC(C(F)(F)F)C4)cc3)ncnc2c1] | L | 43 | 10.18 (s, 1H), 8.97 (d, J = 12.3 Hz, 1H), 8.45 (s, 1H), 7.61 (m 4H), 7.15 (dd, J = 13.8, 2.4 Hz, 1H), 7.06 (d, J = 2.4 Hz, 1H), 4.29 (m, 2H), 3.72 (m, 2H), 3.64 (m, 2H), 3.48 (m, 2H), 3.40 (m, 1H), 3.34 (s, 3H), 3.32 (s, 2H), 2.20 (m, 1H), 2.02 (m, 1H). | 536.2 |
| 154 | 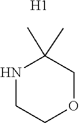 | 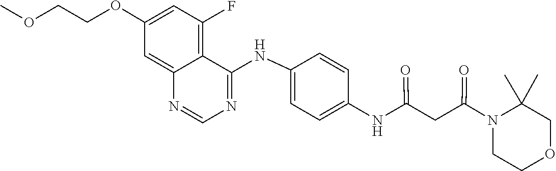 [SMILES: COCCOc1cc(F)c2c(Nc3ccc(NC(=O)CC(=O)N4CCOCC4(C)C)cc3)ncnc2c1] | L | 22 | 10.06 (s, 1H), 8.96 (d, J = 12.4 Hz, 1H), 8.45 (s, 1H), 7.61 (m, 4H), 7.15 (dd, J = 13.8, 2.4 Hz, 1H), 7.06 (d, J = 2.4 Hz, 1H), 4.29 (m, 2H), 3.71 (m, 4H), 3.48 (s, 2H), 3.45 (m, 2H), 3.34 (m, 5H), 1.35 (s, 6H). | 512.2 |
| 155 | 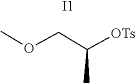 | 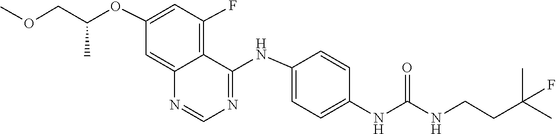 [SMILES: COC[C@@H](C)Oc1cc(F)c2c(Nc3ccc(NC(=O)NCCC(C)(C)F)cc3)ncnc2c1] | O | 11 | 8.85 (d, J = 12.4 Hz, 1H), 8.51 (s, 1H), 8.40 (s, 1H), 7.51 (d, J = 9.2 Hz, 2H), 7.38 (d, J = 9.2 Hz, 2H), 7.09 (dd, J = 14.0, 2.4 Hz, 1H), 7.05 (d, J = 2.0 Hz, 1H), 6.08 (t, J = 5.6 Hz, 1H), 4.89 (m, 1H), 3.49-3.57 (m, | 474.4 |
| | | | | | 2H), 3.30 (s, 3H), 3.17- | |
| | | | | | 3.25 (m, 2H), 1.75-1.83 | |
| | | | | | (m, 2H), 1.35 (d, J = | |
| | | | | | 21.6 Hz, 6H), 1.28 (d, | |
| | | | | | J = 6.4 Hz, 3H). | |
| 156 | 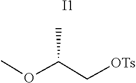 | 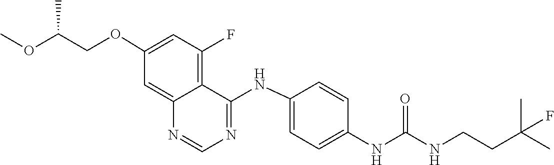 [SMILES: CO[C@H](C)COc1cc(F)c2c(Nc3ccc(NC(=O)NCCC(C)(C)F)cc3)ncnc2c1] | O | 5 | 8.86 (d, J = 12.8 Hz, 1H), 8.51 (s, 1H), 8.41 (s, 1H), 7.51 (d, J = 8.8 Hz, 2H), 7.38 (d, J = 8.8 Hz, 2H), 7.12 (dd, J = 14.0, 2.0 Hz, 1H), 7.03 (d, J = 2.0 Hz, 1H), 6.08 (t, J = 5.6 Hz, 1H), 4.07- 4.16 (m, 2H), 3.72 (m, 1H), 3.33 (s, 3H), 3.21 (m, 2H), 1.75-1.83 (m, | 474.3 |
| | | | | | 2H), 1.35 (d, J = 21.6 | |
| | | | | | Hz, 6H), 1.20 (d, J = 6.0 | |
| | | | | | Hz, 3H). | |
| 157 | 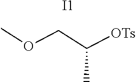 | 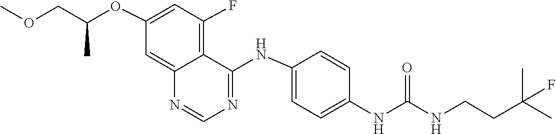 [SMILES: COC[C@H](C)Oc1cc(F)c2c(Nc3ccc(NC(=O)NCCC(C)(C)F)cc3)ncnc2c1] | O | 12 | 8.85 (d, J = 12.4 Hz, 1H), 8.51 (s, 1H), 8.40 (s, 1H), 7.51 (d, J = 9.2 Hz, 2H), 7.38 (d, J = 9.2 Hz, 2H), 7.09 (dd, J = 14.0, 2.4 Hz, 1H), 7.05 (d, J = 2.0 Hz, 1H), 6.08 (t, J = 5.6 Hz, 1H), 4.89 (m, 1H), 3.49-3.57 (m, | 474.4 |
| | | | | | 2H), 3.30 (s, 3H), 3.17- | |
| | | | | | 3.25 (m, 2H), 1.75-1.83 | |
| | | | | | (m, 2H), 1.35 (d, J = | |
| | | | | | 21.6 Hz, 6H), 1.28 (d, | |
| | | | | | J = 6.4 Hz, 3H). | |
| 158 | 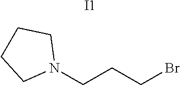 | 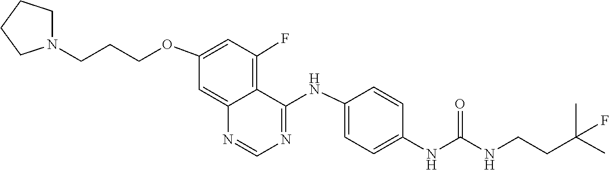 [SMILES: CC(C)(F)CCNC(=O)Nc1ccc(Nc2ncnc3cc(OCCCN4CCCC4)cc(F)c23)cc1] | N | 10 | 8.86 (d, J = 12.4 Hz, 1H), 8.51 (s, 1H), 8.41 (s, 1H), 7.50 (d, J = 8.8 Hz, 2H), 7.38 (d, J = 8.8 Hz, 2H), 7.09 (dd, J = 14.0, 2.4 Hz, 1H), 7.00 (d, J = 2.0 Hz, 1H), 6.08 (t, J = 5.6 Hz, 1H), 4.19 (t, J = 6.0 Hz, 2H), 3.20 (m, 2H), 1.92 (m, 2H), 1.74-1.83 (m, 2H), 1.68 | 513.4 |
| | | | | | (br m, 4H), 1.35 (d, J = | |
| | | | | | 21.6 Hz, 6H). 6Hs are | |
| | | | | | under solvents | |
| 159 | 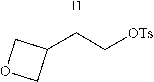 | 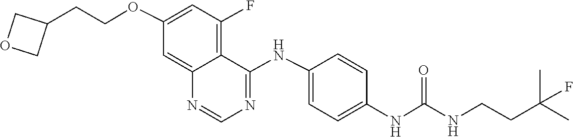 [SMILES: CC(C)(F)CCNC(=O)Nc1ccc(Nc2ncnc3cc(OCCC4COC4)cc(F)c23)cc1] | O | 42 | 8.86 (d, J = 12.4 Hz, 1H), 8.52 (s, 1H), 8.41 (s, 1H), 7.51 (d, J = 8.8 Hz, 2H), 7.38 (d, J = 8.8 Hz, 2H), 7.07 (dd, J = 13.6, 2.4 Hz, 1H), 6.99 (d, J = 2.4 Hz, 1H), 6.08 (t, J = 5.6 Hz, 1H), 4.68 (dd, J = 8.0, 6.0 Hz, | 486.4 |
| | | | | | 2H), 4.37 (t, J = 12.4 | |
| | | | | | Hz, 2H), 4.13 (t, J = 6.4 | |
| | | | | | Hz, 2H), 3.11-3.32 (m, | |
| | | | | | 3H), 2.10-2.15 (m, 2H), | |
| | | | | | 1.75-1.83 (m, 2H), 1.35 | |
| | | | | | (d, J = 21.6 Hz, 6H). | |
| 160 | 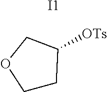 | 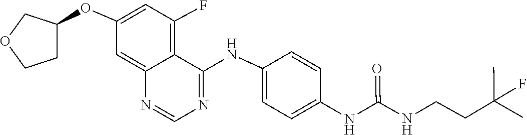 [SMILES: CC(C)(F)CCNC(=O)Nc1ccc(Nc2ncnc3cc(O[C@H]4CCOC4)cc(F)c23)cc1] | O | 4 | 8.88 (d, J = 12.4 Hz, 1H), 8.52 (s, 1H), 8.41 (s, 1H), 7.50 (d, J = 8.8 Hz, 2H), 7.38 (d, J = 8.8 Hz, 2H), 7.10 (dd, J = 14.0, 2.4 Hz, 1H), 7.00 (d, J = 2.0 Hz, 1H), 6.08 (d, J = 5.6 Hz, 1H), 5.29 (br m, 1H), 3.90-3.96 | 472.3 |
| | | | | | (m, 1H), 3.83-3.85 (m, | |
| | | | | | 2H), 3.75-3.80 (m, 1H), | |
| | | | | | 3.18-3.25 (m, 2H), 2.96- | |
| | | | | | 2.36 (m, 1H), 2.02 (m, | |
| | | | | | 1H), 1.75-1.83 (m, 2H), | |
| | | | | | 1.35 (d, J = 21.6 Hz, | |
| | | | | | 6H). | |
| 161 | 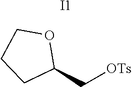 | 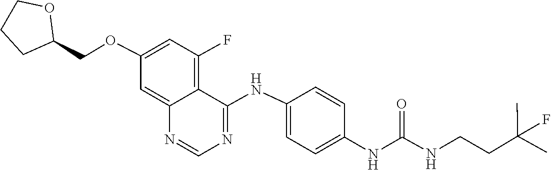 [SMILES: CC(C)(F)CCNC(=O)Nc1ccc(Nc2ncnc3cc(OC[C@H]4CCCO4)cc(F)c23)cc1] | O | 11 | 8.86 (d, J = 12.8 Hz, 1H), 8.51 (s, 1H), 8.41 (s, 1H), 7.51 (d, J = 8.8 Hz, 2H), 7.38 (d, J = 8.8 Hz, 2H), 7.11 (dd, J = 13.6, 2.4 Hz, 1H), 7.03 (d, J = 2.4 Hz, 1H), 6.08 (t, J = 5.6 Hz, 1H), 4.15- 4.24 (m, 2H), 4.08 (m, 1H), 3.80 (m, 1H), 3.69 (m, 1H), 3.17-3.25 (m, | 486.4 |
| | | | | | 2H), 2.03 (m, 1H), 1.89- | |
| | | | | | 1.98 (m, 1H), 1.66-1.88 | |
| | | | | | (m, 4H), 1.35 (d, J = | |
| | | | | | 21.6 Hz, 6H). | |
| 162 | 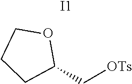 | 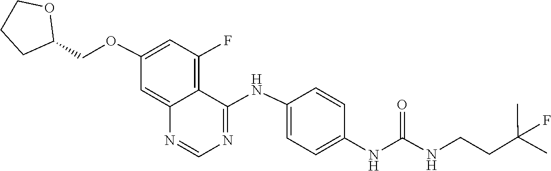 [SMILES: CC(C)(F)CCNC(=O)Nc1ccc(Nc2ncnc3cc(OC[C@@H]4CCCO4)cc(F)c23)cc1] | O | 14 | 8.86 (d, J = 12.8 Hz, 1H), 8.51 (s, 1H), 8.41 (s, 1H), 7.51 (d, J = 9.2 Hz, 2H), 7.38 (d, J = 8.8 Hz, 2H), 7.11 (dd, J = 14.0, 2.4 Hz, 1H), 7.03 (d, J = 2.4 Hz, 1H), 6.08 (t, J = 5.6 Hz, 1H), 4.15- 4.24 (m, 2H), 4.07 (m, 1H), 3.80 (m, 1H), 3.69 (m, 1H), 3.17-3.24 (m, | 486.4 |
| | | | | | 2H), 2.02 (m, 1H), 1.89- | |
| | | | | | 1.98 (m, 1H), 1.66-1.88 | |
| | | | | | (m, 4H), 1.35 (d, J = | |
| | | | | | 21.6 Hz, 6H). | |
| 163 | 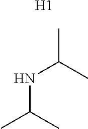 | 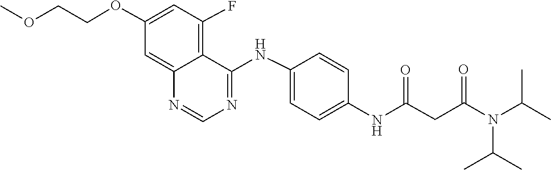 [SMILES: COCCOc1cc(F)c2c(Nc3ccc(NC(=O)CC(=O)N(C(C)C)C(C)C)cc3)ncnc2c1] | L (BTFFH) | 23 | 10.09 (s, 1H), 8.96 (d, J = 12.4 Hz, 1H), 8.45 (s, 1H), 7.61 (m, 4H), 7.15 (dd, J = 13.8, 2.4 Hz, 1H), 7.06 (d, J = 2.4 Hz, 1H), 4.29 (m, 2H), 4.03 (m, 1H), 3.72 (m, 2H), 3.50 (m, 1H), 3.45 (s, 2H), 3.32 (s, 3H), 1.32 (d, J = 6.7 Hz, 6H), 1.17 (d, J = 6.6 Hz, 6H). | 498.2 |
| 164 | 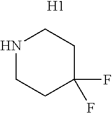 | 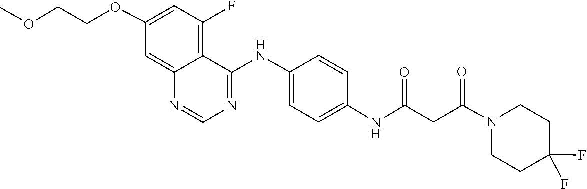 [SMILES: COCCOc1cc(F)c2c(Nc3ccc(NC(=O)CC(=O)N4CCC(F)(F)CC4)cc3)ncnc2c1] | L | 8 | 10.17 (s, 1H), 8.97 (d, J = 12.3 Hz, 1H), 8.45 (s, 1H), 7.61 (m, 4H), 7.15 (dd, J = 13.8, 2.4 Hz, 1H), 7.06 (d, J = 2.4 Hz, 1H), 4.29 (m, 2H), 3.72 (m, 2H), 3.61 (m, 6H), 3.33 (s, 3H), 2.08 (m, 2H), 1.95 (m, 2H). | 518.2 |
| 165 | 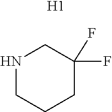 | 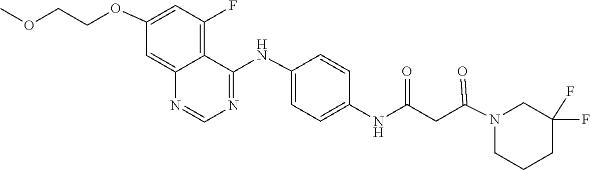 [SMILES: COCCOc1cc(F)c2c(Nc3ccc(NC(=O)CC(=O)N4CCCC(F)(F)C4)cc3)ncnc2c1] | L (BTFFH) | 15 | 10.15 (d, J = 4.9 Hz, 1H), 8.97 (d, J = 12.3 Hz, 1H), 8.45 (s, 1H), 7.61 (m, 4H), 7.15 (dd, J = 13.8, 2.4 Hz, 1H), 7.06 (d, J = 2.4 Hz, 1H), 4.29 (m, 2H), 3.85 (m, 2H), 3.72 (m, 2H), 3.56 (m, 4H), 3.34 (s, 3H), 2.08 (m, 2H), 1.74 (m, 1H), 1.63 (m, 1H). | 518.2 |
| 166 | 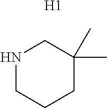 | 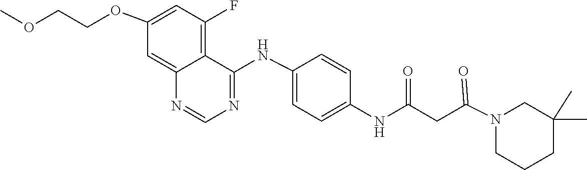 | L | 61 | 10.05 (d, J = 14.3 Hz, 1H), 8.89 (d, J = 12.2 Hz, 1H), 8.37 (s, 1H), 7.55 (d, J = 8.8 Hz, 2H), 7.51 (d, J = 8.7 Hz, 2H), 7.07 (dd, J = 13.7, 2.4 Hz, 1H), 6.98 (d, J = 2.4 Hz, 1H), 4.22 (dd, J = 5.6, 3.3 Hz, 2H), 3.65 (dd, J = 5.5, 3.3 Hz, 2H), 3.47-3.41 (m, | 510.2 |
| | | | | | 2H), 3.39-3.30 (m, | |
| | | | | | 2H), 3.11 (m, 2H), 1.51- | |
| | | | | | 1.40 (m, 2H), 1.30 (dt, | |
| | | | | | J = 8.8, 4.5 Hz, 2H), | |
| | | | | | 0.83 (s, 3H), 0.81 (s, | |
| | | | | | 3H). (rotamers | |
| | | | | | observed). 3Hs are | |
| | | | | | under solvents | |
| 167 | 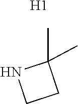 | 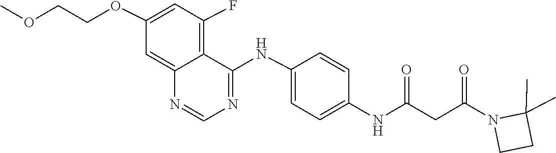 | L | 55 | 10.17 (s, 1H), 8.96 (d, J = 12.3 Hz, 1H), 8.45 (s, 1H), 7.56-7.66 (m, 4H), 7.15 (dd, J = 13.8, 2.3 Hz, 1H), 7.06 (d, J = 2.4 Hz, 1H), 4.29 (dd, J = 5.6, 3.3 Hz, 2H), 4.06 (t, J = 7.7 Hz, 2H), 3.72 (m, 2H), 3.14-3.22 (m, 2H), 2.02 (m, 2H), 1.46- | 482.2 |
| | | | | | 1.52 (m, 6H), (rotamers | |
| | | | | | observed). 3Hs are | |
| | | | | | under solvents | |
| 168 | 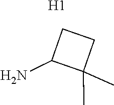 | 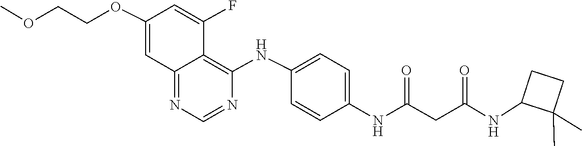 | L (PyAOP) | 7 | 10.11 (s, 1H), 8.98 (d, J = 12.3 Hz, 1H), 8.45 (s, 1H), 7.61 (m, 4H), 7.15 (dd, J = 13.7, 2.4 Hz, 1H), 7.06 (d, J = 2.4 Hz, 1H), 4.29 (m, 2H), 3.95 (q, J = 8.6 Hz, 1H), 3.72 (m, 2H), 3.26 (s, 3H), 2.06 (m, 1H), 1.85 (m, 1H), 1.47 (m, 2H), 1.07 | 496.2 |
| | | | | | (s, 3H), 0.99 (s, 3H). | |
| | | | | | 3Hs are under solvents | |
| 169 | 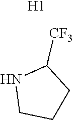 | 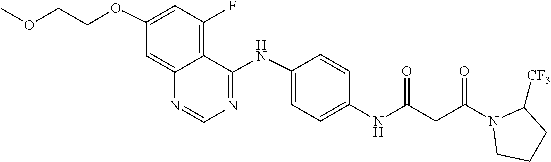 [SMILES: COCCOc1cc(F)c2c(Nc3ccc(NC(=O)CC(=O)N4CCCC4C(F)(F)F)cc3)ncnc2c1] | L (PyAOP) | 21 | 10.17 (d, J = 5.6 Hz, 1H), 8.97 (d, J = 12.3 Hz, 1H), 8.45 (s, 1H), 7.62 (m, 4H), 7.15 (dd, J = 13.8, 2.4 Hz, 1H), 7.06 (d, J = 2.4 Hz, 1H), 4.79 (m, 1H), 4.29 (m, 2H), 3.72 (m, 3H), 3.57 (m, 2H), 3.36 (s, 3H), 3.33 (s, 2H), 2.10 (m, 1H), 2.01 (m, 2H). | 536.2 |
| | | | | | (rotamers observed). | |
| 170 | 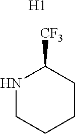 | 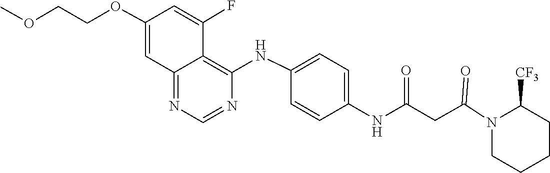 [SMILES: COCCOc1cc(F)c2c(Nc3ccc(NC(=O)CC(=O)N4CCCC[C@@H]4C(F)(F)F)cc3)ncnc2c1] | L (PyAOP) | 73 | 10.18 (d, J = 12.1 Hz, 1H), 8.98 (d, J = 12.3 Hz, 1H), 8.45 (s, 1H), 7.64 (d, J = 8.8 Hz, 2H), 6.61 (d, J = 8.8 Hz, 2H), 7.15 (dd, J = 13.7, 2.4 Hz, 1H), 7.06 (d, J = 2.4 Hz, 1H), 5.22 (m, 1H), 4.29 (m, 2H), 3.90 (m, 1H), 3.72 (m, 2H), 3.60 (m, 1H), 3.34 (s, 3H), | 550.2 |
| | | | | | 3.33 (s, 2H), 3.17 (m, | |
| | | | | | 1H), 1.96 (m, 1H), 1.70 | |
| | | | | | (m, 2H), 1.49 (m, 2H). | |
| 171 | 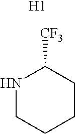 | 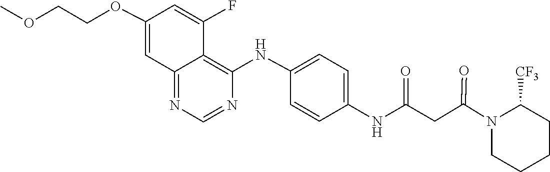 [SMILES: COCCOc1cc(F)c2c(Nc3ccc(NC(=O)CC(=O)N4CCCC[C@H]4C(F)(F)F)cc3)ncnc2c1] | L (PyAOP) | 71 | 10.18 (d, J = 12.1 Hz, 1H), 8.98 (d, J = 12.3 Hz, 1H), 8.45 (s, 1H), 7.64 (d, J = 8.8 Hz, 2H), 6.61 (d, J = 8.8 Hz, 2H), 7.15 (dd, J = 13.7, 2.4 Hz, 1H), 7.06 (d, J = 2.4 Hz, 1H), 5.22 (m, 1H), 4.29 (m, 2H), 3.90 (m, 1H), 3.72 (m, 2H), 3.60 (m, 1H), 3.34 (s, 3H), | 550.2 |
| | | | | | 3.33 (s, 2H), 3.17 (m, | |
| | | | | | 1H), 1.96 (m, 1H), 1.70 | |
| | | | | | (m, 2H), 1.50 (m, 2H). | |
| 172 | 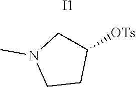 | 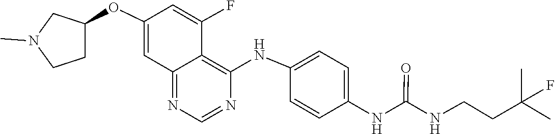 [SMILES: CN1CC[C@H](Oc2cc(F)c3c(Nc4ccc(NC(=O)NCCC(C)(C)F)cc4)ncnc3c2)C1] | N | 6 | 8.86 (d, J = 12.8 Hz, 1H), 8.54 (s, 1H), 8.40 (s, 1H), 7.50 (d, J = 9.2 Hz, 2H), 7.38 (d, J = 9.2 Hz, 2H), 7.05 (dd, J = 14.0, 2.4 Hz, 1H), 6.93 (d, J = 2.4 Hz, 1H), 6.11 (t, J = 5.6 Hz, 1H), 5.06 (m, 1H), 3.21 (m, 2H), | 485.3 |
| | | | | | 2.79-2.69 (m, 3H), 2.33 | |
| | | | | | (m, 2H), 2.27 (s, 3H), | |
| | | | | | 1.74-1.83 (m, 3H), 1.35 | |
| | | | | | (d, J = 21.6 Hz, 6H). | |
| 173 | 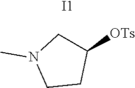 | 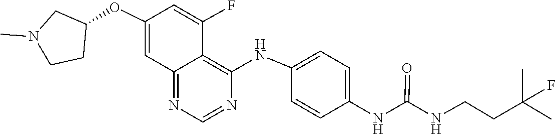 [SMILES: CN1CC[C@@H](Oc2cc(F)c3c(Nc4ccc(NC(=O)NCCC(C)(C)F)cc4)ncnc3c2)C1] | N | 5 | 8.86 (d, J = 12.4 Hz, 1H), 8.55 (s, 1H), 8.40 (s, 1H), 7.50 (d, J = 8.8 Hz, 2H), 7.38 (d, J = 8.8 Hz, 2H), 7.05 (dd, J = 14.0, 2.4 Hz, 1H), 6.92 (d, J = 2.4 Hz, 1H), 6.11 (t, J = 5.6 Hz, 1H), 5.07 (m, 1H), 3.20 (m, 2H), | 485.3 |
| | | | | | 2.78 (m, 1H), 2.70 (m, | |
| | | | | | 2H), 2.37 (m, 2H), 2.27 | |
| | | | | | (s, 3H), 1.74-1.83 (m, | |
| | | | | | 3H), 1.35 (d, J = 22.0 | |
| | | | | | Hz, 6H). | |
| 174 | 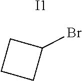 | 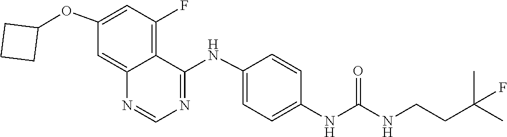 [SMILES: CC(C)(F)CCNC(=O)Nc1ccc(Nc2ncnc3cc(OC4CCC4)cc(F)c23)cc1] | N | 10 | 8.85 (d, J = 12.8 Hz, 1H), 8.51 (s, 1H), 8.40 (s, 1H), 7.50 (d, J = 8.8 Hz, 2H), 7.38 (d, J = 8.8 Hz, 2H), 7.03 (dd, J = 13.6, 2.0 Hz, 1H), 6.86 (d, J = 2.0 Hz, 1H), 6.08 (t, J = 5.6 Hz, 1H), 4.91 (m, 1H), 3.21 (m, 2H), | 456.3 |
| | | | | | 2.08 (m, 2H), 1.66-1.87 | |
| | | | | | (m, 4H), 1.35 (d, J = | |
| | | | | | 21.6 Hz, 6H). 2Hs are | |
| | | | | | under solvents | |
| 175 | 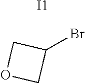 | 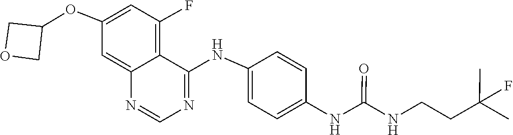 [SMILES: CC(C)(F)CCNC(=O)Nc1ccc(Nc2ncnc3cc(OC4COC4)cc(F)c23)cc1] | N | 10 | 8.91 (d, J = 12.4 Hz, 1H), 8.52 (s, 1H), 8.41 (s, 1H), 7.50 (d, J = 8.8 Hz, 2H), 7.38 (d, J = 8.8 Hz, 2H), 7.11 (dd, J = 13.2, 2.0 Hz, 1H), 6.74 (d, J = 2.4 Hz, 1H), 6.08 (t, J = 5.6 Hz, 1H), 5.50 (m, 1H), 5.00 (m, 2H), | 458.3 |
| | | | | | 4.59 (m, 2H), 3.20 (m, | |
| | | | | | 2H), 1.74-1.83 (m, 2H), | |
| | | | | | 1.35 (d, J = 21.6 Hz, | |
| | | | | | 6H). | |
| 176 | 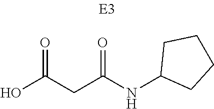 [SMILES: O=C(O)CC(=O)NC1CCCC1] | 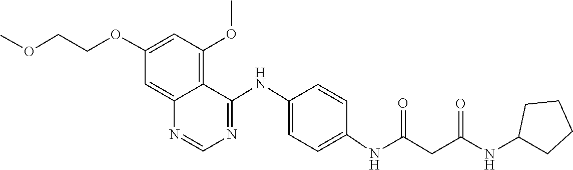 [SMILES: COCCOc1cc(OC)c2c(Nc3ccc(NC(=O)CC(=O)NC4CCCC4)cc3)ncnc2c1] | I (T3P) | 15 | 10.10 (s, 1H), 9.74 (s, 1H), 8.40 (s, 1H), 8.04 (d, J = 7.2 Hz, 1H), 7.72 (d, J = 8.8 Hz, 2H), 7.58 (d, J = 9.2 Hz, 2H), 6.77 (d, J = 2.0 Hz, 1H), 6.72 (d, J = 2.4 Hz, 1H), 4.24 (m, 2H), 4.08 (s, 3H), 4.00 (m, 1H), 3.71 (m, 2H), 3.20 (s, 2H), 1.80 (m, 2H), 1.62 (m, 2H), | 494.3 |
| | | | | | 1.51 (m, 2H), 1.40 (m, | |
| | | | | | 2H). 3Hs are under | |
| | | | | | solvents | |
| 177 | 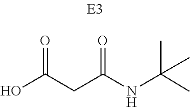 | 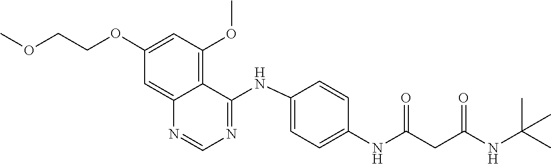 [SMILES: COCCOc1cc(OC)c2c(Nc3ccc(NC(=O)CC(=O)NC(C)(C)C)cc3)ncnc2c1] | I (T3P) | 24 | 10.05 (s, 1H), 9.75 (s, 1H), 8.39 (s, 1H), 7.72 (m, 3H), 7.58 (d, J = 8.4 Hz, 2H), 6.77 (s, 1H), 6.71 (s, 1H), 4.24 (m, 2H), 4.08 (s, 3H), 3.71 (m, 2H), 3.20 (s, 2H), 1.27 (s, 9H). 3Hs are under solvents | 482.4 |
| 178 | 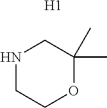 | 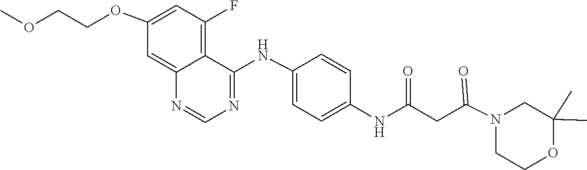 | L | 21 | 10.16 (s, 1H), 8.96 (d, J = 12.3 Hz, 1H), 8.45 (s, 1H), 7.61 (m, 4H), 7.15 (dd, J = 13.9, 2.4 Hz, 1H), 7.06 (d, J = 2.4 Hz, 1H), 4.29 (m, 2H), 3.72 (m, 2H), 3.67 (m, 1H), 3.60 (m, 2H), 3.47 (m, 3H), 3.33 (s, 5H), 1.17 (s, 3H), 1.14 (s, 3H) | 512.2 |
| 179 | 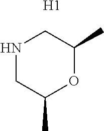 | 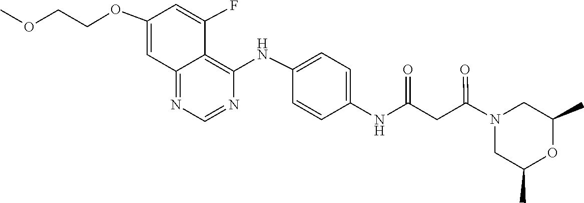 | L | 5 | 10.15 (s, 1H), 8.97 (d, J = 12.3 Hz, 1H), 8.45 (s, 1H), 7.61 (m, 4H), 7.15 (dd, J = 13.9, 2.4 Hz, 1H), 7.06 (d, J = 2.4 Hz, 1H), 4.29 (m, 3H), 3.86 (d, J = 13.1 Hz, 1H), 3.72 (m, 2H), 3.56 (m, 3H), 3.44 (m, 1H), 3.34 (s, 3H), 3.33 (s, 3H), 2.74 (m, 1H), 2.28 (m, 1H), 1.11 (d, J = 6.2, 6H). | 512.2 |
| 180 | 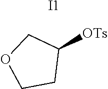 | 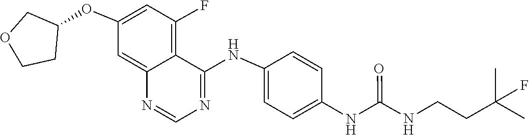 [SMILES: CC(C)(F)CCNC(=O)Nc1ccc(Nc2ncnc3cc(O[C@@H]4CCOC4)cc(F)c23)cc1] | N | 10 | 8.52 (s, 1H), 8.41 (d, J = 2.4 Hz, 1H), 7.50 (d, J = 8.8 Hz, 2H), 7.38 (d, J = 8.8 Hz, 2H), 7.10 (dd, J = 14.0, 2.0 Hz, 1H), 7.00 (d, J = 2.0 Hz, 1H), 6.08 (t, J = 5.6 Hz, 1H), 5.26 (m, 1H), 3.90-3.95 (m, 1H), 3.85 (m, 2H), 3.78 (m, 1H), 3.20 (m, | 472.3 |
| | | | | | 2H), 2.57 (m, 2H), 2.02 | |
| | | | | | (m, 1H), 1.75-1.86 (m, | |
| | | | | | 2H), 1.35 (d, J = 22.0 | |
| | | | | | Hz, 6H). | |
| 181 | 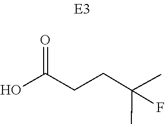 | 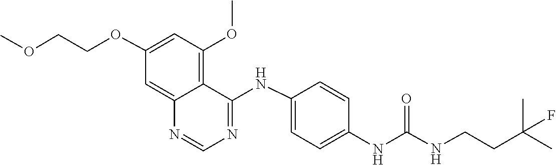 [SMILES: COCCOc1cc(OC)c2c(Nc3ccc(NC(=O)NCCC(C)(C)F)cc3)ncnc2c1] | H | 7 | 9.66 (s, 1H), 8.50 (s, 1H), 8.35 (d, J = 2.4 Hz, 1H), 7.60 (m, 2H), 7.38 (d, J = 8.8 Hz, 2H), 6.75 (d, J = 2.0 Hz, 1H), 6.69 (d, J = 2.0 Hz, 1H), 6.06 (t, J = 5.6 Hz, 1H), 4.24 (m, 2H), 4.07 (s, 3H), 3.71 (m, 2H), 3.32 (s, 3H), 3.20 (m, 2H), 1.74- | 472.3 |
| | | | | | 1.83 (m, 2H), 1.35 (d, | |
| | | | | | J = 21.6 Hz, 6H). | |
| 182 | 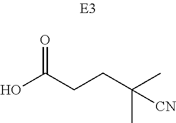 | 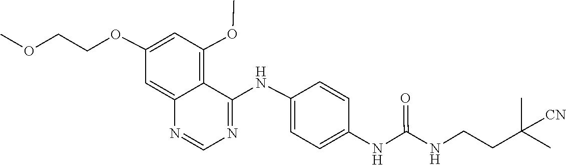 [SMILES: COCCOc1cc(OC)c2c(Nc3ccc(NC(=O)NCCC(C)(C)C#N)cc3)ncnc2c1] | H | 23 | 9.97 (br s, 1H), 8.57 (s, 1H), 8.45 (s, 1H), 7.55 (d, J = 8.8 Hz, 2H), 7.42 (d, J = 8.8 Hz, 2H), 6.76 (m, 2H), 6.20 (t, J = 6.0 Hz, 1H), 4.26 (m, 2H), 4.09 (s, 3H), 3.72 (m, 2H), 3.34 (s, 3H), 3.24 (m, 2H), 1.72 (m, 2H), 1.34 (s, 6H). | 479.2 |
| 183 | 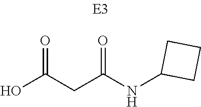 [SMILES: O=C(O)CC(=O)NC1CCC1] | 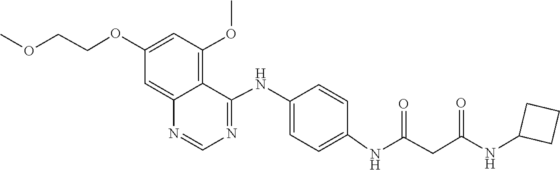 [SMILES: COCCOc1cc(OC)c2c(Nc3ccc(NC(=O)CC(=O)NC4CCC4)cc3)ncnc2c1] | I (T3P) | 16 | 10.08 (s, 1H), 9.75 (s, 1H), 8.39 (s, 1H), 8.31 (d, J = 7.6 Hz, 1H), 7.73 (d, J = 8.8 Hz, 2H), 7.58 (d, J = 8.8 Hz, 2H), 6.77 (d, J = 2.0 Hz, 1H), 6.71 (d, J = 2.0 Hz, 1H), 4.23 (m, 2H), 4.19 (m, 1H), 4.08 (s, 3H), 3.71 (m, 2H), 3.34 (s, 3H), 3.19 (s, 2H), 2.12-2.20 (m, | 480.3 |
| | | | | | 2H), 1.84-1.94 (m, 2H), | |
| | | | | | 1.66 (m, 2H). | |
| 184 | 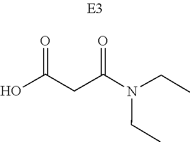 | 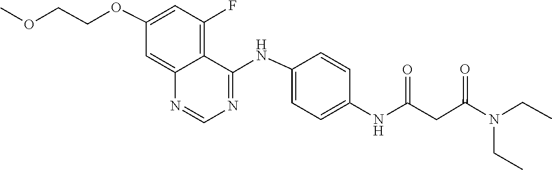 [SMILES: CCN(CC)C(=O)CC(=O)Nc1ccc(Nc2ncnc3cc(OCCOC)cc(F)c23)cc1] | I | 53 | 10.15 (s, 1H), 8.96 (d, J = 12.4 Hz, 1H), 8.45 (s, 1H), 7.66-7.57 (m, 4H), 7.15 (dd, J = 13.8, 2.4 Hz, 1H), 7.06 (d, J = 2.4 Hz, 1H), 4.29 (dd, J = 5.4, 3.4 Hz, 2H), 3.72 (dd, J = 5.6, 3.3 Hz, 2H), 3.47 (s, 2H), 3.37 (q, J = 7.1 Hz, 2H), 3.34 (s, 3H), 3.30 (q, J = 7.0 | 470.2 |
| | | | | | Hz, 2H), 1.15 (t, J = 7.1 | |
| | | | | | Hz, 3H), 1.05 (t, J = 7.0 | |
| | | | | | Hz, 3H). | |
| 185 | 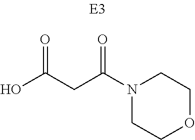 [SMILES: O=C(O)CC(=O)N1CCOCC1] | 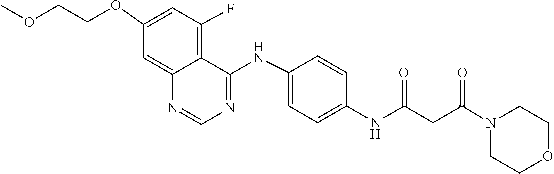 [SMILES: COCCOc1cc(F)c2c(Nc3ccc(NC(=O)CC(=O)N4CCOCC4)cc3)ncnc2c1] | I | 42 | 10.15 (s, 1H), 8.97 (d, J = 12.3 Hz, 1H), 8.45 (s, 1H), 7.63 (d, J = 8.6 Hz, 2H), 7.58 (d, J = 8.8 Hz, 2H), 7.15 (dd, J = 13.7, 2.4 Hz, 1H), 7.06 (d, J = 2.4 Hz, 1H), 4.29 (t, J = 4.4 Hz, 2H), 3.72 (t, J = 4.4 Hz, 2H), 3.65-3.46 (m, 10H), 3.33 (s, 3H). | 484.2 |
| 186 | 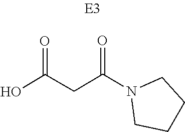 [SMILES: O=C(O)CC(=O)N1CCCC1] | 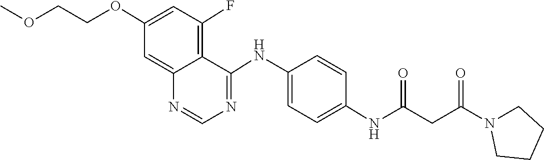 [SMILES: COCCOc1cc(F)c2c(Nc3ccc(NC(=O)CC(=O)N4CCCC4)cc3)ncnc2c1] | I | 69 | 10.19 (s, 1H), 8.96 (d, J = 12.4 Hz, 1H), 8.45 (s, 1H), 7.66-7.57 (m, 4H), 7.15 (dd, J = 13.8, 2.4 Hz, 1H), 7.06 (d, J = 2.4 Hz, 1H), 4.32-4.26 (m, 2H), 3.75-3.69 (m, 2H), 3.50 (t, J = 6.8 Hz, 2H), 3.43 (s, 2H), 3.33 (s, 3H), 1.90 (p, J = 6.8 | 468.2 |
| | | | | | Hz, 2H), 1.81 (p, J = 6.8 | |
| | | | | | Hz, 2H). | |
| 187 | 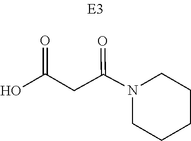 [SMILES: O=C(O)CC(=O)N1CCCCC1] | 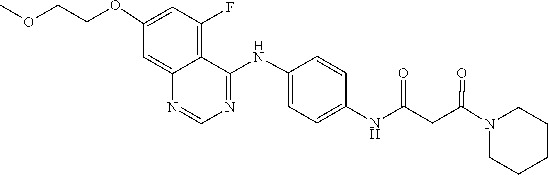 [SMILES: COCCOc1cc(F)c2c(Nc3ccc(NC(=O)CC(=O)N4CCCCC4)cc3)ncnc2c1] | I | 58 | 10.13 (s, 1H), 8.96 (d, J = 12.4 Hz, 1H), 8.45 (s, 1H), 7.63 (d, J = 8.7 Hz, 2H), 7.59 (d, J = 8.7 Hz, 2H), 7.14 (dd, J = 13.9, 2.4 Hz, 1H), 7.06 (d, J = 2.5 Hz, 1H), 4.32-4.26 (m, 2H), 3.72 (dd, J = 5.5, 3.3 Hz, 2H), 3.50 (s, 2H), 3.40 (s, 4H), 3.30 (s, 3H), 1.57 (dq, J = | 482.2 |
| | | | | | 26.1, 5.9 Hz, 4H), 1.45 | |
| | | | | | (p, J = 5.9 Hz, 2H). | |
| 188 | 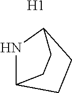 | 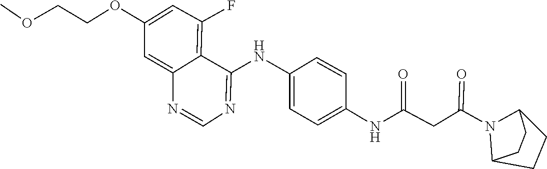 [SMILES: COCCOc1cc(F)c2c(Nc3ccc(NC(=O)CC(=O)N4C5CCC4CC5)cc3)ncnc2c1] | L (T3P) | 16 | 10.15 (s, 1H), 8.96 (d, J = 12.8 Hz, 1H), 8.44 (s, 1H), 7.62 (d, J = 8.8 Hz, 2H), 7.57 (d, J = 8.4 Hz, 2H), 7.13 (d, J = 14.0 Hz, 2.0 Hz, 1H), 7.05 (br s, 1H), 4.40 (m, 2H), 4.28 (m, 2H), 3.71 (m, 2H), 3.41 (s, 2H), 1.78 (m, 2H), 1.62 (m, 2H), 1.43-1.49 (m, 4H). 3Hs | 494.3 |
| | | | | | are under solvents | |
| 189 | 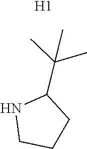 | 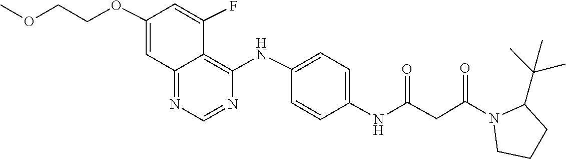 [SMILES: COCCOc1cc(F)c2c(Nc3ccc(NC(=O)CC(=O)N4CCCC4C(C)(C)C)cc3)ncnc2c1] | L (T3P) | 7 | 10.19 (s, 1H), 9.65 (br s, 1H), 8.59 (s, 1H), 7.62 (d, J = 8.8 Hz, 2H), 7.56 (d, J = 8.8 Hz, 2H), 7.29 (br d, J = 14.0 Hz, 1H), 7.05 (d, J = 2.0 Hz, 1H), 4.31 (m, 2H), 3.72 (m, 2H), 3.47-3.54 (m, 2H), 3.33 (s, 3H), 1.80-1.85 (m, 4H), 0.86 (s, 9H). | 524.5 |
| | | | | | 3Hs are under solvent | |
| 191 | 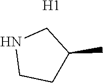 | 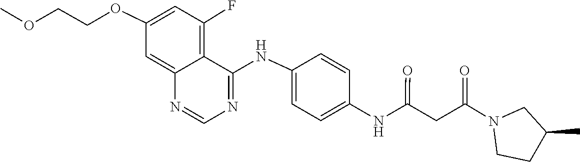 | L (T3P) | 10 | 10.16 (s, 1H), 8.95 (d, J = 12.4 Hz, 1H), 8.44 (s, 1H), 7.62 (d, J = 9.2 Hz, 2H), 7.58 (d, J = 8.8 Hz, 2H), 7.14 (dd, J = 14.0 Hz, 2.4 Hz, 1H), 7.05 (d, J = 2.0 Hz, 1H), 4.28 (m, 2H), 4.14 (m, 0.3H), 4.04 (m, 0.7H), 3.71 (m, 2H), 3.51-3.57 (m, | 482.3 |
| | | | | | 0.6H), 2.43-3.50 (m, | |
| | | | | | 1.4H), 3.41 (m, 2H), | |
| | | | | | 3.32 (s, 3H), 1.90-2.01 | |
| | | | | | (m, 2.1H), 1.80-1.90 (m, | |
| | | | | | 0.9H), 1.62-1.68 (m, | |
| | | | | | 0.3H), 1.50-1.58 (m, | |
| | | | | | 0.7H), 1.17 (d, J = 6.4 | |
| | | | | | Hz, 0.9H), 1.12 (d, J = | |
| | | | | | 6.4 Hz, 2.1H). Note: 2:1 | |
| | | | | | ratio of rotamers. | |
| 192 | 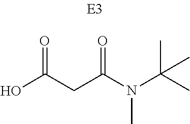 | 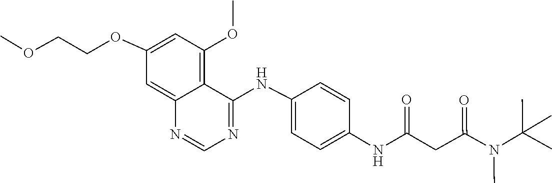 [SMILES: COCCOc1cc(OC)c2c(Nc3ccc(NC(=O)CC(=O)N(C)C(C)(C)C)cc3)ncnc2c1] | L (T3P) | 5 | 10.04 (s, 1H), 8.74 (s, 1H), 8.39 (s, 1H), 7.72 (d, J = 8.8 Hz, 2H), 7.58 (d, J = 8.8 Hz, 2H), 6.77 (d, J = 2.0 Hz, 1H), 6.70 (d, J = 2.4 Hz, 1H), 4.24 (m, 2H), 4.09 (s, 3H), 3.71 (m, 2H), 3.45 (s, 2H), 3.33 (s, 3H), 2.90 (s, 3H), 1.37 (s, 9H). | 496.4 |
| 193 | 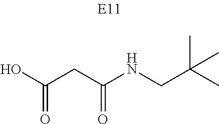 | 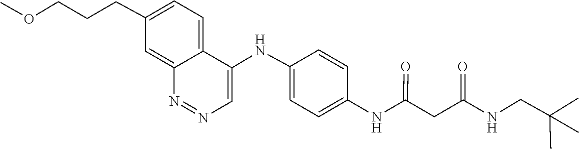 [SMILES: COCCCc1ccc2c(Nc3ccc(NC(=O)CC(=O)NCC(C)(C)C)cc3)cnnc2c1] | I | 30 | 10.19 (s, 1H), 9.16 (s, 1H), 8.67 (s, 1H), 8.28 (d, J = 8.7 Hz, 1H), 7.95 (t, J = 6.3 Hz, 1H), 7.90 (s, 1H), 7.63 (d, J = 8.4 Hz, 2H), 7.55 (d, J = 8.6 Hz, 1H), 7.29 (d, J = 8.4 Hz, 2H), 3.31 (t, J = 6.3 Hz, 2H), 3.27 (s, 2H, hidden under H2O | 464.2 |
| | | | | | peak), 3.19 (s, 3H), 2.87 | |
| | | | | | (d, 2H), 2.80 (t, J = 7.7 | |
| | | | | | Hz, 2H), 1.87 (p, J = 6.9 | |
| | | | | | Hz, 2H), 0.80 (s, 9H). | |
| 194 | 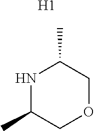 | 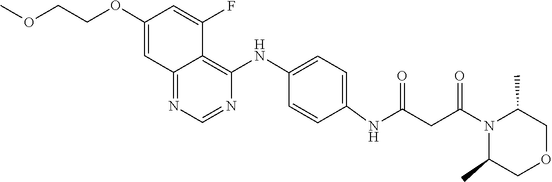 [SMILES: COCCOc1cc(F)c2c(Nc3ccc(NC(=O)CC(=O)N4[C@H](C)COC[C@H]4C)cc3)ncnc2c1] | L (BTFFH) | 20 | 10.16 (s, 1H), 9.03 (d, J = 12.2 Hz, 1H), 8.46 (s, 1H), 7.62 (m, 4H), 7.15 (dd, J = 13.8, 2.4 Hz, 1H), 7.05 (d, J = 2.5 Hz, 1H), 4.29 (m, 2H), 3.98 (m, 3H), 3.71 (m, 2H), 3.60 (m, 2H), 3.33 (s, 5H), 3.13 (m, 1H), 1.25 (q, J = 7.2 Hz, 6H). | 512.2 |
| 195 | 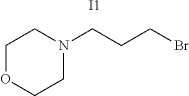 | 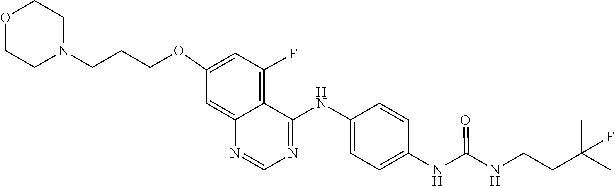 [SMILES: CC(C)(F)CCNC(=O)Nc1ccc(Nc2ncnc3cc(OCCCN4CCOCC4)cc(F)c23)cc1] | N | 15 | 8.86 (d, J = 12.8 Hz, 1H), 8.51 (s, 1H), 8.41 (s, 1H), 7.50 (d, J = 8.8 Hz, 2H), 7.38 (d, J = 8.8 Hz, 2H), 7.10 (dd, J = 13.6, 2.0 Hz, 1H), 7.01 (d, J = 2.0 Hz, 1H), 6.08 (t, J = 5.2 Hz, 1H), 4.19 (t, J = 6.4 Hz, 2H), 3.58 (m, 4H), 3.20 (m, 2H), 2.44 (m, 2H), 2.38 (m, 4H), 1.93 (m 2H), 1.79 | 529.2 |
| | | | | | (m, 2H), 1.35 (d, J = | |
| | | | | | 21.6 Hz, 6H). | |
| 196 | 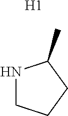 | 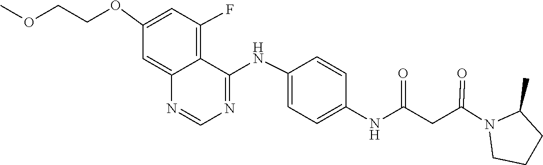 [SMILES: COCCOc1cc(F)c2c(Nc3ccc(NC(=O)CC(=O)N4CCC[C@@H]4C)cc3)ncnc2c1] | L (T3P) | 11 | 10.16 (s, 1H), 8.95 (d, J = 12.4 Hz, 1H), 8.44 (s, 1H), 7.62 (d, J = 8.8 Hz, 2H), 7.58 (d, J = 8.8 Hz, 2H), 7.14 (dd, J = 14.0, 2.4 Hz, 1H), 7.05 (d, J = 2.4 Hz, 1H), 4.28 (m, 2H), 4.02-4.08 (m, 1H), 3.71 (m, 2H), 3.44-3.56 (m, 2H), 3.41 (m, 2H), 3.32 (s, 3H), 1.81-2.01 | 482.3 |
| | | | | | (m, 2H), 1.51-1.65 (m, | |
| | | | | | 1H), 1.17 (d, J = 6.0 Hz, | |
| | | | | | 1H), 1.12 (d, J = 6.0 Hz, | |
| | | | | | 2H). Note: 2:1 ratio of | |
| | | | | | rotamers. | |
| 197 | 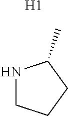 | 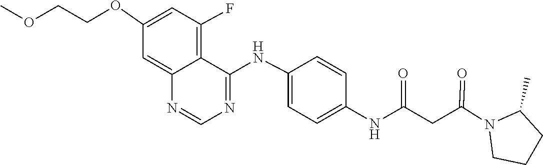 [SMILES: COCCOc1cc(F)c2c(Nc3ccc(NC(=O)CC(=O)N4CCC[C@H]4C)cc3)ncnc2c1] | L (T3P) | 13 | 10.16 (s, 1H), 8.95 (d, J = 12.4 Hz, 1H), 8.44 (s, 1H), 7.63 (d, J = 9.2 Hz, 2H), 7.58 (d, J = 8.8 Hz, 2H), 7.14 (d, J = 13.6 Hz, 2.4 Hz, 1H), 7.05 (d, J = 2.0 Hz, 1H), 4.28 (m, 2H), 4.01-4.16 (m, 1H), 3.71 (m, 2H), 3.47- 3.55 (m, 2H), 3.41 (m, 2H), 3.32 (s, 3H), 1.81- | 482.2 |
| | | | | | 2.00 (m, 3H), 1.51-1.66 | |
| | | | | | (m, 1H), 1.17 (d, J = 6.0 | |
| | | | | | Hz, 1H), 1.12 (d, J = 6.0 | |
| | | | | | Hz, 2H). Note: 2:1 ratio | |
| | | | | | of rotamers. | |
| 198 | 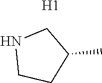 | 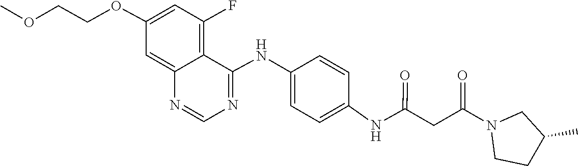 | L (T3P) | 4 | 10.17 (s, 0.5H), 10.16 (s, 0.5H), 8.95 (d, J = 12.0 Hz, 1H), 8.44 (s, 1H), 7.62 (d, J = 8.8 Hz, 2H), 7.57 (d, J = 8.8 Hz, 2H), 7.14 (dd, J = 14.0, 2.4 Hz, 1H), 7.05 (d, J = 2.4 Hz, 1H), 4.28 (m, 2H), 3.71 (m, 2H), 3.58- 3.68 (m, 0.5H), 3.42- 3.56 (m, 2H), 3.40 (s, | 482.1 |
| | | | | | 2H), 3.33 (s, 3H), 3.20- | |
| | | | | | 3.27 (m, 0.5H), 3.02 (m, | |
| | | | | | 0.5H), 2.83 (m, 0.5H), | |
| | | | | | 2.26 (m, 0.5H), 2.19 (m, | |
| | | | | | 0.5H), 2.04 (m, 0.5H), | |
| | | | | | 1.95 (m, 0.5H), 1.54 (m, | |
| | | | | | 0.5H), 1.42 (m, 0.5H), | |
| | | | | | 1.03 (d, J = 6.4 Hz, | |
| | | | | | 1.5H), 1.01 (d, J = 6.4 | |
| | | | | | Hz, 1.5H). Note: 1:1 | |
| | | | | | ratio of rotamers. | |
| 199 | 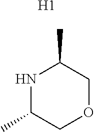 | 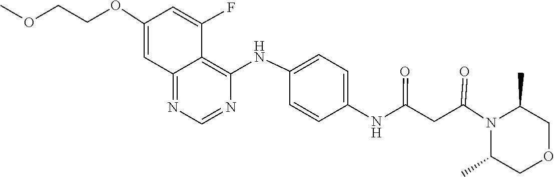 [SMILES: COCCOc1cc(F)c2c(Nc3ccc(NC(=O)CC(=O)N4[C@@H](C)COC[C@@H]4C)cc3)ncnc2c1] | L (BTFFH) | 7 | 10.15 (s, 1H), 8.97 (d, J = 12.4 Hz, 1H), 8.45 (s, 1H), 7.62 (m, 4H), 7.15 (dd, J = 13.8, 2.4 Hz, 1H), 7.06 (d, J = 2.4 Hz, 1H), 4.29 (m, 2H), 3.99 (m, 4H), 3.72 (m, 2H), 3.58 (m, 3H), 3.43 (m, 1H), 3.33 (s, 3H), 1.33 (brs, 6H). | 512.2 |
| 200 | 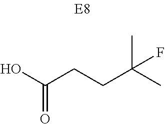 | 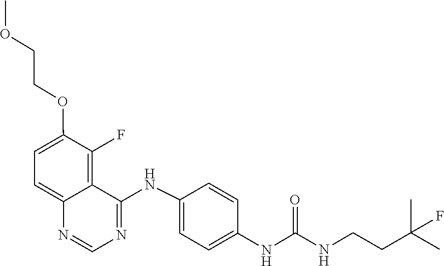 [SMILES: COCCOc1ccc2ncnc(Nc3ccc(NC(=O)NCCC(C)(C)F)cc3)c2c1F] | H | 23 | 8.96 (d, J = 12.8 Hz, 1H), 8.54 (s, 1H), 8.40 (s, 1H), 7.84 (t, J = 9.2 Hz, 1H), 7.61 (d, J = 11.2 Hz, 1H), 7.57 (d, J = 8.8 Hz, 2H), 7.39 (d, J = 8.8 Hz, 2H), 6.10 (t, J = 5.6 Hz, 1H), 4.34 (m, 2H), 3.72 (m, 2H), 3.30 (s, 3H), 3.20 (m, 2H), 1.74-1.84 (m, 2H), 1.35 (d, J = 21.6 Hz, 6H). | 460.1 |
| 201 | 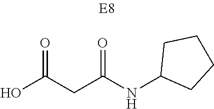 [SMILES: O=C(O)CC(=O)NC1CCCC1] | 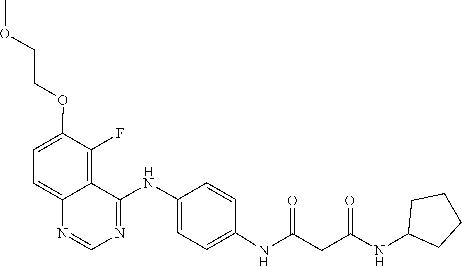 [SMILES: COCCOc1ccc2ncnc(Nc3ccc(NC(=O)CC(=O)NC4CCCC4)cc3)c2c1F] | I (T3P) | 9 | 10.12 (s, 1H), 9.04 (d, J = 13.2 Hz, 1H), 8.44 (s, 1H), 8.05 (d, J = 6.8 Hz, 1H), 7.85 (t, J = 9.2 Hz, 1H), 7.69 (d, J = 9.2 Hz, 2H), 7.63 (d, J = 10.0, Hz, 1H), 7.59 (d, J = 8.8 Hz, 2H), 4.34 (m, 2H), 4.00 (m, 1H), 3.72 (m, 2H), 3.34 (s, 3H), 3.21 (s, 2H), 1.75-1.83 (m, 2H), 1.62-1.68 (m, 2H), 1.49-1.59 (m, 2H), 1.34-1.45 (m, 2H). | 482.1 |
| 202 | 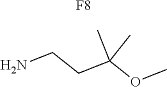 | 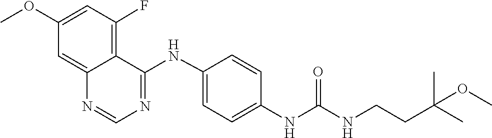 | G | 16 | 8.87 (d, J = 12.4 Hz, 1H), 8.51 (br s, 1H), 8.42 (s, 1H), 7.50 (d, J = 8.8 Hz, 2H), 7.38 (d, J = 8.4 Hz, 2H), 7.10 (dd, J = 14.0, 2.4 Hz, 1H), 7.03 (d, J = 2.0 Hz, 1H), 6.06 (t, J = 5.2 Hz, 1H), 3.92 (s, 3H), 3.13 (m, | 428.2 |
| | | | | | 2H), 3.10 (s, 3H), 1.61 | |
| | | | | | (m, 2H), 1.12 (s, 6H). | |
| 203 | 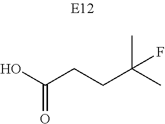 | 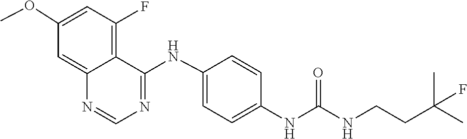 [SMILES: COc1cc(F)c2c(Nc3ccc(NC(=O)NCCC(C)(C)F)cc3)ncnc2c1] | H | 12 | 8.88 (d, J = 12.4 Hz, 1H), 8.53 (s, 1H), 8.42 (s, 1H), 7.51 (d, J = 8.8 Hz, 2H), 7.38 (d, J = 8.8 Hz, 2H), 7.12 (dd, J = 13.6, 2.4 Hz, 1H), 7.03 (d, J = 2.4 Hz, 1H), 6.09 (t, J = 5.6 Hz, 1H), 3.92 (s, 3H), 3.20 (m, 2H), | 416.2 |
| | | | | | 1.74-1.83 (m, 2H), 1.35 | |
| | | | | | (d, J = 21.6 Hz, 6H). | |
| 204 | 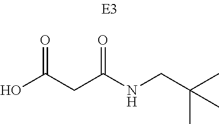 | 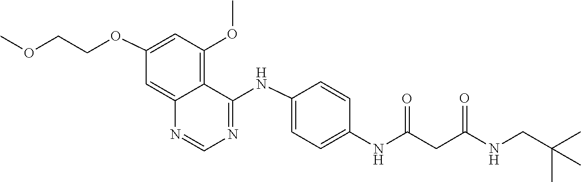 [SMILES: COCCOc1cc(OC)c2c(Nc3ccc(NC(=O)CC(=O)NCC(C)(C)C)cc3)ncnc2c1] | I (T3P) | 15 | 10.10 (s, 1H), 9.75 (s, 1H), 8.39 (s, 1H), 7.97 (t, J = 6.0 Hz, 1H), 7.72 (d, J = 8.8 Hz, 2H), 7.58 (d, J = 9.2 Hz, 2H), 6.77 (d, J = 2.0, Hz, 1H), 6.71 (d, J = 2.4 Hz, 1H), 4.24 (m, 2H), 4.08 (s, 3H), 3.71 (m, 2H), 3.33 (s, 3H), 3.29 (s, 2H), 2.93 (d, J = 6.4 Hz, 2H), 0.86 (s, 9H). | 496.1 |
| 205 | 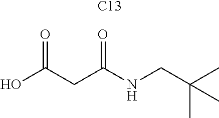 | 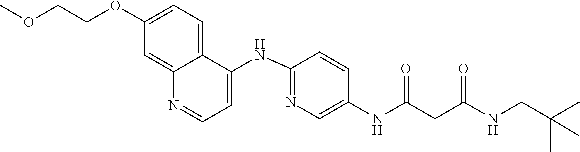 [SMILES: COCCOc1ccc2c(Nc3ccc(NC(=O)CC(=O)NCC(C)(C)C)cn3)ccnc2c1] | I | 31 | 10.21 (s, 1H), 9.33 (s, 1H), 8.51 (dd, J = 16.0, 4.0 Hz, 2H), 8.38 (d, J = 9.4 Hz, 1H), 8.11 (d, J = 5.3 Hz, 1H), 7.99 (dd, J = 8.6, 3.2 Hz, 2H), 7.35-7.28 (m, 2H), 7.22 (dd, J = 9.3, 2.6 Hz, 1H), 4.26 (t, J = 4.5 Hz, 2H), 3.77-3.72 (m, | 466.2 |
| | | | | | 2H), 3.35 (s, 3H), 3.29- | |
| | | | | | 3.35 (m, 2H, under | |
| | | | | | H2O), 2.94 (d, J = 6.2 | |
| | | | | | Hz, 2H), 0.87 (s, 9H). | |
| 206 | 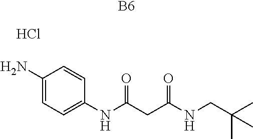 [SMILES: Cl ; "B6" ; CC(C)(C)CNC(=O)CC(=O)Nc1ccc(N)cc1] | 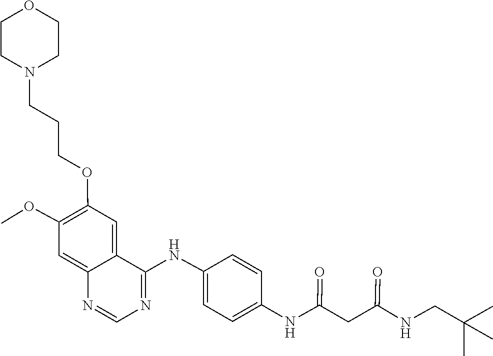 [SMILES: COc1cc2ncnc(Nc3ccc(NC(=O)CC(=O)NCC(C)(C)C)cc3)c2cc1OCCCN1CCOCC1] | Sub- stitution | 13 | 10.13 (s, 1H), 9.50 (s, 1H), 8.43 (s, 1H), 7.99 (t, J = 6.3 Hz, 1H), 7.88 (s, 1H), 7.70 (d, J = 8.6 Hz, 2H), 7.61 (d, J = 8.6 Hz, 2H), 7.19 (s, 1H), 4.26-4.17 (m, 2H), 3.93 (s, 3H), 3.67-3.58 (m, 4H), 3.31 (m, 2H), 3.25-3.12 (m, 2H), 2.94 (d, J = 6.2 Hz, 2H), 2.40-1.90 (m, 4H), 1.30-1.22 (m, 2H), 0.88 (s, 9H). | 565.3 |
| 207 | 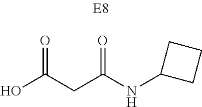 [SMILES: O=C(O)CC(=O)NC1CCC1] | 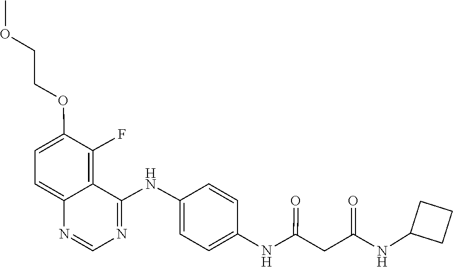 [SMILES: COCCOc1ccc2ncnc(Nc3ccc(NC(=O)CC(=O)NC4CCC4)cc3)c2c1F] | I (T3P) | 5 | 10.11 (s, 1H), 9.03 (d, J = 12.4 Hz, 1H), 8.43 (s, 1H), 8.34 (d, J = 7.6 Hz, 1H), 7.85 (t, J = 9.2 Hz, 1H), 7.68 (d, J = 8.8 Hz, 2H), 7.62 (m, 1H), 7.59 (d, J = 8.8 Hz, 2H), 4.33 (m, 2H), 4.20 (m, 1H), 3.71 (m, 2H), 3.19 (s, 2H), 2.13-2.18 (m, 2H), 1.86-1.92 (m, 2H), 1.59-1.67 (m, 2H). 3Hs, are under solvents | 468.2 |
| 208 | 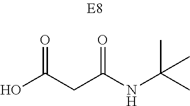 | 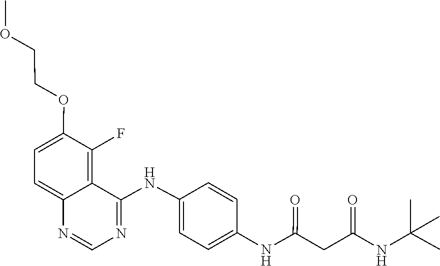 [SMILES: COCCOc1ccc2ncnc(Nc3ccc(NC(=O)CC(=O)NC(C)(C)C)cc3)c2c1F] | I (T3P) | 5 | 10.09 (s, 1H), 9.04 (br d, J = 7.2 Hz, 1H), 8.43 (s, 1H), 7.85 (t, J = 9.2 Hz, 1H), 7.67-7.72 (m, 3H), 7.60 (m, 3H), 4.34 (m, 2H), 3.71 (m, 2H), 3.20 (s, 2H), 1.27 (s, 9H). 3Hs are under solvents | 470.2 |
| 209 | 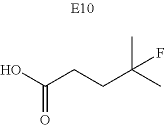 | 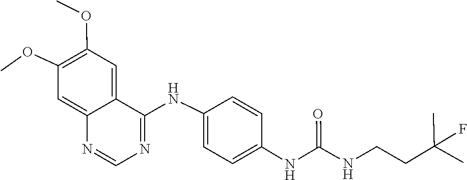 [SMILES: COc1cc2ncnc(Nc3ccc(NC(=O)NCCC(C)(C)F)cc3)c2cc1OC] | H | 2 | 9.37 (s, 1H), 8.53 (s, 1H), 8.38 (s, 1H), 7.82 (s, 1H), 7.57 (d, J = 8.8 Hz, 2H), 7.40 (d, J = 8.8 Hz, 2H), 7.15 (s, 1H), 6.14 (t, J = 5.6 Hz, 1H), 3.94 (s, 3H), 3.92 (s, 3H) 3.20 (m, 2H), 1.74- 1.83 (m, 2H), 1.35 (d, J = 21.6 Hz, 6H). | 428.2 |
| 210 | 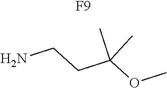 | 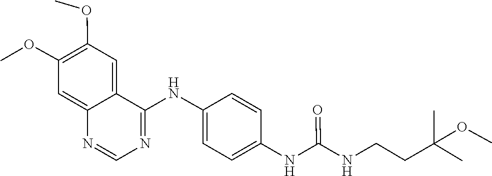 | G | 10 | 9.37 (s, 1H), 8.46 (s, 1H), 8.38 (s, 1H), 7.82 (s, 1H), 7.56 (d, J = 8.4 Hz, 2H), 7.40 (d, J = 8.8 Hz, 2H), 7.40 (d, J = 8.8 Hz, 2H), 7.15 (s, 1H), 6.03 (t, J = 6.0 Hz, 1H), 3.94 (s, 3H), 3.92 (s, 3H), 3.13 (m, 2H), 3.11 (s, 3H), 1.62 (m, 2H), 1.12 (s, 6H). | 440.2 |
| 211 | 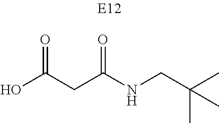 | 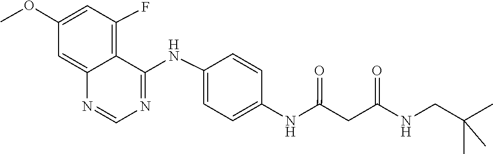 [SMILES: COc1cc(F)c2c(Nc3ccc(NC(=O)CC(=O)NCC(C)(C)C)cc3)ncnc2c1] | I (T3P) | 440.2 | 10.13 (s, 1H), 8.96 (d, J = 11.6 Hz, 1H), 8.45 (s, 1H), 7.98 (t, J = 6.0 Hz, 1H), 7.63 (d, J = 8.8 Hz, 2H), 7.58 (d, J = 8.8 Hz, 2H), 7.12 (dd, J = 2.4 Hz, 1H), 7.05 (d, J = 2.4, Hz, 1H), 3.93 (s, 3H), 3.30 (s, 2H), 2.93 (d, J = 6.4 Hz, 2H), 0.86 (s, 9H). | 23 |
| 212 | 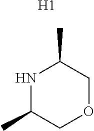 | 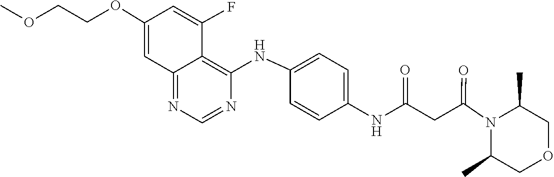 [SMILES: COCCOc1cc(F)c2c(Nc3ccc(NC(=O)CC(=O)N4[C@H](C)COC[C@@H]4C)cc3)ncnc2c1] | L (PyAOP) | 49 | 10.16 (s, 1H), 8.97 (d, J = 12.3 Hz, 1H), 8.45 (s, 1H), 7.61 (m, 4H), 7.15 (dd, J = 13.7, 2.4 Hz, 1H), 7.06 (d, J = 2.4 Hz, 1H), 4.29 (m, 2H), 3.98 (m, 1H), 3.71 (m, 4H), 3.44 (m, 5H), 3.34 (s, 3H), 1.28 (d, J = 6.2 Hz, 6H). | 512.2 |
| 213 | 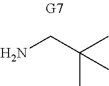 | 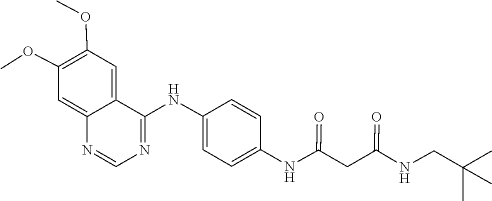 [SMILES: COc1cc2ncnc(Nc3ccc(NC(=O)CC(=O)NCC(C)(C)C)cc3)c2cc1OC] | M | 62 | 10.11 (s, 1H), 9.49 (s, 1H), 8.43 (s, 1H), 7.99 (t, J = 6.3 Hz, 1H), 7.85 (s, 1H), 7.70 (d, J = 8.5 Hz, 2H), 7.61 (d, J = 8.5 Hz, 2H), 7.18 (s, 1H), 3.96 (s, 3H), 3.94 (s, 3H), 3.31 (s, 2H), 2.94 (d, J = 6.2 Hz, 2H), 0.88 (s, 9H). | 452.2 |
| 214 | 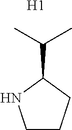 | 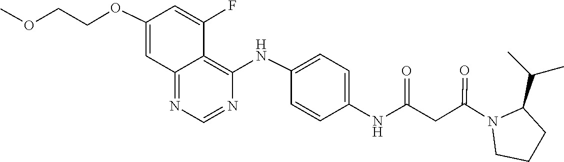 [SMILES: COCCOc1cc(F)c2c(Nc3ccc(NC(=O)CC(=O)N4CCC[C@@H]4C(C)C)cc3)ncnc2c1] | L (T3P) | 16 | 10.15 (d, J = 3.6 Hz, 1H), 8.96 (d, J = 12.0 Hz, 1H), 8.44 (s, 1H), 7.62 (d, J = 9.2 Hz, 2H), 7.57 (d, J = 9.2 Hz, 2H), 7.14 (dd, J = 13.6, 2.4 Hz, 1H), 7.05 (d, J = 2.4 Hz, 1H), 4.28 (m, 2H), 3.87 (m, 1H), 3.71 (m, 2H), 3.64-3.56 (m, 1H), 3.38 (m, 1H), | 510.2 |
| | | | | | 2.23 (m, 1H), 1.71-1.90 | |
| | | | | | (m, 4H), 0.82 (d, J = 6.8 | |
| | | | | | Hz, 3H), 0.76 (d, J = 6.8 | |
| | | | | | Hz, 3H). 3Hs are under | |
| | | | | | solvents. 2Hs are | |
| | | | | | missing. | |
| 215 | 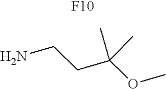 | 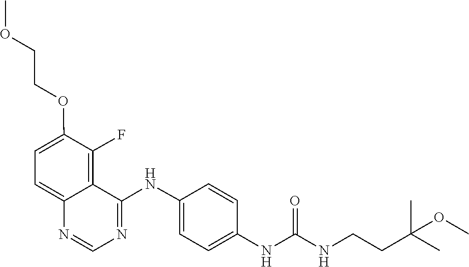 | G | 40 | 8.95 (d, J = 13.6 Hz, 1H), 8.50 (s, 1H), 8.40 (s, 1H), 7.84 (t, J = 9.2 Hz, 1H), 7.65 (d, J = 9.2 Hz, 1H), 7.56 (d, J = 8.8 Hz, 2H), 7.40 (d, J = 8.8 Hz, 2H), 6.02 (t, J = 5.6 Hz, 1H), 4.34 (m, 2H), 3.72 (m, 2H), 3.33 (s, 3H), 3.14-3.08 (m, 2H), 3.11 (s, 3H), 1.63 (m, 2H), 1.12 (s, 6H). | 472.2 |
| 216 | 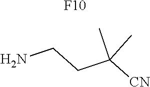 | 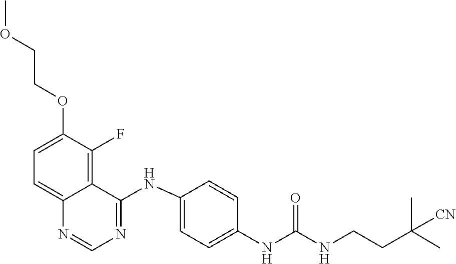 [SMILES: COCCOc1ccc2ncnc(Nc3ccc(NC(=O)NCCC(C)(C)C#N)cc3)c2c1F] | G | 7 | 8.96 (d, J = 13.2 Hz, 1H), 8.54 (s, 1H), 8.40 (s, 1H), 7.84 (t, J = 9.2 Hz, 1H), 7.61 (m, 1H), 7.57 (d, J = 8.8 Hz, 2H), 7.40 (d, J = 8.8 Hz, 2H), 6.20 (t, J = 5.6 Hz, 1H), 4.33 (m, 2H), 3.71 (m, 2H), 3.34 (s, 3H), 3.24 (m, 2H), 1.72 (m, 2H), 1.33 (s, 6H). | 467.2 |
| 217 | 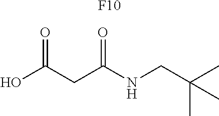 | 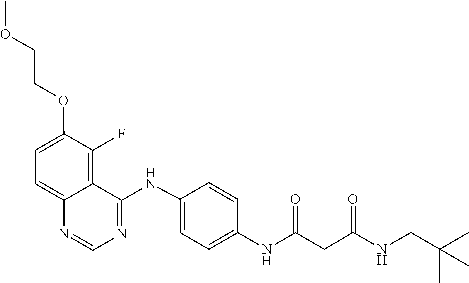 [SMILES: COCCOc1ccc2ncnc(Nc3ccc(NC(=O)CC(=O)NCC(C)(C)C)cc3)c2c1F] | I (T3P) | 10 | 10.14 (s, 1H), 9.04 (d, J = 12.4 Hz, 1H), 8.43 (s, 1H), 7.98 (t, J = 6.4 Hz, 1H), 7.85 (t, J = 6.0 Hz, 1H), 7.69 (d, J = 8.8 Hz, 2H), 7.62 (m, 1H), 7.60 (d, J = 8.8 Hz, 2H), 4.34 (m, 2H), 3.71 (m, 2H), 3.34 (s, 3H), 3.30 (s, 2H), 2.93 (d, J = 6.0 Hz, 2H), 0.86 (s, 9H). | 184.3 |
| 218 | 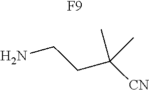 | 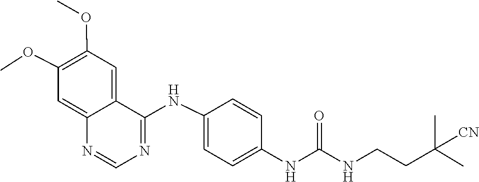 [SMILES: COc1cc2ncnc(Nc3ccc(NC(=O)NCCC(C)(C)C#N)cc3)c2cc1OC] | G | 6 | 9.34 (s, 1H), 8.54 (s, 1H), 8.38 (s, 1H), 7.82 (s, 1H), 7.58 (s, 1H), 7.56 (s, 1H), 7.42 (s, 1H), 7.39 (s, 1H), 7.15 (s, 1H), 6.23 (t, J = 5.6 Hz, 1H), 3.94 (s, 3H), 3.92 (s, 3H), 3.24 (m, 2H), 1.72 (m, 2H), 1.34 (s, 6H). | 435.2 |
| 219 | 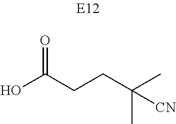 | 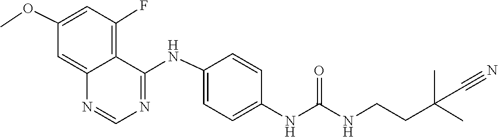 [SMILES: COc1cc(F)c2c(Nc3ccc(NC(=O)NCCC(C)(C)C#N)cc3)ncnc2c1] | H | 7 | 8.88 (d, J = 12.4 Hz, 1H), 8.54 (s, 1H), 8.42 (s, 1H), 7.51 (d, J = 8.8 Hz, 2H), 7.39 (d, J = 8.8 Hz, 2H), 7.11 (dd, J = 13.6, 2.4 Hz, 1H), 7.03 (d, J = 2.0 Hz, 1H), 6.20 (t, J = 5.6 Hz, 1H), 3.92 (s, 3H), 3.23 (m, 2H), | 423.2 |
| | | | | | 1.72 (m, 2H), 1.33 (s, | |
| | | | | | 6H). | |
| 220 | 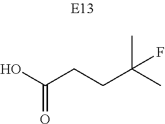 | 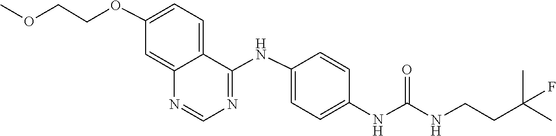 [SMILES: COCCOc1ccc2c(Nc3ccc(NC(=O)NCCC(C)(C)F)cc3)ncnc2c1] | H | 14 | 10.17 (br s, 1H), 8.59 (d, J = 6.0 Hz, 2H), 8.49 (d, J = 8.4 Hz, 1H), 7.56 (br d, J = 7.2 Hz, 2H), 7.42 (br d, J = 7.2 Hz, 2H), 7.33 (br d, J = 5.2 Hz, 1H), 7.17 (br m, 1H), 6.13 (br s, 1H), 4.29 (br m, 2H), 3.73 (br m, 2H), | 442.2 |
| | | | | | 3.33 (s, 3H), 3.21 (br m, | |
| | | | | | 2H), 1.77-1.81 (br m, | |
| | | | | | 2H), 1.34 (br d, J = 21.6 | |
| | | | | | Hz, 6H). | |
| 221 | 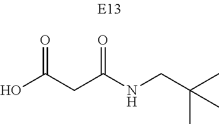 | 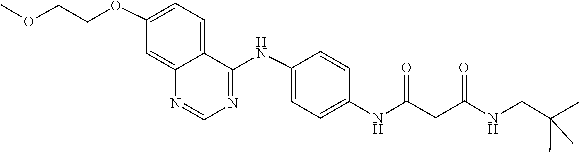 [SMILES: COCCOc1ccc2c(Nc3ccc(NC(=O)CC(=O)NCC(C)(C)C)cc3)ncnc2c1] | I (T3P) | 9 | 10.11 (s, 1H), 9.63 (s, 1H), 8.48 (s, 1H), 8.43 (d, J = 9.2 Hz, 1H), 7.98 (t, J = 6.4 Hz, 1H), 7.34 (d, J = 8.8 Hz, 2H), 7.58 (d, J = 9.2 Hz, 2H), 7.24 (dd, J = 9.2, 2.4 Hz, 1H), 7.16 (d, J = 24 Hz, 1H), 4.26 (m, 2H), 3.72 (m, 2H), 3.33 (s, 3H), | 466.3 |
| | | | | | 3.29 (s, 2H), 2.93 (d, J = | |
| | | | | | 6.4 Hz, 2H), 0.86 (s, | |
| | | | | | 9H). | |
| 222 | 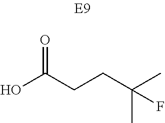 | 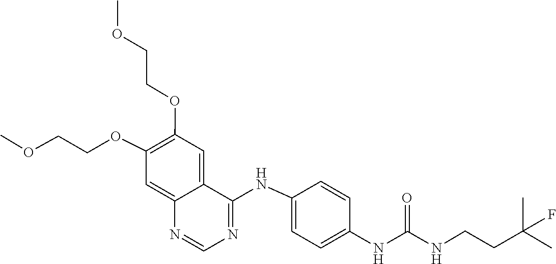 [SMILES: COCCOc1cc2ncnc(Nc3ccc(NC(=O)NCCC(C)(C)F)cc3)c2cc1OCCOC] | H | 5 | 9.49 (br s, 1H), 8.51 (s, 1H), 8.41 (s, 1H), 7.86 (s, 1H), 7.56 (d, J = 8.8 Hz, 2H), 7.40 (d, J = 8.8 Hz, 2H), 7.18 (s, 1H), 6.09 (s, 1H), 4.27 (m, 4H), 3.73-3.78 (m, 4H), 3.36 (s, 3H), 3.35 (s, 3H), 3.20 (m, 2H), 1.75- 1.83 (m, 2H), 1.34 (d, J = 21.6 Hz, 6H). | 516.3 |
| 223 | 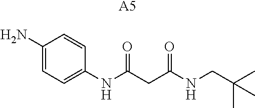 [SMILES: "A5" ; CC(C)(C)CNC(=O)CC(=O)Nc1ccc(N)cc1] | 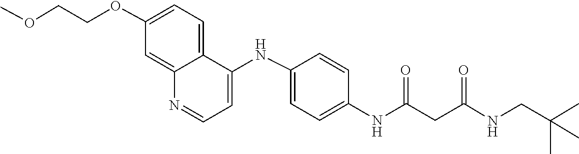 [SMILES: COCCOc1ccc2c(Nc3ccc(NC(=O)CC(=O)NCC(C)(C)C)cc3)ccnc2c1] | P | 26 | 10.07 (s, 1H), 8.86 (s, 1H), 8.26 (d, J = 5.4 Hz, 1H), 8.22 (d, J = 9.3 Hz, 1H), 7.91 (t, J = 6.3 Hz, 1H), 7.57 (d, J = 8.4 Hz, 2H), 7.22 (d, J = 8.5 Hz, 2H), 7.17 (d, J = 2.7 Hz, 1H), 7.10 (dd, J = 9.2, 2.6 Hz, 1H), 6.60 (d, J = 5.4 Hz, 1H), 4.17 (t, J = | 465.2 |
| | | | | | 4.5 Hz, 2H), 3.67 (dd, | |
| | | | | | J = 5.4, 3.5 Hz, 2H), 3.27 | |
| | | | | | (s, 3H), 3.24 (s, 2H), | |
| | | | | | 2.87 (d, J = 6.2 Hz, 2H), | |
| | | | | | 0.80 (s, 9H). | |
| 224 | 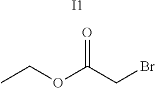 | 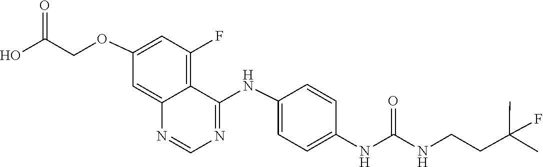 [SMILES: CC(C)(F)CCNC(=O)Nc1ccc(Nc2ncnc3cc(OCC(=O)O)cc(F)c23)cc1] | N & hydrolysis with LiOH | 15 | 13.30 (s, 1H), 8.89 (d, J = 12.4 Hz, 1H), 8.57 (s, 1H), 8.40 (s, 1H), 7.49 (d, J = 8.8 Hz, 2H), 7.37 (d, J = 8.8 Hz, 2H), 7.13 (d, J = 13.6 Hz, 1H), 6.93 (s, 1H), 6.14 (t, J = 5.6 Hz, 1H), 4.83 (s, 2H), 3.20 (m, 2H), 1.74- 1.83 (m, 2H), 1.34 (d, J = 21.6 Hz, 6H) | 460.1 |
| 225 | 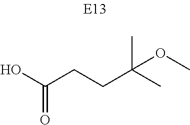 | 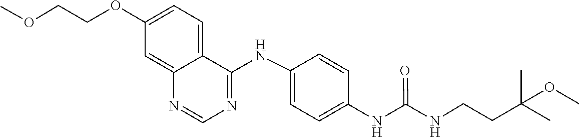 | H | 11 | 9.54 (s, 1H), 8.45 (m, 2H), 8.41 (d, 9.2 Hz, 1H), 7.61 (d, J = 8.8 Hz, 2H), 7.38 (d, J = 8.4 Hz, 2H), 7.22 (dd, J = 9.2, 2.4 Hz, 1H), 7.41 (d, J = 2.4 Hz, 1H), 6.03 (d, J = 5.2 Hz, 1H), 4.26 (m, 2H), 3.72 (m, 2H), 3.11 | 454.5 |
| | | | | | (s, 3H), 3.07-3.20 (m, | |
| | | | | | 2H), 1.61 (m, 2H), 1.11 | |
| | | | | | (s, 6H). 3Hs are nder | |
| | | | | | solvents | |
| 226 | 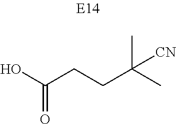 | 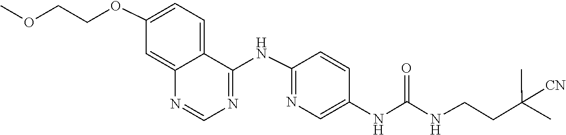 [SMILES: COCCOc1ccc2c(Nc3ccc(NC(=O)NCCC(C)(C)C#N)cn3)ncnc2c1] | H | 11 | 10.11 (s, 1H), 8.76 (s, 1H), 8.57 (m, 2H), 8.49 (d, J = 2.4 Hz, 1H), 8.19 (d, J = 8.8 Hz, 1H), 7.82 (dd, J = 8.8, 2.4 Hz, 1H), 7.19 (m, 2H), 6.41 (t, J = 4.8 Hz, 1H), 4.27 (m, 2H), 3.72 (m, 2H), 3.26 (m, 2H), 1.73 (m, | 450.3 |
| | | | | | 2H), 1.34 (s, 6H). 3H | |
| | | | | | are under solvents. | |
| 227 | 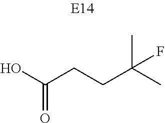 | 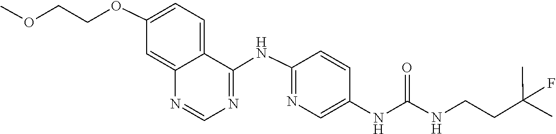 [SMILES: COCCOc1ccc2c(Nc3ccc(NC(=O)NCCC(C)(C)F)cn3)ncnc2c1] | H | 33 | 10.11 (s, 1H), 8.67 (s, 1H), 8.56 (m, 2H), 8.48 (d, J = 2.4 Hz, 1H), 8.19 (d, J = 8.8 Hz, 1H), 7.82 (dd, J = 6.4, 2.4 Hz, 1H), 7.19 (m, 2H), 6.22 (t, J = 5.6 Hz, 1H), 4.27 (m, 2H), 3.72 (m, 2H), 3.21 (m, 2H), 1.75-1.84 | 443.2 |
| | | | | | (m, 2H), 1.35 (d, J = | |
| | | | | | 21.6 Hz, 6H). 3H are | |
| | | | | | under solvents. | |
| 228 | 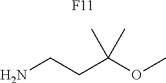 | 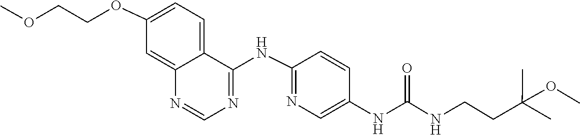 | G | 33 | 10.09 (s, 1H), 8.65 (s, 1H), 8.56 (m, 2H), 8.48 (d, J = 2.4 Hz, 1H), 8.18 (d, J = 8.8 Hz, 1H), 7.81 (dd, J = 8.8, 2.8 Hz, 1H), 7.19 (m, 2H), 6.19 (t, J = 5.6 Hz, 1H), 4.27 (m, 2H), 3.72 (m, 2H), 3.33 (s, 3H), 3.11 (s, | 455.2 |
| | | | | | 3H), 3.08-3.16 (m, 2H), | |
| | | | | | 1.62 (m, 2H), 1.12 (s, | |
| | | | | | 6H). | |
| 229 | 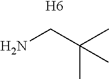 | 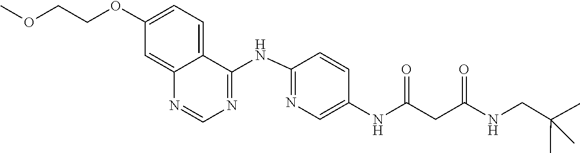 [SMILES: COCCOc1ccc2c(Nc3ccc(NC(=O)CC(=O)NCC(C)(C)C)cn3)ncnc2c1] | L (T3P) | 22 | 10.28 (s, 1H), 10.22 (s, 1H), 8.66 (d, J = 2.4 Hz, 1H), 8.58 (m, 2H), 8.31 (d, J = 9.2 Hz, 1H), 7.98 (m, 2H), 7.21 (m, 2H), 4.28 (m, 2H), 3.72 (m, 2H), 3.33 (s, 3H), 2.93 (d, J = 6.4 Hz, 2H), 0.86 (s, 9H). 2Hs are under solvent peaks | 467.3 |

### Biological Assays

The following assay was used to measure the effects of compounds of the disclosure.

Biochemical Assay for uKIT

Activity of c-KIT kinase (SEQ. ID NO. 1 or SEQ. ID NO. 2) was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942). Assays were conducted in 384-well plates (100 μL final volume) using 16 nM (Decode, SEQ ID No. 1) or 4.36 nM (Signal Chem, SEQ. ID No. 2), 0.7 mg/mL PE4Y substrate, 1.5 units pyruvate kinase, 2.1 units lactate dehydrogenase, 1 mM phosphoenol pyruvate, 0.28 mM NADH and 1 mM ATP in assay buffer (100 mM Tris, pH 7.5, 15 mM MgCl₂, 0.5 mM DTT, 0.1% octyl-glucoside, 0.002% (w/v) BSA, and 0.002% Triton X-100). Inhibition of KIT was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 2-3 h time frame. The reaction rate at each concentration of compound was converted to percent inhibition using controls (i.e., reaction with no test compound and reaction with a known inhibitor) and IC₅₀ values were calculated using software routines in Prism (GraphPad software).

c-KIT protein sequence (residues T544-V976 with N-
terminal GST fusion)
SEQ ID No. 1
MEHHHHHHHHEYMPMEMAPILGYWKIKGLVQPTRLLLEYLEEKYEEHLYE
RDEGDKWRNKKFELGLEFPNLPYYIDGDVKLTQSMAIIRYIADKHNMLGG
CPKERAEISMLEGAVDIRYGVSRIAYSKDFETLKVDFLSKLPEMLKMFED
RLCHKTYLNGDHVTHPDFMLYDALDVVLYMDPMCLDAFPKLVCFKKRIEA
IPQIDKYLKSSKYIWPLQGWQATFGGGDHPPKSDLVPRHNQTSLYKKAGS
AAAVLEENLYFQGTYKYLQKPMYEVQWKVVEEINGNNYVYIDPTQLPYDH
KWEFPRNRLSFGKTLGAGAFGKVVEATAYGLIKSDAAMTVAVKMLKPSAH
LTEREALMSELKVLSYLGNHMNIVNLLGACTIGGPTLVITEYCCYGDLLN
FLRRKRDSFICSKQEDHAEAALYKNLLHSKESSCSDSTNEYMDMKPGVSY
VVPTKADKRRSVRIGSYIERDVTPAIMEDDELALDLEDLLSFSYQVAKGM
AFLASKNCIHRDLAARNILLTHGRITKICDFGLARDIKNDSNYVVKGNAR
LPVKWMAPESIFNCVYTFESDVWSYGIFLWELFSLGSSPYPGMPVDSKFY
KMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
c-KIT protein sequence (residues 544-end with N-
terminal GST-tag and Thrombin site)
SEQ ID No. 2
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSTYKYLQKPMYEVQWKVVEEINGNN
YVYIDPTQLPYDHKWEFPRNRLSFGKTLGAGAFGKVVEATAYGLIKSDAA
MTVAVKMLKPSAHLTEREALMSELKVLSYLGNHMNIANLLGACTIGGPTL
VITEYCCYGDLLNFLRRKRDSFICSKQEDHAEAALYKNLLHSKESSCSDS
TNEYMDMKPGVSYVVPTKADKRRSVRIGSYIERDVTPAIMEDDELALDLE
DLLSFSYQVAKGMAFLASKNCIHRDLAARNILLTHGRITKICDFGLARDI
KNDSNYVVKGNARLPVKWMAPESIFNCVYTFESDVWSYGIFLWELFSLGS
SPYPGMPVDSKFYKMIKEGFRMLSPEHAPAEMYDIMKTCWDADPLKRPTF
KQIVQLIEKQISESTNHIYSNLANCSPNRQKPVVDHSVRINSVGSTASSS
QPLLVHDDV

TABLE I
Inhibition of biochemical activity of uKIT kinases by exemplary
compounds shown in Table H.

| Example | |
|---|---|
| (Compound) | |
| Number | uKIT IC₅₀ |
| 1 | + |
| 2 | + |
| 3 | ++ |
| 4 | ++ |
| 5 | ++ |
| 6 | + |
| 7 | + |
| 8 | + |
| 9 | + |
| 10 | + |
| 11 | ++ |
| 12 | ++ |
| 13 | ++ |
| 14 | ++ |
| 15 | + |
| 16 | + |
| 17 | + |
| 18 | + |
| 19 | + |
| 20 | + |
| 21 | + |
| 22 | + |
| 23 | +++ |
| 24 | +++ |
| 25 | +++ |
| 26 | + |
| 27 | +++ |
| 28 | ++ |
| 29 | +++ |
| 30 | + |
| 31 | + |
| 32 | + |
| 33 | ++ |
| 34 | + |
| 35 | + |
| 36 | + |
| 37 | ++ |
| 38 | ++ |
| 39 | + |
| 40 | ++++ |
| 41 | + |
| 42 | + |
| 43 | ++ |
| 44 | ++ |
| 45 | + |
| 46 | + |
| 47 | + |
| 48 | +++ |
| 49 | + |
| 50 | +++ |
| 51 | ++ |
| 52 | ++ |
| 53 | ++++ |
| 54 | ++ |
| 55 | +++ |
| 56 | + |
| 57 | +++ |
| 58 | ++ |
| 59 | ++ |
| 60 | ++ |
| 61 | ++ |
| 62 | ++++ |
| 63 | ++ |
| 64 | ++ |
| 65 | + |
| 66 | ++ |
| 67 | ++ |
| 68 | + |
| 69 | + |
| 70 | ++++ |
| 71 | ++ |
| 72 | ++++ |
| 73 | ++ |
| 74 | ++ |
| 75 | ++ |
| 76 | ++ |
| 77 | ++ |
| 78 | +++ |
| 79 | ++ |
| 80 | ++++ |
| 81 | ++ |
| 82 | + |
| 83 | ++++ |
| 84 | ++++ |
| 85 | + |
| 86 | ++ |
| 87 | ++ |
| 88 | ++ |
| 89 | ++ |
| 90 | + |
| 91 | + |
| 92 | + |
| 93 | +++ |
| 94 | +++ |
| 95 | + |
| 96 | ++ |
| 97 | + |
| 98 | ++ |
| 99 | + |
| 100 | ++ |
| 101 | +++ |
| 102 | ++ |
| 103 | ++ |
| 104 | + |
| 105 | + |
| 106 | + |
| 107 | ++ |
| 108 | ++ |
| 109 | ++ |
| 110 | ++ |
| 111 | + |
| 112 | ++ |
| 113 | ++ |
| 114 | +++ |
| 115 | ++ |
| 116 | +++ |
| 117 | +++ |
| 118 | + |
| 119 | ++ |
| 120 | + |
| 121 | +++ |
| 122 | ++ |
| 123 | + |
| 124 | ++ |
| 125 | ++ |
| 126 | +++ |
| 127 | ++ |
| 128 | ++ |
| 129 | ++ |
| 130 | ++ |
| 131 | ++ |
| 132 | ++ |
| 133 | + |
| 134 | ++++ |
| 135 | + |
| 136 | ++ |
| 137 | ++ |
| 138 | + |
| 139 | ++ |
| 140 | ++ |
| 141 | ++ |
| 142 | ++ |
| 143 | ++ |
| 144 | +++ |
| 145 | ++ |
| 146 | +++ |
| 147 | ++ |
| 148 | ++ |
| 149 | + |
| 150 | + |
| 151 | + |
| 152 | ++ |
| 153 | ++ |
| 154 | ++ |
| 155 | + |
| 156 | + |
| 157 | ++ |
| 158 | + |
| 159 | + |
| 160 | + |
| 161 | + |
| 162 | + |
| 163 | +++ |
| 164 | ++ |
| 165 | ++ |
| 166 | ++ |
| 167 | ++ |
| 168 | ++ |
| 169 | ++ |
| 170 | ++ |
| 171 | ++ |
| 172 | ++ |
| 173 | + |
| 174 | + |
| 175 | + |
| 176 | ++ |
| 177 | ++ |
| 179 | +++ |
| 180 | ++++ |
| 181 | + |
| 182 | ++ |
| 183 | ++ |
| 184 | ++ |
| 185 | +++ |
| 186 | +++ |
| 187 | +++ |
| 188 | ++ |
| 189 | ++ |
| 190 | ++ |
| 191 | ++ |
| 192 | ++ |
| 193 | ++ |
| 195 | + |
| 196 | ++ |
| 197 | ++ |
| 198 | ++ |
| 199 | +++ |
| 200 | +++ |
| 201 | +++ |
| 202 | ++ |
| 203 | + |
| 204 | ++ |
| 205 | ++++ |
| 206 | +++ |
| 207 | +++ |
| 208 | ++++ |
| 209 | ++ |
| 210 | ++ |
| 211 | ++ |
| 213 | ++ |
| 214 | ++ |
| 215 | ++ |
| 216 | ++ |
| 217 | +++ |
| 219 | ++ |
| 220 | + |
| 221 | ++ |
| 222 | ++ |
| 223 | +++ |
| 224 | ++ |
| 225 | + |
| 226 | +++ |
| 227 | +++ |
| 228 | ++ |
| 229 | +++ |

For Table I, “+” refers to an IC₅₀ less than or equal to 10 nM; “++” refers to an IC₅₀ greater than 10 nM and less than or equal to 100 nM; “+++” refers to an IC₅₀ greater than 100 nM and less than or equal to 1000 nM; and “++++” refers to an IC₅₀ greater than 1000 nM.

### Ba/F3 KIT Mutant Cell Line Transduction and Expansion

### Cloning and Vector Packaging

Several different KIT cDNA encoding various primary and secondary mutations were cloned into a pLV(Exp]-CMV>(cDNA):IRES:puro vector. These vectors were constructed and packaged by VectorBuilder.

Exponentially grown Ba/F3 cells (1×10⁶ cells in 1 ml medium) were infected with 60 μl of viral suspension in a 6-well plate in the presence of mIL-3 (10 ng/ml) and polybrene (7.5 μg/ml) (Vector Builder) and incubated for 16 h. After 16 h, the cells were centrifuged, and viral supernatant was discarded. The cells were then re-suspended in fresh medium and allowed to recover for another day. After 72 h post infection, the cells were seeded with complete medium with mIL-3 and with 1 μg/ml puromycin added to medium for selection. After 48 h, cells without viral infection die, whereas successfully transduced cells were collected and changed to fresh medium with 5 ng/ml of IL-3. After a week or two with gradual withdrawal of mIL3 the cells that can continue to proliferate in the absence of mIL3 were exponentially expanded and were frozen down for banking.

Ba/F3 mutant KIT Cell Proliferation Assays

A serial dilution of test compound was dispensed into a 384-well black clear bottom plate (Corning, Corning, NY). Six hundred and twenty-five cells were added per well in 50 μL complete growth medium. Plates were incubated for 68 h at 37° C., 5% CO₂, 95% humidity. At the end of the incubation period 10 μL of a 440 μM solution of resazurin (Sigma, St. Louis, MO) in PBS was added to each well and incubated for an additional 5 h at 37° C., 5% CO₂, 95% humidity. Plates were read on a Synergy2 reader (Biotek, Winooski, VT) using an excitation of 540 nM and an emission of 600 nM. Data was analyzed using Prism software (Graphpad, San Diego, CA) to calculate IC₅₀ values.

### GIST 430 Cell Proliferation Assay

A serial dilution of test compound was dispensed into a 96-well black clear bottom plate (Corning, Corning, NY). Five thousand cells were added per well in 200 μL complete growth medium. Plates were incubated for 115 h at 37 degrees Celsius, 5% CO₂, 95% humidity. At the end of the incubation period 40 μL of a 440 μM solution of resazurin (Sigma, St. Louis, MO) in PBS was added to each well and incubated for an additional 5 h at 37 degrees Celsius, 5% CO₂, 95% humidity. Plates were read on a Synergy2 reader (Biotek, Winooski, VT) using an excitation of 540 nM and an emission of 600 nM. Data was analyzed using Prism software (Graphpad, San Diego, CA) to calculate IC₅₀ values.

TABLE J
Inhibition of cell proliferation in GIST430 and BaF3 mutants by
exemplary compounds (“Ex. No.”).

| | | exon11del- | exon11del- | | AYdup- | AYdup- |
|---|---|---|---|---|---|---|
| | GIST430 | V654A | Y823D | AYdup | V654A | D816G |
| Ex. No. | IC₅₀ | IC₅₀ | IC₅₀ | IC₅₀ | IC₅₀ | IC₅₀ |
| 1 | + | | | | | |
| 2 | ++ | ++ | +++ | + | ++ | ++ |
| 3 | +++ | | | | | |
| 4 | + | ++ | ++ | ++ | ++ | ++ |
| 5 | ++ | ++ | ++++ | | ++ | |
| 6 | ++ | + | +++ | | +++ | |
| 7 | ++ | ++ | ++ | | ++ | |
| 8 | ++ | ++ | +++ | + | ++ | ++ |
| 9 | + | + | ++ | + | + | + |
| 10 | + | + | ++ | + | ++ | + |
| 11 | +++ | | | | | |
| 12 | +++ | | | | | |
| 13 | ++ | | | | | |
| 14 | +++ | | | | | |
| 15 | ++ | ++ | +++ | | ++ | ++ |
| 16 | + | + | ++ | | ++ | + |
| 17 | + | | | | | |
| 18 | ++ | + | ++ | + | + | + |
| 19 | ++ | ++ | +++ | + | + | + |
| 20 | ++ | + | ++ | | + | + |
| 21 | + | | | | | |
| 22 | + | | | | | |
| 23 | +++ | | | | | |
| 24 | +++ | | | | | |
| 25 | ++++ | ++++ | | | ++++ | |
| 26 | ++ | ++ | ++ | + | ++ | + |
| 27 | ++++ | ++++ | | | ++++ | |
| 28 | ++ | ++ | +++ | | ++ | ++ |
| 29 | +++ | ++++ | ++++ | | ++++ | ++++ |
| 30 | + | ++ | ++ | | ++ | + |
| 31 | ++ | ++ | +++ | ++ | ++ | + |
| 32 | ++ | ++ | ++ | + | ++ | + |
| 33 | ++ | +++ | +++ | | ++ | ++ |
| 34 | ++ | + | ++ | | ++ | + |
| 35 | ++ | ++ | ++ | + | ++ | ++ |
| 36 | ++ | + | +++ | | ++ | ++ |
| 37 | + | ++ | ++ | + | ++ | ++ |
| 38 | + | + | + | + | ++ | + |
| 39 | + | ++ | ++ | + | ++ | ++ |
| 40 | ++++ | ++++ | | | | |
| 41 | ++ | ++ | ++ | + | ++ | ++ |
| 42 | ++ | ++ | ++ | + | ++ | ++ |
| 43 | +++ | +++ | +++ | | +++ | +++ |
| 44 | +++ | +++ | +++ | ++ | +++ | +++ |
| 45 | + | ++ | +++ | + | ++ | ++ |
| 46 | ++ | ++ | +++ | + | +++ | ++ |
| 47 | + | ++ | ++ | + | ++ | ++ |
| 48 | +++ | +++ | +++ | ++ | +++ | +++ |
| 49 | ++ | ++ | +++ | + | +++ | ++ |
| 50 | +++ | ++++ | ++++ | +++ | +++ | ++++ |
| 51 | + | ++ | ++ | ++ | ++ | ++ |
| 52 | ++ | ++ | +++ | + | +++ | ++ |
| 53 | +++ | ++++ | | +++ | ++++ | ++++ |
| 54 | ++ | +++ | +++ | ++ | +++ | +++ |
| 55 | +++ | ++ | +++ | | +++ | |
| 56 | + | ++ | ++ | | ++ | |
| 57 | ++ | ++ | ++++ | | +++ | |
| 58 | ++ | +++ | ++++ | | +++ | |
| 59 | ++ | ++ | ++++ | | +++ | |
| 60 | ++ | ++ | +++ | | ++ | |
| 61 | + | + | ++ | + | ++ | + |
| 62 | ++++ | +++ | | | +++ | |
| 63 | ++ | ++ | ++++ | | +++ | |
| 64 | ++ | ++ | +++ | | +++ | |
| 65 | ++ | + | +++ | | ++ | |
| 66 | ++ | ++ | ++++ | | ++ | |
| 67 | +++ | ++ | ++ | | +++ | |
| 68 | + | + | +++ | | ++ | |
| 69 | + | ++ | ++ | ++ | ++ | + |
| 70 | ++++ | +++ | +++ | | +++ | |
| 71 | +++ | ++ | ++++ | | +++ | |
| 72 | +++ | +++ | | | ++++ | |
| 73 | ++ | ++ | +++ | | ++ | |
| 74 | ++ | ++ | +++ | | ++ | |
| 75 | +++ | ++ | ++++ | | +++ | |
| 76 | + | + | +++ | | ++ | |
| 77 | ++ | + | +++ | | ++ | |
| 78 | +++ | ++ | ++++ | | +++ | |
| 79 | ++ | ++ | +++ | | ++ | |
| 80 | | ++++ | | | | |
| 81 | ++ | ++ | ++++ | | ++ | |
| 82 | ++ | + | +++ | | ++ | |
| 83 | ++++ | ++++ | | | | |
| 84 | ++++ | +++ | | | | |
| 85 | + | + | +++ | | ++ | |
| 86 | ++ | ++ | ++++ | | ++ | |
| 87 | | +++ | | | +++ | |
| 88 | + | + | ++ | + | ++ | + |
| 89 | ++ | ++ | +++ | | ++ | |
| 90 | ++ | + | +++ | | ++++ | |
| 91 | ++ | ++ | +++ | | ++ | |
| 92 | + | + | ++ | + | ++ | ++ |
| 93 | ++++ | +++ | | | ++++ | |
| 94 | +++ | +++ | | | +++ | |
| 95 | + | + | ++ | + | ++ | ++ |
| 96 | ++ | ++ | ++++ | | ++ | |
| 97 | + | + | ++ | + | ++ | ++ |
| 98 | +++ | ++ | ++++ | | +++ | |
| 99 | + | + | ++ | | ++ | |
| 100 | ++ | ++ | +++ | | ++ | |
| 101 | +++ | ++ | ++++ | | +++ | |
| 102 | + | ++ | ++ | + | ++ | ++ |
| 103 | + | ++ | +++ | | ++ | |
| 104 | + | + | + | + | ++ | + |
| 105 | + | + | +++ | | ++ | |
| 106 | + | + | ++ | | ++ | |
| 107 | +++ | ++ | ++++ | | +++ | |
| 108 | + | + | ++ | + | ++ | + |
| 109 | ++ | ++ | ++++ | | +++ | |
| 110 | ++ | ++ | ++++ | | +++ | |
| 111 | + | + | ++ | + | ++ | ++ |
| 112 | + | + | ++ | + | ++ | ++ |
| 113 | ++ | ++ | +++ | | ++ | |
| 114 | +++ | +++ | ++++ | | +++ | |
| 115 | ++ | ++ | +++ | | ++ | |
| 116 | +++ | ++ | | | +++ | |
| 117 | ++ | ++ | ++++ | | ++ | |
| 118 | ++ | + | ++ | | ++ | |
| 119 | ++ | ++ | +++ | | ++ | |
| 120 | ++ | + | +++ | | ++ | |
| 121 | +++ | ++ | ++++ | | +++ | |
| 122 | +++ | ++ | ++++ | | +++ | |
| 123 | ++ | + | ++ | | ++ | |
| 124 | ++ | ++ | +++ | | ++ | |
| 125 | ++ | ++ | | | ++ | |
| 126 | +++ | +++ | ++++ | | +++ | |
| 127 | ++ | ++ | ++++ | | ++ | |
| 128 | ++ | + | +++ | | ++ | |
| 129 | ++ | + | ++++ | | ++ | |
| 130 | ++ | + | ++ | + | ++ | ++ |
| 131 | ++ | + | ++ | + | ++ | ++ |
| 132 | +++ | ++ | | | +++ | |
| 133 | ++ | + | ++ | | + | |
| 134 | | +++ | ++++ | | ++++ | |
| 135 | + | + | ++ | | + | |
| 136 | ++ | + | +++ | | ++ | |
| 137 | ++ | ++ | ++++ | | ++ | |
| 138 | ++ | + | +++ | + | ++ | ++ |
| 139 | ++ | + | ++++ | + | ++ | ++ |
| 140 | ++ | ++ | +++ | | ++ | |
| 141 | ++ | + | ++ | + | ++ | ++ |
| 142 | ++ | ++ | ++ | | ++ | |
| 143 | + | + | ++ | + | ++ | ++ |
| 144 | +++ | +++ | | | +++ | |
| 145 | ++ | + | ++ | + | ++ | ++ |
| 146 | +++ | ++ | | | +++ | |
| 147 | +++ | ++ | ++++ | | +++ | |
| 148 | +++ | ++ | | | +++ | |
| 149 | ++ | ++ | ++ | | ++ | |
| 150 | ++ | ++ | +++ | + | ++ | ++ |
| 151 | ++ | ++ | +++ | | ++ | |
| 152 | ++ | ++ | +++ | | ++ | |
| 153 | ++ | +++ | +++ | | ++ | |
| 154 | ++ | +++ | +++ | | +++ | |
| 155 | +++ | ++ | +++ | | ++ | |
| 156 | ++ | ++ | ++ | + | ++ | ++ |
| 157 | +++ | +++ | +++ | | +++ | |
| 158 | ++ | ++ | ++ | + | + | + |
| 159 | ++ | ++ | ++ | | ++ | |
| 160 | ++ | ++ | ++ | + | ++ | ++ |
| 161 | ++ | ++ | ++ | + | + | ++ |
| 162 | ++ | ++ | ++ | + | ++ | ++ |
| 163 | +++ | ++++ | +++ | | +++ | |
| 164 | ++ | +++ | +++ | | +++ | |
| 165 | ++ | +++ | +++ | | ++ | |
| 166 | ++ | ++ | ++ | | ++ | |
| 167 | ++ | +++ | +++ | | ++ | |
| 168 | + | ++ | ++ | | ++ | |
| 169 | ++ | +++ | +++ | | ++ | |
| 170 | ++ | ++ | ++ | | ++ | |
| 171 | ++ | ++ | + | | ++ | |
| 172 | ++ | ++ | ++ | | ++ | |
| 173 | ++ | ++ | ++ | | ++ | |
| 174 | +++ | ++ | ++ | | ++ | |
| 175 | ++ | ++ | ++ | | ++ | |
| 176 | ++ | +++ | ++ | ++ | ++ | ++ |
| 177 | ++ | +++ | +++ | | ++ | |
| 179 | +++ | +++ | +++ | | +++ | |
| 180 | ++++ | ++++ | +++ | | ++++ | |
| 181 | ++ | ++ | + | | ++ | |
| 182 | ++ | ++ | ++ | | ++ | |
| 183 | ++ | ++ | ++ | | + | |
| 184 | ++ | +++ | +++ | | + | |
| 185 | +++ | ++++ | ++++ | | ++++ | |
| 186 | ++++ | ++++ | ++++ | | ++++ | |
| 187 | +++ | ++++ | ++++ | | +++ | |
| 188 | +++ | +++ | +++ | | +++ | |
| 189 | +++ | +++ | +++ | | +++ | |
| 190 | ++ | ++ | ++ | | ++ | |
| 191 | +++ | +++ | +++ | | +++ | |
| 192 | ++ | +++ | ++ | | ++ | |
| 193 | | | | | | |
| 195 | + | ++ | ++ | | ++ | |
| 196 | ++ | +++ | +++ | | ++ | |
| 197 | ++ | +++ | +++ | | ++ | |
| 198 | ++ | +++ | +++ | | ++ | |
| 199 | | ++++ | ++++ | | ++++ | |
| 200 | | ++++ | ++++ | | ++++ | |
| 201 | | ++++ | ++++ | | +++ | |
| 202 | | ++ | ++ | | ++ | |
| 203 | | ++ | ++ | | ++ | |
| 204 | | +++ | ++ | | ++ | |
| 205 | | ++++ | ++++ | | ++++ | |
| 206 | | ++++ | ++++ | | +++ | |
| 207 | | ++++ | ++++ | | +++ | |
| 208 | | ++++ | ++++ | | ++++ | |
| 209 | | +++ | ++++ | | ++++ | |
| 210 | | ++++ | ++++ | | ++++ | |
| 211 | | ++ | ++ | | ++ | |
| 212 | | ++++ | ++++ | | +++ | |
| 213 | | ++++ | ++++ | | +++ | |
| 214 | | ++ | ++ | | ++ | |
| 215 | | +++ | +++ | | +++ | |
| 216 | | +++ | +++ | | +++ | |
| 217 | | ++++ | ++++ | | +++ | |
| 218 | | ++++ | ++++ | | +++ | |
| 219 | | ++ | ++ | | ++ | |
| 220 | | +++ | ++ | | +++ | |
| 221 | | +++ | +++ | | ++ | |

For Table J, “+” refers to an IC₅₀ less than or equal to 10 nM; “++” refers to an IC₅₀ greater than 10 nM and less than or equal to 100 nM; “+++” refers to an IC₅₀ greater than 100 nM and less than or equal to 1000 nM; and “++++” refers to an IC₅₀ greater than 1000 nM.

All cited references are herein expressly incorporated by reference in their entirety.

The present disclosure is not to be limited in scope by the specific embodiments disclosed in the examples, which are intended as illustrations of aspects of the disclosure and any embodiments that are functionally equivalent are within the scope of this disclosure. Indeed, various modifications in addition to those shown and described herein will become apparent to those skilled in the art and are intended to fall within the scope of the appended claims.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

1. A compound represented by Formula I: 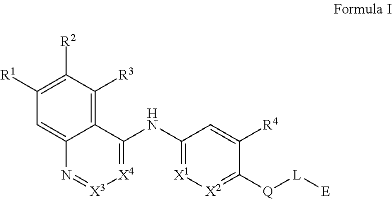
or a pharmaceutically acceptable salt, enantiomer, stereoisomer, or tautomer thereof, wherein:
X¹ and X² are each independently selected from the group consisting of N, CH, and CF;
X³ and X⁴ are each independently CH or N, provided that not more that one of X³ and X⁴ is N;
Q is selected from the group consisting of 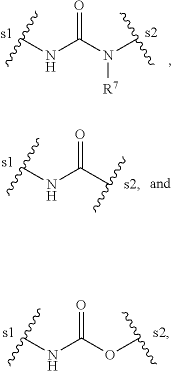
wherein s1 is the site covalently linked to the ring; and s2 is the site covalently linked to L;
L is selected from the group consisting of a direct bond and a C₁-C₆ alkyl optionally substituted with (E¹¹)ₘ, or when Q 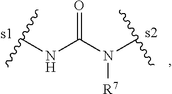
L taken together with R⁷ and the N atom to which L and R⁷ are attached form an optionally substituted heterocyclyl having from 4 to 10 atoms in the ring structure wherein the optional substituent is selected from the group consisting of alkyl, halogen, haloalkyl, hydroxyl, alkoxy, haloalkoxy, and oxo;
E is selected from the group consisting of H, alkenyl, cyano, haloalkoxy, haloalkyl, halogen,
optionally substituted alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone, and
optionally substituted alkoxy wherein the optionally substituted substituent, at each occurrence, is independently selected from the group consisting of cycloalkyl and heterocyclyl; or
Q-L-E taken together is selected from the group consisting of 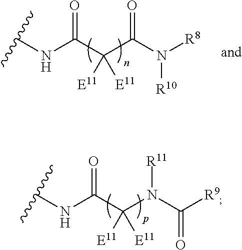
E¹¹, at each occurrence, is independently selected from the group consisting of H, alkyl, and halogen, or two occurrences of E¹¹ when taken together with the C atom to which they are attached form an optionally substituted cycloalkyl having from 3 to 4 atoms in the ring structure, wherein the optional substituent is selected from the group consisting of C₁-C₆ alkyl, halogen, C₁-C₆ haloalkyl, C₁-C₆ alkoxy, C₁-C₆ haloalkoxy, hydroxy, and oxo;
R¹ is selected from the group consisting of H, haloalkyl,
optionally substituted alkoxy wherein the optionally substituted substituent, at each occurrence, is independently selected from the group consisting of alkoxy, acetamido, amine, haloalkyl, haloalkoxy, halogen, hydroxy, cyano, carboxylic acid,
optionally substituted amido, and optionally substituted heterocyclyl, wherein
the substituent of the optionally substituted amido, at each occurrence, is independently, C₁-C₆ alkyl, and
the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo,
optionally substituted heterocyclyloxy wherein the optionally substituted substituent, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo, and
optionally substituted alkyl wherein the optionally substituted substituent, at each occurrence, is independently selected from the group consisting of alkoxy, amine, haloalkoxy, and hydroxy;
R² is selected from the group consisting of H and optionally substituted alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selected from the group consisting of C₁-C₆ alkyl and halogen;
R³ is selected from the group consisting of F and alkoxy;
R⁴ is selected from the group consisting of H, F, alkyl, and alkoxy;
R⁷ is selected independently from the group consisting of H, alkyl, haloalkyl, or cycloalkyl;
R⁸ is selected from the group consisting of optionally substituted alkyl, optionally substituted heterocyclyl, and optionally substituted cycloalkyl, wherein
the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹⁰ is selected from the group consisting of H and alkyl, or taken together with R⁸ and the N atom to which R⁸ and R¹⁰ are attached form an optionally substituted heterocyclyl having from 4 to 8 atoms in the ring structure wherein the optional substituent at each occurrence, is independently selected from the group consisting of alkyl, halogen, haloalkyl, hydroxyl, alkoxy, haloalkoxy, and oxo;
R⁹ is selected from the group consisting of optionally substituted alkyl, optionally substituted heterocyclyl, and optionally substituted cycloalkyl, wherein
the substituent of the optionally substituted alkyl, at each occurrence, is independently selected from the group consisting of C₁-C₆ alkyl, alkoxy, amine, halogen, haloalkyl, haloalkoxy, hydroxy, cyano, cycloalkyl, and heterocyclyl,
the substituent of the optionally substituted heterocyclyl, at each occurrence, is independently selected from the group consisting of C₁-C₆ alkyl, alkoxy, amine, halogen, haloalkyl, haloalkoxy, hydroxy, oxo, and cyano, and
the substituent of the optionally substituted cycloalkyl at each occurrence, is independently selected from the group consisting of C₁-C₆ alkyl, alkoxy, amine, halogen, haloalkyl, haloalkoxy, hydroxy, and cyano;
R¹¹ is selected from the group consisting of H and alkyl, or taken together with R⁹ and the atoms to which R⁹ and R¹¹ are respectively attached form an optionally substituted heterocyclyl having from 4 to 7 atoms in the ring structure wherein the optional substituent, at each occurrence, is independently selected from the group consisting of alkyl, halogen, haloalkyl, hydroxyl, alkoxy, and haloalkoxy;
each occurrence of m is independently 0, 1, 2, 3, or 4;
n is 0, 1, 2, 3, or 4; and
p is 1, 2, or 3;
provided that the compound is not:

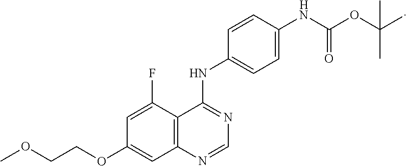

2-4. (canceled)

5. The compound of claim 1 represented by Formula I-I: 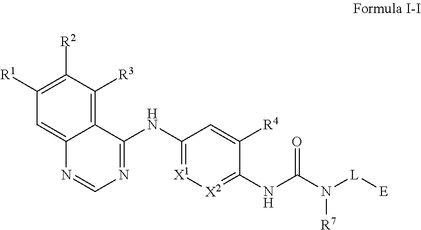
or a pharmaceutically acceptable salt, enantiomer, stereoisomer, or tautomer thereof.

6. The compound of claim 1 represented by Formula I-J: 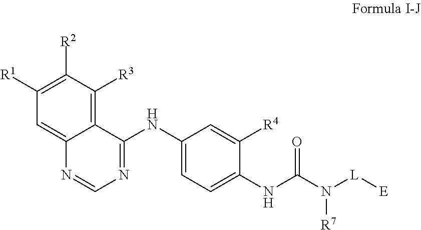
or a pharmaceutically acceptable salt, enantiomer, stereoisomer, or tautomer thereof.

7. The compound of claim 1 represented by Formula I-K: 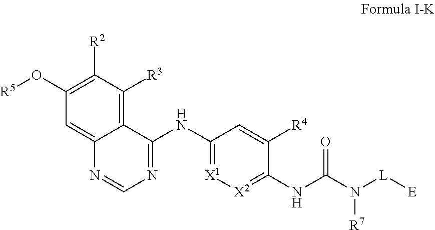
or a pharmaceutically acceptable salt, enantiomer, stereoisomer, or tautomer thereof.

8. The compound of claim 1 represented by Formula I-L: 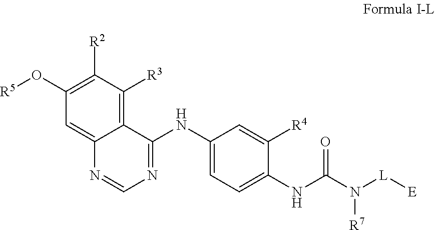
or a pharmaceutically acceptable salt, enantiomer, stereoisomer, or tautomer thereof.

9. The compound of claim 1 represented by Formula I-O: 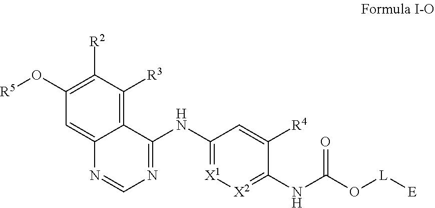
or a pharmaceutically acceptable salt, enantiomer, stereoisomer, or tautomer thereof.

10. (canceled)

11. The compound of claim 1 represented by Formula I-T: 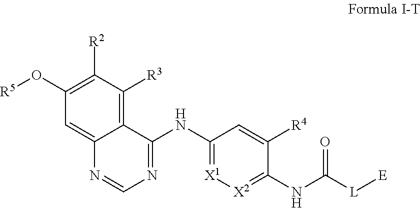
or a pharmaceutically acceptable salt, enantiomer, stereoisomer, or tautomer thereof.

12. The compound of claim 1 represented by Formula I-U: 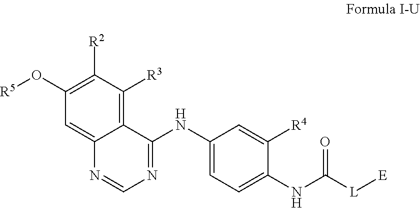
or a pharmaceutically acceptable salt, enantiomer, stereoisomer, or tautomer thereof.

13. (canceled)

14. The compound of claim 1 represented by Formula I-W: 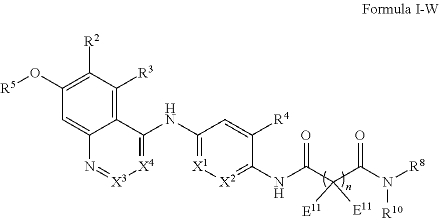
or a pharmaceutically acceptable salt, enantiomer, stereoisomer, or tautomer thereof.

15. The compound of claim 1 represented by Formula I-X: 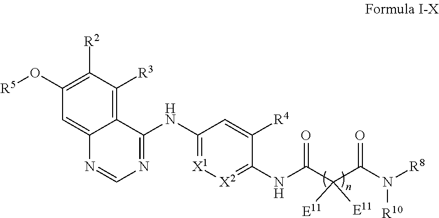
or a pharmaceutically acceptable salt, enantiomer, stereoisomer, or tautomer thereof.

16. The compound of claim 1 represented by Formula I-Y: 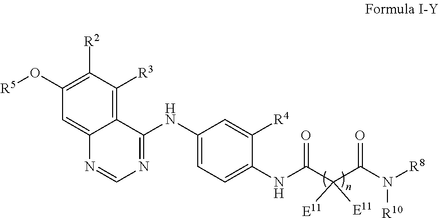
or a pharmaceutically acceptable salt, enantiomer, stereoisomer, or tautomer thereof.

17-18. (canceled)

19. The compound of claim 1, wherein R¹ is selected from the group consisting of H, haloalkyl,
optionally substituted C₁-C₆ alkoxy wherein the optionally substituted substituent, at each occurrence, is independently selected from the group consisting of alkoxy, acetamido, amine, haloalkyl, haloalkoxy, halogen, hydroxy, cyano, carboxylic acid, optionally substituted amido, and optionally substituted heterocyclyl, wherein
the substituent of the optionally substituted amido, at each occurrence, is independently C₁-C₆ alkyl,
the substituent of the optionally substituted heterocyclyl, at each occurrence, is independently C₁-C₆ alkyl, amine, cyano, halogen, C₁-C₆ haloalkyl, C₁-C₆ alkoxy, C₁-C₆ haloalkoxy, hydroxy, and oxo,
optionally substituted C₄-C₆ cycloalkyloxy wherein the optionally substituted substituent, at each occurrence, is independently selected from the group consisting of independently, C₁-C₆ alkyl, amine, halogen, C₁-C₆ haloalkyl, C₁-C₆ alkoxy, C₁-C₆ haloalkoxy, hydroxy, and oxo, and
optionally substituted C₁-C₆ alkyl wherein the optionally substituted substituent, at each occurrence, is independently selected from the group consisting of alkoxy, amine, haloalkoxy, and hydroxy.

20. The compound of claim 1, wherein R¹ is selected from the group consisting of H,

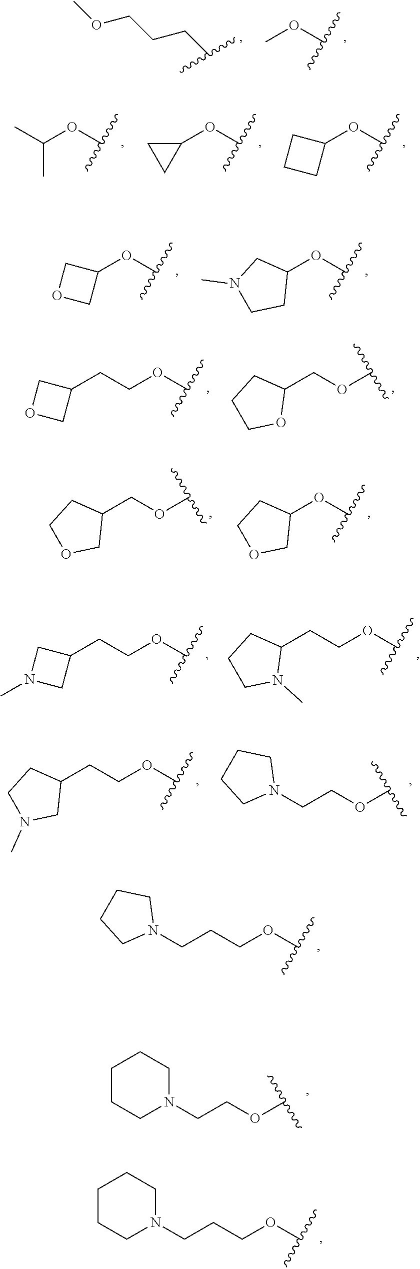

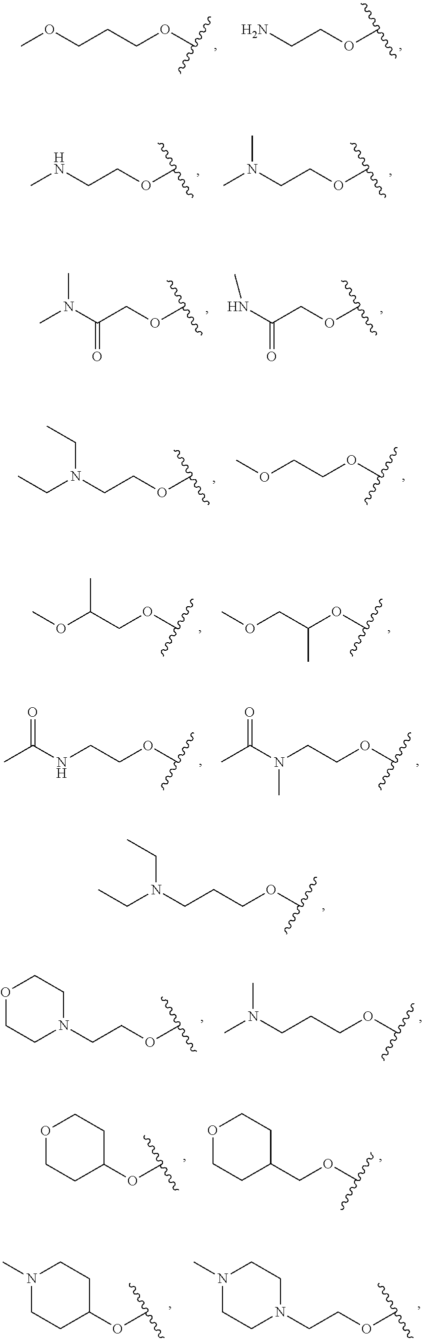

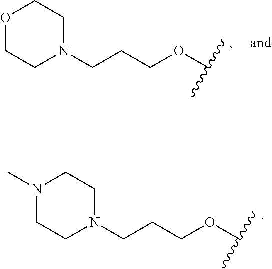 [SMILES: *OCCCN1CCOCC1 ; *OCCCN1CCN(C)CC1]

21. The compound of claim 1, wherein R¹ is selected from the group consisting of H,

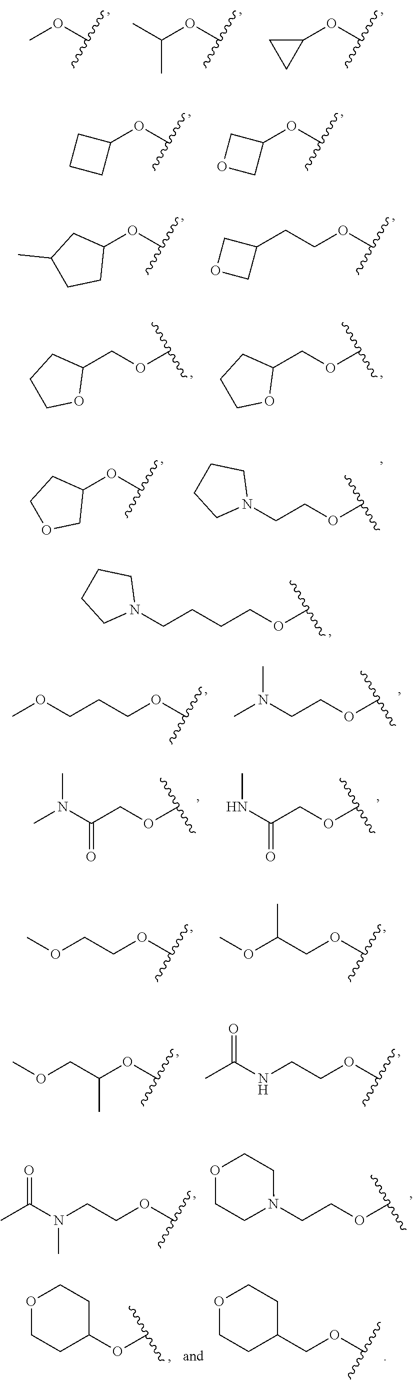

22-24. (canceled)

25. The compound of claim 1, wherein R² is selected from the group consisting of H, and optionally substituted C₁-C₆ alkoxy wherein the optionally substituted substituent, at each occurrence, is independently, alkoxy, amine, heterocyclyl, or when taken together with R¹ form an optionally substituted heterocyclyl having from 5 to 6 atoms in the ring structure wherein the optionally substituted substituent, at each occurrence, is independently selecting from the group consisting of C₁-C₆ alkyl and halogen.

26. The compound of claim 1, wherein R² is selected from the group consisting of H,

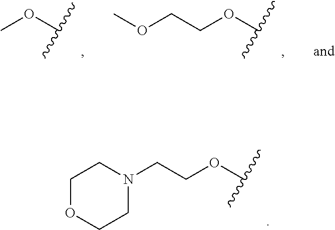

27. The compound of claim 1, wherein R² is H.

28-30. (canceled)

31. The compound of claim 1, wherein R³ is F.

32. The compound of claim 1, wherein R⁴ is selected from the group consisting of H, F, C₁-C₆ alkyl, and alkoxy.

33. (canceled)

34. The compound of claim 1, wherein X¹, X², X³, and X⁴ are independently CH or N, provided that not more that one of X³ and X⁴ is N.

35. The compound of claim 1, wherein X¹ is CH and X² is CH.

36-38. (canceled)

39. The compound of claim 1, wherein 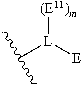
is selected from the group consisting of

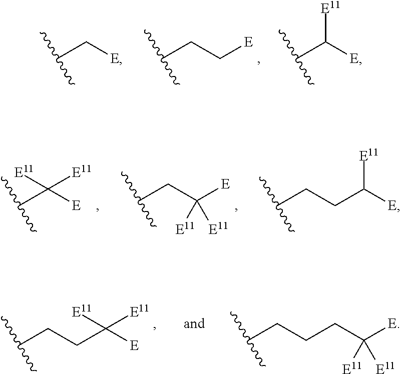

40. The compound of claim 38, wherein E¹¹, at each occurrence, is independently selected from the group consisting of H, C₁-C₆ alkyl, C₃-C₅ cycloalkyl, and halogen.

41. (canceled)

42. The compound of claim 1, wherein 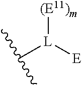
is selected from the group consisting of

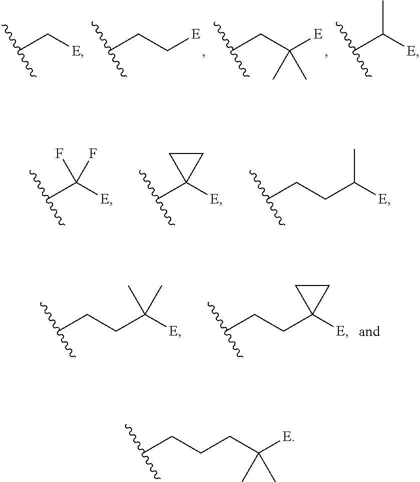

43. The compound of claim 1, wherein E is selected from the group consisting of H, C₁-C₆ alkenyl, cyano, haloalkoxy, haloalkyl, halogen,
optionally substituted C₁-C₆ alkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted C₃-C₆ cycloalkyl wherein the optionally substituted substituent, at each occurrence, is independently selected from the group consisting of C₁-C₆ alkyl, alkoxy, amine, halogen, haloalkyl, haloalkoxy, hydroxy, and cyano,
optionally substituted heterocyclyl wherein the optionally substituted substituent, at each occurrence, is independently selected from the group consisting of C₁-C₆ alkyl, alkoxy, amide, amine, acyl, alkoxyalkyl, halogen, haloalkyl, haloalkoxy, hydroxy, hydroxyalkyl, oxo, cyano, cyanoalkyl, and sulfone; and
optionally substituted alkoxy wherein the optionally substituted substituent, at each occurrence, is independently, selected from the group consisting of cycloalkyl, and heterocyclyl.

44. The compound of claim 1, wherein E is selected from the group consisting of H, fluoro, methyl, trifluoromethyl, methoxy, tert-butoxy, trifluoromethoxy, cyano, alkenyl,

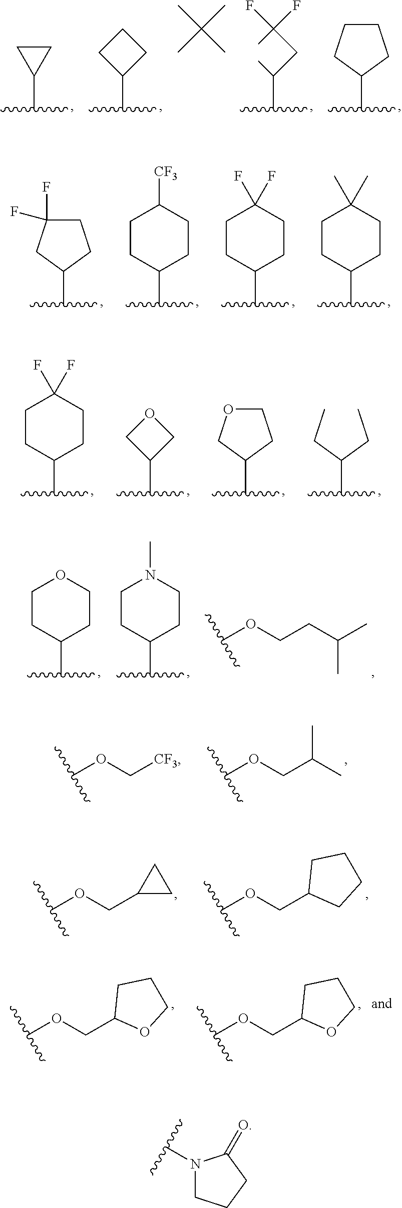

45-48. (canceled)

49. The compound of claim 1, wherein 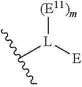
is selected from the group consisting of

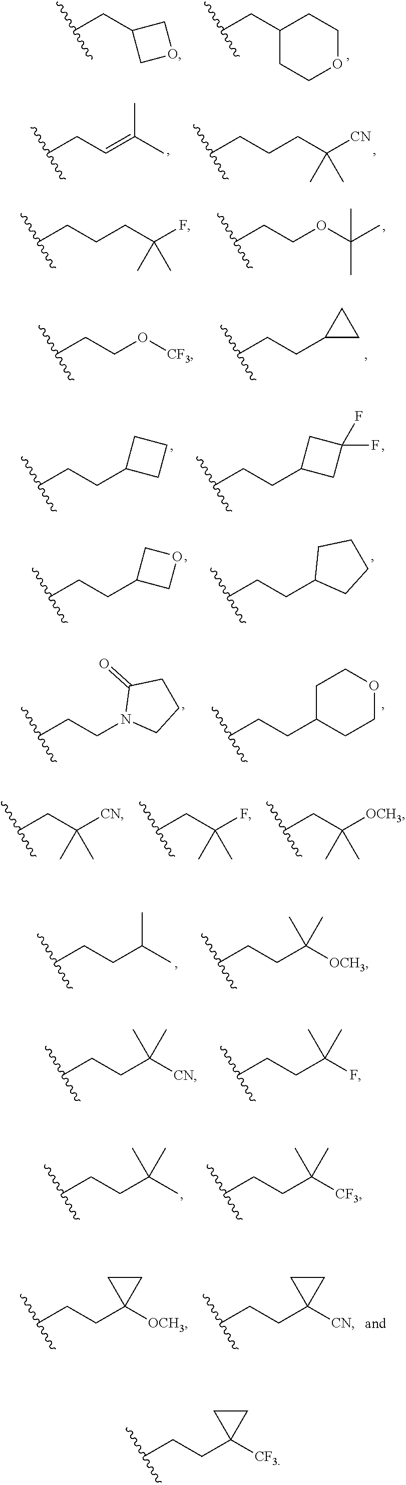

50. The compound of claim 1, wherein when L is a direct bond, 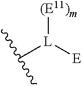
is selected from the group consisting of

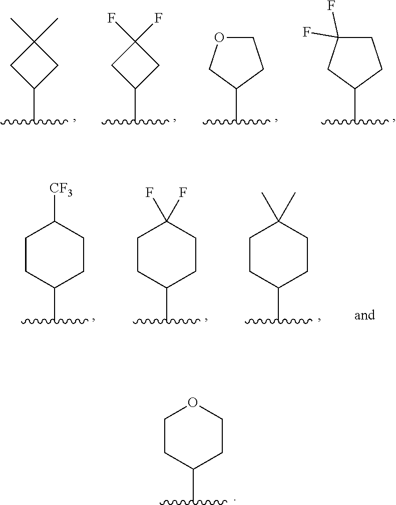

51. The compound of claim 1, wherein R⁷ is H, alkyl, or haloalkyl.

52-62. (canceled)

63. The compound of claim 1, wherein R⁸ is selected from the group consisting of alkyl, cycloalkyl, and heterocyclyl, and R¹⁰ is selected from the group consisting of H and alkyl.

64. The compound of claim 1, wherein R⁸ is selected from the group consisting of 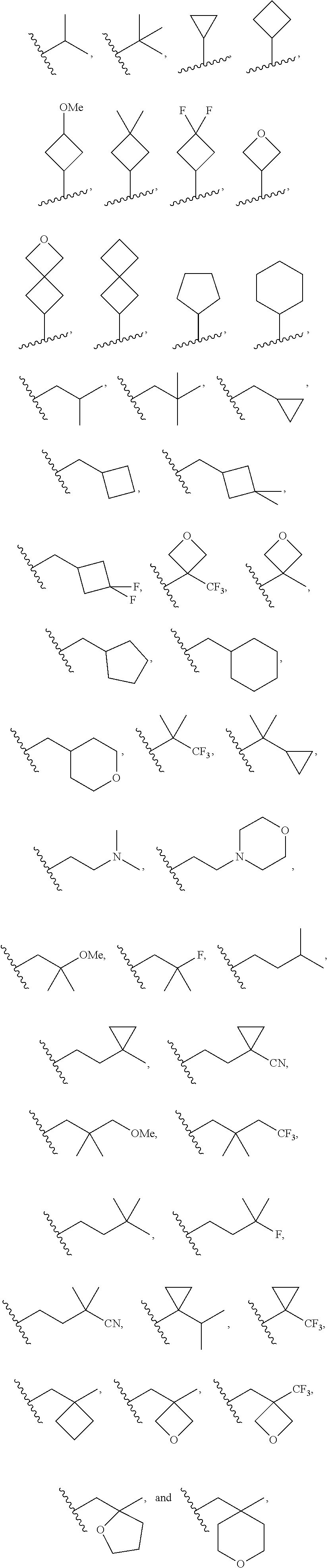
and R¹⁰ is selected from the group consisting of H, methyl, ethyl, and isopropyl.

65. The compound of claim 1, wherein R⁸ taken together with R¹⁰ and the N atom to which R⁸ and R¹⁰ are attached form a ring structure selected from the group consisting of

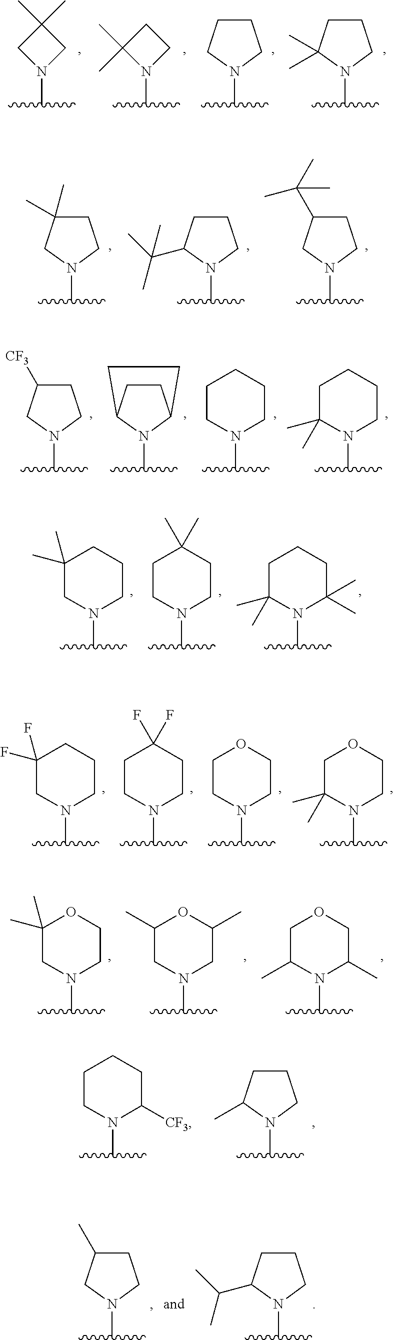

66. (canceled)

67. A compound selected from the group consisting of: 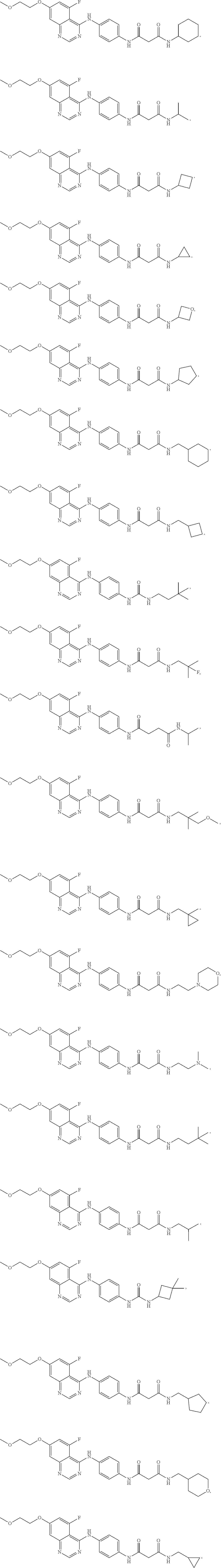 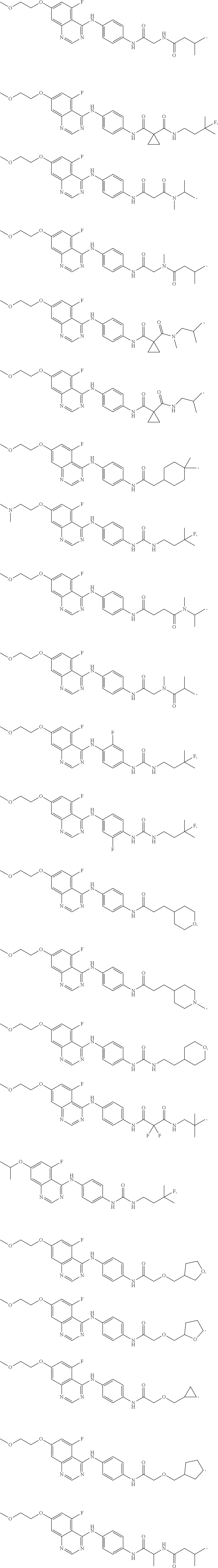 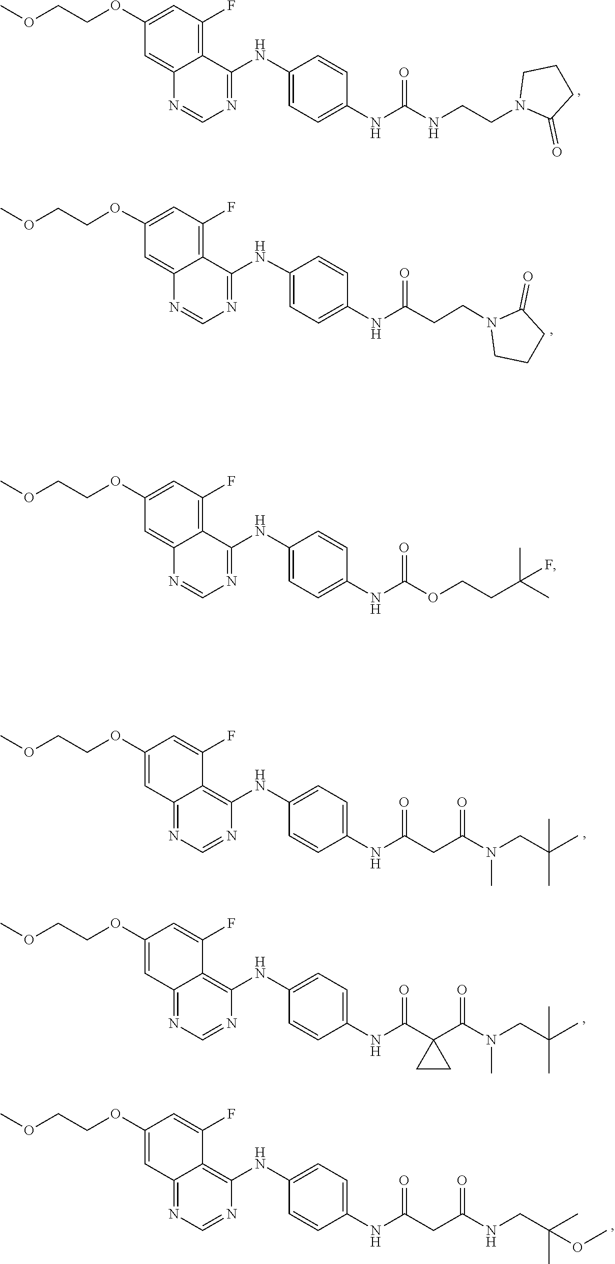 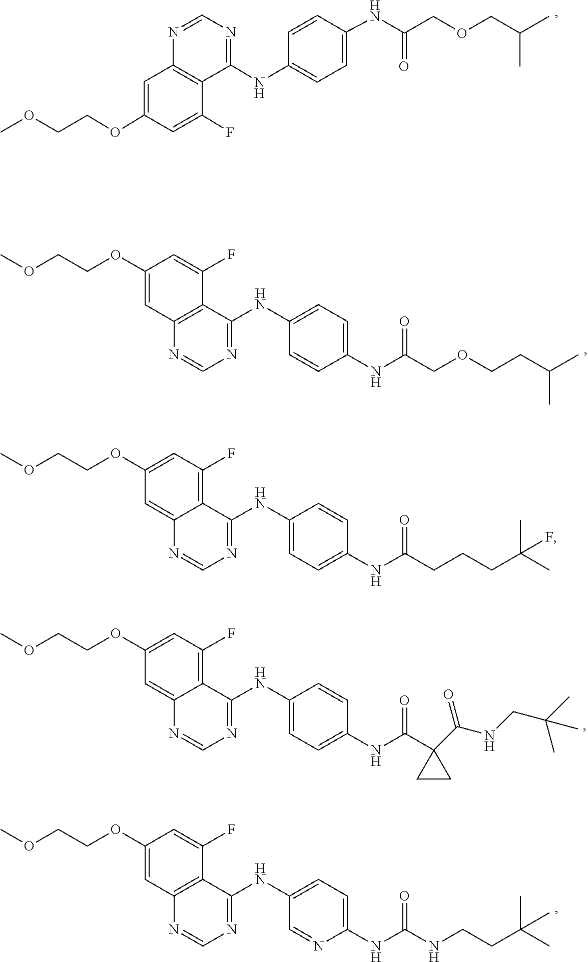 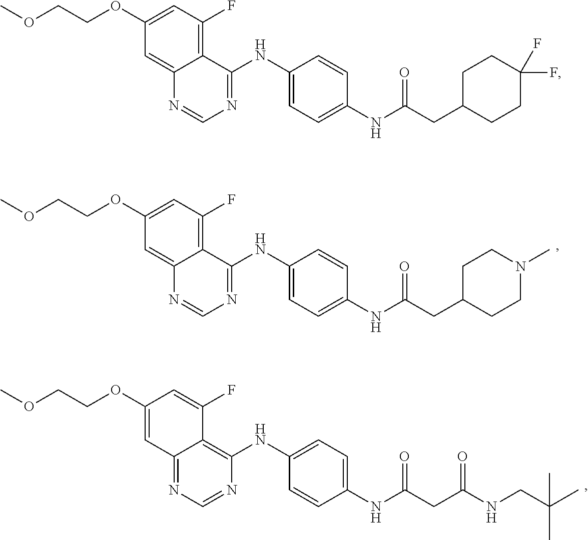 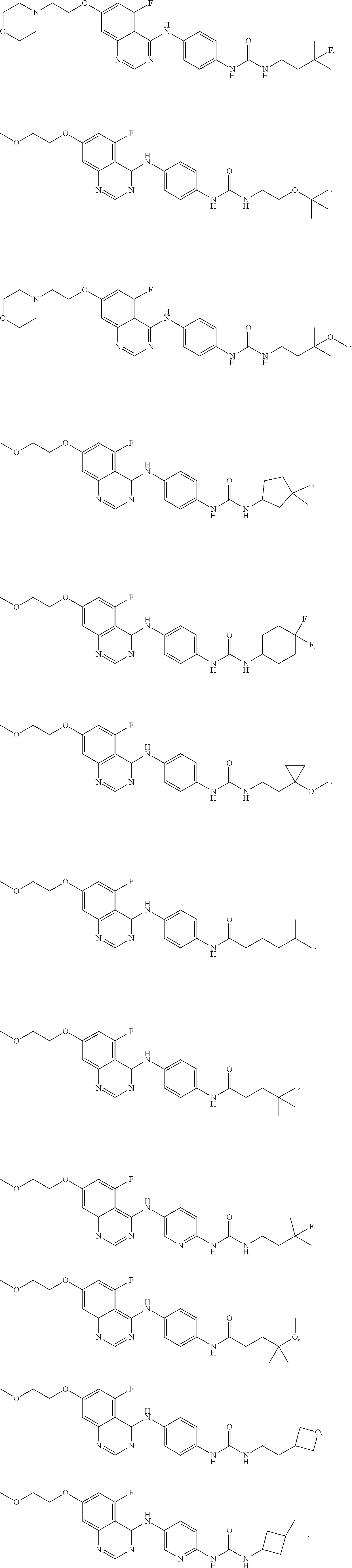 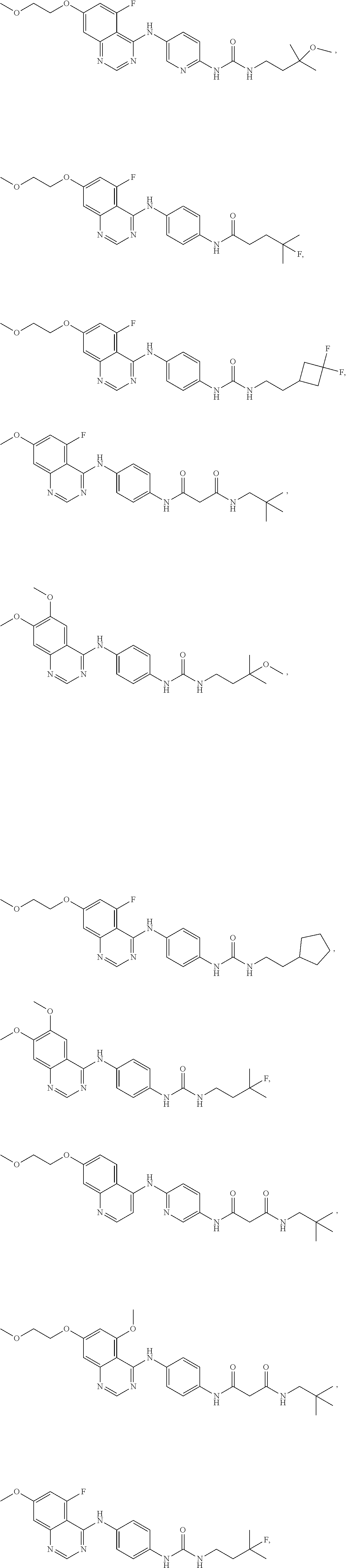 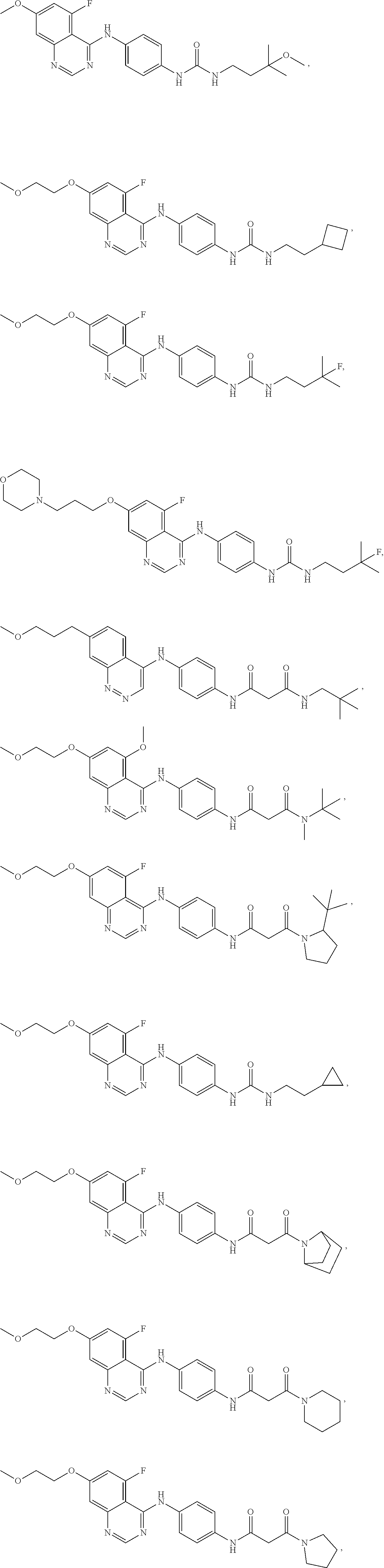 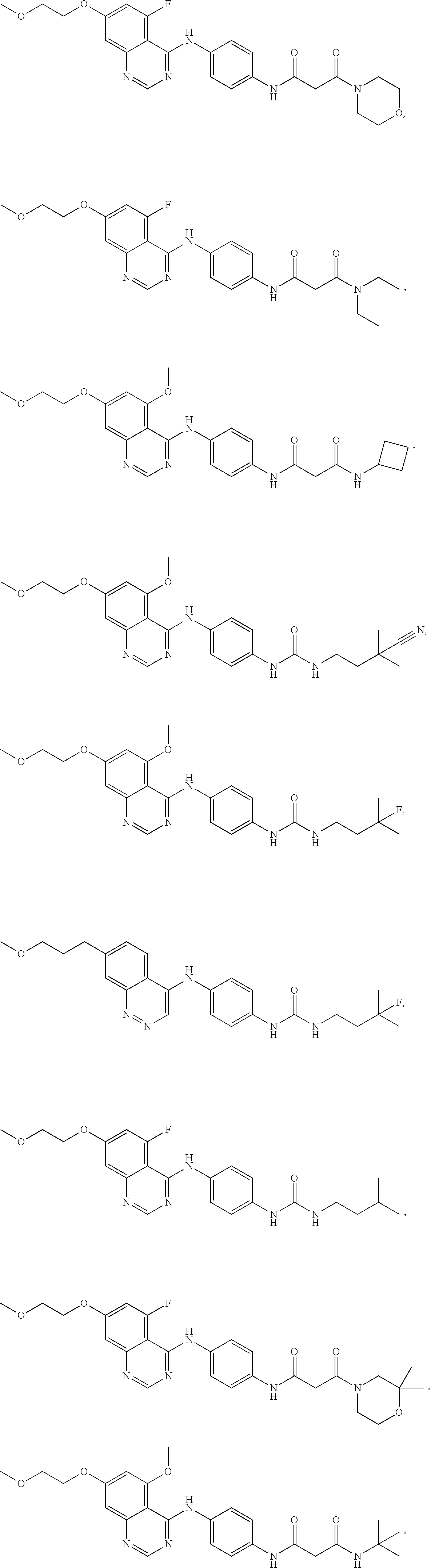 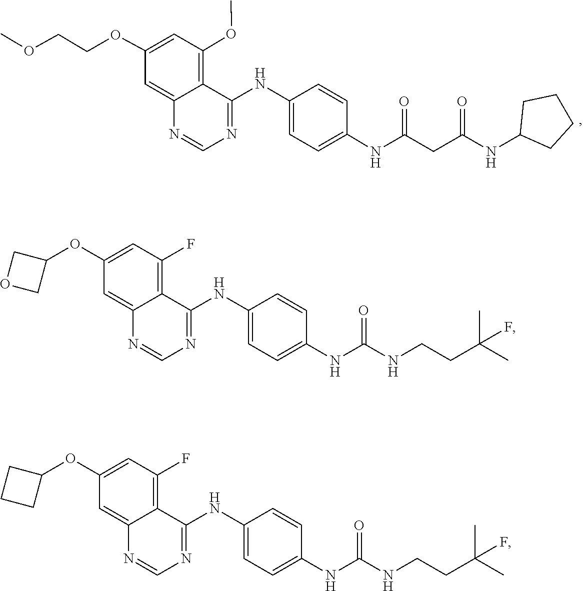 [SMILES: COCCOc1cc(OC)c2c(Nc3ccc(NC(=O)CC(=O)NC4CCCC4)cc3)ncnc2c1 ; "," ; CC(C)(F)CCNC(=O)Nc1ccc(Nc2ncnc3cc(OC4COC4)cc(F)c23)cc1 ; CC(C)(F)CCNC(=O)Nc1ccc(Nc2ncnc3cc(OC4CCC4)cc(F)c23)cc1] 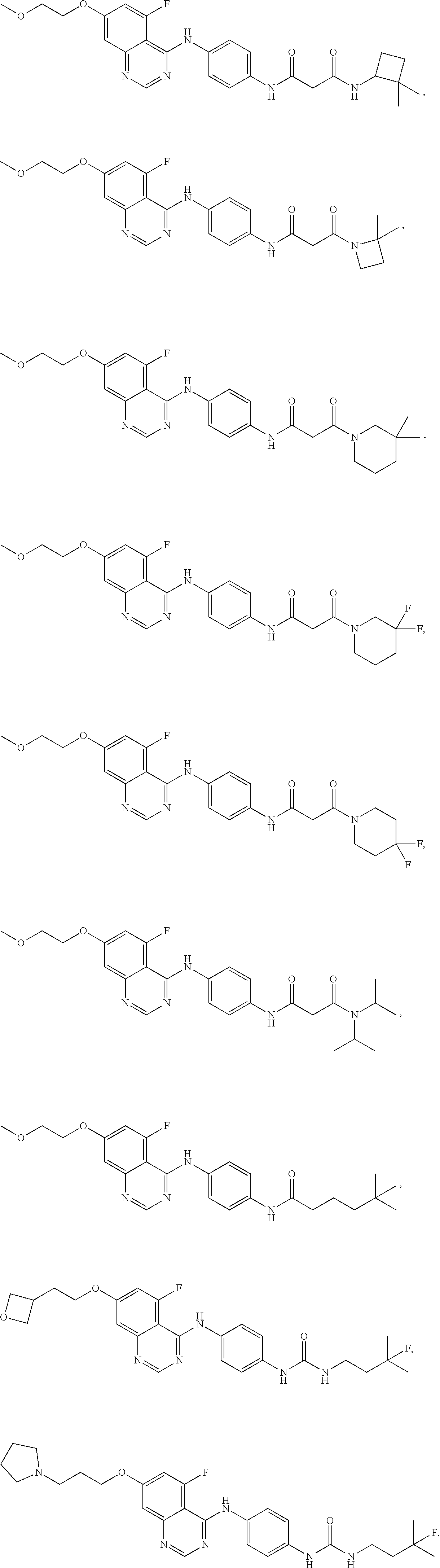 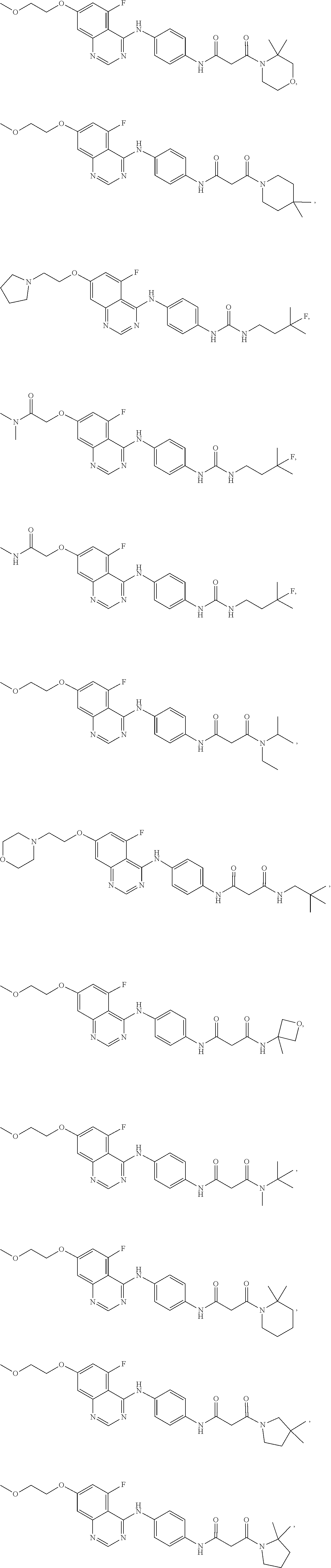 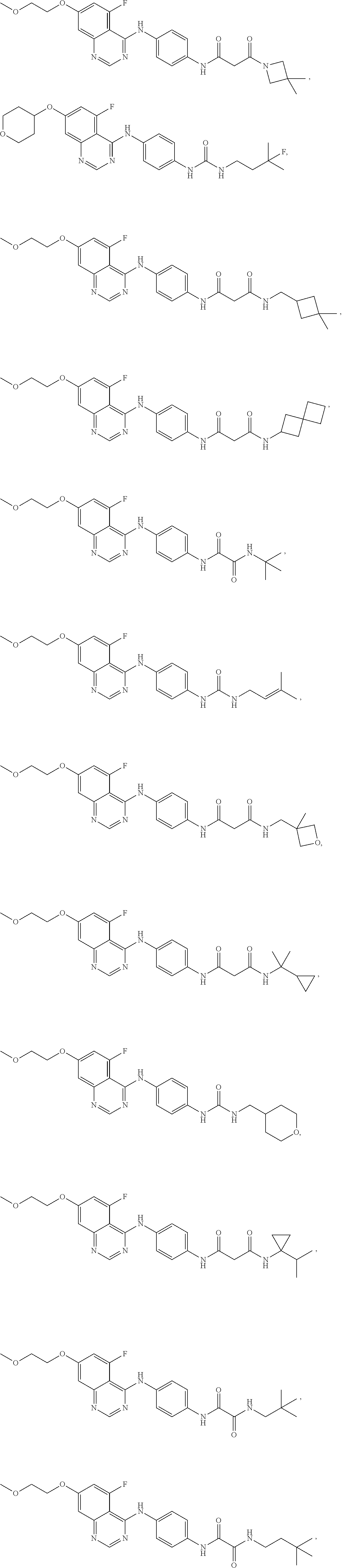 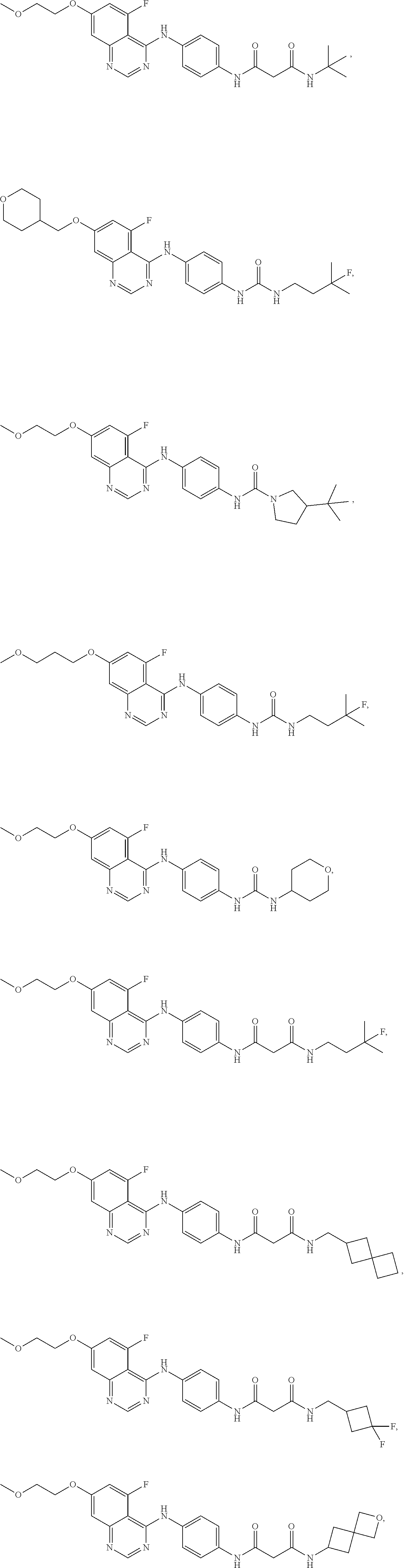 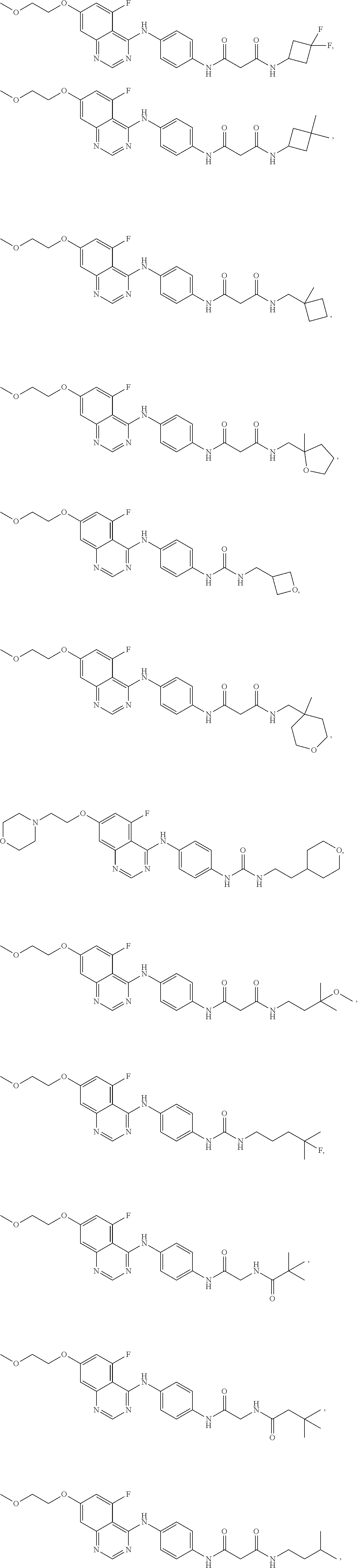 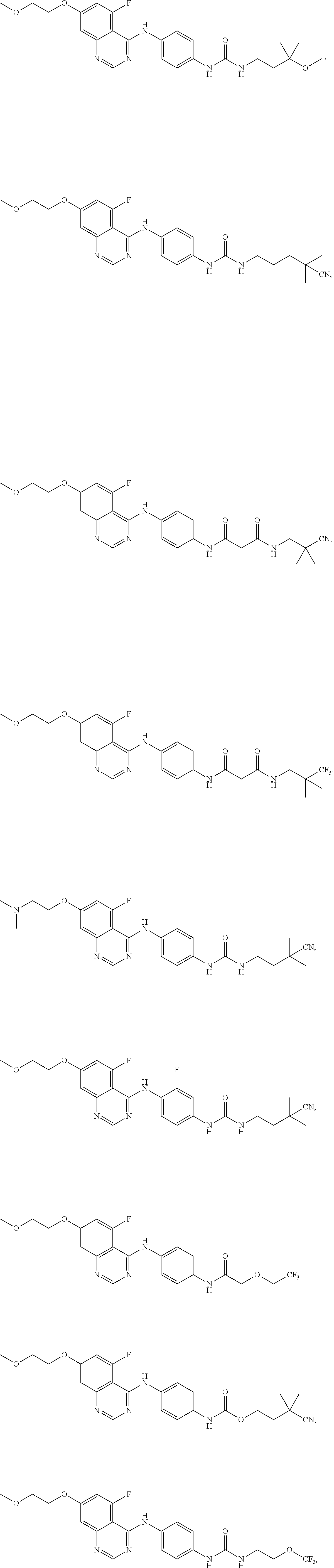 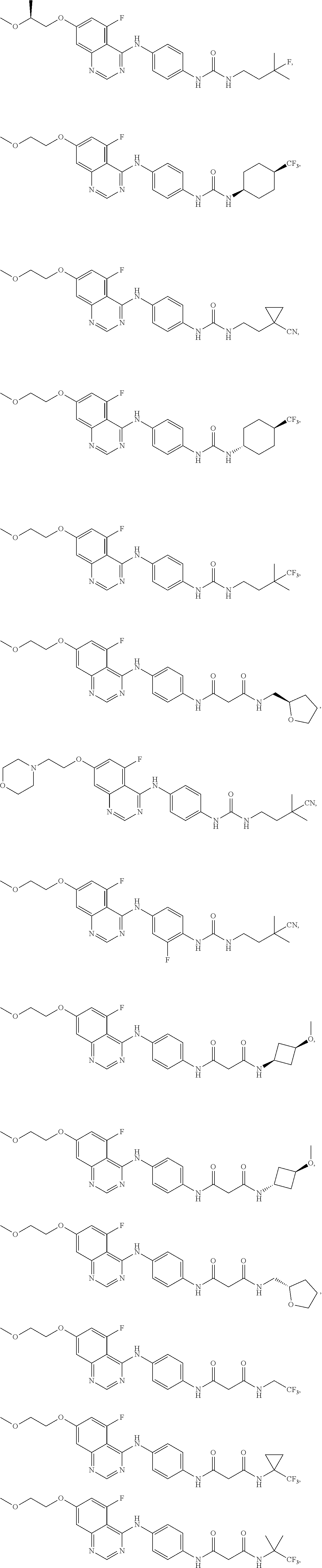 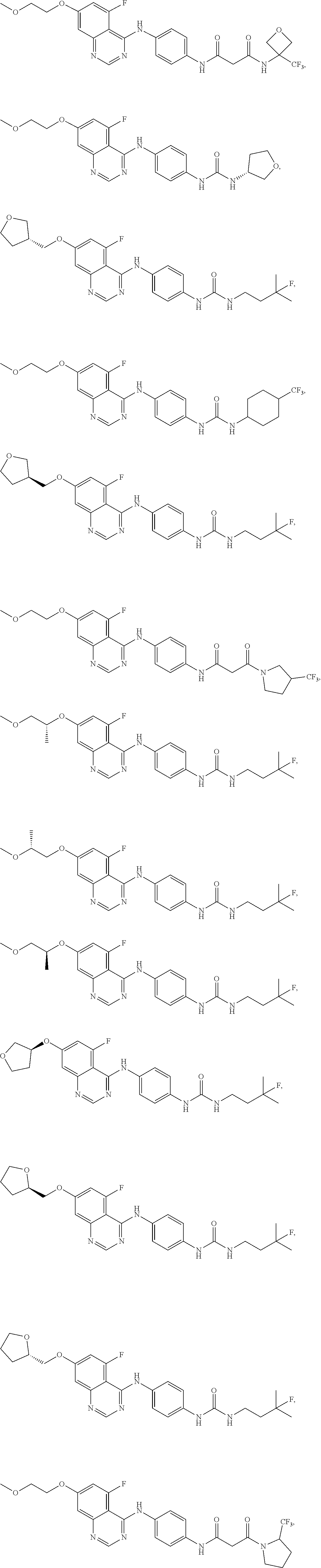 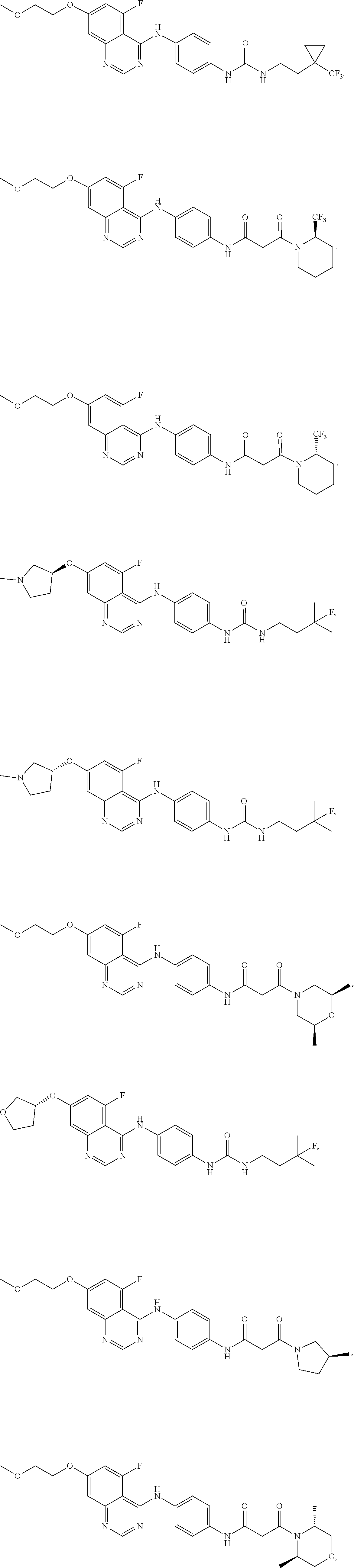 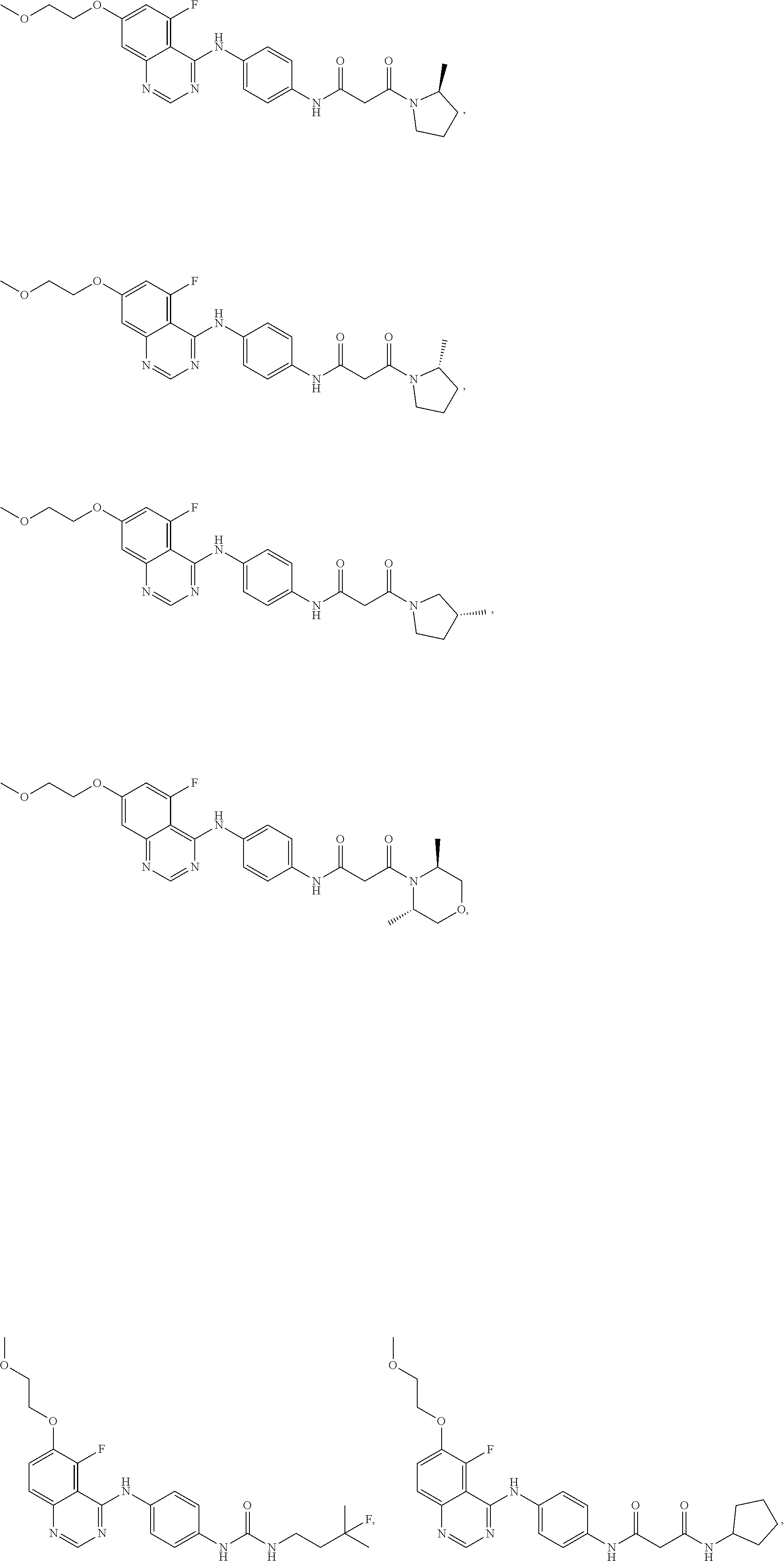 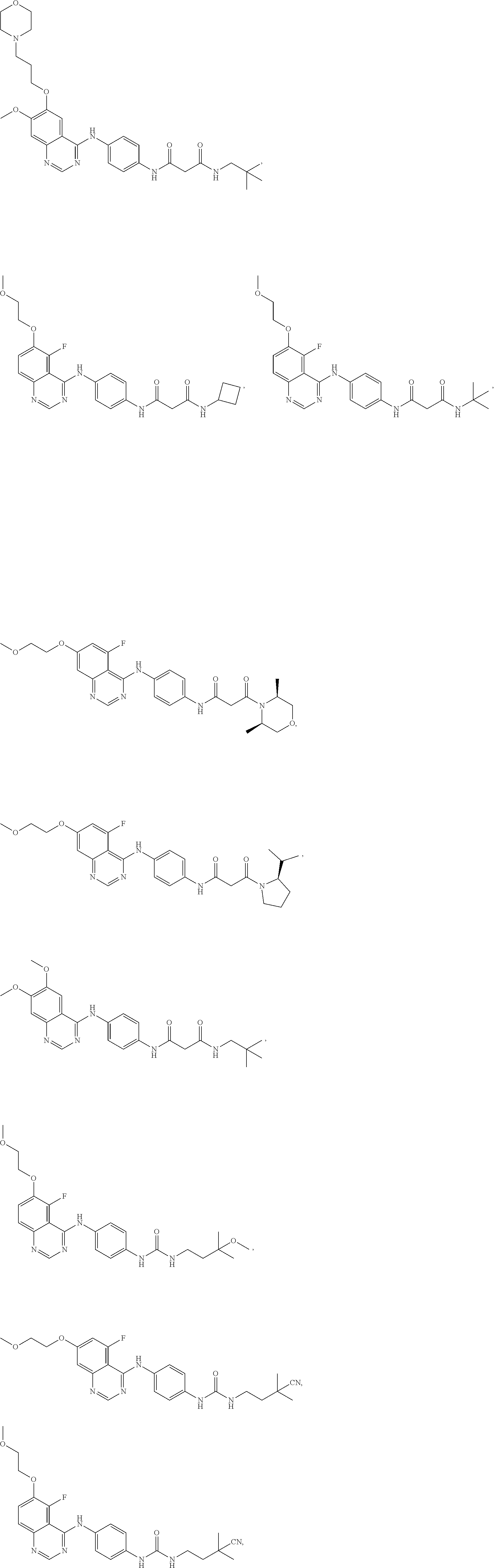 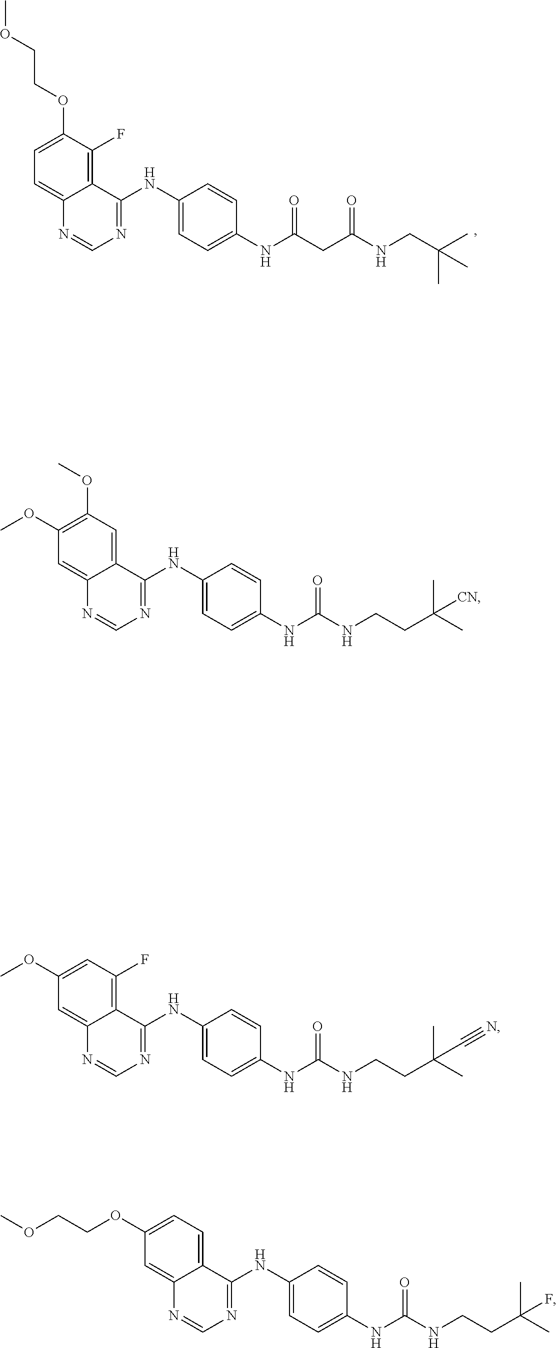 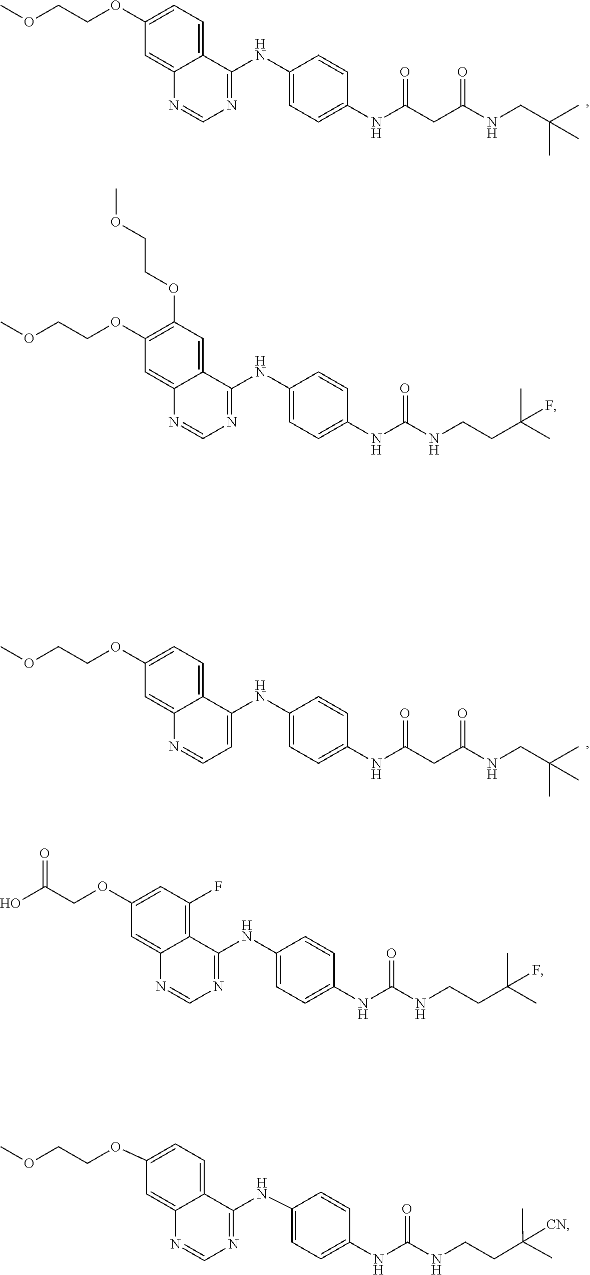 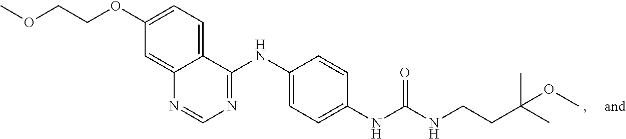
and pharmaceutically acceptable salts, enantiomers, stereoisomers, or tautomers thereof.

68. A pharmaceutical composition comprising the compound according to claim 1, or a pharmaceutically acceptable salt, enantiomer, stereoisomer, or tautomer thereof, and a pharmaceutically acceptable carrier or excipient.

69. A method of treating a disease selected from the group consisting of gastrointestinal stromal tumors (GIST), NF-1-deficient gastrointestinal stromal tumors, succinate dehydrogenase (SDH)-deficient gastrointestinal stromal tumors, KIT activated gastrointestinal stromal tumors, PDGFRa activated gastrointestinal stromal tumors (GIST), melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, and non-small cell lung cancer in a patient in need thereof, comprising administering to the patient a therapeutically effective amount of the compound of claim 1, or a pharmaceutically acceptable salt, enantiomer, stereoisomer, or tautomer thereof.

70. The method of claim 69, wherein the disease is gastrointestinal stromal tumors (GIST).

71. The method of claim 70, wherein the disease is a KIT activated gastrointestinal stromal tumors (GIST).

72. The method of claim 71, wherein the KIT activated gastrointestinal stromal tumors (GIST) has a baseline mutation selected from the group consisting of a KIT exon 9 mutation, a KIT exon 11 mutation, a KIT exon 13 mutation, a KIT exon 17 mutation, and a KIT exon 18 mutation.

73. The method of claim 69, wherein the disease is a PDGFRa activated gastrointestinal stromal tumors (GIST).

74. The method of claim 73, wherein the PDGFRa activated gastrointestinal stromal tumors (GIST) has a baseline mutation selected from PDGFRA D842V.

75. The method of claim 69, wherein the disease is melanoma.

76. The method of claim 75, wherein the melanoma is a KIT activated melanoma.

77. The method of claim 75, wherein the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation.

78-92. (canceled)
